SEQUENCE LISTING (52 sequences)

SEQ ID NO: 1 - RNA, 4443 nt, Artificial Sequence
  Synthetic polynucleotide
augcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaa

SEQ ID NO: 2 - RNA, 4443 nt, Artificial Sequence
  Synthetic polynucleotide
augcagcgguccccgcucgaaaaggccagugucguguccaaacucuucuucucauggacucggccuauccuuagaaagggguaucggcagaggcuugaguugucugacaucuaccagauccccucgguagauucggcggauaaccucucggagaagcucgaacgggaaugggaccgcgaacucgcgucuaagaaaaacccgaagcucaucaacgcacugagaaggugcuucuucuggcgguucauguucuacgguaucuucuuguaucucggggaggucacaaaagcaguccaaccccuguuguugggucgcauuaucgccucguacgaccccgauaacaaagaagaacggagcaucgcgaucuaccucgggaucggacuguguuugcuuuucaucgucagaacacuuuuguugcauccagcaaucuucggccuccaucacaucgguaugcagaugcgaaucgcuauguuuagcuugaucuacaaaaagacacugaaacucucgucgcggguguuggauaagauuuccaucggucaguuggugucccugcuuaguaauaaccucaacaaauucgaugagggacuggcgcuggcacauuucguguggauugccccguugcaagucgcccuuuugaugggccuuauuugggagcuguugcaggcaucugccuuuuguggccugggauuucugauuguguuggcauuguuucaggcugggcuugggcggaugaugaugaaguaucgcgaccagagagcggguaaaaucucggaaagacucgucaucacuucggaaaugaucgaaaacauccagucggucaaagccuauugcugggaagaagcuauggagaagaugauugaaaaccuccgccaaacugagcugaaacugacccgcaaggcggcguauguccgguauuucaauucgucagcguucuucuuuuccggguucuucguugucuuucucucgguuuugccuuaugccuugauuaaggggauuauccuccgcaagauuuucaccacgauuucguucugcauuguauugcgcauggcagugacacggcaauuuccgugggccgugcagacaugguaugacucgcuuggagcgaucaacaaaauccaagacuucuugcaaaagcaagaguacaagacccuggaguacaaucuuacuacuacggagguaguaauggagaaugugacggcuuuuugggaagaggguuuuggagaacuguuugagaaagcaaagcagaauaacaacaaccgcaagaccucaaauggggacgauucccuguuuuucucgaacuucucccugcucggaacacccguguugaaggacaucaauuucaagauugagaggggacagcuucucgcgguagcgggaagcacuggugcgggaaaaacuagccucuugauggugauuaugggggagcuugagcccagcgaggggaagauuaaacacuccgggcguaucucauucuguagccaguuuucauggaucaugcccggaaccauuaaagagaacaucauuuucggaguauccuaugaugaguaccgauacagaucggucauuaaggcgugccaguuggaagaggacauuucuaaguucgccgagaaggauaacaucgucuugggagaaggggguauuacauugucgggagggcagcgagcgcggaucagccucgcgagagcgguauacaaagaugcagauuuguaucugcuugauucaccguuuggauaccucgacguauugacagaaaaagaaaucuucgagucgugcguguguaaacuuauggcuaauaagacgagaauccuggugacaucaaaaauggaacaccuuaagaaggcggacaagauccugauccuccacgaaggaucguccuacuuuuacggcacuuucucagaguugcaaaacuugcagccggacuucucaagcaaacucauggggugugacucauucgaccaguucagcgcggaacggcggaacucgaucuugacggaaacgcugcaccgauucucgcuugagggugaugccccgguaucguggaccgagacaaagaagcagucguuuaagcagacaggagaauuuggugagaaaagaaagaacaguaucuugaauccuauuaacucaauucgcaaguucucaaucguccagaaaacuccacugcagaugaauggaauugaagaggauucggacgaaccccuggagcgcaggcuuagccucgugccggauucagagcaaggggaggccauucuuccccggauuucggugauuucaaccggaccuacacuucaggcgaggcgaaggcaauccgugcucaaccucaugacgcauucgguaaaccaggggcaaaacauucaccgcaaaacgacggccucaacgagaaaagugucacuugcaccccaggcgaauuugacugaacucgacaucuacagccguaggcuuucgcaagaaaccggacuugagaucagcgaagaaaucaaugaagaagauuugaaagaguguuucuuugaugacauggaaucaaucccagcggugacaacguggaacacauacuugcguuacaucacggugcacaaguccuugauuuucguccucaucuggugucucgugaucuuucucgcugaggucgcagcgucacuugugguccucuggcugcuugguaauacgcccuugcaagacaaaggcaauucuacacacucaagaaacaauuccuaugccgugauuaucacuucuacaagcucguauuacguguuuuacaucuacguaggaguggccgacacucugcucgcgauggguuucuuccgaggacucccacucguucacacgcuuaucacugucuccaagauucuccaccauaagaugcuucauagcguacugcaggcucccauguccaccuugaauacgcucaaggcgggagguauuuugaaucgcuucucaaaagauauugcaauuuuggaugaccuucugccccugacgaucuucgacuucauccaguuguugcugaucgugauuggggcuauugcaguagucgcuguccuccagccuuacauuuuugucgcgaccguuccggugaucguggcguuuaucaugcugcgggccuauuucuugcagacgucacagcagcuuaagcaacuggagucugaagggaggucgccuaucuuuacgcaucuugugaccaguuugaagggauuguggacguugcgcgccuuuggcaggcagcccuacuuugaaacacuguuccacaaagcgcugaaucuccauacggcaaauugguuuuuguauuugaguacccuccgaugguuucagaugcgcauugagaugauuuuugugaucuucuuuaucgcggugacuuuuaucuccaucuugaccacgggagagggcgagggacgggucgguauuauccugacacucgccaugaacauuaugagcacuuugcagugggcagugaacagcucgauugauguggauagccugaugagguccguuucgagggucuuuaaguucaucgacaugccgacggagggaaagcccacaaaaaguacgaaacccuauaagaaugggcaauugaguaagguaaugaucaucgagaacagucacgugaagaaggaugacaucuggccuagcgggggucagaugaccgugaaggaccugacggcaaaauacaccgagggagggaacgcaauccuugaaaacaucucguucagcauuagccccggucagcgugugggguugcucgggaggaccgggucaggaaaaucgacguugcugucggccuucuugagacuucugaauacagagggugagauccagaucgacggcguuucgugggauagcaucaccuugcagcaguggcggaaagcguuuggaguaaucccccaaaaggucuuuaucuuuagcggaaccuuccgaaagaaucucgauccuuaugaacaguggucagaucaagagauuuggaaagucgcggacgagguuggccuucggaguguaaucgagcaguuuccgggaaaacucgacuuuguccuuguagaugggggaugcguccugucgcaugggcacaagcagcucaugugccuggcgcgauccguccucucuaaagcgaaaauucuucucuuggaugaaccuucggcccaucuggacccgguaacguaucagaucaucagaaggacacuuaagcaggcguuugccgacugcacggugauucucugugagcaucguaucgaggccaugcucgaaugccagcaauuucuugucaucgaagagaauaagguccgccaguacgacuccauccagaagcugcuuaaugagagaucauuguuccggcaggcgauuucaccauccgauagggugaaacuuuuuccacacagaaauucgucgaagugcaaguccaaaccgcagaucgcggccuugaaagaagagacugaagaagaaguucaagacacgcgucuuuaa

SEQ ID NO: 3 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 4 - RNA, 140 nt, Artificial Sequence
  Synthetic polynucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 5 - RNA, 105 nt, Artificial Sequence
  Synthetic polynucleotide
cggguggcaucccugugaccccuccccagugccucuccuggcccuggaaguugccacuccagugcccaccagccuuguccuaauaaaauuaaguugcaucaagcu

SEQ ID NO: 6 - RNA, 105 nt, Artificial Sequence
  Synthetic polynucleotide
ggguggcaucccugugaccccuccccagugccucuccuggcccuggaaguugccacuccagugcccaccagccuuguccuaauaaaauuaaguugcaucaaagcu

SEQ ID NO: 7 - RNA, 4688 nt, Artificial Sequence
  Synthetic polynucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacgaugcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaacggguggcaucccugugaccccuccccagugccucuccuggcccuggaaguugccacuccagugcccaccagccuuguccuaauaaaauuaaguugcaucaagcu

SEQ ID NO: 8 - RNA, 4688 nt, Artificial Sequence
  Synthetic polynucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacgaugcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaaggguggcaucccugugaccccuccccagugccucuccuggcccuggaaguugccacuccagugcccaccagccuuguccuaauaaaauuaaguugcaucaaagcu

SEQ ID NO: 9 - protein, 874 aa, Unknown
  bacterial
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 10 - DNA, 18 bp, Artificial Sequence
  Synthetic polynucleotide
atttaggtgacactatag

SEQ ID NO: 11 - DNA, 23 bp, Artificial Sequence
  Synthetic polynucleotide
atttaggggacactatagaagag

SEQ ID NO: 12 - DNA, 22 bp, Artificial Sequence
  Synthetic polynucleotide
atttaggggacactatagaagg

SEQ ID NO: 13 - DNA, 23 bp, Artificial Sequence
  Synthetic polynucleotide
atttaggggacactatagaaggg

SEQ ID NO: 14 - DNA, 20 bp, Artificial Sequence
  Synthetic polynucleotide
atttaggtgacactatagaa

SEQ ID NO: 15 - DNA, 22 bp, Artificial Sequence
  Synthetic polynucleotide
atttaggtgacactatagaaga

SEQ ID NO: 16 - DNA, 23 bp, Artificial Sequence
  Synthetic polynucleotide
atttaggtgacactatagaagag

SEQ ID NO: 17 - DNA, 22 bp, Artificial Sequence
  Synthetic polynucleotide
atttaggtgacactatagaagg

SEQ ID NO: 18 - DNA, 23 bp, Artificial Sequence
  Synthetic polynucleotide
atttaggtgacactatagaaggg

SEQ ID NO: 19 - DNA, 23 bp, Artificial Sequence
  Synthetic polynucleotide
  misc_feature (22)..(22): n is a, c, g, or t
atttaggtgacactatagaagng

SEQ ID NO: 20 - DNA, 24 bp, Artificial Sequence
  Synthetic polynucleotide
catacgatttaggtgacactatag

SEQ ID NO: 21 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 22 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 23 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 24 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 25 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 26 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 27 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaagccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 28 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 29 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagagtctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 30 - DNA, 4443 bp, Artificial Sequence
  Synthetic polynucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 31 - DNA, 4443 bp, Artificial Sequence
  Synthetic polynucleotide
atgcagagaagccccctggagaaggcctctgtggtgagcaagctgttcttcagctggaccagacccatcctgagaaagggctacagacagagactggagctgtctgacatctaccagatcccctctgtggactctgccgacaacctgtctgagaagctggagagagagtgggacagagagctggccagcaagaagaaccccaagctgatcaatgccctgagaagatgcttcttctggagattcatgttctatggcatcttcctgtacctgggagaggtgaccaaggccgtgcagcccctgctgctgggcaggatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggccggcctgggcagaatgatgatgaagtacagagaccagagagccggcaagatctctgagagactggtgatcacctctgagatgattgagaacatccagtctgtgaaggcctactgctgggaggaggccatggagaagatgattgagaacctgagacagacagagctgaagctgaccaggaaggccgcctatgtgagatacttcaacagctctgccttcttcttctctggcttctttgtggtgttcctgtctgtgctgccctatgccctgatcaagggcatcatcctgaggaagatcttcaccaccatcagcttctgcattgtgctgaggatggccgtgaccaggcagttcccctgggccgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggagagctgtttgagaaggccaagcagaacaacaacaacagaaagaccagcaatggagatgacagcctgttcttcagcaacttcagcctgctgggcacccctgtgctgaaggacatcaacttcaagattgagaggggccagctgctggccgtggccggcagcacaggagccggcaagaccagcctgctgatggtgatcatgggagagctggagccctctgagggcaagatcaagcactctggcagaatcagcttctgcagccagttcagctggatcatgcctggcaccatcaaggagaacatcatctttggggtgagctatgatgagtacaggtacagatctgtgatcaaggcctgccagctggaggaggacatctccaagtttgccgagaaggacaacattgtgctgggggagggaggcatcaccctgtctgggggccagagagccagaatcagcctggccagagccgtgtacaaggatgccgacctgtacctgctggacagcccctttggctacctggatgtgctgacagagaaggagatctttgagagctgtgtgtgcaagctgatggccaacaagaccaggatcctggtgaccagcaagatggagcacctgaagaaggccgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacaggttcagcctggagggggatgcccctgtgagctggacagagaccaagaagcagagcttcaagcagacaggagagtttggggagaagaggaagaacagcatcctgaaccccatcaacagcatcaggaagttcagcattgtgcagaagacccccctgcagatgaatggcattgaggaggactctgatgagcccctggagagaagactgagcctggtgccagactctgagcagggagaggccatcctgcccaggatctctgtgatcagcacaggccccaccctgcaggccagaagaagacagtctgtgctgaacctgatgacccactctgtgaaccagggccagaatatccacagaaagaccacagccagcaccagaaaggtgagcctggccccccaggccaacctgacagagctggacatctacagcagaaggctgagccaggagacaggcctggagatctctgaggagatcaatgaggaggacctgaaggagtgcttctttgatgacatggagagcatccctgccgtgaccacctggaacacctacctgagatacatcacagtgcacaagagcctgatctttgtgctgatctggtgcctggtgatcttcctggccgaggtggccgccagcctggtggtgctgtggctgctgggcaacacccccctgcaggacaagggcaacagcacccacagcagaaacaacagctatgctgtgatcatcaccagcaccagcagctactatgtgttctacatctatgtgggagtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctggaggcatcctgaacagattcagcaaggacattgccatcctggatgacctgctgcccctgaccatctttgacttcatccagctgctgctgattgtgattggagccattgccgtggtggccgtgctgcagccctacatctttgtggccacagtgcctgtgattgtggccttcatcatgctgagggcctacttcctgcagaccagccagcagctgaagcagctggagtctgagggcagaagccccatcttcacccacctggtgaccagcctgaagggcctgtggaccctgagggcctttggcagacagccctactttgagaccctgttccacaaggccctgaacctgcacacagccaactggttcctgtacctgagcaccctgagatggttccagatgaggattgagatgatctttgtgatcttcttcattgccgtgaccttcatcagcatcctgaccacaggggagggcgagggcagagtgggcatcatcctgaccctggccatgaacatcatgagcaccctgcagtgggccgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctggaggccagatgacagtgaaggacctgacagccaagtacacagaggggggcaatgccatcctggagaacatcagcttcagcatcagccctggccagagggtgggcctgctgggcagaacaggctctggcaagagcaccctgctgtctgccttcctgaggctgctgaacacagagggagagatccagattgatggggtgagctgggacagcatcaccctgcagcagtggaggaaggcctttggggtgatcccccagaaggtgttcatcttctctggcaccttcaggaagaacctggacccctatgagcagtggtctgaccaggagatctggaaggtggccgatgaggtgggcctgagatctgtgattgagcagttccctggcaagctggactttgtgctggtggatggaggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgccgactgcacagtgatcctgtgtgagcacagaattgaggccatgctggagtgccagcagttcctggtgattgaggagaacaaggtgaggcagtatgacagcatccagaagctgctgaatgagagaagcctgttcagacaggccatcagcccctctgacagagtgaagctgttcccccacaggaacagcagcaagtgcaagagcaagccccagattgccgccctgaaggaggagacagaggaggaggtgcaggacaccagactgtga

SEQ ID NO: 32 - DNA, 4443 bp, Artificial Sequence
  Synthetic polynucleotide
atgcagaggagccccctggagaaggccagcgtggtgagcaagctgttcttcagctggaccaggcccatcctgaggaagggctacaggcagaggctggagctgagcgacatctaccagatccccagcgtggacagcgccgacaacctgagcgagaagctggagagggagtgggacagggagctggccagcaagaagaaccccaagctgatcaacgccctgaggaggtgcttcttctggaggttcatgttctacggcatcttcctgtacctgggcgaggtgaccaaggccgtgcagcccctgctgctgggcaggatcatcgccagctacgaccccgacaacaaggaggagaggagcatcgccatctacctgggcatcggcctgtgcctgctgttcatcgtgaggaccctgctgctgcaccccgccatcttcggcctgcaccacatcggcatgcagatgaggatcgccatgttcagcctgatctacaagaagaccctgaagctgagcagcagggtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcaggatgatgatgaagtacagggaccagagggccggcaagatcagcgagaggctggtgatcaccagcgagatgatcgagaacatccagagcgtgaaggcctactgctgggaggaggccatggagaagatgatcgagaacctgaggcagaccgagctgaagctgaccaggaaggccgcctacgtgaggtacttcaacagcagcgccttcttcttcagcggcttcttcgtggtgttcctgagcgtgctgccctacgccctgatcaagggcatcatcctgaggaagatcttcaccaccatcagcttctgcatcgtgctgaggatggccgtgaccaggcagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacaggaagaccagcaacggcgacgacagcctgttcttcagcaacttcagcctgctgggcacccccgtgctgaaggacatcaacttcaagatcgagaggggccagctgctggccgtggccggcagcaccggcgccggcaagaccagcctgctgatggtgatcatgggcgagctggagcccagcgagggcaagatcaagcacagcggcaggatcagcttctgcagccagttcagctggatcatgcccggcaccatcaaggagaacatcatcttcggcgtgagctacgacgagtacaggtacaggagcgtgatcaaggcctgccagctggaggaggacatcagcaagttcgccgagaaggacaacatcgtgctgggcgagggcggcatcaccctgagcggcggccagagggccaggatcagcctggccagggccgtgtacaaggacgccgacctgtacctgctggacagccccttcggctacctggacgtgctgaccgagaaggagatcttcgagagctgcgtgtgcaagctgatggccaacaagaccaggatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagaggaggaacagcatcctgaccgagaccctgcacaggttcagcctggagggcgacgcccccgtgagctggaccgagaccaagaagcagagcttcaagcagaccggcgagttcggcgagaagaggaagaacagcatcctgaaccccatcaacagcatcaggaagttcagcatcgtgcagaagacccccctgcagatgaacggcatcgaggaggacagcgacgagcccctggagaggaggctgagcctggtgcccgacagcgagcagggcgaggccatcctgcccaggatcagcgtgatcagcaccggccccaccctgcaggccaggaggaggcagagcgtgctgaacctgatgacccacagcgtgaaccagggccagaacatccacaggaagaccaccgccagcaccaggaaggtgagcctggccccccaggccaacctgaccgagctggacatctacagcaggaggctgagccaggagaccggcctggagatcagcgaggagatcaacgaggaggacctgaaggagtgcttcttcgacgacatggagagcatccccgccgtgaccacctggaacacctacctgaggtacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcaggaacaacagctacgccgtgatcatcaccagcaccagcagctactacgtgttctacatctacgtgggcgtggccgacaccctgctggccatgggcttcttcaggggcctgcccctggtgcacaccctgatcaccgtgagcaagatcctgcaccacaagatgctgcacagcgtgctgcaggcccccatgagcaccctgaacaccctgaaggccggcggcatcctgaacaggttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagggcctacttcctgcagaccagccagcagctgaagcagctggagagcgagggcaggagccccatcttcacccacctggtgaccagcctgaagggcctgtggaccctgagggccttcggcaggcagccctacttcgagaccctgttccacaaggccctgaacctgcacaccgccaactggttcctgtacctgagcaccctgaggtggttccagatgaggatcgagatgatcttcgtgatcttcttcatcgccgtgaccttcatcagcatcctgaccaccggcgagggcgagggcagggtgggcatcatcctgaccctggccatgaacatcatgagcaccctgcagtgggccgtgaacagcagcatcgacgtggacagcctgatgaggagcgtgagcagggtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagggtgggcctgctgggcaggaccggcagcggcaagagcaccctgctgagcgccttcctgaggctgctgaacaccgagggcgagatccagatcgacggcgtgagctgggacagcatcaccctgcagcagtggaggaaggccttcggcgtgatcccccagaaggtgttcatcttcagcggcaccttcaggaagaacctggacccctacgagcagtggagcgaccaggagatctggaaggtggccgacgaggtgggcctgaggagcgtgatcgagcagttccccggcaagctggacttcgtgctggtggacggcggctgcgtgctgagccacggccacaagcagctgatgtgcctggccaggagcgtgctgagcaaggccaagatcctgctgctggacgagcccagcgcccacctggaccccgtgacctaccagatcatcaggaggaccctgaagcaggccttcgccgactgcaccgtgatcctgtgcgagcacaggatcgaggccatgctggagtgccagcagttcctggtgatcgaggagaacaaggtgaggcagtacgacagcatccagaagctgctgaacgagaggagcctgttcaggcaggccatcagccccagcgacagggtgaagctgttcccccacaggaacagcagcaagtgcaagagcaagccccagatcgccgccctgaaggaggagaccgaggaggaggtgcaggacaccaggctgtga

SEQ ID NO: 33 - DNA, 4443 bp, Artificial Sequence
  Synthetic polynucleotide
atgcagagatcccctctggagaaggcctcagtggtgtccaagcttttcttctcctggaccaggcccattttaagaaagggctacaggcagagacttgagctgtctgacatctatcagatcccttctgtggattctgctgacaatcttagtgaaaaattggaaagggagtgggacagagagctggcaagtaaaaagaaccccaagctgattaatgccctgaggcgctgctttttttggagattcatgttctatggcatattcctctaccttggagaagtaaccaaagctgtacagcctctcctccttggcagaatcattgcctcctatgatcctgataacaaggaggagagaagcatagccatctacctgggcattgggctgtgcctcttgtttattgtgaggacccttctcttgcaccctgccatctttggccttcatcacattggcatgcaaatgagaatagcaatgtttagtcttatttacaaaaaaacattaaaactctcttccagggtgttggacaagatcagtattggacaactggtcagcctgctgagcaacaacctgaacaagtttgatgaaggactggccctggcccactttgtctggattgccccccttcaggtggctcttttgatgggcctgatctgggaactcctgcaggcctctgccttctgtgggttaggcttcctgatagtgctagctctctttcaggcagggttgggtagaatgatgatgaagtacagagaccagagggctgggaagatatctgagaggctggtcattacttctgaaatgatagaaaacatccagtctgttaaagcttactgctgggaggaggctatggaaaagatgattgagaacttgaggcaaacagagctcaagctgactaggaaggcagcctatgtcaggtatttcaacagcagtgctttcttcttctcaggctttttcgtggtcttcttgagtgttctgccctatgccctcatcaaggggataattttgagaaagattttcaccactatttccttttgcattgtcctgaggatggctgtcaccaggcaattcccctgggctgtgcagacatggtatgactctctgggggccatcaacaaaatccaagatttcctgcagaagcaggagtacaagaccctggaatacaacctcaccaccacagaagttgtgatggagaatgtgactgcattctgggaggaaggatttggggagctgtttgagaaagcaaaacaaaacaataataacaggaaaaccagcaatggagatgactccctgttcttttccaacttctctttgttgggcacccctgtcctgaaagatataaactttaaaattgaaagagggcagctgttggcagttgctggctccacaggagctggaaaaacttcactactgatggtgatcatgggggagttagaaccctctgaagggaaaataaaacattctgggaggattagtttctgcagccagttcagctggatcatgcctgggaccattaaagaaaatattatatttggagtgagctatgatgaatatagatataggagtgtcatcaaagcctgtcagttggaggaagacatcagcaaatttgcagagaaagacaacattgttctgggtgaaggtggcatcaccctgtcaggagggcaaagggccaggatcagcttggccagagcagtctataaagatgctgatctgtacctcctggatagcccttttggctatctggatgttttgacagagaaggaaatttttgagtcctgtgtctgcaagttaatggcaaataaaacaaggatacttgtgacctcaaaaatggaacacctgaagaaggctgacaaaattctgatcctgcatgagggcagcagctacttttatggaacattttctgaactgcagaatttgcaaccagacttttcatcaaagctcatgggatgtgacagttttgatcagttttctgcagaaaggagaaactccattttgactgagaccctgcacaggttcagtctggagggggatgccccagtgagttggactgagacaaagaaacagagcttcaagcagactggagagtttggagaaaagaggaaaaactcaattctcaatcccatcaatagcatcaggaagttcagcatagttcagaagactcctttgcagatgaatgggattgaagaggactcagatgagcccctggaaaggagactctccttggtgccagattcagagcagggggaagccatactgccaaggatctctgtgatttctacagggcccaccctccaagcaagaaggagacagtcagttttaaacctgatgacccactctgtcaaccagggacagaacattcatagaaagacaacagcatctacaagaaaagtttcactggcccctcaagccaatttaactgaactagatatctacagcaggaggctcagccaagaaacaggcctggagatctcagaagaaataaatgaggaggatttgaaggaatgcttctttgatgatatggagagcatcccagctgtcacaacctggaacacctacctgagatacatcacagtgcacaaatccctcatctttgtacttatatggtgccttgtcatcttcttagctgaggtggctgcttccctggtggtgctgtggctgctgggaaacacacccctccaggataaagggaactctactcacagcaggaacaacagttatgctgtgatcatcaccagtacctcctcctactatgtgttctacatttatgttggagttgcagacacattgcttgccatgggtttttttagaggactccccctggtgcatactctcatcactgtttccaaaatccttcaccacaagatgctgcacagtgtactacaggctcccatgagcaccctcaacactcttaaagcaggaggaatcttgaacagatttagcaaggacattgcaattcttgatgacctgcttccactgaccatctttgacttcatccagcttctgctcattgtaattggtgccattgctgtggtagcagtgctccagccatatatttttgtggccactgtgcctgttattgtggccttcattatgttgagagcctacttcctgcagacctctcagcagctcaagcaacttgaaagtgagggcaggagccccatatttacacacttggtcacttccctcaaaggcctctggacactcagagcttttggaagacaaccttattttgaaactctcttccacaaggctctgaatctccacacagccaactggtttctgtatctttcaacactgcgctggttccagatgaggattgagatgatctttgttatcttcttcatagctgttaccttcatctctattctgacaactggtgagggggaagggagagtaggcatcatcctcacactagccatgaacataatgtctaccttacaatgggccgtgaacagctccatagatgtggacagcctcatgagaagtgtgtcaagagttttcaaattcattgacatgcccacagaaggcaaaccaaccaagagcacaaaaccctacaagaatggccagctgagtaaggtcatgatcattgaaaattctcatgtgaagaaggatgatatttggcccagtgggggccagatgacagtcaaggacctcactgccaaatacacagagggtggaaatgctatcctagagaacatctccttctccatctccccaggccaaagagttggcttgctgggcaggactggcagtggcaagtccaccttgctctcagcatttctcaggcttttaaatacagagggagagattcaaattgatggggtgtcttgggatagtataacacttcaacagtggaggaaagcctttggtgtgattcctcagaaagtgtttatcttctctggcactttcagaaaaaatctggacccctatgaacagtggagtgaccaggaaatctggaaggtggcagatgaagtgggcctaagatcagtcatagagcagtttcctggaaagttggattttgtgcttgtagatggaggctgtgtgctgtcccatggccataaacagctaatgtgcctggctaggtcagtgctgagcaaggccaagatcctgctgttagatgagccttcagcccatctggaccctgtgacataccagattatcagaagaactctgaagcaggcctttgctgactgcactgtcatcctgtgtgagcacagaattgaggccatgctggagtgccagcagttccttgttatagaagagaataaggttaggcagtatgacagcattcagaaactgctaaatgaaagatctctcttcaggcaagctatttcaccatctgatagagtgaaactttttccccacagaaattcctctaaatgtaaatctaagccccagatagctgccttgaaagaggagactgaagaagaagtccaggacaccagactgtga

SEQ ID NO: 34 - DNA, 4443 bp, Artificial Sequence
  Synthetic polynucleotide
atgcagagatccccgctggagaaggcatctgtggtgtcaaaactgttctttagctggacaaggcccatccttaggaaagggtacagacagaggttggagctgtcagacatatatcagatcccttcagtggactctgcagacaacctctctgaaaagctggagagggaatgggacagggaactggccagcaaaaaaaaccctaaactgattaatgccctgaggaggtgcttcttttggagattcatgttctatgggatcttcctttacctgggggaggtgactaaagctgttcagcctcttcttctggggaggattattgcctcctatgacccagacaacaaagaagaaagaagcatagccatttacttaggcataggcctctgcttgctcttcatagttagaaccctcctactccacccagccatctttggtctccaccacataggtatgcagatgagaatagcaatgttctccttgatctacaagaagaccctcaagctgtccagcagggtgctggacaagatctccataggccagttagtcagtctactgtccaataacttaaataagtttgatgagggactggcactggcacattttgtgtggattgcccccctccaagtggcccttcttatgggccttatctgggagctgttgcaggcctctgctttctgtggcctgggtttcctcatagtcctagccttattccaggctggactgggcagaatgatgatgaagtatagggaccaaagagcagggaagatttctgaaaggctggttataacttctgagatgattgagaacattcagtcagtgaaagcttactgctgggaagaagctatggaaaaaatgattgaaaatctcagacagactgaattaaagttgaccaggaaagctgcttatgtcagatacttcaactcctcagccttctttttttctggcttctttgttgtattcctttcagtcctcccctatgccctgattaagggcattatcttgaggaaaattttcacaaccatctccttttgtattgtcctcaggatggctgttacaaggcaatttccttgggctgtgcaaacttggtatgatagccttggagcaatcaacaagatccaggatttcctgcaaaagcaggagtacaagacattggaatacaaccttaccaccactgaggtggtgatggaaaatgtgactgccttctgggaggaggggtttggagagctgtttgagaaagccaaacagaacaacaacaatagaaagacctctaatggtgatgattccctgttcttttctaactttagtcttctggggaccccagttctgaaagatattaactttaaaattgaaaggggacagttgctggctgtggctgggtccactggggctgggaagacaagcctgctcatggtgatcatgggagagctggaacccagtgaaggaaagatcaaacactcaggcaggatctccttctgcagccagttctcatggattatgccaggcactattaaagaaaatatcatctttggtgtaagctatgatgagtacaggtatagatctgtaattaaagcctgccagctggaggaagacatctctaagtttgctgagaaggataacattgtgttgggggaagggggcatcaccctttctggtgggcagagggctaggatctcccttgctagggcagtatacaaggatgctgacttgtacctcttggatagtccttttggctacctagatgtgctgacagagaaagaaatatttgaaagctgtgtgtgtaagctcatggctaacaagaccaggatcctggtcaccagtaaaatggaacacctcaaaaaagcagacaagatccttattctccatgagggctcctcctacttctatgggaccttcagtgagctgcagaatctgcagccagacttctcctcaaaacttatgggctgtgactcctttgaccaattctctgcagaaagaaggaatagcatactgacagaaacactgcatagattctccctggaaggagatgccccagtgagttggacagaaaccaaaaagcagagcttcaagcagactggtgagtttggtgaaaagaggaagaattctatcctgaaccccatcaatagcatcaggaaatttagcatagtccaaaagacccccctccagatgaatggaatagaggaggatagtgatgagcctcttgagagaaggctgtccctggttccagacagtgaacagggtgaagccattcttccgaggatcagtgtcatctccactgggcccacattgcaggccagaagaagacagtctgttctgaatttgatgacacattctgtgaatcaaggccagaatatccatagaaaaaccactgccagcaccagaaaagtttctctagccccccaggctaacctgactgagttagacatctacagcagaaggctgagccaagagactggcttggaaatatctgaggagatcaatgaggaggacctcaaggagtgcttctttgatgacatggagtcaatccctgcagtcactacatggaacacttacctaaggtacatcacagttcataagagcctcatctttgtcctcatatggtgtctggtcatctttttagcagaagtggctgccagcctagttgtgctgtggttactgggcaatacacctcttcaggacaaaggcaatagcacacacagcagaaacaactcctatgcagtgatcatcacctctacaagctcttactatgtattctatatatatgtgggagtggcagatactctcctggccatgggattcttcaggggattacctctagttcacacattgatcacagtgtcaaaaattctccaccacaagatgttacacagtgtcctgcaagccccaatgtctactctgaacacacttaaggcaggtggaattttgaataggtttagcaaggacatagctatcctggatgatctcctccctctgaccatctttgacttcatccagttactgctcattgtaattggagccattgcagtggtagcagtcctacagccttacatttttgtggctactgttcctgttattgtggccttcattatgctaagagcttacttcctgcaaacaagccaacagttgaaacagctagaaagtgagggaaggtcccccatcttcacccacctggtgacatcactcaaggggctatggactcttagggcttttgggagacagccgtactttgagaccttattccataaggcccttaacctccatacagcaaactggttcttatacctgagtactctgaggtggtttcaaatgaggattgaaatgatttttgtgatcttcttcattgctgtgaccttcatctcaatcttgaccacaggagagggggagggcagggtgggcatcatactgaccttggccatgaacattatgtcaaccctgcagtgggctgtcaatagctccattgatgtggacagtctgatgaggagtgtctccagggtcttcaagtttattgacatgccaactgagggcaaacccaccaaaagcactaagccatataaaaatggccaactgtccaaagtgatgatcattgaaaattcacatgtaaagaaggatgatatctggccctctggaggacagatgacagtgaaagacctgactgccaagtacacagagggtggtaatgccattcttgagaacattagtttcagtatttccccggggcaaagggtgggcctccttggcagaacaggctctggcaagagtaccctgctgtcagcctttttaagactgttgaacactgagggagaaattcagattgatggtgtctcctgggatagcatcaccctccagcagtggagaaaagcttttggagtgatcccgcaaaaggttttcatcttttcaggcaccttccggaagaacctggacccctatgagcagtggtctgaccaggaaatatggaaggtagctgatgaagttgggcttaggtcagtcatagagcagttcccaggcaaactggactttgtcctggtggatggtggatgtgtactgagtcatgggcacaaacagctgatgtgcctagccaggtctgtgctcagcaaggcaaagatattgctgcttgatgaacccagtgcccatctggacccagtcacatatcagatcatcagaagaacattgaagcaggcctttgctgattgcacagttatcctctgtgagcacaggattgaggccatgctggagtgccagcagtttctggtgattgaggagaataaagtaaggcagtatgactccatccagaagctgctcaatgaaagaagcctctttagacaagctatctccccctcagacagggtcaaattgttccctcacagaaacagcagcaagtgcaagagcaagccccaaattgcagccttgaaagaggagacagaggaagaggtgcaggacaccagactctga

SEQ ID NO: 35 - DNA, 4443 bp, Artificial Sequence
  Synthetic polynucleotide
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagagaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcagaagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 36 - DNA, 4443 bp, Artificial Sequence
  Synthetic polynucleotide
atgcagcgcagccccctggagaaggccagcgtggtgagcaagctgttcttcagctggacccgccccatcctgcgcaagggctaccgccagcgcctggagctgagcgacatctaccagatccccagcgtggacagcgccgacaacctgagcgagaagctggagcgcgagtgggaccgcgagctggccagcaagaagaaccccaagctgatcaacgccctgcgccgctgcttcttctggcgcttcatgttctacggcatcttcctgtacctgggcgaggtgaccaaggccgtgcagcccctgctgctgggccgcatcatcgccagctacgaccccgacaacaaggaggagcgcagcatcgccatctacctgggcatcggcctgtgcctgctgttcatcgtgcgcaccctgctgctgcaccccgccatcttcggcctgcaccacatcggcatgcagatgcgcatcgccatgttcagcctgatctacaagaagaccctgaagctgagcagccgcgtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggccgcatgatgatgaagtaccgcgaccagcgcgccggcaagatcagcgagcgcctggtgatcaccagcgagatgatcgagaacatccagagcgtgaaggcctactgctgggaggaggccatggagaagatgatcgagaacctgcgccagaccgagctgaagctgacccgcaaggccgcctacgtgcgctacttcaacagcagcgccttcttcttcagcggcttcttcgtggtgttcctgagcgtgctgccctacgccctgatcaagggcatcatcctgcgcaagatcttcaccaccatcagcttctgcatcgtgctgcgcatggccgtgacccgccagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaaccgcaagaccagcaacggcgacgacagcctgttcttcagcaacttcagcctgctgggcacccccgtgctgaaggacatcaacttcaagatcgagcgcggccagctgctggccgtggccggcagcaccggcgccggcaagaccagcctgctgatggtgatcatgggcgagctggagcccagcgagggcaagatcaagcacagcggccgcatcagcttctgcagccagttcagctggatcatgcccggcaccatcaaggagaacatcatcttcggcgtgagctacgacgagtaccgctaccgcagcgtgatcaaggcctgccagctggaggaggacatcagcaagttcgccgagaaggacaacatcgtgctgggcgagggcggcatcaccctgagcggcggccagcgcgcccgcatcagcctggcccgcgccgtgtacaaggacgccgacctgtacctgctggacagccccttcggctacctggacgtgctgaccgagaaggagatcttcgagagctgcgtgtgcaagctgatggccaacaagacccgcatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagcgccgcaacagcatcctgaccgagaccctgcaccgcttcagcctggagggcgacgcccccgtgagctggaccgagaccaagaagcagagcttcaagcagaccggcgagttcggcgagaagcgcaagaacagcatcctgaaccccatcaacagcatccgcaagttcagcatcgtgcagaagacccccctgcagatgaacggcatcgaggaggacagcgacgagcccctggagcgccgcctgagcctggtgcccgacagcgagcagggcgaggccatcctgccccgcatcagcgtgatcagcaccggccccaccctgcaggcccgccgccgccagagcgtgctgaacctgatgacccacagcgtgaaccagggccagaacatccaccgcaagaccaccgccagcacccgcaaggtgagcctggccccccaggccaacctgaccgagctggacatctacagccgccgcctgagccaggagaccggcctggagatcagcgaggagatcaacgaggaggacctgaaggagtgcttcttcgacgacatggagagcatccccgccgtgaccacctggaacacctacctgcgctacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagccgcaacaacagctacgccgtgatcatcaccagcaccagcagctactacgtgttctacatctacgtgggcgtggccgacaccctgctggccatgggcttcttccgcggcctgcccctggtgcacaccctgatcaccgtgagcaagatcctgcaccacaagatgctgcacagcgtgctgcaggcccccatgagcaccctgaacaccctgaaggccggcggcatcctgaaccgcttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgcgcgcctacttcctgcagaccagccagcagctgaagcagctggagagcgagggccgcagccccatcttcacccacctggtgaccagcctgaagggcctgtggaccctgcgcgccttcggccgccagccctacttcgagaccctgttccacaaggccctgaacctgcacaccgccaactggttcctgtacctgagcaccctgcgctggttccagatgcgcatcgagatgatcttcgtgatcttcttcatcgccgtgaccttcatcagcatcctgaccaccggcgagggcgagggccgcgtgggcatcatcctgaccctggccatgaacatcatgagcaccctgcagtgggccgtgaacagcagcatcgacgtggacagcctgatgcgcagcgtgagccgcgtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagcgcgtgggcctgctgggccgcaccggcagcggcaagagcaccctgctgagcgccttcctgcgcctgctgaacaccgagggcgagatccagatcgacggcgtgagctgggacagcatcaccctgcagcagtggcgcaaggccttcggcgtgatcccccagaaggtgttcatcttcagcggcaccttccgcaagaacctggacccctacgagcagtggagcgaccaggagatctggaaggtggccgacgaggtgggcctgcgcagcgtgatcgagcagttccccggcaagctggacttcgtgctggtggacggcggctgcgtgctgagccacggccacaagcagctgatgtgcctggcccgcagcgtgctgagcaaggccaagatcctgctgctggacgagcccagcgcccacctggaccccgtgacctaccagatcatccgccgcaccctgaagcaggccttcgccgactgcaccgtgatcctgtgcgagcaccgcatcgaggccatgctggagtgccagcagttcctggtgatcgaggagaacaaggtgcgccagtacgacagcatccagaagctgctgaacgagcgcagcctgttccgccaggccatcagccccagcgaccgcgtgaagctgttcccccaccgcaacagcagcaagtgcaagagcaagccccagatcgccgccctgaaggaggagaccgaggaggaggtgcaggacacccgcctgtaa

SEQ ID NO: 37 - DNA, 4443 bp, Artificial Sequence
  Synthetic polynucleotide
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagggaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcaggagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 38 - DNA, 4443 bp, Artificial Sequence
  Synthetic polynucleotide
atgcagaggtcacctctggaaaaggctagcgtggtcagcaagctatttttttcctggacccgcccgatactcaggaagggctaccgacagcggctggagctgagtgacatttatcagattccctccgtcgattccgctgacaacctgtctgagaaactggagcgggaatgggatagggaactggcgtccaaaaaaaaccccaaactcatcaatgcactccgcagatgcttcttctggcggtttatgttttatggcatattcctgtatctgggggaggtgacgaaagccgtgcagccgctgctgcttggtcgcattatcgcgtcatacgatccagataacaaggaggaaagaagtatcgctatctatctcgggatagggctgtgcctgctcttcattgtgcggactcttctcttgcaccccgccattttcggtctgcatcatataggtatgcagatgagaattgcgatgttctcattgatttacaaaaaaacgcttaagctaagttcaagggtgctagataagatatcgatcggccagctggtgtctctgcttagcaacaacctcaataaattcgacgaaggccttgcactggcccacttcgtgtggatcgcccctctgcaggtggctctgctgatggggttaatatgggagctgttgcaggcctccgctttttgtggcctggggtttctcatcgtgttggccttgtttcaggcagggctgggacgtatgatgatgaaatatagggatcagagggctggcaaaatctctgagcgcctggttattacgagtgaaatgattgagaacatccagtcagtgaaggcctattgctgggaggaggccatggaaaaaatgattgagaacctacgccagactgagctgaagttaaccagaaaagccgcctatgtgcgctactttaacagtagcgcatttttcttctccggttttttcgtggtgtttcttagtgtgttgccgtatgccttaatcaagggaataatactccggaagattttcactaccatcagcttctgtatcgtgttgcggatggccgtcacccggcagtttccctgggcagtacagacttggtacgattctctcggagcaattaacaaaatccaagactttctacaaaagcaggagtacaagaccctggagtacaatctgaccaccacagaagtcgtaatggagaatgtaactgccttctgggaagagggctttggcgaactctttgaaaaggccaagcagaacaataacaaccggaagacctccaacggggacgacagcttatttttcagcaatttttctttgctcgggacccctgtactgaaagatattaactttaagatcgagcgcggacaactcctggctgtcgccggcagcactggagctggaaaaacatcactgcttatggtgataatgggagaactcgaaccaagcgagggaaaaataaagcactctggacggattagtttttgctcccagttctcgtggataatgcctggcaccattaaggagaatatcatctttggagtgagttacgacgaataccggtaccggtccgttatcaaggcttgtcaactcgaggaggacatttctaaattcgccgaaaaagataatatagtgctgggcgaaggaggcattacactgagcgggggtcagagagctcgaattagcctcgcccgagcagtctataaagacgccgatctttacctgctggattccccttttgggtatttggatgttctgacagagaaggaaatctttgaatcatgtgtctgtaaactgatggccaataagactaggattctagtgacttcgaaaatggagcacctgaaaaaagcggacaaaattctgatactccatgaagggtcttcctacttctacggcaccttctcagagttgcagaacttacaacctgatttttcatctaagcttatggggtgcgactcgtttgaccagttctccgctgaaagacgaaacagcatcttaacggaaactcttcacaggttctcattagagggagatgcgccggtgtcctggacagagacaaaaaaacagtctttcaaacagacaggagagtttggcgagaagagaaaaaactcaatcctcaatcccatcaattctattagaaagtttagcatcgtccaaaaaacaccattgcagatgaatgggattgaggaggacagtgatgagcctttggaacgaagactgtccctggtacccgatagcgaacagggtgaggccatccttcctaggatctcggtcataagtacagggcccacactgcaggccaggcgacgtcaaagtgtcctcaatcttatgacgcacagtgtgaatcaggggcagaacatccatcgtaagacgacagcttcaactcgaaaggtcagtctagctccacaagccaatcttacagagctggacatttattcccgccgcctcagtcaggagaccggattggaaatatcagaggaaattaatgaagaggatctgaaggaatgcttctttgatgacatggaatcgatccccgctgttactacctggaacacatatctgagatatattaccgtccataagagcttaatctttgtactgatatggtgcttggtgattttcctggcagaggttgcggcgagtttggtcgtgctatggctccttggaaacactcccctgcaggataaggggaactccactcatagcaggaataacagctatgccgtgatcatcacctctacctcctcttattacgtgttttacatatacgtcggtgttgcggataccctgttggcaatggggttctttagaggactacccctagttcacaccctgatcaccgtttcgaagatcttgcaccacaagatgcttcatagcgttctccaagctcctatgagcacccttaatacactgaaagcaggaggtatccttaaccgcttttccaaagacatcgctatactcgacgatttgctcccattgaccatcttcgacttcattcagctgctcctcattgtgatcggcgccattgccgtggtcgcagtgttacagccatatattttcgtagccaccgtgcccgtcatcgtggcatttatcatgctgcgcgcatatttcttacagacatctcagcaactgaagcagctggaatctgagggcagatctcctatttttacacacctggttaccagcctgaagggcctgtggaccctgcgtgctttcggtcgccaaccctactttgagactctcttccataaggctctgaatttacatactgccaattggttcctataccttagtacccttcggtggttccagatgcggatagaaatgatcttcgtgattttcttcatcgcagtcactttcatctctattttgacgaccggtgagggcgagggcagggtgggcatcattctgactttggccatgaacattatgtcaacactccagtgggccgttaattcaagcattgatgtggattccttgatgcgttccgtcagcagggtatttaaattcatagacatgcccaccgagggcaagccaacaaaatctaccaagccatacaaaaatggccaactaagcaaggtcatgattatcgagaattctcatgtgaaaaaggacgacatttggccttccgggggtcaaatgactgtaaaggacctgacggctaaatacactgagggcggtaatgctatcttggagaacatctctttcagcatctcccctggccagagagtgggactgctcgggcggacaggctccggaaagtctacgctcctttcagcattccttagacttctgaacaccgaaggtgagattcagattgacggggtctcttgggactccatcacacttcagcaatggaggaaggcattcggtgtaatcccccaaaaggtttttatcttctccggaacatttcgtaagaatctggacccgtacgagcagtggtcagatcaggagatctggaaagtagcagacgaggtcgggctacggagcgttattgaacagtttcctggcaaactggacttcgttttggtggacggaggctgtgtgctgagtcacggccataaacaactgatgtgcttagctaggtctgttctcagcaaggcaaagattttactgctggatgaaccaagcgcccaccttgatccagtgacatatcaaatcatcagaagaactcttaaacaggcgttcgccgactgcacagtgatcctgtgtgagcacagaatagaagccatgctggaatgtcaacagtttctcgtgattgaggagaacaaggtgcgccagtacgatagcatccagaagttactcaatgaaaggtcactcttcaggcaggccatctcacccagcgaccgcgttaagctgtttccacaccgaaacagttccaagtgcaaaagtaagccacagattgctgcactgaaggaagagacagaagaagaagttcaggacactcggctctga

SEQ ID NO: 39 - DNA, 4443 bp, Artificial Sequence; identical to SEQ ID NO: 21
  Synthetic polynucleotide

SEQ ID NO: 40 - DNA, 4443 bp, Artificial Sequence
  Synthetic polynucleotide
atgcaacggagtcctctggaaaaagcctctgtcgtatctaagcttttcttcagttggacacgcccgattttgagaaagggttatcggcaacgcttggaacttagtgacatctaccaaattccaagtgtagactcagccgataacttgagcgaaaagctcgaacgagagtgggatcgagaactggctagcaaaaaaaatcccaaactcataaatgccctgcgacgctgtttcttttggcgatttatgttttacggtattttcctttatttgggtgaggtcacgaaggctgtacagccactgctgctgggtcgcatcattgcctcttacgaccctgacaacaaagaggagcggtcaatagctatctaccttggtataggactttgcttgctcttcatagtccgcacgttgcttctccaccctgctatatttggtctccatcacattgggatgcaaatgcggatcgcgatgttcagtcttatatataaaaagactcttaaactttccagccgggttctggataagatctctattggtcaactggtatctcttttgtctaacaacctgaataagttcgacgagggccttgcattggcccattttgtatggattgcccctttgcaagtcgccctcctgatgggattgatctgggaactcctgcaagctagtgctttttgcggattgggattcctcatagtccttgcgctctttcaggcgggacttggacgcatgatgatgaagtatcgcgaccaacgagctggcaagatcagtgaacggcttgtaataaccagtgaaatgatagagaacatccagagcgtaaaagcttactgttgggaagaagcgatggaaaagatgattgagaaccttcgccagacagaacttaaacttacacgaaaggccgcttatgtccggtacttcaactcttcagcatttttttttagtggcttctttgtagtgttcctgtccgtccttccgtatgcacttatcaagggtataatacttaggaaaatcttcacaacaatcagtttttgcatagtccttcgcatggcagtaactcgccaatttccctgggcagttcagacgtggtacgactcacttggcgcaattaacaaaattcaagatttcctccaaaagcaagagtataaaaccttggaatacaaccttaccaccacagaagttgtaatggaaaatgtcacagccttctgggaggaaggtttcggcgaactttttgagaaggcgaagcaaaataacaataatcggaaaacatcaaacggtgacgattcactgttcttttctaactttagccttcttgggacgcccgtcctgaaggacataaactttaagattgaacggggtcaacttctcgcggtcgcagggagtactggagcggggaaaacgagcctgctgatggtgataatgggggagttggagccctcagaaggcaagatcaagcatagtggtagaattagcttctgcagtcaatttagttggattatgccgggcacgatcaaagaaaatataatctttggggtatcctacgatgaatacaggtaccgatcagtgataaaagcgtgccagcttgaagaagacatttcaaagtttgctgagaaggataatatcgtacttggagaaggaggtatcaccctgtctgggggtcaacgagcgaggatctccctggcacgcgccgtctacaaggacgcggacctctatctgttggattcaccgttcggatatttggacgtgcttacggagaaagaaatatttgagagctgtgtttgcaagctcatggcaaataaaaccagaatattggttacaagcaagatggagcatcttaagaaagcagataaaatcctgatattgcacgagggctcttcatacttctacgggacgttttctgagttgcagaacctccagccggatttcagctctaagctgatgggctgtgattcctttgatcagtttagtgcggaaagacgaaacagtatactcaccgaaacactgcacaggttctctctggagggcgacgccccggtttcctggacagagacgaagaagcagtccttcaaacagacaggcgagtttggggagaaaaggaaaaatagcatactcaacccgattaacagcattcgcaagttcagtatagtacaaaagaccccgttgcagatgaacggtatagaggaagattctgatgagccactggaaagacggctttctctcgttccggacagtgaacagggagaggcaatactgcctcggatcagcgttatctctacaggacctactttgcaagctcggcgccgacagtcagtcttgaatcttatgactcatagtgttaatcaaggccagaatatccatcgcaagaccaccgcaagtacaaggaaagtgagcttggcacctcaagcaaaccttactgaacttgatatctactcacggcgactttcacaggagaccggacttgaaattagtgaagaaattaacgaggaggacctcaaggagtgcttcttcgatgacatggaatcaatccccgcagtcacaacctggaacacttatctgaggtatataacagttcacaagagcctcatttttgtacttatttggtgtttggtaattttcctggcggaggttgctgcttctttggtcgtcctttggctcctcgggaatacaccgctccaagacaaaggcaactctacccatagtaggaacaattcatatgcagtgattataaccagtacatcatcttattacgttttctatatttatgtcggggtagctgacacgctgttggcgatgggcttctttaggggcctccccttggtacacacccttatcacggtgagtaaaatcctgcatcacaaaatgcttcattctgtactccaagcgccgatgagtacgcttaatacgctgaaagcaggagggatactgaatcggttcagcaaggacatcgccattctggatgacctgcttccattgacaatatttgatttcattcagctccttctcatagttattggagccatagcggtggtggctgtgcttcagccttatatattcgttgccacagttcccgttatagtggcatttataatgctcagggcctactttctccagacttcccagcagttgaagcaactcgaatcagaaggaaggtcacctattttcacacatcttgtgacttccttgaagggcttgtggacgctgcgggccttcggaagacaaccatattttgaaactctcttccacaaagctttgaatcttcatactgcgaactggttcctgtatttgagtactttgcgctggttccagatgaggatagaaatgatattcgttatcttctttatcgcggttacgttcataagtatcctcactacgggggagggtgagggtagagtgggcataatactgaccctcgccatgaacattatgtccaccctgcagtgggcggtaaacagcagcatagatgtggattctttgatgcgcagtgtgagcagggtttttaagtttatcgatatgccgacggaaggaaagcccactaaaagcacgaaaccctataaaaatggacagcttagcaaagtaatgataatcgagaatagccatgtgaaaaaggatgacatatggccttccggaggccaaatgactgttaaagatctgaccgctaaatataccgagggcggcaacgcaatactcgaaaacataagcttttccataagccccggccaacgcgtgggtcttctggggaggactggctccggaaaatcaacgttgcttagcgcgtttttgcggctccttaacactgaaggtgagatccaaatagatggcgttagttgggactctataacactgcaacaatggcggaaagctttcggcgtcatacctcagaaggtgttcatctttagcggaacgttcaggaagaacttggatccctacgaacaatggagtgatcaagaaatatggaaagtggcagatgaggtaggcttgcgcagtgtcattgaacaattcccagggaaactcgactttgtactggtggacggcggttgcgtcttgtcacacgggcacaaacagttgatgtgtttggcccgcagtgttttgtctaaggcgaagattctgttgctcgacgaaccgagtgctcatcttgatcccgtcacctaccaaatcatcagaaggacgttgaagcaagctttcgccgactgcactgtaatcctttgtgagcataggatcgaagcaatgctcgagtgccaacagttcttggttatagaggagaataaggttcggcaatacgactcaatacagaaactgcttaatgagcggtcactctttcgacaagctatctctcctagtgacagggtaaagctttttcctcatcggaattccagcaagtgtaagagtaaaccacagatcgccgcccttaaagaggagaccgaagaagaggtgcaggatacgagactttag

SEQ ID NO: 41 - protein, 1480 aa, Artificial Sequence
  Synthetic polypeptide
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNDILTETLHRFSLEGDAPVSWTETKKQSFKQTGEFGEKRKNSILNPINSIRKFSIVQKTPLQMNGIEEDSDEPLERRLDLVPDSEQGEAILPRISVISTGPTLQARRRQSVLNLMTHSVNQGQNIHRKTTASTRKVDLAPQANLTELDIYSRRLDQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 42 - protein, 1480 aa, Artificial Sequence
  Synthetic polypeptide
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNDILTETLHRFSLEGDAPVSWTETKKQDFKQTGEFGEKRKNDILNPINSIRKFSIVQKTPLQMNGIEEDSDEPLERRLDLVPDSEQGEAILPRISVISTGPTLQARRRQSVLNLMTHSVNQGQNIHRKTTASTRKVDLAPQANLTELDIYSRRLDQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 43 - protein, 1480 aa, Artificial Sequence
  Synthetic polypeptide
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSCDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 44 - protein, 1480 aa, Artificial Sequence
  Synthetic polypeptide
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDQPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 45 - DNA, 4443 bp, Artificial Sequence
  Synthetic polynucleotide
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaacgatatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctggatctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtggatctggcacctcaagcgaatcttaccgagctcgacatctactcccggagactggatcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 46 - DNA, 4443 bp, Artificial Sequence
  Synthetic polynucleotide
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaacgatatcctgacggaaaccttgcaccgcttctctttggaaggcgacgcccctgtgtcatggaccgagactaagaagcaggatttcaagcagaccggggaattcggcgaaaagaggaagaacgacatcttgaaccccattaactccatccgcaagttctcaatcgtgcaaaagacgccactgcagatgaacggcattgaggaggactccgacgaaccccttgagaggcgcctggatctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtggatctggcacctcaagcgaatcttaccgagctcgacatctactcccggagactggatcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 47 - DNA, 4443 bp, Artificial Sequence
  Synthetic polynucleotide
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctcctgcgacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggacgaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 48 - DNA, 4443 bp, Artificial Sequence
  Synthetic polynucleotide
atgcaacgctctcctcttgaaaaggcctcggtggtgtccaagctcttcttctcgtggactagacccatcctgagaaaggggtacagacagcgcttggagctgtccgatatctatcaaatcccttccgtggactccgcggacaacctgtccgagaagctcgagagagaatgggacagagaactcgcctcaaagaagaacccgaagctgattaatgcgcttaggcggtgctttttctggcggttcatgttctacggcatcttcctctacctgggagaggtcaccaaggccgtgcagcccctgttgctgggacggattattgcctcctacgaccccgacaacaaggaagaaagaagcatcgctatctacttgggcatcggtctgtgcctgcttttcatcgtccggaccctcttgttgcatcctgctattttcggcctgcatcacattggcatgcagatgagaattgccatgttttccctgatctacaagaaaactctgaagctctcgagccgcgtgcttgacaagatttccatcggccagctcgtgtccctgctctccaacaatctgaacaagttcgacgagggcctcgccctggcccacttcgtgtggatcgcccctctgcaagtggcgcttctgatgggcctgatctgggagctgctgcaagcctcggcattctgtgggcttggattcctgatcgtgctggcactgttccaggccggactggggcggatgatgatgaagtacagggaccagagagccggaaagatttccgaacggctggtgatcacttcggaaatgatcgaaaacatccagtcagtgaaggcctactgctgggaagaggccatggaaaagatgattgaaaacctccggcaaaccgagctgaagctgacccgcaaggccgcttacgtgcgctatttcaactcgtccgctttcttcttctccgggttcttcgtggtgtttctctccgtgctcccctacgccctgattaagggaatcatcctcaggaagatcttcaccaccatttccttctgtatcgtgctccgcatggccgtgacccggcagttcccatgggccgtgcagacttggtacgactccctgggagccattaacaagatccaggacttccttcaaaagcaggagtacaagaccctcgagtacaacctgactactaccgaggtcgtgatggaaaacgtcaccgccttttgggaggagggatttggcgaactgttcgagaaggccaagcagaacaacaacaaccgcaagacctcgaacggtgacgactccctcttcttttcaaacttcagcctgctcgggacgcccgtgctgaaggacattaacttcaagatcgaaagaggacagctcctggcggtggccggatcgaccggagccggaaagacttccctgctgatggtgatcatgggagagcttgaacctagcgagggaaagatcaagcactccggccgcatcagcttctgtagccagttttcctggatcatgcccggaaccattaaggaaaacatcatcttcggcgtgtcctacgatgaataccgctaccggtccgtgatcaaagcctgccagctggaagaggatatttcaaagttcgcggagaaagataacatcgtgctgggcgaagggggtattaccttgtcggggggccagcgggctagaatctcgctggccagagccgtgtataaggacgccgacctgtatctcctggactcccccttcggatacctggacgtcctgaccgaaaaggagatcttcgaatcgtgcgtgtgcaagctgatggctaacaagactcgcatcctcgtgacctccaaaatggagcacctgaagaaggcagacaagattctgattctgcatgaggggtcctcctacttttacggcaccttctcggagttgcagaacttgcagcccgacttctcatcgaagctgatgggttgcgacagcttcgaccagttctccgccgaaagaaggaactcgatcctgacggaaaccttgcaccgcttctctttggaaggcgacgcccctgtgtcatggaccgagactaagaagcagagcttcaagcagaccggggaattcggcgaaaagaggaagaacagcatcttgaaccccattaactccatccgcaagttctcaatcgtgcaaaagacgccactgcagatgaacggcattgaggaggactccgacgaaccccttgagaggcgcctgtccctggtgccggacagcgagcagggagaagccatcctgcctcggatttccgtgatctccactggtccgacgctccaagcccggcggcggcagtccgtgctgaacctgatgacccacagcgtgaaccagggccaaaacattcaccgcaagactaccgcatccacccggaaagtgtccctggcacctcaagcgaatcttaccgagctcgacatctactcccggagactgtcgcaggaaaccgggctcgaaatttccgaagaaatcaacgaggaggatctgaaagagtgcttcttcgacgatatggagtcgatacccgccgtgacgacttggaacacttatctgcggtacatcactgtgcacaagtcattgatcttcgtgctgatttggtgcctggtgattttcctggccgaggtcgcggcctcactggtggtgctctggctgttgggaaacacgcctctgcaagacaagggaaactccacgcactcgagaaacaacagctatgccgtgattatcacttccacctcctcttattacgtgttctacatctacgtcggagtggcggataccctgctcgcgatgggtttcttcagaggactgccgctggtccacaccttgatcaccgtcagcaagattcttcaccacaagatgttgcatagcgtgctgcaggcccccatgtccaccctcaacactctgaaggccggaggcattctgaacagattctccaaggacatcgctatcctggacgatctcctgccgcttaccatctttgacttcatccagctgctgctgatcgtgattggagcaatcgcagtggtggcggtgctgcagccttacattttcgtggccactgtgccggtcattgtggcgttcatcatgctgcgggcctacttcctccaaaccagccagcagctgaagcaactggaatccgagggacgatcccccatcttcactcaccttgtgacgtcgttgaagggactgtggaccctccgggctttcggacggcagccctacttcgaaaccctcttccacaaggccctgaacctccacaccgccaattggttcctgtacctgtccaccctgcggtggttccagatgcgcatcgagatgattttcgtcatcttcttcatcgcggtcacattcatcagcatcctgactaccggagagggagagggacgggtcggaataatcctgaccctcgccatgaacattatgagcaccctgcagtgggcagtgaacagctcgatcgacgtggacagcctgatgcgaagcgtcagccgcgtgttcaagttcatcgacatgcctactgagggaaaacccactaagtccactaagccctacaaaaatggccagctgagcaaggtcatgatcatcgaaaactcccacgtgaagaaggacgatatttggccctccggaggtcaaatgaccgtgaaggacctgaccgcaaagtacaccgagggaggaaacgccattctcgaaaacatcagcttctccatttcgccgggacagcgggtcggccttctcgggcggaccggttccgggaagtcaactctgctgtcggctttcctccggctgctgaataccgagggggaaatccaaattgacggcgtgtcttgggattccattactctgcagcagtggcggaaggccttcggcgtgatcccccagaaggtgttcatcttctcgggtaccttccggaagaacctggatccttacgagcagtggagcgaccaagaaatctggaaggtcgccgacgaggtcggcctgcgctccgtgattgaacaatttcctggaaagctggacttcgtgctcgtcgacgggggatgtgtcctgtcgcacggacataagcagctcatgtgcctcgcacggtccgtgctctccaaggccaagattctgctgctggaccaaccttcggcccacctggatccggtcacctaccagatcatcaggaggaccctgaagcaggcctttgccgattgcaccgtgattctctgcgagcaccgcatcgaggccatgctggagtgccagcagttcctggtcatcgaggagaacaaggtccgccaatacgactccattcaaaagctcctcaacgagcggtcgctgttcagacaagctatttcaccgtccgatagagtgaagctcttcccgcatcggaacagctcaaagtgcaaatcgaagccgcagatcgcagccttgaaggaagagactgaggaagaggtgcaggacacccggctttaa

SEQ ID NO: 49 - DNA, 4443 bp, Artificial Sequence
  Synthetic polynucleotide
atgcagcgctcgcctctggaaaaggcgagcgtcgtgtcacggctattcttttcttggacccggcccattctcaggaagggctacaggcagaggctggagttgagcgacatctatcagattccttccgtggacagcgccgacaacctgagcgagaagctggaaagggagtgggaccgcgaactggcaagcaaaaagaaccccaagctgatcaatgccctgagaaggtgtttcttttggagattcatgttctacgggatctttctgtatctgggcgaggttacaaaggctgtgcagcccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctggcttcttcgttgtgtttctgagcgtgctgccatacgctctcatcaaaggcatcatcctgcggaagatcttcaccaccatcagcttttgcatcgtgcttagaatggccgtgacccggcagttcccatgggccgtgcaaacttggtatgattccctgggcgccatcaacaaaatccaggatttcctgcagaagcaggaatacaagacactcgaatataatctcacaactactgaggtggttatggagaacgtgactgccttctgggaggaggggttcggagagctttttgagaaggcaaaacagaataacaacaaccgcaaaaccagcaacggcgacgacagcctgttcttctccaatttttctctcctgggaacacccgtcctcaaagacatcaactttaagatcgagaggggacagctgctcgcagtcgccggatccacaggcgccggcaagacctctctgctgatggttatcatgggcgaactggagccatccgagggcaagattaagcacagtggaagaatctccttttgtagccagttcagttggattatgcccggcactattaaggagaatatcatttttggggtgagctatgatgagtatcggtatcggagcgttatcaaagcctgtcagctggaggaggatatcagcaaattcgcagagaaggataatatcgtgctgggggaggggggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaataagacccggatcctagtgaccagtaagatggagcacctgaagaaggcagacaagatcttgattctgcacgagggatcctcttacttttacggcacctttagcgagctgcagaatctccagcccgatttctcatctaagctgatgggctgtgatagcttcgaccagttctctgccgagcgcagaaacagcatcctgacagagacactgcaccggttttcactggagggcgacgcccctgtcagctggaccgagaccaaaaagcagtctttcaagcagacaggcgagttcggcgagaagcgcaaaaacagcatcctgaatccaatcaactctataaggaagtttagcatcgtgcagaagacacccctccagatgaacggcatcgaagaggacagtgacgagcccctggagcggcgcctgagcctcgtgcctgacagcgaacagggcgaggccatcctgcctaggatcagcgtgatttcaaccgggccaacactgcaggctaggagaagacagtcagtgcttaacctgatgacacatagcgtgaatcagggacagaacatccatcgaaaaaccacagcctctactcgcaaagtgtcactggctcctcaggctaatctgacagagctggacatctatagcaggaggctgagccaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggtttctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgtccacactgcagtgggccgtgaatagttcaatcgacgtggacagtctgatgaggtccgtgagccgggtgttcaagttcattgacatgcccacagaggggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggaccaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctctttccccacagaaactcctcaaagtgcaagtccaagccccagatcgccgccctgaaggaggagaccgaggaggaggtgcaggacaccaggctgtga

SEQ ID NO: 50 - DNA, 4443 bp, Artificial Sequence
  Synthetic polynucleotide
atgcagcgctcgcctctggaaaaggcgagcgtcgtgtcacggctattcttttcttggacccggcccattctcaggaagggctacaggcagaggctggagttgagcgacatctatcagattccttccgtggacagcgccgacaacctgagcgagaagctggaaagggagtgggaccgcgaactggcaagcaaaaagaaccccaagctgatcaatgccctgagaaggtgtttcttttggagattcatgttctacgggatctttctgtatctgggcgaggttacaaaggctgtgcagcccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctggcttcttcgttgtgtttctgagcgtgctgccatacgctctcatcaaaggcatcatcctgcggaagatcttcaccaccatcagcttttgcatcgtgcttagaatggccgtgacccggcagttcccatgggccgtgcaaacttggtatgattccctgggcgccatcaacaaaatccaggatttcctgcagaagcaggaatacaagacactcgaatataatctcacaactactgaggtggttatggagaacgtgactgccttctgggaggaggggttcggagagctttttgagaaggcaaaacagaataacaacaaccgcaaaaccgagaacggcgacgacagcctgttcttctccaatttttctctcctgggaacacccgtcctcaaagacatcaactttaagatcgagaggggacagctgctcgcagtcgccggatccacaggcgccggcaagacctctctgctgatggttatcatgggcgaactggagccatccgagggcaagattaagcacagtggaagaatctccttttgtagccagttcagttggattatgcccggcactattaaggagaatatcatttttggggtgagctatgatgagtatcggtatcggagcgttatcaaagcctgtcagctggaggaggatatcagcaaattcgcagagaaggataatatcgtgctgggggaggggggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaataagacccggatcctagtgaccagtaagatggagcacctgaagaaggcagacaagatcttgattctgcacgagggatcctcttacttttacggcacctttagcgagctgcagaatctccagcccgatttctcatctaagctgatgggctgtgatagcttcgaccagttctctgccgagcgcagaaacgaaatcctgacagagacactgcaccggtttgagctggagggcgacgcccctgtcagctggaccgagaccaaaaagcaggaattcaagcaggagggcgagttcggcgagaagcgcaaaaacgaaatcctgaatccaatcaactctataaggaagtttgaaatcgtgcagaagacacccctccagatgaacggcatcgaagaggacagtgacgagcccctggagcggcgcctgagcctcgtgcctgacagcgaacagggcgaggccatcctgcctaggatcgaggtgatttcaaccgggccaacactgcaggctaggagaagacagtcagtgcttaacctgatgacacatagcgtgaatcagggacagaacatccatcgaaaagaagaagccgaaactcgcaaagtggagctggctcctcaggctaatctgacagagctggacatctatagcaggaggctggaacaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggtttctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgtccacactgcagtgggccgtgaatagttcaatcgacgtggacagtctgatgaggtccgtgagccgggtgttcaagttcattgacatgcccacagaggggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggacgaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctctttccccacagaaactcctcaaagtgcaagtccaagccccagatcgccgccctgaaggaggagaccgaggaggaggtgcaggacaccaggctgtga

SEQ ID NO: 51 - DNA, 4443 bp, Artificial Sequence
  Synthetic polynucleotide
atgcagcgctcgcctctggaaaaggcgagcgtcgtgtcacggctattcttttcttggacccggcccattctcaggaagggctacaggcagaggctggagttgagcgacatctatcagattccttccgtggacagcgccgacaacctgagcgagaagctggaaagggagtgggaccgcgaactggcaagcaaaaagaaccccaagctgatcaatgccctgagaaggtgtttcttttggagattcatgttctacgggatctttctgtatctgggcgaggttacaaaggctgtgcagcccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctggcttcttcgttgtgtttctgagcgtgctgccatacgctctcatcaaaggcatcatcctgcggaagatcttcaccaccatcagcttttgcatcgtgcttagaatggccgtgacccggcagttcccatgggccgtgcaaacttggtatgattccctgggcgccatcaacaaaatccaggatttcctgcagaagcaggaatacaagacactcgaatataatctcacaactactgaggtggttatggagaacgtgactgccttctgggaggaggggttcggagagctttttgagaaggcaaaacagaataacaacaaccgcaaaaccgagaacggcgacgacagcctgttcttctccaatttttctctcctgggaacacccgtcctcaaagacatcaactttaagatcgagaggggacagctgctcgcagtcgccggatccacaggcgccggcaagacctctctgctgatggttatcatgggcgaactggagccatccgagggcaagattaagcacagtggaagaatctccttttgtagccagttcagttggattatgcccggcactattaaggagaatatcatttttggggtgagctatgatgagtatcggtatcggagcgttatcaaagcctgtcagctggaggaggatatcagcaaattcgcagagaaggataatatcgtgctgggggaggggggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaataagacccggatcctagtgaccagtaagatggagcacctgaagaaggcagacaagatcttgattctgcacgagggatcctcttacttttacggcacctttagcgagctgcagaatctccagcccgatttctcatctaagctgatgggctgtgatagcttcgaccagttctctgccgagcgcagaaacgaaatcctgacagagacactgcaccggtttgagctggagggcgacgcccctgtcagctggaccgagaccaaaaagcaggaattcaagcaggagggcgagttcggcgagaagcgcaaaaacgaaatcctgaatccaatcaactctataaggaagtttgaaatcgtgcagaagacacccctccagatgaacggcatcgaagaggacagtgacgagcccctggagcggcgcctggaactcgtgcctgacagcgaacagggcgaggccatcctgcctaggatcgaggtgatttcaaccgggccaacactgcaggctaggagaagacaggaagtgcttaacctgatgacacatagcgtgaatcagggacagaacatccatcgaaaagaagaagccgaaactcgcaaagtggagctggctcctcaggctaatctgacagagctggacatctatagcaggaggctggaacaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggtttctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgtccacactgcagtgggccgtgaatagttcaatcgacgtggacagtctgatgaggtccgtgagccgggtgttcaagttcattgacatgcccacagaggggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggacgaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctctttccccacagaaactcctcaaagtgcaagtccaagccccagatcgccgccctgaaggaggagaccgaggaggaggtgcaggacaccaggctgtga

SEQ ID NO: 52 - DNA, 4443 bp, Artificial Sequence
  Synthetic polynucleotide
atgcagcgctcgcctctggaaaaggcgagcgtcgtgtcaaagctattcttttcttggacccggcccattctcaggaagggctacaggcagaggctggagttgagcgacatctatcagattccttccgtggacagcgccgacaacctgagcgagaagctggaaagggagtgggaccgcgaactggcaagcaaaaagaaccccaagctgatcaatgccctgagaaggtgtttcttttggagattcatgttctacgggatctttctgtatctgggcgaggttacaaaggctgtgcagcccctgctgctcggcagaatcatcgcctcatacgatccagacaacaaggaagaaagaagcatcgccatctacctgggcattggcctctgcctcctgtttattgtgcggactctgctgctgcacccagcaattttcgggttgcatcatattggcatgcagatgcgcattgctatgttttccctcatctacaaaaagacactgaaactcagctcccgggtgctggacaagatctccatcggccaactggtgtctctcctgagcaataacttgaataagttcgacgaagggctggccctggcacacttcgtgtggattgcccccctgcaggtggccctgctgatgggactgatttgggaactgctgcaggctagcgctttctgcggcctggggttcctgatcgtgctggcactgtttcaggcaggcctgggccgtatgatgatgaagtacagagaccagagggccgggaagatctccgaacggctcgttattacctctgagatgatcgagaacattcagtctgtgaaagcctactgctgggaggaggctatggagaagatgatcgagaatctgagacagaccgagctgaagctgaccagaaaggccgcctacgtgaggtacttcaacagcagtgccttcttcttctctggcttcttcgttgtgtttctgagcgtgctgccatacgctctcatcaaaggcatcatcctgcggaagatcttcaccaccatcagcttttgcatcgtgcttagaatggccgtgacccggcagttcccatgggccgtgcaaacttggtatgattccctgggcgccatcaacaaaatccaggatttcctgcagaagcaggaatacaagacactcgaatataatctcacaactactgaggtggttatggagaacgtgactgccttctgggaggaggggttcggagagctttttgagaaggcaaaacagaataacaacaaccgcaaaaccgagaacggcgacgacagcctgttcttctccaatttttctctcctgggaacacccgtcctcaaagacatcaactttaagatcgagaggggacagctgctcgcagtcgccggatccacaggcgccggcaagacctctctgctgatggttatcatgggcgaactggagccatccgagggcaagattaagcacagtggaagaatctccttttgtagccagttcagttggattatgcccggcactattaaggagaatatcatttttggggtgagctatgatgagtatcggtatcggagcgttatcaaagcctgtcagctggaggaggatatcagcaaattcgcagagaaggataatatcgtgctgggggaggggggaatcaccctgagcggaggccagagagccagaatctcactggcccgggccgtctacaaggacgccgacctttaccttctggacagtccctttggatatctggatgtgctgactgaaaaggagatcttcgagtcttgtgtgtgcaagctgatggctaataagacccggatcctagtgaccagtaagatggagcacctgaagaaggcagacaagatcttgattctgcacgagggatcctcttacttttacggcacctttagcgagctgcagaatctccagcccgatttctcatctaagctgatgggctgtgatagcttcgaccagttctctgccgagcgcagaaacgaaatcctgacagagacactgcaccggtttgagctggagggcgacgcccctgtcagctggaccgagaccaaaaagcaggaattcaagcaggagggcgagttcggcgagaagcgcaaaaacgaaatcctgaatccaatcaactctataaggaagtttgaaatcgtgcagaagacacccctccagatgaacggcatcgaagaggacagtgacgagcccctggagcggcgcctgagcctcgtgcctgacagcgaacagggcgaggccatcctgcctaggatcgaggtgatttcaaccgggccaacactgcaggctaggagaagacagtcagtgcttaacctgatgacacatagcgtgaatcagggacagaacatccatcgaaaagaagaagccgaaactcgcaaagtggagctggctcctcaggctaatctgacagagctggacatctatagcaggaggctggaacaggagacaggcctggagatcagtgaggagatcaacgaagaggacctgaaggagtgctttttcgatgacatggagagtatccccgccgtcaccacctggaatacctacctccggtacatcacagtgcacaagtccctcatctttgtgctgatttggtgcctcgtgatctttctcgcagaagtggccgcctccctggtggtgctgtggctgttggggaatactccactgcaggacaaaggcaattctacacacagcaggaataattcctatgccgtgattatcaccagcacatcctcttactacgtgttctacatctacgtgggagtggcagatactctgcttgcaatgggcttcttcagggggctgcccctggtgcacacactgatcacagtgtccaagatcctccaccataaaatgctccacagcgtgctgcaggcacccatgagcaccctgaacacactgaaggccggcggcatcctgaatcgcttttccaaagacatcgccatcctcgacgatctcctgccactgaccatcttcgattttatccagctgctgctgatcgtgatcggggccatcgccgtggtggccgtgctgcagccatacattttcgtggctacagtgcccgtgatcgttgcctttatcatgctgagagcctacttcctgcagacttctcagcagctgaagcagctggagagcgaagggagaagccccatcttcactcacctggtgacaagcctgaagggactctggaccctgagagccttcggccggcagccctatttcgagaccctgtttcacaaggccctcaacctgcacacagccaactggtttctctacctgtccaccctgaggtggttccagatgaggattgaaatgatcttcgtgatttttttcatcgccgtgacattcattagcattctgaccaccggcgagggggaggggagagtgggcatcatcctgacccttgccatgaacattatgtccacactgcagtgggccgtgaatagttcaatcgacgtggacagtctgatgaggtccgtgagccgggtgttcaagttcattgacatgcccacagaggggaaacccaccaaaagcaccaagccctacaagaacgggcagctgtccaaggttatgatcatcgagaactctcacgtgaagaaggacgacatttggcccagcggcggccagatgacagtgaaagatctgaccgccaaatacaccgagggaggcaacgccatcctcgaaaacattagcttctctatcagccctggacagagggtgggcctgctgggccggacaggctcagggaagagtactctgctgtcagcattcctgaggctcctgaacacagagggcgagatccagattgacggcgtgtcctgggactccatcaccctgcagcagtggcggaaggctttcggggtgatcccccagaaggtgttcatctttagcggcactttcagaaagaatctggacccttatgagcagtggagtgaccaggagatctggaaagtggccgatgaggtcggactgaggagcgtgatcgagcagtttccagggaagctggactttgtgctggtggatggcggatgcgtgctgtctcacggccataaacagctgatgtgtctggcccggtccgtgctgtctaaggccaagatcctgctgctggacgaaccctccgcccacctggaccccgtgacataccagatcatcaggagaactctcaagcaggccttcgccgactgtaccgtgattctgtgcgagcaccgcattgaagctatgctggagtgtcagcagttcctggtgatcgaggaaaataaggtgaggcagtacgacagcatccagaagctgctgaacgagcgctccctgttccgccaggctatctccccatcagaccgggtgaagctctttccccacagaaactcctcaaagtgcaagtccaagccccagatcgccgccctgaaggaggagaccgaggaggaggtgcaggacaccaggctgtga

(19) United States
(12) Patent Application Publication
(10) Pub. No.: US 2024/0197825 A1
(43) Pub. Date: Jun. 20, 2024

(54) mRNA Encoding Engineered CFTR

(71) Applicant: Translate Bio, Inc., Lexington, MA (US)

(72) Inventors: Richard Wooster, Lexington, MA (US); Frank DeRosa, Lexington, MA (US); Lianne Boeglin, Lexington, MA (US); Priyaanka Nanduri, Lexington, MA (US); Anusha Dias, Lexington, MA (US); Khang Anh Tran, Lexington, MA (US)

(21) Appl. No.: 17/761,893

(22) PCT Filed: Sep. 17, 2020
(86) PCT No.: PCT/US2020/051277

§ 371 Date: Mar. 18, 2022

Related U.S. Application Data
(60) Provisional application No. 62/903,047, filed on Sep. 20, 2019.
(60) Provisional application No. 62/984,632, filed on Mar. 3, 2020.
(60) Provisional application No. 63/021,263, filed on May 7, 2020.

(51) Int. Cl. A61K 38/17; A61K 48/00

(52) U.S. Cl. CPC A61K 38/177; A61K 48/0033; A61K 48/005

(57) ABSTRACT

The present invention provides, among other things, improved methods and pharmaceutical compositions for treating cystic fibrosis based on codon optimized mRNA encoding an engineered or mutant Cystic Fibrosis Transmembrane Conductance Regulator (CFTR) protein.

### CROSS-REFERENCE TO RELATED APPLICATIONS

This application is a 35 U.S.C § 371 National Stage Application of International Application No. PCT/US2020/051277 filed on Sep. 17, 2020, which claims the benefit of, and priority to U.S. Provisional Patent Application Ser. No. 62/903,047 filed on Sep. 20, 2019, U.S. Provisional Patent Application Ser. No. 62/984,632, filed on Mar. 3, 2020, and U.S. Provisional Patent Application Ser. No. 63/021,263, filed on May 7, 2020, the contents of each of which are incorporated herein in its entirety.

### INCORPORATION-BY-REFERENCE OF SEQUENCE LISTING

The contents of the text file named “MRT-2115US_SL.txt”, which was created on Oct. 11, 2022 and is 261 KB in size, are hereby incorporated by reference in its entirety.

### BACKGROUND

Cystic fibrosis is an autosomal inherited disorder resulting from mutation of the CFTR gene, which encodes a chloride ion channel believed to be involved in regulation of multiple other ion channels and transport systems in epithelial cells. Loss of function of CFTR results in chronic lung disease, aberrant mucus production, and dramatically reduced life expectancy. See generally Rowe et al., New Engl. J. Med. 352, 1992-2001 (2005).

Currently there is no cure for cystic fibrosis. The literature has documented numerous difficulties encountered in attempting to induce expression of CFTR in the lung. For example, viral vectors comprising CFTR DNA triggered immune responses and CF symptoms persisted after administration. Conese et al., J. Cyst. Fibros. 10 Suppl 2, S114-28 (2011); Rosenecker et al., Curr. Opin. Mol. Ther. 8, 439-45 (2006). Non-viral delivery of DNA, including CFTR DNA, has also been reported to trigger immune responses. Alton et al., Lancet 353, 947-54 (1999); Rosenecker et al., J Gene Med. 5, 49-60 (2003). Furthermore, non-viral DNA vectors encounter the additional problem that the machinery of the nuclear pore complex does not ordinarily import DNA into the nucleus, where transcription would occur. Pearson, Nature 460, 164-69 (2009).

### SUMMARY OF THE INVENTION

The present invention provides, among other things, pharmaceutical compositions comprising messenger RNA (mRNA) encoding an engineered or mutant Cystic Fibrosis Transmembrane Conductance Regulator (CFTR) protein and methods of making and using thereof. Notably, engineered or mutant CFTR proteins described herein contain mutations that enhance the activity or stability of the protein. These pharmaceutical compositions can be used for improved treatment of cystic fibrosis.

In one aspect, the present invention provides a pharmaceutical composition for treating cystic fibrosis, comprising an mRNA encoding a Cystic Fibrosis Transmembrane Conductance Regulator (CFTR), wherein the CFTR comprises one or more mutations that produce an activated CFTR protein.

In one aspect, a pharmaceutical composition is provided herein for treating cystic fibrosis, comprising an mRNA encoding a Cystic Fibrosis Transmembrane Conductance Regulator (CFTR), wherein the CFTR comprises one or more mutations that produce a CFTR protein that is more stable than a wild type CFTR protein.

For example, in some embodiments, the mutant CFTR protein has a greater half-life in comparison to wild type CFTR. In some embodiments, the mutant CFTR protein has about the same half-life as a wild type CFTR. In some embodiments, the mutant CFTR protein has about 5 minutes, 10 minutes, 15 minutes, 30 minutes, 45 minutes, 60 minutes, 90 minutes, 2 hours, 4 hours, 6 hours, 8 hours, 10 hours, 12 hours, 14 hours, 16 hours, 18 hours, 20 hours, 22 hours, or 24 hours greater half-life in comparison to wild type CFTR. In some embodiments, the mutant CFTR protein has more than 24 hours half-life in comparison to wild type CFTR.

In some embodiments, one or more mutations are located within the CFTR regulatory domain, a membrane-spanning domain (MSD), and/or a nucleotide-binding domain (NBD). In some embodiments, one or more mutations are located within the CFTR regulatory domain amino acid residues 634-835. In some embodiments, mutations are located within different domains.

In some embodiments, the engineered CFTR protein comprising one or more mutations results in increased activity in comparison to wild type CFTR protein.

In some embodiments, the engineered CFTR protein comprising one or more mutations results in increased trafficking in comparison to wild type CFTR protein.

In some embodiments, the engineered CFTR protein comprising one or more mutations results in increased conductivity in comparison to wild type CFTR protein. In some embodiments, the engineered CFTR protein comprising one or more mutations results in increased conductivity in the presence of modulators in comparison to wild type CFTR protein. For example, the engineered CFTR protein has increased conductivity in the presence of a potentiator, corrector and/or activator in comparison to conductivity in a wild type CFTR protein.

In some embodiments, the engineered CFTR protein comprising one or more mutations has improved pharmacokinetic properties in comparison to wild type CFTR protein.

In some embodiments, the engineered CFTR protein comprising one or more mutations produces an activated CFTR protein that is more stable than a wild type CFTR protein.

In some embodiments, the engineered CFTR protein comprising one or more mutations produces a CFTR protein that is more therapeutically effective for the treatment of cystic fibrosis in comparison to a wild type CFTR protein. For example, the engineered CFTR protein has one or more of improved activity, trafficking, synergistic conductivity in the presence of modulators (e.g., potentiators, correctors and/or activators), pharmacokinetic properties, and stability in comparison to wild type CFTR protein.

In some embodiments, the CFTR comprises at least 2 mutations in amino acid residues in comparison to wild type CFTR protein. In some embodiments, the CFTR comprises at least 3 mutations in amino acid residues in comparison to wild type CFTR protein. In some embodiments, the CFTR comprises at least 4 mutations in amino acid residues in comparison to wild type CFTR protein. In some embodiments, the CFTR comprises at least 5 mutations in amino acid residues in comparison to wild type CFTR protein. In some embodiments, the CFTR comprises at least 6 mutations in amino acid residues in comparison to wild type CFTR protein. In some embodiments, the CFTR comprises at least 7 mutations in amino acid residues in comparison to wild type CFTR protein. In some embodiments, the CFTR comprises at least 10 mutations in amino acid residues in comparison to wild type CFTR protein. In some embodiments, the CFTR comprises at least 11 mutations in amino acid residues in comparison to wild type CFTR protein. In some embodiments, the CFTR comprises at least 12 mutations in amino acid residues in comparison to wild type CFTR protein. In some embodiments, the CFTR comprises at least 13 mutations in amino acid residues in comparison to wild type CFTR protein. In some embodiments, the CFTR comprises at least 14 mutations in amino acid residues in comparison to wild type CFTR protein. In some embodiments, the CFTR comprises at least 15 mutations in amino acid residues in comparison to wild type CFTR protein. In some embodiments, the CFTR comprises at least 16 mutations in amino acid residues in comparison to wild type CFTR protein. In some embodiments, the CFTR comprises at least 17 mutations in amino acid residues in comparison to wild type CFTR protein. In some embodiments, the CFTR comprises at least 18 mutations in amino acid residues in comparison to wild type CFTR protein. In some embodiments, the CFTR comprises at least 19 mutations in amino acid residues in comparison to wild type CFTR protein. In some embodiments, the CFTR comprises at least 20 mutations in amino acid residues in comparison to wild type CFTR protein.

In some embodiments, the CFTR comprises between 1 and 20 mutations in amino acid residues in comparison to wild type CFTR protein. In some embodiments, the CFTR comprises between 2 and 15 mutations in amino acid residues in comparison to wild type CFTR protein. In some embodiments, the CFTR comprises between 3 and 12 mutations in amino acid residues in comparison to wild type CFTR protein. In some embodiments, the CFTR comprises between 5 and 10 mutations in amino acid residues in comparison to wild type CFTR protein.

In some embodiments, the mutation is an amino acid substitution. In some embodiments, the mutation is a deletion of one or more amino acids. In some embodiments, the mutation is an insertion of one or more amino acids. In some embodiments, the mutation is an amino acid inversion. In some embodiments, the mutation is a combination of amino acid substitution, a deletion of one or more amino acids, an insertion of one or more amino acids, an amino acid inversion.

In some embodiments, one or more mutations are made at residues K14, K68, S422, S660, S670, S686, T690, S700, K710, S712, K716, S737, S753, S768, T787, T788, S790, S795, S813, K978, K1041, K1080, K1218, E1371, K190, G550, R553, R555, K1250, K464, W401, F409, F430, and combinations thereof. In some embodiments, a mutation is made at residue K14. In some embodiments, a mutation is made at residue K68. In some embodiments, a mutation is made at residue S422. In some embodiments, a mutation is made at residue S660. In some embodiments, a mutation is made at residue S670. In some embodiments, a mutation is made at residue S686. In some embodiments, a mutation is made at residue S700. In some embodiments, a mutation is made at residue K710. In some embodiments, a mutation is made at residue S712. In some embodiments, a mutation is made at residue K716. In some embodiments, a mutation is made at residue S737. In some embodiments, a mutation is made at residue S753. In some embodiments, a mutation is made at residue S768. In some embodiments, a mutation is made at residue T787. In some embodiments, a mutation is made at residue T788. In some embodiments, a mutation is made at residue S790. In some embodiments, a mutation is made at residue S795. In some embodiments, a mutation is made at residue S813. In some embodiments, a mutation is made at residue K978. In some embodiments, a mutation is made at residue K1041. In some embodiments, a mutation is made at residue K1080. In some embodiments, a mutation is made at residue K1218. In some embodiments, a mutation is made at residue E1371. In some embodiments, a mutation is made at residue K190. In some embodiments, a mutation is made at residue G550. In some embodiments, a mutation is made at residue R553. In some embodiments, a mutation is made at residue R555. In some embodiments, a mutation is made at residue K1250. In some embodiments, a mutation is made at residue K464. In some embodiments, a mutation is made at residue W401. In some embodiments, a mutation is made at residue F409. In some embodiments, a mutation is made at residue F430. Accordingly, in some embodiments, one or more CFTR lysine residues are mutated. In some embodiments, one or more CFTR serine residues are mutated. In some embodiments, one or more CFTR glycine residues are mutated. In some embodiments, one or more CFTR threonine residues are mutated. In some embodiments, one or more CFTR arginine residues are mutated. In some embodiments, one or more CFTR tryptophan residues are mutated. In some embodiments, one or more CFTR threonine residues are mutated. In some embodiments, one or more CFTR glutamic acid residues are mutated. In some embodiments, one or more CFTR phenylalanine residues are mutated.

In some embodiments, CFTR comprises one or more ATP gating cycle mutations. In some embodiments, CFTR comprises mutations at residues W401 and E1371. In some embodiments, CFTR comprises mutations at residues K464 and E1371. In some embodiments, CFTR comprises mutations at W401, F409, and E1371. In some embodiments, CFTR comprises mutations at W401, F409, F430 and E1371. In some embodiments, CFTR comprises a mutation at K978. In some embodiments, CFTR comprises a mutation at E1371. In some embodiments, CFTR comprises mutations at residues W401 and E1371. In some embodiments, CFTR comprises mutations at W401, F409, and E1371. In some embodiments, CFTR comprises mutations at W401, F409, F430, and E1371. In some embodiments, CFTR comprises mutations at K464 and E1371.

In some embodiments, the one or more mutations result in a substitution to an alanine residue. In some embodiments, the one or more mutations result in a substitution to an arginine residue. In some embodiments, the one or more mutations result in a substitution to an asparagine residue. In some embodiments, the one or more mutations result in a substitution to an aspartate residue. In some embodiments, the one or more mutations result in a substitution to a cysteine residue. In some embodiments, the one or more mutations result in a substitution to a glutamate residue. In some embodiments, the one or more mutations result in a substitution to a glutamine residue. In some embodiments, the one or more mutations result in a substitution to a glycine residue. In some embodiments, the one or more mutations result in a substitution to a histidine residue. In some embodiments, the one or more mutations result in a substitution to an isoleucine residue. In some embodiments, the one or more mutations result in a substitution to a leucine residue. In some embodiments, the one or more mutations result in a substitution to a lysine residue. In some embodiments, the one or more mutations result in a substitution to a methionine residue. In some embodiments, the one or more mutations result in a substitution to a phenylalanine residue. In some embodiments, the one or more mutations result in a substitution to a proline residue. In some embodiments, the one or more mutations result in a substitution to a serine residue. In some embodiments, the one or more mutations result in a substitution to a threonine residue. In some embodiments, the one or more mutations result in a substitution to a tryptophan residue. In some embodiments, the one or more mutations result in a substitution to a tyrosine residue.

In some embodiments, one or more mutations are selected from K14R, K68R, K710R, K716R, K1041R, K1080R, K1218R, and combinations thereof.

In some embodiments, the CFTR comprises mutations selected from K14R, K68R, K1218R, K14R/K68R, K14R/K1218R, K68R/K1218R, and K14R/K68R/K1218R. Accordingly, in some embodiments, the one or more mutations is a K14R mutation. In some embodiments, the one or more mutations is a K68R mutation. In some embodiments, the one or more mutations is a K1218R mutation. In some embodiments, the one or more mutations is a K14R/K68R mutation. In some embodiments, the one or more mutations is a K14R/K1218R mutation. In some embodiments, the one or more mutations is a K68R/K1218R mutation. In some embodiments, the one or more mutations is a K14R/K68R/K1218R mutation.

In some embodiments, the CFTR comprises mutations S660D/S737D/S795D/S813D, S660DD/S686D/S700D/S737D/S795D/S813D, K978C, or E1371Q. In some embodiments, the CFTR comprises mutations S660D/S737D/S795D/S813D. In some embodiments, the CFTR comprises mutations S660DD/S686D/S700D/S737D/S795D/S813D. In some embodiments, the one or more mutations is a K978C mutation. In some embodiments, the one or more mutations is a E1371Q mutation. In some embodiments, the CFTR comprises mutations S422D/S660D/S670D/S686D/T690D/S700D/S712D/S753D/T787D/T788D/S790D/S795D/S813 D. In some embodiments, the CFTR comprises mutations S422D/S660D/S670D/S686D/T690D/S700D/S712D/S737D/S753D/S768D/T787D/T788D/S790 D/S795D/S813D.

In some embodiments, the one or more mutations made at residues S422, S660, S670, S686, T690, S700, S712, S737, S753, 5768, T787, T788, 5790, S795, 5813 is an amino acid substitution to glutamic acid (E). In some embodiments, the CFTR comprises mutations S422E/S660E/S670E/S686E/T690E/S700E/S712E/S753E/T787E/T788E/S790E/S795E/S813E. In some embodiments, the CFTR comprises mutations S422E/S660E/S670E/S686E/T690E/S700E/S712E/S737E/S753E/S768E/T787E/T788E/S790E/S795E/S813E.

In some embodiments, the CFTR further comprises K14R mutation. In some embodiments, the CFTR further comprises E1371Q mutation. In some embodiments, the CFTR comprises mutations K14R/S422D/S660D/S670D/S686D/T690D/S700D/S712D/S753D/T787D/T788D/S790D/S795D/S813D. In some embodiments, the CFTR comprises mutations K14R/S422D/S660D/S670D/S686D/T690D/S700D/S712D/S737D/S753D/S768D/T787D/T788 D/S790D/S795D/S813D. In some embodiments, the CFTR comprises mutations K14R/S422E/S660E/S670E/S686E/T690E/S700E/S712E/S753E/T787E/T788E/S790E/S795E/S813E. In some embodiments, the CFTR comprises mutations K14R/S422E/S660E/S670E/S686E/T690E/S700E/S712E/S737E/S753E/S768E/T787E/T788E/S790E/S795E/S813E.

In some embodiments, the mRNA encoding CFTR is codon optimized. In some embodiments, the mRNA encoding CFTR is not codon optimized.

In some embodiments, the codon optimized CFTR mRNA further comprises a 5′ untranslated region (UTR) sequence of SEQ ID NO: 4.

In some embodiments, the codon optimized CFTR mRNA further comprises a 3′ untranslated region (UTR) sequence of SEQ ID NO: 5 of SEQ ID NO: 6.

In some embodiments, the codon optimized CFTR mRNA is encapsulated within a nanoparticle. In some embodiments, the nanoparticle is a liposome. In some embodiments, the liposome comprises one or more cationic lipids, one or more non-cationic lipids, one or more cholesterol-based lipids and one or more PEG-modified lipids. In some embodiments, the liposome comprises no more than three distinct lipid components. In some embodiments, one distinct lipid component is a sterol-based cationic lipid. In some embodiments, a sterol-based cationic lipid is imidazole cholesterol ester (ICE).

In some embodiments, the liposome has a size of less than about 200 nm. In some embodiments, the liposome has a size of less than about 150 nm. In some embodiments, the liposome has a size of less than about 120 nm. In some embodiments, the liposome has a size of less than about 110 nm. In some embodiments, the liposome has a size of less than about 100 nm. In some embodiments, the liposome has a size of less than about 80 nm. In some embodiments, the liposome has a size of less than about 60 nm. In some embodiments, the liposome has a size of less than about 50 nm.

In some embodiments, the pharmaceutical composition further comprises a CFTR potentiator. In some embodiments, the pharmaceutical composition further comprises a CFTR corrector. In some embodiments, the pharmaceutical composition further comprises a CFTR activator. In some embodiments, the pharmaceutical composition further comprises a CFTR potentiator, corrector and/or activator. Suitable CFTR potentiators, correctors and/or activators include ivacaftor (trade name Kalydeco®), lumacaftor (trade name Orkambi®), tezacaftor, vX-659, VX-445, VX-152, VX-440, VX-371, VX-561, GLPG1837, GLPG2222, GLPG2737, GLPG2451, GLPG1837, PTI-428, PTI-801, PTI-808, and eluforsen. In some embodiments, the pharmaceutical composition further comprises ivacaftor. In some embodiments, the pharmaceutical composition further comprises lumacaftor. In some embodiments, the pharmaceutical composition further comprises tezacaftor. In some embodiments, the pharmaceutical composition further comprises ivacaftor, lumacaftor, tezacaftor, or a combination. In some embodiments, the pharmaceutical composition further comprises VX-659. In some embodiments, the pharmaceutical composition further comprises VX-445. In some embodiments, the pharmaceutical composition further comprises VX-152. In some embodiments, the pharmaceutical composition further comprises VX-440. In some embodiments, the pharmaceutical composition further comprises VX-371. In some embodiments, the pharmaceutical composition further comprises VX-561. In some embodiments, the pharmaceutical composition further comprises GLPG1837. In some embodiments, the pharmaceutical composition further comprises GLPG2222. In some embodiments, the pharmaceutical composition further comprises GLPG2737. In some embodiments, the pharmaceutical composition further comprises GLPG2451. In some embodiments, the pharmaceutical composition further comprises GLPG1837. In some embodiments, the pharmaceutical composition further comprises PTI-428. In some embodiments, the pharmaceutical composition further comprises PTI-801. In some embodiments, the pharmaceutical composition further comprises PTI-808. In some embodiments, the pharmaceutical composition further comprises eluforsen. In some embodiments, the pharmaceutical composition further comprises any combination of CFTR potentiators, correctors, and/or activators.

In one aspect, the invention provides, among other things, a polynucleotide comprising a sequence complementary to the sequence of the mRNA of the present invention.

In some embodiments, the polynucleotide is a linear or circular polynucleotide comprising deoxyribonucleotide residues.

In one aspect, the invention provides, among other things, a cultured cell comprising the polynucleotide of the present invention.

In one aspect, the invention provides a method of inducing CFTR expression in epithelial cells in a lung of a mammal comprising a step of contacting the epithelial cells in the lung of the mammal with a pharmaceutical composition of the present invention.

In some embodiments, the codon optimized CFTR mRNA is administered via pulmonary delivery. In some embodiments, the codon optimized CFTR mRNA is administered via intravenous delivery. In some embodiments, the codon optimized CFTR mRNA is administered via oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, intraperitoneal, and/or intranasal administration.

In some embodiments, the pulmonary delivery is nebulization. In some embodiments, the codon optimized CFTR mRNA is administered via aerosolization.

In some embodiments, treating the subject is achieved at a lower therapeutically effective dose in comparison to treating the subject with an mRNA encoding a wild type CFTR.

In some embodiments, treating the subject in need results in shorter nebulization times to administer a therapeutically effective dose in comparison to treating with an mRNA encoding wild type CFTR.

In one aspect, the invention provides, among other things, a nucleic acid encoding a modified Cystic Fibrosis Transmembrane Conductance Regulator (CFTR) protein, wherein the modified CFTR protein comprises one or more amino acid substitutions to glutamic acid.

In some embodiments, one or more amino acid substitutions occur at serine residues. In some embodiments, one or more amino acid substitutions occur at threonine residues.

In some embodiments, one or more amino acid substitutions are located within the CFTR regulatory domain. In some embodiments, one or more amino acid substitutions are located at amino acid residues 440-820. In some embodiments, one or more amino acid substitutions occur at residue S422, S660, S670, S686, T690, S700, S712, S737, S753, S768, T787, T788, S790, S795, and/or S813.

In some embodiments, a modified CFTR protein comprises one or more mutations of S422E, S660E, S670E, S686E, T690E, S700E, S712E, S737E, S753E, S768E, T787E, T788E, S790E, S795E, and/or S813E. In some embodiments, a modified CFTR protein comprises mutations S422E/S660E/S670E/S686E/T690E/S700E/S712E/S753E/T787E/T788E/S790E/S795E/S813E. In some embodiments, a modified CFTR protein comprises mutations S422E/S660E/S670E/S686E/T690E/S700E/S712E/S737E/S753E/S768E/T787E/T788E/S790E/S795E/S813E.

In some embodiments, a modified CFTR protein further comprises K14R mutation. In some embodiments, a modified CFTR protein further comprises E1371Q mutation.

In one aspect, the invention provides, among other things, a nucleic acid encoding a modified Cystic Fibrosis Transmembrane Conductance Regulator (CFTR) protein, wherein the modified CFTR protein comprises K14R and E1371Q mutations.

In some embodiments, a modified CFTR protein further comprises one or more mutations in the CFTR regulatory domain, a membrane-spanning domain (MSD), and/or a nucleotide-binding domain.

In some embodiments, a nucleic acid is DNA, cDNA, RNA, mRNA or PNA. In some embodiments, a nucleic acid is DNA. In some embodiments, a nucleic acid is cDNA. In some embodiments, a nucleic acid is RNA. In some embodiments, a nucleic acid is mRNA. In some embodiments, a nucleic acid is PNA. In some embodiments, a nucleic acid is PCNA. In some embodiments, a nucleic acid is MCNA.

In one aspect, the invention provides, among other things, an adeno-associated virus (AAV) vector comprising a nucleic acid encoding a modified Cystic Fibrosis Transmembrane Conductance Regulator (CFTR) protein.

In some embodiments, an AAV vector is packaged in a virus or viral particle. In some embodiments, a virus particle is pseudotyped. In some embodiments, a virus is a retrovirus, adenovirus, adeno-associate virus, herpes virus, or lentivirus.

In one aspect, the invention provides, among other things, a modified CFTR protein comprising one or more amino acid substitutions to glutamic acid.

In one aspect, the invention provides, among other things, a method of treating cystic fibrosis comprising administering to a subject in need of treatment a modified CFTR protein.

In one aspect, the invention provides, among other things, a method of inducing CFTR expression in a subject, the method comprising administering the AAV vector. In one aspect, the invention provides, among other things, a method of treating cystic fibrosis comprising administering to a subject in need of treatment the AAV vector.

### BRIEF DESCRIPTION OF THE DRAWING

The drawings are for illustration purposes only not for limitation.

FIG. 1 shows a schematic that illustrates the regulation of Cystic Fibrosis Transmembrane Conductance Regulator (CFTR) channel activity.

FIG. 2A and FIG. 2B show schematics of exemplary engineered CFTR proteins with mutations that activate and/or stabilize and/or enhance CFTR protein expression. FIG. 2A shows a schematic model and exemplary R domain phosphomimetic mutations. FIG. 2B shows a schematic model of ATP-gating mutations. FIG. 2C shows exemplary stability/expression-enhancing mutations. FIG. 2D shows exemplary engineered CFTR stability mutant protein expression relative to WT.

FIG. 3A is a gel that shows mutated CFTR constructs are expressed to similar levels as compared with wild type CFTR in cultured HEK cells.

FIG. 3B shows an exemplary graph of short circuit current depicting chloride ion flux over time through WT CFTR and engineered CFTR mutant protein channels in an Ussing Chamber Assay at a high dose of 0.5 μg.

FIG. 4 shows an exemplary graph of short circuit current depicting chloride ion flux over time through WT CFTR and engineered CFTR mutant protein channels in an Ussing Chamber Assay at a low dose of 0.35 μg.

FIG. 5 shows exemplary results of a graph of a time course of short-circuit current, a measure of chloride ion transport through engineered CFTR protein E at doses ranging from 0.05-0.35 μg.

FIG. 6 shows exemplary results of a graph of a time course of short-circuit current, a measure of chloride ion transport through WT CFTR protein upon ecsin permeabilization of the membrane using doses from 10-25 μM ecsin in the presence and absence of ATP.

FIG. 7 shows exemplary results of a graph of a time course of short-circuit current, a measure of chloride ion transport through WT CFTR protein at a dose of 10 μM ecsin in an Ussing Chamber Assay.

FIG. 8 shows an exemplary graph of short circuit current depicting chloride ion flux over time through WT CFTR and engineered phosphomimetic CFTR mutant protein channels in an Ussing Chamber Assay.

FIG. 9A shows exemplary graph of short-circuit conductivity measured by Ussing chamber using FRT cells transfected with wild-type, 13D, and 13E CFTR mRNA. FIG. 9B shows exemplary graph of short-circuit conductivity measured by Ussing chamber using FRT cells transfected with wild-type, 15D, and 15E CFTR mRNA. Arrowheads indicate addition of forskolin (f), VX-770 Ivacaftor (v), or CFTR inhibitor 172. Error bars represent s.e.m. with N=3 or 4.

FIG. 10 shows an exemplary bar graph depicting maximum activated current for various trafficking CFTR mutant proteins listed in Table 10, as determined by an Ussing Chamber assay.

FIG. 11A shows an exemplary a Western blot illustrating expression of CFTR proteins (15E, 13E, E1371Q variants, and wild-type CFTR) in HEK293 cells. FIG. 11B is a quantification of expression levels as shown in FIG. 11A FIG. 11C shows exemplary graph of short-circuit conductivity measured by Ussing chamber using FRT cells transfected with wild-type, 15E, 15E/K14R, 13D, 13D/K14R, 13E and 13E/K14R CFTR mRNAs. FIG. 11D is a bar graph representation of maximum activated current for various CFTR constructs tested in FIG. 11C. FIG. 11E is a series of graphs that show short-circuit conductivity measured by Ussing chamber using FRT cells that were transfected with WT, 13D, 13E, 15D or 15E CFTR mRNAs as indicated.

FIG. 12A is a table of currents depicting chloride ion flux at baseline and after forskolin, VX-770, and CFTRinh-172 additions. FIG. 12B is an exemplary graph of short-circuit conductivity illustrating, among other things, that alanine substitution at phosphomimetic sites reduces forskolin-induced chloride conductance in FRT cells.

FIG. 13A shows exemplary graph of short-circuit conductivity measured by Ussing chamber using FRT cells transfected with wild-type, E1371Q and E1371Q/K14R CFTR mRNAs. Arrowheads indicate addition of forskolin (f), VX-770 Ivacaftor (v), or CFTR inhibitor 172. Error bars represent s.e.m. with N=3 or 4. FIG. 13B is a bar graph representation of activity and expression of E1371Q CFTR mutant and E1371Q/K14R CFTR mutant compared to the wild-type CFTR protein.

FIG. 14A and FIG. 14B is a bar graph representation of maximum activation current for various CFTR constructs in Ussing chamber assay at 22 and 44 hours. FIG. 14C is an exemplary bar graphs showing that removal of ubiquitination site at N terminus alters channel activity when combined with other substitutions.

FIG. 15A depicts a schematic that shows CFTR that contains mutations at the indicated amino acid residues. FIG. 15B depicts a series of graphs that shows channel relaxation times for the indicated CFTR mutants. FIG. 15C depicts a series of graphs that show measurements of short-circuit conductivity measured by Ussing chamber of CFTR variants.

FIG. 16A depicts a graph that shows short-circuit conductivity measured by Ussing chamber using FRT cells that were transfected with CFTR mRNAs that had phosphomimetic CFTR mutations. FIG. 16B is a graph that shows forskolin EC50 (nM) in CFTR mRNAs that had varying number of phosphomimetic mutations.

FIG. 17 depicts a graph that shows forskolin sensitivity of CFTR mRNAs that had specific amino acid substitutions as assessed by short-circuit conductivity measured by Ussing chamber using FRT cells that were transfected with CFTR mRNAs identified in the graph.

FIG. 18A is a gel that depicts the banding patterns of various CFTR sequences.

FIG. 18B is a bar graph that depicts CFTR mRNA potency of various CFTR sequences. FIG. 18C is a bar graph that depicts the relative expression of various CFTR sequences in the C band.

FIG. 19 is a bar graph that depicts the results of an assay to assess cytotoxicity of various CFTR mRNA sequences.

FIGS. 20A, 20B, 20C, 20D, 20E, and 20F are a series of graphs that show short-circuit conductivity measured by Ussing chamber of various codon-optimized and non-codon optimized CFTR constructs.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “not in a single batch” would not mean mRNA synthesized at different times that are combined to achieve the desired amount.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or grammatical equivalent, refers to the process of confining an mRNA molecule within a nanoparticle.

Engineered or mutant: As used herein, the terms “engineered” or “mutant”, or grammatical equivalents refer to a nucleotide or protein sequence comprising one or more modifications compared to its naturally-occurring sequence, including but not limited to deletions, insertions of heterologous nucleic acids or amino acids, inversions, substitutions, or combinations thereof.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated. Impurities: As used herein, the term “impurities” refers to substances inside a confined amount of liquid, gas, or solid, which differ from the chemical composition of the target material or compound. Impurities are also referred to as contaminants.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Stable: As used herein, the term “stable” protein or its grammatical equivalents refer to protein that retains its physical stability and/or biological activity. In one embodiment, protein stability is determined based on the percentage of monomer protein in the solution, at a low percentage of degraded (e.g., fragmented) and/or aggregated protein. In one embodiment, a stable engineered protein retains or exhibits an enhanced half-life as compared to a wild-type protein. In one embodiment, a stable engineered protein is less prone to ubiquitination that leads to proteolysis as compared to a wild-type protein.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, improved methods and pharmaceutical compositions for treating cystic fibrosis using messenger RNA (mRNA) encoding an engineered or mutant Cystic Fibrosis Transmembrane Conductance Regulator (CFTR) protein. In some embodiments, the mRNA is a codon-optimized mRNA. In particular embodiments, the engineered or mutant CFTR proteins achieve higher activity or stability than the wild-type CFTR protein. mRNAs disclosed herein encoding engineered or mutant CFTR proteins are particularly useful for treating cystic fibrosis by mRNA therapeutics.

### Cystic Fibrosis

The present invention may be used to treat a subject who is suffering from or susceptible to cystic fibrosis. Cystic fibrosis is a genetic disorder characterized by mutations in the gene for Cystic Fibrosis Transmembrane Conductance Regulator (CFTR). The CFTR protein functions as a channel across the membrane of cells that produce mucus, sweat, saliva, tears, and digestive enzymes. The channel transports negatively charged particles called chloride ions into and out of cells. The transport of chloride ions helps control the movement of water in tissues, which is necessary for the production of thin, freely flowing mucus. Mucus is a slippery substance that lubricates and protects the lining of the airways, digestive system, reproductive system, and other organs and tissues.

Respiratory symptoms of cystic fibrosis include: a persistent cough that produces thick mucus (sputum), wheezing, breathlessness, exercise intolerance, repeated lung infections and inflamed nasal passages or a stuffy nose. Digestive symptoms of cystic fibrosis include: foul-smelling, greasy stools, poor weight gain and growth, intestinal blockage, particularly in newborns (meconium ileus), and severe constipation.

Codon Optimized mRNA Encoding CFTR

In some embodiments, the present invention provides methods and compositions for delivering codon optimized mRNA encoding CFTR to a subject for the treatment of cystic fibrosis. A suitable codon optimized CFTR mRNA encodes any full length, fragment or portion of a CFTR protein which can be substituted for naturally-occurring CFTR protein activity and/or reduce the intensity, severity, and/or frequency of one or more symptoms associated with cystic fibrosis.

In some embodiments, a suitable codon optimized mRNA sequence is an mRNA sequence encoding a human CFTR (hCFTR) protein. Exemplary codon optimized CFTR mRNA coding sequence and the corresponding amino acid sequence are shown in Table 1:

TABLE 1
Exemplary Codon-Optimized Human CFTR

| | |
|---|---|
| SEQ ID | AUGCAACGCUCUCCUCUUGAAAAGGCCUCGGUGGUGUCCAAGCUCUU |
| NO: 1 | CUUCUCGUGGACUAGACCCAUCCUGAGAAAGGGGUACAGACAGCGCU |
| | UGGAGCUGUCCGAUAUCUAUCAAAUCCCUUCCGUGGACUCCGCGGAC |
| | AACCUGUCCGAGAAGCUCGAGAGAGAAUGGGACAGAGAACUCGCCUC |
| | AAAGAAGAACCCGAAGCUGAUUAAUGCGCUUAGGCGGUGCUUUUUC |
| | UGGCGGUUCAUGUUCUACGGCAUCUUCCUCUACCUGGGAGAGGUCAC |
| | CAAGGCCGUGCAGCCCCUGUUGCUGGGACGGAUUAUUGCCUCCUACG |
| | ACCCCGACAACAAGGAAGAAAGAAGCAUCGCUAUCUACUUGGGCAUC |
| | GGUCUGUGCCUGCUUUUCAUCGUCCGGACCCUCUUGUUGCAUCCUGC |
| | UAUUUUCGGCCUGCAUCACAUUGGCAUGCAGAUGAGAAUUGCCAUG |
| | UUUUCCCUGAUCUACAAGAAAACUCUGAAGCUCUCGAGCCGCGUGCU |
| | UGACAAGAUUUCCAUCGGCCAGCUCGUGUCCCUGCUCUCCAACAAUC |
| | UGAACAAGUUCGACGAGGGCCUCGCCCUGGCCCACUUCGUGUGGAUC |
| | GCCCCUCUGCAAGUGGCGCUUCUGAUGGGCCUGAUCUGGGAGCUGCU |
| | GCAAGCCUCGGCAUUCUGUGGGCUUGGAUUCCUGAUCGUGCUGGCAC |
| | UGUUCCAGGCCGGACUGGGGCGGAUGAUGAUGAAGUACAGGGACCA |
| | GAGAGCCGGAAAGAUUUCCGAACGGCUGGUGAUCACUUCGGAAAUG |
| | AUCGAAAACAUCCAGUCAGUGAAGGCCUACUGCUGGGAAGAGGCCAU |
| | GGAAAAGAUGAUUGAAAACCUCCGGCAAACCGAGCUGAAGCUGACCC |
| | GCAAGGCCGCUUACGUGCGCUAUUUCAACUCGUCCGCUUUCUUCUUC |
| | UCCGGGUUCUUCGUGGUGUUUCUCUCCGUGCUCCCCUACGCCCUGAU |
| | UAAGGGAAUCAUCCUCAGGAAGAUCUUCACCACCAUUUCCUUCUGUA |
| | UCGUGCUCCGCAUGGCCGUGACCCGGCAGUUCCCAUGGGCCGUGCAG |
| | ACUUGGUACGACUCCCUGGGAGCCAUUAACAAGAUCCAGGACUUCCU |
| | UCAAAAGCAGGAGUACAAGACCCUCGAGUACAACCUGACUACUACCG |
| | AGGUCGUGAUGGAAAACGUCACCGCCUUUUGGGAGGAGGGAUUUGG |
| | CGAACUGUUCGAGAAGGCCAAGCAGAACAACAACAACCGCAAGACCU |
| | CGAACGGUGACGACUCCCUCUUCUUUUCAAACUUCAGCCUGCUCGGG |
| | ACGCCCGUGCUGAAGGACAUUAACUUCAAGAUCGAAAGAGGACAGCU |
| | CCUGGCGGUGGCCGGAUCGACCGGAGCCGGAAAGACUUCCCUGCUGA |
| | UGGUGAUCAUGGGAGAGCUUGAACCUAGCGAGGGAAAGAUCAAGCA |
| | CUCCGGCCGCAUCAGCUUCUGUAGCCAGUUUUCCUGGAUCAUGCCCG |
| | GAACCAUUAAGGAAAACAUCAUCUUCGGCGUGUCCUACGAUGAAUAC |
| | CGCUACCGGUCCGUGAUCAAAGCCUGCCAGCUGGAAGAGGAUAUUUC |
| | AAAGUUCGCGGAGAAAGAUAACAUCGUGCUGGGCGAAGGGGGUAUU |
| | ACCUUGUCGGGGGGCCAGCGGGCUAGAAUCUCGCUGGCCAGAGCCGU |
| | GUAUAAGGACGCCGACCUGUAUCUCCUGGACUCCCCCUUCGGAUACC |
| | UGGACGUCCUGACCGAAAAGGAGAUCUUCGAAUCGUGCGUGUGCAA |
| | GCUGAUGGCUAACAAGACUCGCAUCCUCGUGACCUCCAAAAUGGAGC |
| | ACCUGAAGAAGGCAGACAAGAUUCUGAUUCUGCAUGAGGGGUCCUCC |
| | UACUUUUACGGCACCUUCUCGGAGUUGCAGAACUUGCAGCCCGACUU |
| | CUCAUCGAAGCUGAUGGGUUGCGACAGCUUCGACCAGUUCUCCGCCG |
| | AAAGAAGGAACUCGAUCCUGACGGAAACCUUGCACCGCUUCUCUUUG |
| | GAAGGCGACGCCCCUGUGUCAUGGACCGAGACUAAGAAGCAGAGCUU |
| | CAAGCAGACCGGGGAAUUCGGCGAAAAGAGGAAGAACAGCAUCUUG |
| | AACCCCAUUAACUCCAUCCGCAAGUUCUCAAUCGUGCAAAAGACGCC |
| | ACUGCAGAUGAACGGCAUUGAGGAGGACUCCGACGAACCCCUUGAGA |
| | GGCGCCUGUCCCUGGUGCCGGACAGCGAGCAGGGAGAAGCCAUCCUG |
| | CCUCGGAUUUCCGUGAUCUCCACUGGUCCGACGCUCCAAGCCCGGCG |
| | GCGGCAGUCCGUGCUGAACCUGAUGACCCACAGCGUGAACCAGGGCC |
| | AAAACAUUCACCGCAAGACUACCGCAUCCACCCGGAAAGUGUCCCUG |
| | GCACCUCAAGCGAAUCUUACCGAGCUCGACAUCUACUCCCGGAGACU |
| | GUCGCAGGAAACCGGGCUCGAAAUUUCCGAAGAAAUCAACGAGGAG |
| | GAUCUGAAAGAGUGCUUCUUCGACGAUAUGGAGUCGAUACCCGCCGU |
| | GACGACUUGGAACACUUAUCUGCGGUACAUCACUGUGCACAAGUCAU |
| | UGAUCUUCGUGCUGAUUUGGUGCCUGGUGAUUUUCCUGGCCGAGGU |
| | CGCGGCCUCACUGGUGGUGCUCUGGCUGUUGGGAAACACGCCUCUGC |
| | AAGACAAGGGAAACUCCACGCACUCGAGAAACAACAGCUAUGCCGUG |
| | AUUAUCACUUCCACCUCCUCUUAUUACGUGUUCUACAUCUACGUCGG |
| | AGUGGCGGAUACCCUGCUCGCGAUGGGUUUCUUCAGAGGACUGCCGC |
| | UGGUCCACACCUUGAUCACCGUCAGCAAGAUUCUUCACCACAAGAUG |
| | UUGCAUAGCGUGCUGCAGGCCCCCAUGUCCACCCUCAACACUCUGAA |
| | GGCCGGAGGCAUUCUGAACAGAUUCUCCAAGGACAUCGCUAUCCUGG |
| | ACGAUCUCCUGCCGCUUACCAUCUUUGACUUCAUCCAGCUGCUGCUG |
| | AUCGUGAUUGGAGCAAUCGCAGUGGUGGCGGUGCUGCAGCCUUACA |
| | UUUUCGUGGCCACUGUGCCGGUCAUUGUGGCGUUCAUCAUGCUGCGG |
| | GCCUACUUCCUCCAAACCAGCCAGCAGCUGAAGCAACUGGAAUCCGA |
| | GGGACGAUCCCCCAUCUUCACUCACCUUGUGACGUCGUUGAAGGGAC |
| | UGUGGACCCUCCGGGCUUUCGGACGGCAGCCCUACUUCGAAACCCUC |
| | UUCCACAAGGCCCUGAACCUCCACACCGCCAAUUGGUUCCUGUACCU |
| | GUCCACCCUGCGGUGGUUCCAGAUGCGCAUCGAGAUGAUUUUCGUCA |
| | UCUUCUUCAUCGCGGUCACAUUCAUCAGCAUCCUGACUACCGGAGAG |
| | GGAGAGGGACGGGUCGGAAUAAUCCUGACCCUCGCCAUGAACAUUAU |
| | GAGCACCCUGCAGUGGGCAGUGAACAGCUCGAUCGACGUGGACAGCC |
| | UGAUGCGAAGCGUCAGCCGCGUGUUCAAGUUCAUCGACAUGCCUACU |
| | GAGGGAAAACCCACUAAGUCCACUAAGCCCUACAAAAAUGGCCAGCU |
| | GAGCAAGGUCAUGAUCAUCGAAAACUCCCACGUGAAGAAGGACGAU |
| | AUUUGGCCCUCCGGAGGUCAAAUGACCGUGAAGGACCUGACCGCAAA |
| | GUACACCGAGGGAGGAAACGCCAUUCUCGAAAACAUCAGCUUCUCCA |
| | UUUCGCCGGGACAGCGGGUCGGCCUUCUCGGGCGGACCGGUUCCGGG |
| | AAGUCAACUCUGCUGUCGGCUUUCCUCCGGCUGCUGAAUACCGAGGG |
| | GGAAAUCCAAAUUGACGGCGUGUCUUGGGAUUCCAUUACUCUGCAGC |
| | AGUGGCGGAAGGCCUUCGGCGUGAUCCCCCAGAAGGUGUUCAUCUUC |
| | UCGGGUACCUUCCGGAAGAACCUGGAUCCUUACGAGCAGUGGAGCGA |
| | CCAAGAAAUCUGGAAGGUCGCCGACGAGGUCGGCCUGCGCUCCGUGA |
| | UUGAACAAUUUCCUGGAAAGCUGGACUUCGUGCUCGUCGACGGGGG |
| | AUGUGUCCUGUCGCACGGACAUAAGCAGCUCAUGUGCCUCGCACGGU |
| | CCGUGCUCUCCAAGGCCAAGAUUCUGCUGCUGGACGAACCUUCGGCC |
| | CACCUGGAUCCGGUCACCUACCAGAUCAUCAGGAGGACCCUGAAGCA |
| | GGCCUUUGCCGAUUGCACCGUGAUUCUCUGCGAGCACCGCAUCGAGG |
| | CCAUGCUGGAGUGCCAGCAGUUCCUGGUCAUCGAGGAGAACAAGGUC |
| | CGCCAAUACGACUCCAUUCAAAAGCUCCUCAACGAGCGGUCGCUGUU |
| | CAGACAAGCUAUUUCACCGUCCGAUAGAGUGAAGCUCUUCCCGCAUC |
| | GGAACAGCUCAAAGUGCAAAUCGAAGCCGCAGAUCGCAGCCUUGAAG |
| | GAAGAGACUGAGGAAGAGGUGCAGGACACCCGGCUUUAA |
| SEQ ID | AUGCAGCGGUCCCCGCUCGAAAAGGCCAGUGUCGUGUCCAAACUCUU |
| NO: 2 | CUUCUCAUGGACUCGGCCUAUCCUUAGAAAGGGGUAUCGGCAGAGGC |
| | UUGAGUUGUCUGACAUCUACCAGAUCCCCUCGGUAGAUUCGGCGGAU |
| | AACCUCUCGGAGAAGCUCGAACGGGAAUGGGACCGCGAACUCGCGUC |
| | UAAGAAAAACCCGAAGCUCAUCAACGCACUGAGAAGGUGCUUCUUCU |
| | GGCGGUUCAUGUUCUACGGUAUCUUCUUGUAUCUCGGGGAGGUCAC |
| | AAAAGCAGUCCAACCCCUGUUGUUGGGUCGCAUUAUCGCCUCGUACG |
| | ACCCCGAUAACAAAGAAGAACGGAGCAUCGCGAUCUACCUCGGGAUC |
| | GGACUGUGUUUGCUUUUCAUCGUCAGAACACUUUUGUUGCAUCCAGC |
| | AAUCUUCGGCCUCCAUCACAUCGGUAUGCAGAUGCGAAUCGCUAUGU |
| | UUAGCUUGAUCUACAAAAAGACACUGAAACUCUCGUCGCGGGUGUU |
| | GGAUAAGAUUUCCAUCGGUCAGUUGGUGUCCCUGCUUAGUAAUAAC |
| | CUCAACAAAUUCGAUGAGGGACUGGCGCUGGCACAUUUCGUGUGGA |
| | UUGCCCCGUUGCAAGUCGCCCUUUUGAUGGGCCUUAUUUGGGAGCUG |
| | UUGCAGGCAUCUGCCUUUUGUGGCCUGGGAUUUCUGAUUGUGUUGG |
| | CAUUGUUUCAGGCUGGGCUUGGGCGGAUGAUGAUGAAGUAUCGCGA |
| | CCAGAGAGCGGGUAAAAUCUCGGAAAGACUCGUCAUCACUUCGGAAA |
| | UGAUCGAAAACAUCCAGUCGGUCAAAGCCUAUUGCUGGGAAGAAGC |
| | UAUGGAGAAGAUGAUUGAAAACCUCCGCCAAACUGAGCUGAAACUG |
| | ACCCGCAAGGCGGCGUAUGUCCGGUAUUUCAAUUCGUCAGCGUUCUU |
| | CUUUUCCGGGUUCUUCGUUGUCUUUCUCUCGGUUUUGCCUUAUGCCU |
| | UGAUUAAGGGGAUUAUCCUCCGCAAGAUUUUCACCACGAUUUCGUUC |
| | UGCAUUGUAUUGCGCAUGGCAGUGACACGGCAAUUUCCGUGGGCCGU |
| | GCAGACAUGGUAUGACUCGCUUGGAGCGAUCAACAAAAUCCAAGACU |
| | UCUUGCAAAAGCAAGAGUACAAGACCCUGGAGUACAAUCUUACUACU |
| | ACGGAGGUAGUAAUGGAGAAUGUGACGGCUUUUUGGGAAGAGGGUU |
| | UUGGAGAACUGUUUGAGAAAGCAAAGCAGAAUAACAACAACCGCAA |
| | GACCUCAAAUGGGGACGAUUCCCUGUUUUUCUCGAACUUCUCCCUGC |
| | UCGGAACACCCGUGUUGAAGGACAUCAAUUUCAAGAUUGAGAGGGG |
| | ACAGCUUCUCGCGGUAGCGGGAAGCACUGGUGCGGGAAAAACUAGCC |
| | UCUUGAUGGUGAUUAUGGGGGAGCUUGAGCCCAGCGAGGGGAAGAU |
| | UAAACACUCCGGGCGUAUCUCAUUCUGUAGCCAGUUUUCAUGGAUCA |
| | UGCCCGGAACCAUUAAAGAGAACAUCAUUUUCGGAGUAUCCUAUGA |
| | UGAGUACCGAUACAGAUCGGUCAUUAAGGCGUGCCAGUUGGAAGAG |
| | GACAUUUCUAAGUUCGCCGAGAAGGAUAACAUCGUCUUGGGAGAAG |
| | GGGGUAUUACAUUGUCGGGAGGGCAGCGAGCGCGGAUCAGCCUCGCG |
| | AGAGCGGUAUACAAAGAUGCAGAUUUGUAUCUGCUUGAUUCACCGU |
| | UUGGAUACCUCGACGUAUUGACAGAAAAAGAAAUCUUCGAGUCGUG |
| | CGUGUGUAAACUUAUGGCUAAUAAGACGAGAAUCCUGGUGACAUCA |
| | AAAAUGGAACACCUUAAGAAGGCGGACAAGAUCCUGAUCCUCCACGA |
| | AGGAUCGUCCUACUUUUACGGCACUUUCUCAGAGUUGCAAAACUUGC |
| | AGCCGGACUUCUCAAGCAAACUCAUGGGGUGUGACUCAUUCGACCAG |
| | UUCAGCGCGGAACGGCGGAACUCGAUCUUGACGGAAACGCUGCACCG |
| | AUUCUCGCUUGAGGGUGAUGCCCCGGUAUCGUGGACCGAGACAAAGA |
| | AGCAGUCGUUUAAGCAGACAGGAGAAUUUGGUGAGAAAAGAAAGAA |
| | CAGUAUCUUGAAUCCUAUUAACUCAAUUCGCAAGUUCUCAAUCGUCC |
| | AGAAAACUCCACUGCAGAUGAAUGGAAUUGAAGAGGAUUCGGACGA |
| | ACCCCUGGAGCGCAGGCUUAGCCUCGUGCCGGAUUCAGAGCAAGGGG |
| | AGGCCAUUCUUCCCCGGAUUUCGGUGAUUUCAACCGGACCUACACUU |
| | CAGGCGAGGCGAAGGCAAUCCGUGCUCAACCUCAUGACGCAUUCGGU |
| | AAACCAGGGGCAAAACAUUCACCGCAAAACGACGGCCUCAACGAGAA |
| | AAGUGUCACUUGCACCCCAGGCGAAUUUGACUGAACUCGACAUCUAC |
| | AGCCGUAGGCUUUCGCAAGAAACCGGACUUGAGAUCAGCGAAGAAA |
| | UCAAUGAAGAAGAUUUGAAAGAGUGUUUCUUUGAUGACAUGGAAUC |
| | AAUCCCAGCGGUGACAACGUGGAACACAUACUUGCGUUACAUCACGG |
| | UGCACAAGUCCUUGAUUUUCGUCCUCAUCUGGUGUCUCGUGAUCUUU |
| | CUCGCUGAGGUCGCAGCGUCACUUGUGGUCCUCUGGCUGCUUGGUAA |
| | UACGCCCUUGCAAGACAAAGGCAAUUCUACACACUCAAGAAACAAUU |
| | CCUAUGCCGUGAUUAUCACUUCUACAAGCUCGUAUUACGUGUUUUAC |
| | AUCUACGUAGGAGUGGCCGACACUCUGCUCGCGAUGGGUUUCUUCCG |
| | AGGACUCCCACUCGUUCACACGCUUAUCACUGUCUCCAAGAUUCUCC |
| | ACCAUAAGAUGCUUCAUAGCGUACUGCAGGCUCCCAUGUCCACCUUG |
| | AAUACGCUCAAGGCGGGAGGUAUUUUGAAUCGCUUCUCAAAAGAUA |
| | UUGCAAUUUUGGAUGACCUUCUGCCCCUGACGAUCUUCGACUUCAUC |
| | CAGUUGUUGCUGAUCGUGAUUGGGGCUAUUGCAGUAGUCGCUGUCC |
| | UCCAGCCUUACAUUUUUGUCGCGACCGUUCCGGUGAUCGUGGCGUUU |
| | AUCAUGCUGCGGGCCUAUUUCUUGCAGACGUCACAGCAGCUUAAGCA |
| | ACUGGAGUCUGAAGGGAGGUCGCCUAUCUUUACGCAUCUUGUGACCA |
| | GUUUGAAGGGAUUGUGGACGUUGCGCGCCUUUGGCAGGCAGCCCUAC |
| | UUUGAAACACUGUUCCACAAAGCGCUGAAUCUCCAUACGGCAAAUUG |
| | GUUUUUGUAUUUGAGUACCCUCCGAUGGUUUCAGAUGCGCAUUGAG |
| | AUGAUUUUUGUGAUCUUCUUUAUCGCGGUGACUUUUAUCUCCAUCU |
| | UGACCACGGGAGAGGGCGAGGGACGGGUCGGUAUUAUCCUGACACUC |
| | GCCAUGAACAUUAUGAGCACUUUGCAGUGGGCAGUGAACAGCUCGA |
| | UUGAUGUGGAUAGCCUGAUGAGGUCCGUUUCGAGGGUCUUUAAGUU |
| | CAUCGACAUGCCGACGGAGGGAAAGCCCACAAAAAGUACGAAACCCU |
| | AUAAGAAUGGGCAAUUGAGUAAGGUAAUGAUCAUCGAGAACAGUCA |
| | CGUGAAGAAGGAUGACAUCUGGCCUAGCGGGGGUCAGAUGACCGUG |
| | AAGGACCUGACGGCAAAAUACACCGAGGGAGGGAACGCAAUCCUUGA |
| | AAACAUCUCGUUCAGCAUUAGCCCCGGUCAGCGUGUGGGGUUGCUCG |
| | GGAGGACCGGGUCAGGAAAAUCGACGUUGCUGUCGGCCUUCUUGAG |
| | ACUUCUGAAUACAGAGGGUGAGAUCCAGAUCGACGGCGUUUCGUGG |
| | GAUAGCAUCACCUUGCAGCAGUGGCGGAAAGCGUUUGGAGUAAUCCC |
| | CCAAAAGGUCUUUAUCUUUAGCGGAACCUUCCGAAAGAAUCUCGAUC |
| | CUUAUGAACAGUGGUCAGAUCAAGAGAUUUGGAAAGUCGCGGACGA |
| | GGUUGGCCUUCGGAGUGUAAUCGAGCAGUUUCCGGGAAAACUCGAC |
| | UUUGUCCUUGUAGAUGGGGGAUGCGUCCUGUCGCAUGGGCACAAGC |
| | AGCUCAUGUGCCUGGCGCGAUCCGUCCUCUCUAAAGCGAAAAUUCUU |
| | CUCUUGGAUGAACCUUCGGCCCAUCUGGACCCGGUAACGUAUCAGAU |
| | CAUCAGAAGGACACUUAAGCAGGCGUUUGCCGACUGCACGGUGAUUC |
| | UCUGUGAGCAUCGUAUCGAGGCCAUGCUCGAAUGCCAGCAAUUUCUU |
| | GUCAUCGAAGAGAAUAAGGUCCGCCAGUACGACUCCAUCCAGAAGCU |
| | GCUUAAUGAGAGAUCAUUGUUCCGGCAGGCGAUUUCACCAUCCGAUA |
| | GGGUGAAACUUUUUCCACACAGAAAUUCGUCGAAGUGCAAGUCCAA |
| | ACCGCAGAUCGCGGCCUUGAAAGAAGAGACUGAAGAAGAAGUUCAA |
| | GACACGCGUCUUUAA |
| Human | MQRSPLEKASVVSKLFFSWTRPILRKGYRQRLELSDIYQIPSVDSADNLSEK |
| CFTR | LEREWDRELASKKNPKLINALRRCFFWRFMFYGIFLYLGEVTKAVQPLLL |
| Protein | GRIIASYDPDNKEERSIAIYLGIGLCLLFIVRTLLLHPAIFGLHHIGMQMRIA |
| Sequence | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNSILTETLHR |
| | FSLEGDAPVSWTETKKQSFKQTGEFGEKRKNSILNPINSIRKFSIVQKTPLQ |
| | MNGIEEDSDEPLERRLSLVPDSEQGEAILPRISVISTGPTLQARRRQSVLNL |
| | MTHSVNQGQNIHRKTTASTRKVSLAPQANLTELDIYSRRLSQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSK |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHKALN |
| | LHTANWFLYLSTLRWFQMRIEMIFVIFFIAVTFISILTTGEGEGRVGIILTLA |
| | MNIMSTLQWAVNSSIDVDSLMRSVSRVFKFIDMPTEGKPTKSTKPYKNGQ |
| | LSKVMIIENSHVKKDDIWPSGGQMTVKDLTAKYTEGGNAILENISFSISPGQ |
| | RVGLLGRTGSGKSTLLSAFLRLLNTEGEIQIDGVSWDSITLQQWRKAFGVIP |
| | QKVFIFSGTFRKNLDPYEQWSDQEIWKVADEVGLRSVIEQFPGKLDFVLVD |
| | GGCVLSHGHKQLMCLARSVLSKAKILLLDEPSAHLDPVTYQIIRRTLKQAF |
| | ADCTVILCEHRIEAMLECQQFLVIEENKVRQYDSIQKLLNERSLFRQAISPS |
| | DRVKLFPHRNSSKCKSKPQIAALKEETEEEVQDTRL (SEQ ID NO: 3) |

Additional exemplary codon optimized mRNA sequences are described below, for example, SEQ ID NO: 7 and SEQ ID NO: 8, both of which include 5′ and 3′ untranslated regions framing a codon-optimized hCFTR-encoding mRNA and SEQ ID NO: 27 to SEQ ID NO: 40.

### Engineered or Mutant CFTR Proteins

In some embodiments, a suitable mRNA sequence encodes an engineered or mutant CFTR protein. In some embodiments, the mRNA sequence may be codon optimized. In some embodiments, an engineered or mutant CFTR protein may be a modified CFTR protein containing one or more amino acid substitutions, deletions, and/or insertions that increases the activity and/or stability as compared to a wild-type or naturally-occurring CFTR protein. Accordingly, in some embodiments, the modified CFTR amino acid sequence has 70%, 75%, 80%, 85%, 90%, 95, 98%, 99% or more identity to wild type CFTR. Furthermore, in some embodiments, the modified CFTR is encoded by an mRNA that has 70%, 75%, 80%, 85%, 90%, 95, 98%, 99% or more identity to an mRNA that encodes a wild type CFTR protein.

### Activated CFTR Protein Mutations

CFTR is a single polypeptide containing an N-terminal lasso motif, two transmembrane domains (TMDs), and two nucleotide-binding domains (NBDs). Distinct from other ATP-binding cassette (ABC) transporters, CFTR also contains an ˜200-residue cytoplasmic regulatory (R) domain that regulates the activity of CFTR. The activity of CFTR is regulated by protein kinase A-dependent phosphorylation and ATP. The R domain contains 19 predicted sites for protein kinase A (PKA); up to six have been found phosphorylated in vivo. Phosphorylation of the R domain increases the open probability of the CFTR channel and also stimulates ATP hydrolysis by CFTR. Additionally, eliminating ATP hydrolysis prolongs the lifetime of the open CFTR channel (activated CFTR), and mutating the cytosolic loops promotes CFTR activity independent of ATP.

In some embodiments, an engineered CFTR protein may contain one or more modifications that provide constitutive CFTR activity. In some embodiments, the one or more modifications provide a more CFTR activity in comparison to wild type CFTR protein activity. In particular, an engineered CFTR protein may contain one or more modifications that mimic phosphorylated residue in the R domain (R Domain Phosphomimetic mutation). In some embodiments, serine (S) or threonine (T) residues are substituted with aspartic acid (D) residues to mimic phosphorylation states. In some embodiments, serine (S) or threonine (T) residues are substituted with glutamic acid (E) residues. In some embodiments, an engineered CFTR protein may contain one or more modifications at residues S422, S660, S670, S686, T690, S700, S712, S737, S753, S768, T787, T788, S790, S795 and S813. In some embodiments, an engineered CFTR protein may contain one or more modifications that include S422D, S660D, S670D, S686D, T690D, S700D, S712D, S737D, S753D, S768D, T787D, T788D, S790D, S795D and S813D. In some embodiments, an engineered CFTR protein contains modifications of S660D, S737D, S795D and S813D. In some embodiments, an engineered CFTR protein contains modifications of S660D, S686D, S700D, S737D, S795D and S813D. In some embodiments, an engineered CFTR protein contains modifications of S660D, S686D, S700D, S712D, S737D, S768D, S795D and S813D. In some embodiments, an engineered CFTR protein contains modifications of S422D, S660D, S670D, S686D, T690D, S700D, S712D, S753D, T787D, T788D, S790D, S795D and S813D. In some embodiments, an engineered CFTR protein contains modifications of S422D, S660D, S670D, S686D, T690D, S700D, S712D, S737D, S753D, S768D, T787D, T788D, S790D, S795D and S813D.

In some embodiments, increasing the number of phosphomimetic CFTR mutations results in increased sensitivity to activation. In some embodiments, increasing the number of phosphomimetic CFTR mutations results in increased sensitivity to activation by forskolin. In some embodiments, the number of phosphomimetic CFTR mutations include 1, 2, 3, 4, 5, 6, 7, 8, 9, 10, 11, 12, 13, 14, 15, 16, 17, 18, 19 or 20 amino acid substitutions.

In some embodiments, an engineered CFTR protein has low or no immunogenic potential. Accordingly, in some embodiments, an engineered CFTR protein has low immunogenic potential. In some embodiments, an engineered CFTR protein has no immunogenic potential. In some embodiments, an engineered CFTR protein does not create or enhance the potential of cytotoxic T cell lymphocyte epitopes. Accordingly, in some embodiments, an engineered CFTR protein does not create the potential of cytotoxic T cell lymphocyte epitopes. In some embodiments, an engineered CFTR protein does not enhance the potential of cytotoxic T cell lymphocyte epitopes. In some embodiments, an engineered CFTR protein does not cause increased cytotoxicity in comparison to a vehicle control.

In some embodiments, engineered, codon-optimized CFTR has increased activity in comparison to non-codon optimized CFTR.

In some embodiments, an engineered CFTR protein contains modifications at residues S660 and S737, S795 and S813. In some embodiments an engineered CFTR protein contains modifications at residues S660, S686, S700, S737, and S795. In some embodiments an engineered CFTR protein contains modifications at residues S660, S686, S700, S737, S795, and S813. In some embodiments an engineered CFTR protein contains modifications at residues S422, S660, S670, S686, S690, S700, S712, S737, S753, S768, S787, S788, S790, S795, and S813. In some embodiments an engineered CFTR protein contains modifications at residues S422, S660, S670, S686, S690, S700, S712, S753, S787, S788, S790, S795, and S813. In some embodiments an engineered CFTR protein contains modifications at residues S660, S686, S700, S712, S737, S768, S795, and S813. In some embodiments an engineered CFTR protein contains modifications at residue K978. In some embodiments an engineered CFTR protein contains modifications at residue E1371. In some embodiments an engineered CFTR protein contains modifications at residues S422, S660, S670, S686, S690, S700, S712, S753, S787, S788, S790, S795, S813, and E1371.In some embodiments, an engineered CFTR protein contains S660D and S737D, S795D and S813D mutations. In some embodiments an engineered CFTR protein contains S660D, S686D, S700D, S737D, and S795D mutations. In some embodiments an engineered CFTR protein contains S660D, S686D, S700D, S737D, S795D, and S813D mutations. In some embodiments an engineered CFTR protein contains S422D, S660D, S670D, S686D, S690D, S700D, S712D, S737D, S753D, S768D, S787D, S788D, S790D, S795D, and S813D mutations. In some embodiments an engineered CFTR protein contains S422A, S660A, S670A, S686A, S690A, S700A, S712A, S737A, S753A, S768A, S787A, S788A, S790A, S795A, and S813A mutations. In some embodiments an engineered CFTR protein contains S422D, S660D, S670D, S686D, S690D, S700D, S712D, S753D, S787D, S788D, S790D, S795D, and S813D mutations. In some embodiments an engineered CFTR protein contains S660D, S686D, S700D, S712D, S737D, S768D, S795D, and S813D mutations. In some embodiments an engineered CFTR protein contains K978C mutation. In some embodiments an engineered CFTR protein contains E1371Q mutation. In some embodiments an engineered CFTR protein contains S422A, S660A, S670A, S686A, S690A, S700A, S712A, S753A, S787A, S788A, S790A, S795A, S813A, and E1371Q mutations. In some embodiments an engineered CFTR protein contains S422D, S660D, S670D, S686D, S690D, S700D, S712D, S753D, S787D, S788D, S790D, S795D, S813D, and E1371Q mutations.

In some embodiments, an engineered CFTR protein may contain one more modifications to abolish its ATPase activity (ATP Hydrolysis-Deficient mutation). In some embodiments, a catalytic residue in NBD2 domain of CFTR is replaced to abolish its ATPase activity. In particular embodiments, a catalytic residue is E1371. In some embodiment, an engineered CFTR protein contains an E1371Q modification.

In some embodiments, an engineered CFTR protein may contain a modification in the cytosolic loops that promote CFTR channel activity in the absence of ATP (ATP-Independent Activity mutation). In some embodiments, K978 residue in the cytosolic loop is modified to achieve constitutive activity. In some embodiments, K978 is substituted with cysteine, serine, or proline. In particular embodiments, an engineered CFTR protein contains a K978C mutation.

In some embodiments, an engineered CFTR protein may contain any combinations of modifications to achieve a CFTR protein that has greater activity as compared to wild type CFTR protein.

### Stable Trafficking CFTR Protein Mutations

Posttranslational modification can occur at multiple intracellular sites and modify the fate of native and damaged proteins. Both wild-type and mutant CFTR proteins undergo ubiquitination at multiple lysines in the proteins and in one or more subcellular locations. There are several potential lysines to which ubiquitin can be added in CFTR to stabilize the protein. There are additional sites on the CFTR protein that are involved in proteolysis initiation. For example, the ubiquitinated K710, K716, and K1041 residues stabilize wild-type CFTR, protecting it from proteolysis. Modifications K14R and K68R lead to increased mature band C CFTR, which can be augmented by proteasomal (but not lysosomal) inhibition, allowing trafficking to the surface. The amount of CFTR in the K1041R mutant was drastically reduced and consisted of bands A/B. The K1218R mutant increases total and cell surface CFTR, which is further accumulated by proteasomal and lysosomal inhibition.

In some embodiments, an engineered CFTR protein may contain one or more modifications that provide an increased stability for CFTR protein compared to the naturally-occurring CFTR protein (Stability mutant). In particular, an engineered CFTR protein may contain one or more modifications that remove an ubiquitination site in a CFTR protein. In some embodiments, an ubiquitination site is K14, K68, K710, K716, K1041, K1080 or K1218. In some embodiments, a lysine residue is substituted with an arginine residue to remove ubiquitination sites. In some embodiments an engineered CFTR protein contains one or more modifications of K14R, K68R, K710R, K716R, K1041R, K1080R, and K1218R.

In some embodiments, an engineered CFTR protein contains modifications at residue K14. In some embodiments, an engineered CFTR protein contains modifications at residue K68. In some embodiments, an engineered CFTR protein contains modifications at residues K14 and K68. In some embodiments, an engineered CFTR protein contains modifications at residue K1218. In some embodiments, an engineered CFTR protein contains modifications at residues K68 and K1218. In some embodiments, an engineered CFTR protein contains modifications at residues K14 and K1218. In some embodiments, an engineered CFTR protein contains modifications at residues K14, K68, and K1218. In some embodiments, an engineered CFTR protein contains modifications at residue K710. In some embodiments, an engineered CFTR protein contains modifications at residue K716. In some embodiments, an engineered CFTR protein contains modifications at residues K710 and K716. In some embodiments, an engineered CFTR protein contains modifications at residue K1041. In some embodiments, an engineered CFTR protein contains modifications at residues K710, R716, and K1041.

In some embodiments, an engineered CFTR protein contains K14R mutation. In some embodiments, an engineered CFTR protein contains K68R mutation. In some embodiments, an engineered CFTR protein contains K14R and K68R mutations. In some embodiments, an engineered CFTR protein contains K1218R mutation. In some embodiments, an engineered CFTR protein contains K68R and K1218R mutations. In some embodiments, an engineered CFTR protein contains K14R and K1218R mutations. In some embodiments, an engineered CFTR protein contains K14R, K68R, and K1218R mutations. In some embodiments, an engineered CFTR protein contains K710R mutation. In some embodiments, an engineered CFTR protein contains K716R mutation. In some embodiments, an engineered CFTR protein contains K710R and K716R mutations. In some embodiments, an engineered CFTR protein contains K1041R mutation. In some embodiments, an engineered CFTR protein contains K710R, R716R, and K1041R mutations.

In some embodiments, an engineered CFTR protein may contain any combination of Phosphorylated R Domain Mimic, ATP-Independent Activity, ATP Hydrolysis-Deficient, and Stability mutant modifications. In some embodiments, an engineered CFTR protein comprises one or more mutations at residues K14, K68, S422, S660, S670, S686, S700, K710, S712, K716, S737, S753, S768, T787, T786, S790, S795, S813, K978, K1041, K1080, K1218, E1371, and combinations thereof. In particular embodiments, an engineered CFTR protein comprises a combination of mutations listed in Table 2.

In some embodiments, an engineered CFTR protein contains modifications at residues S660, S737, S795, S813, and K14. In some embodiments, an engineered CFTR protein contains modifications at residues S660, S737, S795, S813, and K68. In some embodiments, an engineered CFTR protein contains modifications at residues S660, S737, S795, S813, and K1218. In some embodiments, an engineered CFTR protein contains modifications at residues S660, S737, S795, S813, K14, and K68. In some embodiments, an engineered CFTR protein contains modifications at residues S660, S737, S795, S813, K14, and K1218. In some embodiments, an engineered CFTR protein contains modifications at residues S660, S737, S795, S813, K68, and K1218. In some embodiments, an engineered CFTR protein contains modifications at residues S660, S737, S795, S813, K14, K68, and K1218. In some embodiments, an engineered CFTR protein contains modifications at residues S660, S686, S700, S737, S795, and K14. In some embodiments, an engineered CFTR protein contains modifications at residues S660, S686, S700, S737, S795, and K68. In some embodiments, an engineered CFTR protein contains modifications at residues S660, S686, S700, S737, S795, and K1218. In some embodiments, an engineered CFTR protein contains modifications at residues S660, S686, S700, S737, S795, K14, and K68. In some embodiments, an engineered CFTR protein contains modifications at residues S660, S686, S700, S737, S795, K14, and K1218. In some embodiments, an engineered CFTR protein contains modifications at residues S660, S686, S700, S737, S795, K68, and K1218. In some embodiments, an engineered CFTR protein contains modifications at residues S660, S686, S700, S737, S795, K14, K68, and K1218. In some embodiments, an engineered CFTR protein contains modifications at residues K978, and K14. In some embodiments, an engineered CFTR protein contains modifications at residues K978, and K68. In some embodiments, an engineered CFTR protein contains modifications at residues K978 and K1218. In some embodiments, an engineered CFTR protein contains modifications at residues K978, K14, and K68. In some embodiments, an engineered CFTR protein contains modifications at residues K978, K14, and K1218. In some embodiments, an engineered CFTR protein contains modifications at residues K978, K68, and K1218. In some embodiments, an engineered CFTR protein contains modifications at residues K978, K14, K68, and K1218. In some embodiments, an engineered CFTR protein contains modifications at residues E1371, K14, K68, and K1218. In some embodiments, an engineered CFTR protein contains modifications at residues E1371, and K14. In some embodiments, an engineered CFTR protein contains modifications at residues E1371, and K68. In some embodiments, an engineered CFTR protein contains modifications at residues E1371, and K1218. In some embodiments, an engineered CFTR protein contains modifications at residues E1371, K14, and K68. In some embodiments, an engineered CFTR protein contains modifications at residues E1371, K14, and K1218. In some embodiments, an engineered CFTR protein contains modifications at residues E1371, K68, and K1218. In some embodiments, an engineered CFTR protein contains modifications at residues S660, S686, S700, S737, S795, and E1371. In some embodiments, an engineered CFTR protein contains modifications at residues S660, S686, S700, S737, S795, E1371, and K14. In some embodiments, an engineered CFTR protein contains modifications at residues S660, S686, S700, S737, S795, E1371, and K68. In some embodiments, an engineered CFTR protein contains modifications at residues S660, S686, S700, S737, S795, E1371, and K1218. In some embodiments, an engineered CFTR protein contains modifications at residues S660, S686, S700, S737, S795, E1371, K14, and K68. In some embodiments, an engineered CFTR protein contains modifications at residues S660, S686, S700, S737, S795, E1371, K14, and K1218. In some embodiments, an engineered CFTR protein contains modifications at residues S660, S686, S700, S737, S795, E1371, K68, and K1218. In some embodiments, an engineered CFTR protein contains modifications at residues S660, S686, S700, S737, S795, E1371, K14, K68, and K1218. In some embodiments, an engineered CFTR protein contains modifications at residues S660, S686, S700, S737, S795, K978, E1371, and K14. In some embodiments, an engineered CFTR protein contains modifications at residues S660, S686, S700, S737, S795, K978, E1371, and K68. In some embodiments, an engineered CFTR protein contains modifications at residues S660, S686, S700, S737, S795, K978, E1371, and K1218. In some embodiments, an engineered CFTR protein contains modifications at residues S660, S686, S700, S737, S795, K978, E1371, K14, and K68. In some embodiments, an engineered CFTR protein contains modifications at residues S660, S686, S700, S737, S795, K978, E1371, K14, and K1218. In some embodiments, an engineered CFTR protein contains modifications at residues S660, S686, S700, S737, S795, K978, E1371, K68, and K1218. In some embodiments, an engineered CFTR protein contains modifications at residues S660, S686, S700, S737, S795, E1371, K14, K68, K978, and K1218.

In some embodiments, an engineered CFTR protein contains S660D, S73D7, S795D, S813D, and K14R mutations. In some embodiments, an engineered CFTR protein contains S660D, S737D, S795D, S813D, and K68R mutations. In some embodiments, an engineered CFTR protein contains S660D, S737D, S795D, S813D, and K1218R mutations. In some embodiments, an engineered CFTR protein contains S660D, S737D, S795D, S813D, K14R, and K68R mutations. In some embodiments, an engineered CFTR protein contains S660D, S737D, S795D, S813D, K14R, and K1218R mutations. In some embodiments, an engineered CFTR protein contains S660D, S737D, S795D, S813D, K68R, and K1218R mutations. In some embodiments an engineered CFTR protein contains S660D, S737D, S795D, S813D, K14R, K68R, and K1218R mutations. In some embodiments, an engineered CFTR protein contains S660D, S686D, S700D, S737D, S795D, and K14R mutations. In some embodiments, an engineered CFTR protein contains S660D, S686D, S700D, S737D, S795D, and K68R mutations. In some embodiments, an engineered CFTR protein contains S660D, S686D, S700D, S737D, S795D, and K1218R mutations. In some embodiments, an engineered CFTR protein contains S660D, S686D, S700D, S737D, S795D, K14R, and K68R mutations. In some embodiments, an engineered CFTR protein contains S660D, S686D, S700D, S737D, S795D, K14R, and K1218R mutations. In some embodiments, an engineered CFTR protein contains S660D, S686D, S700D, S737D, S795D, K68R, and K1218R mutations. In some embodiments, an engineered CFTR protein contains S660D, S686D, S700D, S737D, S795D, K14R, K68R, and K1218R mutations. In some embodiments, an engineered CFTR protein contains K978C, and K14R mutations. In some embodiments, an engineered CFTR protein contains K978C, and K68R mutations. In some embodiments, an engineered CFTR protein contains K978C and K1218R mutations. In some embodiments, an engineered CFTR protein contains K978C, K14R, and K68R mutations. In some embodiments, an engineered CFTR protein contains K978C, K14R, and K1218R mutations. In some embodiments, an engineered CFTR protein contains K978C, K68R, and K1218R mutations. In some embodiments, an engineered CFTR protein contains K978C, K14R, K68R, and K1218R mutations. In some embodiments, an engineered CFTR protein contains E1371Q, and K14R mutations. In some embodiments, an engineered CFTR protein contains E1371Q, and K68R mutations. In some embodiments, an engineered CFTR protein contains E1371Q, and K1218R mutations. In some embodiments, an engineered CFTR protein contains E1371Q, K14R, and K68R mutations. In some embodiments, an engineered CFTR protein contains E1371Q, K14R, and K1218R mutations. In some embodiments, an engineered CFTR protein contains E1371Q, K68R, and K1218R mutations. In some embodiments, an engineered CFTR protein contains E1371Q, K14R, K68R, and K1218R mutations. In some embodiments, an engineered CFTR protein contains S660D, S686D, S700D, S737D, S795D, E1371Q, and K14R mutations. In some embodiments, an engineered CFTR protein contains S660D, S686D, S700D, S737D, S795D, E1371Q, and K68R mutations. In some embodiments, an engineered CFTR protein contains S660D, S686D, S700D, S737D, S795D, E1371Q, and K1218R mutations. In some embodiments, an engineered CFTR protein contains S660D, S686D, S700D, S737D, S795D, E1371Q, K14R, and K68R mutations. In some embodiments, an engineered CFTR protein contains S660D, S686D, S700D, S737D, S795D, E1371Q, K14R, and K1218R mutations. In some embodiments, an engineered CFTR protein contains S660D, S686D, S700D, S737D, S795D, E1371D, K68R, and K1218R mutations. In some embodiments, an engineered CFTR protein contains S660D, S686D, S700D, S737D, S795D, and E1371Q mutations. In some embodiments, an engineered CFTR protein contains S660D, S686D, S700D, S737D, S795D, K978C, E1371Q, and K14R mutations. In some embodiments, an engineered CFTR protein contains S660D, S686D, S700D, S737D, S795D, K978C, E1371Q, and K68R mutations. In some embodiments, an engineered CFTR protein contains S660D, S686D, S700D, S737D, S795D, K978C, E1371Q, and K1218R mutations. In some embodiments, an engineered CFTR protein contains S660D, S686D, S700D, S737D, S795D, K978C, E1371Q, K14R, and K68R mutations. In some embodiments, an engineered CFTR protein contains S660D, S686D, S700D, S737D, S795D, K978C, E1371Q, K14R, and K1218R mutations. In some embodiments, an engineered CFTR protein contains S660D, S686D, S700D, S737D, S795D, K978C, E1371Q, K68R, and K1218R mutations. In some embodiments, an engineered CFTR protein contains S660D, S686D, S700D, S737D, S795D, E1371Q, K14R, K68R, and K1218R mutations. In some embodiments, an engineered CFTR protein contains S660D, S686D, S700D, S737D, S795D, E1371Q, K14R, K68R, K978C, and K1218R mutations.

TABLE 2
Engineered CFTR Proteins

| Engineered | Activated CFTR Mutation | | | | | | | | Stable CFTR Mutation | | |
|---|---|---|---|---|---|---|---|---|---|---|---|
| CFTR | S660 | S686 | S700 | S737 | S795 | S813 | K978 | E1371 | K14 | K68 | K1218 |
| A | | | | | | | | | | | |
| B | D | | | D | D | D | | | | | |
| C | D | D | D | D | D | | | | | | |
| D | | | | | | | C | | | | |
| E | | | | | | | | Q | | | |
| F | | | | | | | | | R | | |
| G | | | | | | | | | | R | |
| H | | | | | | | | | R | R | |
| I | | | | | | | | | | | R |
| J | | | | | | | | | | R | R |
| K | | | | | | | | | R | | R |
| L | | | | | | | | | R | R | R |
| M | D | | | D | D | D | | | R | R | R |
| N | D | D | D | D | D | | | | R | R | R |
| O | | | | | | | C | | R | R | R |
| P | | | | | | | | Q | R | R | R |
| Q | D | D | D | D | D | D | | Q | | | |
| R | D | D | D | D | D | D | | Q | R | R | R |
| S | D | D | D | D | D | D | C | Q | R | R | R |
| T | X | X | X | X | X | X | X | X | X | X | X |

*X = any amino acid

In some embodiments, an engineered CFTR protein comprises S660D, S737D, S795D and S813D mutations. In some embodiments, an mRNA suitable for the present invention encodes an amino acid sequence at least 80%, 85%, 90%, 91%, 92%, 93%, 94%, 95%, 96%, 97%, 98%, 99% or more homologous to SEQ ID NO: 41. In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 45.

In some embodiments, an engineered CFTR protein comprises S660D, S686D, S700D, S737D, S795D and S813D mutations. In some embodiments, an mRNA suitable for the present invention encodes an amino acid sequence at least 80%, 85%, 90%, 91%, 92%, 93%, 94%, 95%, 96%, 97%, 98%, 99% or more homologous to SEQ ID NO: 42. In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 46.

In some embodiments, an engineered CFTR protein comprises K978C mutation. In some embodiments, an mRNA suitable for the present invention encodes an amino acid sequence at least 80%, 85%, 90%, 91%, 92%, 93%, 94%, 95%, 96%, 97%, 98%, 99% or more homologous to SEQ TD NO: 43. In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ TD NO: 47.

In some embodiments, an engineered CFTR protein comprises E1371Q mutation. In some embodiments, an mRNA suitable for the present invention encodes an amino acid sequence at least 80%, 85%, 90%, 91%, 92%, 93%, 94%, 95%, 96%, 97%, 98%, 99% or more homologous to SEQ TD NO: 44. In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ TD NO: 48.

TABLE 3
Exemplary Engineered CFTR Protein Sequences

| | |
|---|---|
| SEQ ID | MQRSPLEKASVVSKLFFSWTRPILRKGYRQRLELSDIYQIPSVDSADNLSEK |
| NO: 41 | LEREWDRELASKKNPKLINALRRCFFWRFMFYGIFLYLGEVTKAVQPLLL |
| | GRIIASYDPDNKEERSIAIYLGIGLCLLFIVRTLLLHPAIFGLHHIGMQMRIA |
| | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNDILTETLHR |
| | FSLEGDAPVSWTETKKQSFKQTGEFGEKRKNSILNPINSIRKFSIVQKTPLQ |
| | MNGIEEDSDEPLERRLDLVPDSEQGEAILPRISVISTGPTLQARRRQSVLNL |
| | MTHSVNQGQNIHRKTTASTRKVDLAPQANLTELDIYSRRLDQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSK |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHK |
| | ALNLHTANWFLYLSTLRWFQMRIEMIFVIFFIAVTFISILTTGEGEGRVGIIL |
| | TLAMNIMSTLQWAVNSSIDVDSLMRSVSRVFKFIDMPTEGKPTKSTKPYK |
| | NGQLSKVMIIENSHVKKDDIWPSGGQMTVKDLTAKYTEGGNAILENISFSI |
| | SPGQRVGLLGRTGSGKSTLLSAFLRLLNTEGEIQIDGVSWDSITLQQWRKA |
| | FGVIPQKVFIFSGTFRKNLDPYEQWSDQEIWKVADEVGLRSVIEQFPGKLD |
| | FVLVDGGCVLSHGHKQLMCLARSVLSKAKILLLDEPSAHLDPVTYQIIRRT |
| | LKQAFADCTVILCEHRIEAMLECQQFLVIEENKVRQYDSIQKLLNERSLFR |
| | QAISPSDRVKLFPHRNSSKCKSKPQIAALKEETEEEVQDTRL (SEQ ID NO: |
| | 41) |
| SEQ ID | MQRSPLEKASVVSKLFFSWTRPILRKGYRQRLELSDIYQIPSVDSADNLSEK |
| NO: 42 | LEREWDRELASKKNPKLINALRRCFFWRFMFYGIFLYLGEVTKAVQPLLL |
| | GRIIASYDPDNKEERSIAIYLGIGLCLLFIVRTLLLHPAIFGLHHIGMQMRIA |
| | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNDILTETLHR |
| | FSLEGDAPVSWTETKKQDFKQTGEFGEKRKNDILNPINSIRKFSIVQKTPLQ |
| | MNGIEEDSDEPLERRLDLVPDSEQGEAILPRISVISTGPTLQARRRQSVLNL |
| | MTHSVNQGQNIHRKTTASTRKVDLAPQANLTELDIYSRRLDQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSK |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHKALN |
| | LHTANWFLYLSTLRWFQMRIEMIFVIFFIAVTFISILTTGEGEGRVGIILTLA |
| | MNIMSTLQWAVNSSIDVDSLMRSVSRVFKFIDMPTEGKPTKSTKPYKNGQ |
| | LSKVMIIENSHVKKDDIWPSGGQMTVKDLTAKYTEGGNAILENISFSISPGQ |
| | RVGLLGRTGSGKSTLLSAFLRLLNTEGEIQIDGVSWDSITLQQWRKAFGVIP |
| | QKVFIFSGTFRKNLDPYEQWSDQEIWKVADEVGLRSVIEQFPGKLDFVLVD |
| | GGCVLSHGHKQLMCLARSVLSKAKILLLDEPSAHLDPVTYQIIRRTLKQAF |
| | ADCTVILCEHRIEAMLECQQFLVIEENKVRQYDSIQKLLNERSLFRQAISPS |
| | DRVKLFPHRNSSKCKSKPQIAALKEETEEEVQDTRL (SEQ ID NO: 42) |
| SEQ ID | MQRSPLEKASVVSKLFFSWTRPILRKGYRQRLELSDIYQIPSVDSADNLSEK |
| NO: 43 | LEREWDRELASKKNPKLINALRRCFFWRFMFYGIFLYLGEVTKAVQPLLL |
| | GRIIASYDPDNKEERSIAIYLGIGLCLLFIVRTLLLHPAIFGLHHIGMQMRIA |
| | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNSILTETLHR |
| | FSLEGDAPVSWTETKKQSFKQTGEFGEKRKNSILNPINSIRKFSIVQKTPLQ |
| | MNGIEEDSDEPLERRLSLVPDSEQGEAILPRISVISTGPTLQARRRQSVLNL |
| | MTHSVNQGQNIHRKTTASTRKVSLAPQANLTELDIYSRRLSQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSC |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHKALN |
| | LHTANWFLYLSTLRWFQMRIEMIFVIFFIAVTFISILTTGEGEGRVGIILTLA |
| | MNIMSTLQWAVNSSIDVDSLMRSVSRVFKFIDMPTEGKPTKSTKPYKNGQ |
| | LSKVMIIENSHVKKDDIWPSGGQMTVKDLTAKYTEGGNAILENISFSISPGQ |
| | RVGLLGRTGSGKSTLLSAFLRLLNTEGEIQIDGVSWDSITLQQWRKAFGVIP |
| | QKVFIFSGTFRKNLDPYEQWSDQEIWKVADEVGLRSVIEQFPGKLDFVLVD |
| | GGCVLSHGHKQLMCLARSVLSKAKILLLDEPSAHLDPVTYQIIRRTLKQAF |
| | ADCTVILCEHRIEAMLECQQFLVIEENKVRQYDSIQKLLNERSLFRQAISPS |
| | DRVKLFPHRNSSKCKSKPQIAALKEETEEEVQDTRL (SEQ ID NO: 43) |
| SEQ ID | MQRSPLEKASVVSKLFFSWTRPILRKGYRQRLELSDIYQIPSVDSADNLSEK |
| NO: 44 | LEREWDRELASKKNPKLINALRRCFFWRFMFYGIFLYLGEVTKAVQPLLL |
| | GRIIASYDPDNKEERSIAIYLGIGLCLLFIVRTLLLHPAIFGLHHIGMQMRIA |
| | MFSLIYKKTLKLSSRVLDKISIGQLVSLLSNNLNKFDEGLALAHFVWIAPLQ |
| | VALLMGLIWELLQASAFCGLGFLIVLALFQAGLGRMMMKYRDQRAGKIS |
| | ERLVITSEMIENIQSVKAYCWEEAMEKMIENLRQTELKLTRKAAYVRYFN |
| | SSAFFFSGFFVVFLSVLPYALIKGIILRKIFTTISFCIVLRMAVTRQFPWAVQT |
| | WYDSLGAINKIQDFLQKQEYKTLEYNLTTTEVVMENVTAFWEEGFGELFE |
| | KAKQNNNNRKTSNGDDSLFFSNFSLLGTPVLKDINFKIERGQLLAVAGSTG |
| | AGKTSLLMVIMGELEPSEGKIKHSGRISFCSQFSWIMPGTIKENIIFGVSYDE |
| | YRYRSVIKACQLEEDISKFAEKDNIVLGEGGITLSGGQRARISLARAVYKD |
| | ADLYLLDSPFGYLDVLTEKEIFESCVCKLMANKTRILVTSKMEHLKKADKI |
| | LILHEGSSYFYGTFSELQNLQPDFSSKLMGCDSFDQFSAERRNSILTETLHR |
| | FSLEGDAPVSWTETKKQSFKQTGEFGEKRKNSILNPINSIRKFSIVQKTPLQ |
| | MNGIEEDSDEPLERRLSLVPDSEQGEAILPRISVISTGPTLQARRRQSVLNL |
| | MTHSVNQGQNIHRKTTASTRKVSLAPQANLTELDIYSRRLSQETGLEISEEI |
| | NEEDLKECFFDDMESIPAVTTWNTYLRYITVHKSLIFVLIWCLVIFLAEVAA |
| | SLVVLWLLGNTPLQDKGNSTHSRNNSYAVIITSTSSYYVFYIYVGVADTLL |
| | AMGFFRGLPLVHTLITVSKILHHKMLHSVLQAPMSTLNTLKAGGILNRFSK |
| | DIAILDDLLPLTIFDFIQLLLIVIGAIAVVAVLQPYIFVATVPVIVAFIMLRAY |
| | FLQTSQQLKQLESEGRSPIFTHLVTSLKGLWTLRAFGRQPYFETLFHKALN |
| | LHTANWFLYLSTLRWFQMRIEMIFVIFFIAVTFISILTTGEGEGRVGIILTLA |
| | MNIMSTLQWAVNSSIDVDSLMRSVSRVFKFIDMPTEGKPTKSTKPYKNGQ |
| | LSKVMIIENSHVKKDDIWPSGGQMTVKDLTAKYTEGGNAILENISFSISPGQ |
| | RVGLLGRTGSGKSTLLSAFLRLLNTEGEIQIDGVSWDSITLQQWRKAFGVIP |
| | QKVFIFSGTFRKNLDPYEQWSDQEIWKVADEVGLRSVIEQFPGKLDFVLVD |
| | GGCVLSHGHKQLMCLARSVLSKAKILLLDQPSAHLDPVTYQIIRRTLKQAF |
| | ADCTVILCEHRIEAMLECQQFLVIEENKVRQYDSIQKLLNERSLFRQAISPS |
| | DRVKLFPHRNSSKCKSKPQIAALKEETEEEVQDTRL (SEQ ID NO: 44) |

TABLE 4
Exemplary Codon Optimized mRNA Sequences Encoding an Engineered CFTR
Protein

| | |
|---|---|
| SEQ ID | ATGCAACGCTCTCCTCTTGAAAAGGCCTCGGTGGTGTCCAAGCTCTTCT |
| NO: 45 | TCTCGTGGACTAGACCCATCCTGAGAAAGGGGTACAGACAGCGCTTGG |
| | AGCTGTCCGATATCTATCAAATCCCTTCCGTGGACTCCGCGGACAACCT |
| | GTCCGAGAAGCTCGAGAGAGAATGGGACAGAGAACTCGCCTCAAAGA |
| | AGAACCCGAAGCTGATTAATGCGCTTAGGCGGTGCTTTTTCTGGCGGTT |
| | CATGTTCTACGGCATCTTCCTCTACCTGGGAGAGGTCACCAAGGCCGTG |
| | CAGCCCCTGTTGCTGGGACGGATTATTGCCTCCTACGACCCCGACAACA |
| | AGGAAGAAAGAAGCATCGCTATCTACTTGGGCATCGGTCTGTGCCTGC |
| | TTTTCATCGTCCGGACCCTCTTGTTGCATCCTGCTATTTTCGGCCTGCAT |
| | CACATTGGCATGCAGATGAGAATTGCCATGTTTTCCCTGATCTACAAGA |
| | AAACTCTGAAGCTCTCGAGCCGCGTGCTTGACAAGATTTCCATCGGCCA |
| | GCTCGTGTCCCTGCTCTCCAACAATCTGAACAAGTTCGACGAGGGCCTC |
| | GCCCTGGCCCACTTCGTGTGGATCGCCCCTCTGCAAGTGGCGCTTCTGA |
| | TGGGCCTGATCTGGGAGCTGCTGCAAGCCTCGGCATTCTGTGGGCTTGG |
| | ATTCCTGATCGTGCTGGCACTGTTCCAGGCCGGACTGGGGCGGATGAT |
| | GATGAAGTACAGGGACCAGAGAGCCGGAAAGATTTCCGAACGGCTGG |
| | TGATCACTTCGGAAATGATCGAAAACATCCAGTCAGTGAAGGCCTACT |
| | GCTGGGAAGAGGCCATGGAAAAGATGATTGAAAACCTCCGGCAAACC |
| | GAGCTGAAGCTGACCCGCAAGGCCGCTTACGTGCGCTATTTCAACTCGT |
| | CCGCTTTCTTCTTCTCCGGGTTCTTCGTGGTGTTTCTCTCCGTGCTCCCCT |
| | ACGCCCTGATTAAGGGAATCATCCTCAGGAAGATCTTCACCACCATTTC |
| | CTTCTGTATCGTGCTCCGCATGGCCGTGACCCGGCAGTTCCCATGGGCC |
| | GTGCAGACTTGGTACGACTCCCTGGGAGCCATTAACAAGATCCAGGAC |
| | TTCCTTCAAAAGCAGGAGTACAAGACCCTCGAGTACAACCTGACTACT |
| | ACCGAGGTCGTGATGGAAAACGTCACCGCCTTTTGGGAGGAGGGATTT |
| | GGCGAACTGTTCGAGAAGGCCAAGCAGAACAACAACAACCGCAAGAC |
| | CTCGAACGGTGACGACTCCCTCTTCTTTTCAAACTTCAGCCTGCTCGGG |
| | ACGCCCGTGCTGAAGGACATTAACTTCAAGATCGAAAGAGGACAGCTC |
| | CTGGCGGTGGCCGGATCGACCGGAGCCGGAAAGACTTCCCTGCTGATG |
| | GTGATCATGGGAGAGCTTGAACCTAGCGAGGGAAAGATCAAGCACTCC |
| | GGCCGCATCAGCTTCTGTAGCCAGTTTTCCTGGATCATGCCCGGAACCA |
| | TTAAGGAAAACATCATCTTCGGCGTGTCCTACGATGAATACCGCTACCG |
| | GTCCGTGATCAAAGCCTGCCAGCTGGAAGAGGATATTTCAAAGTTCGC |
| | GGAGAAAGATAACATCGTGCTGGGCGAAGGGGGTATTACCTTGTCGGG |
| | GGGCCAGCGGGCTAGAATCTCGCTGGCCAGAGCCGTGTATAAGGACGC |
| | CGACCTGTATCTCCTGGACTCCCCCTTCGGATACCTGGACGTCCTGACC |
| | GAAAAGGAGATCTTCGAATCGTGCGTGTGCAAGCTGATGGCTAACAAG |
| | ACTCGCATCCTCGTGACCTCCAAAATGGAGCACCTGAAGAAGGCAGAC |
| | AAGATTCTGATTCTGCATGAGGGGTCCTCCTACTTTTACGGCACCTTCT |
| | CGGAGTTGCAGAACTTGCAGCCCGACTTCTCATCGAAGCTGATGGGTT |
| | GCGACAGCTTCGACCAGTTCTCCGCCGAAAGAAGGAACGATATCCTGA |
| | CGGAAACCTTGCACCGCTTCTCTTTGGAAGGCGACGCCCCTGTGTCATG |
| | GACCGAGACTAAGAAGCAGAGCTTCAAGCAGACCGGGGAATTCGGCG |
| | AAAAGAGGAAGAACAGCATCTTGAACCCCATTAACTCCATCCGCAAGT |
| | TCTCAATCGTGCAAAAGACGCCACTGCAGATGAACGGCATTGAGGAGG |
| | ACTCCGACGAACCCCTTGAGAGGCGCCTGGATCTGGTGCCGGACAGCG |
| | AGCAGGGAGAAGCCATCCTGCCTCGGATTTCCGTGATCTCCACTGGTCC |
| | GACGCTCCAAGCCCGGCGGCGGCAGTCCGTGCTGAACCTGATGACCCA |
| | CAGCGTGAACCAGGGCCAAAACATTCACCGCAAGACTACCGCATCCAC |
| | CCGGAAAGTGGATCTGGCACCTCAAGCGAATCTTACCGAGCTCGACAT |
| | CTACTCCCGGAGACTGGATCAGGAAACCGGGCTCGAAATTTCCGAAGA |
| | AATCAACGAGGAGGATCTGAAAGAGTGCTTCTTCGACGATATGGAGTC |
| | GATACCCGCCGTGACGACTTGGAACACTTATCTGCGGTACATCACTGTG |
| | CACAAGTCATTGATCTTCGTGCTGATTTGGTGCCTGGTGATTTTCCTGG |
| | CCGAGGTCGCGGCCTCACTGGTGGTGCTCTGGCTGTTGGGAAACACGC |
| | CTCTGCAAGACAAGGGAAACTCCACGCACTCGAGAAACAACAGCTATG |
| | CCGTGATTATCACTTCCACCTCCTCTTATTACGTGTTCTACATCTACGTC |
| | GGAGTGGCGGATACCCTGCTCGCGATGGGTTTCTTCAGAGGACTGCCG |
| | CTGGTCCACACCTTGATCACCGTCAGCAAGATTCTTCACCACAAGATGT |
| | TGCATAGCGTGCTGCAGGCCCCCATGTCCACCCTCAACACTCTGAAGGC |
| | CGGAGGCATTCTGAACAGATTCTCCAAGGACATCGCTATCCTGGACGA |
| | TCTCCTGCCGCTTACCATCTTTGACTTCATCCAGCTGCTGCTGATCGTGA |
| | TTGGAGCAATCGCAGTGGTGGCGGTGCTGCAGCCTTACATTTTCGTGGC |
| | CACTGTGCCGGTCATTGTGGCGTTCATCATGCTGCGGGCCTACTTCCTC |
| | CAAACCAGCCAGCAGCTGAAGCAACTGGAATCCGAGGGACGATCCCCC |
| | ATCTTCACTCACCTTGTGACGTCGTTGAAGGGACTGTGGACCCTCCGGG |
| | CTTTCGGACGGCAGCCCTACTTCGAAACCCTCTTCCACAAGGCCCTGAA |
| | CCTCCACACCGCCAATTGGTTCCTGTACCTGTCCACCCTGCGGTGGTTC |
| | CAGATGCGCATCGAGATGATTTTCGTCATCTTCTTCATCGCGGTCACAT |
| | TCATCAGCATCCTGACTACCGGAGAGGGAGAGGGACGGGTCGGAATAA |
| | TCCTGACCCTCGCCATGAACATTATGAGCACCCTGCAGTGGGCAGTGA |
| | ACAGCTCGATCGACGTGGACAGCCTGATGCGAAGCGTCAGCCGCGTGT |
| | TCAAGTTCATCGACATGCCTACTGAGGGAAAACCCACTAAGTCCACTA |
| | AGCCCTACAAAAATGGCCAGCTGAGCAAGGTCATGATCATCGAAAACT |
| | CCCACGTGAAGAAGGACGATATTTGGCCCTCCGGAGGTCAAATGACCG |
| | TGAAGGACCTGACCGCAAAGTACACCGAGGGAGGAAACGCCATTCTCG |
| | AAAACATCAGCTTCTCCATTTCGCCGGGACAGCGGGTCGGCCTTCTCGG |
| | GCGGACCGGTTCCGGGAAGTCAACTCTGCTGTCGGCTTTCCTCCGGCTG |
| | CTGAATACCGAGGGGGAAATCCAAATTGACGGCGTGTCTTGGGATTCC |
| | ATTACTCTGCAGCAGTGGCGGAAGGCCTTCGGCGTGATCCCCCAGAAG |
| | GTGTTCATCTTCTCGGGTACCTTCCGGAAGAACCTGGATCCTTACGAGC |
| | AGTGGAGCGACCAAGAAATCTGGAAGGTCGCCGACGAGGTCGGCCTGC |
| | GCTCCGTGATTGAACAATTTCCTGGAAAGCTGGACTTCGTGCTCGTCGA |
| | CGGGGGATGTGTCCTGTCGCACGGACATAAGCAGCTCATGTGCCTCGC |
| | ACGGTCCGTGCTCTCCAAGGCCAAGATTCTGCTGCTGGACGAACCTTCG |
| | GCCCACCTGGATCCGGTCACCTACCAGATCATCAGGAGGACCCTGAAG |
| | CAGGCCTTTGCCGATTGCACCGTGATTCTCTGCGAGCACCGCATCGAGG |
| | CCATGCTGGAGTGCCAGCAGTTCCTGGTCATCGAGGAGAACAAGGTCC |
| | GCCAATACGACTCCATTCAAAAGCTCCTCAACGAGCGGTCGCTGTTCA |
| | GACAAGCTATTTCACCGTCCGATAGAGTGAAGCTCTTCCCGCATCGGA |
| | ACAGCTCAAAGTGCAAATCGAAGCCGCAGATCGCAGCCTTGAAGGAAG |
| | AGACTGAGGAAGAGGTGCAGGACACCCGGCTTTAA (SEQ ID NO: 45) |
| SEQ ID | ATGCAACGCTCTCCTCTTGAAAAGGCCTCGGTGGTGTCCAAGCTCTTCT |
| NO: 46 | TCTCGTGGACTAGACCCATCCTGAGAAAGGGGTACAGACAGCGCTTGG |
| | AGCTGTCCGATATCTATCAAATCCCTTCCGTGGACTCCGCGGACAACCT |
| | GTCCGAGAAGCTCGAGAGAGAATGGGACAGAGAACTCGCCTCAAAGA |
| | AGAACCCGAAGCTGATTAATGCGCTTAGGCGGTGCTTTTTCTGGCGGTT |
| | CATGTTCTACGGCATCTTCCTCTACCTGGGAGAGGTCACCAAGGCCGTG |
| | CAGCCCCTGTTGCTGGGACGGATTATTGCCTCCTACGACCCCGACAACA |
| | AGGAAGAAAGAAGCATCGCTATCTACTTGGGCATCGGTCTGTGCCTGC |
| | TTTTCATCGTCCGGACCCTCTTGTTGCATCCTGCTATTTTCGGCCTGCAT |
| | CACATTGGCATGCAGATGAGAATTGCCATGTTTTCCCTGATCTACAAGA |
| | AAACTCTGAAGCTCTCGAGCCGCGTGCTTGACAAGATTTCCATCGGCCA |
| | GCTCGTGTCCCTGCTCTCCAACAATCTGAACAAGTTCGACGAGGGCCTC |
| | GCCCTGGCCCACTTCGTGTGGATCGCCCCTCTGCAAGTGGCGCTTCTGA |
| | TGGGCCTGATCTGGGAGCTGCTGCAAGCCTCGGCATTCTGTGGGCTTGG |
| | ATTCCTGATCGTGCTGGCACTGTTCCAGGCCGGACTGGGGCGGATGAT |
| | GATGAAGTACAGGGACCAGAGAGCCGGAAAGATTTCCGAACGGCTGG |
| | TGATCACTTCGGAAATGATCGAAAACATCCAGTCAGTGAAGGCCTACT |
| | GCTGGGAAGAGGCCATGGAAAAGATGATTGAAAACCTCCGGCAAACC |
| | GAGCTGAAGCTGACCCGCAAGGCCGCTTACGTGCGCTATTTCAACTCGT |
| | CCGCTTTCTTCTTCTCCGGGTTCTTCGTGGTGTTTCTCTCCGTGCTCCCCT |
| | ACGCCCTGATTAAGGGAATCATCCTCAGGAAGATCTTCACCACCATTTC |
| | CTTCTGTATCGTGCTCCGCATGGCCGTGACCCGGCAGTTCCCATGGGCC |
| | GTGCAGACTTGGTACGACTCCCTGGGAGCCATTAACAAGATCCAGGAC |
| | TTCCTTCAAAAGCAGGAGTACAAGACCCTCGAGTACAACCTGACTACT |
| | ACCGAGGTCGTGATGGAAAACGTCACCGCCTTTTGGGAGGAGGGATTT |
| | GGCGAACTGTTCGAGAAGGCCAAGCAGAACAACAACAACCGCAAGAC |
| | CTCGAACGGTGACGACTCCCTCTTCTTTTCAAACTTCAGCCTGCTCGGG |
| | ACGCCCGTGCTGAAGGACATTAACTTCAAGATCGAAAGAGGACAGCTC |
| | CTGGCGGTGGCCGGATCGACCGGAGCCGGAAAGACTTCCCTGCTGATG |
| | GTGATCATGGGAGAGCTTGAACCTAGCGAGGGAAAGATCAAGCACTCC |
| | GGCCGCATCAGCTTCTGTAGCCAGTTTTCCTGGATCATGCCCGGAACCA |
| | TTAAGGAAAACATCATCTTCGGCGTGTCCTACGATGAATACCGCTACCG |
| | GTCCGTGATCAAAGCCTGCCAGCTGGAAGAGGATATTTCAAAGTTCGC |
| | GGAGAAAGATAACATCGTGCTGGGCGAAGGGGGTATTACCTTGTCGGG |
| | GGGCCAGCGGGCTAGAATCTCGCTGGCCAGAGCCGTGTATAAGGACGC |
| | CGACCTGTATCTCCTGGACTCCCCCTTCGGATACCTGGACGTCCTGACC |
| | GAAAAGGAGATCTTCGAATCGTGCGTGTGCAAGCTGATGGCTAACAAG |
| | ACTCGCATCCTCGTGACCTCCAAAATGGAGCACCTGAAGAAGGCAGAC |
| | AAGATTCTGATTCTGCATGAGGGGTCCTCCTACTTTTACGGCACCTTCT |
| | CGGAGTTGCAGAACTTGCAGCCCGACTTCTCATCGAAGCTGATGGGTT |
| | GCGACAGCTTCGACCAGTTCTCCGCCGAAAGAAGGAACGATATCCTGA |
| | CGGAAACCTTGCACCGCTTCTCTTTGGAAGGCGACGCCCCTGTGTCATG |
| | GACCGAGACTAAGAAGCAGGATTTCAAGCAGACCGGGGAATTCGGCG |
| | AAAAGAGGAAGAACGACATCTTGAACCCCATTAACTCCATCCGCAAGT |
| | TCTCAATCGTGCAAAAGACGCCACTGCAGATGAACGGCATTGAGGAGG |
| | ACTCCGACGAACCCCTTGAGAGGCGCCTGGATCTGGTGCCGGACAGCG |
| | AGCAGGGAGAAGCCATCCTGCCTCGGATTTCCGTGATCTCCACTGGTCC |
| | GACGCTCCAAGCCCGGCGGCGGCAGTCCGTGCTGAACCTGATGACCCA |
| | CAGCGTGAACCAGGGCCAAAACATTCACCGCAAGACTACCGCATCCAC |
| | CCGGAAAGTGGATCTGGCACCTCAAGCGAATCTTACCGAGCTCGACAT |
| | CTACTCCCGGAGACTGGATCAGGAAACCGGGCTCGAAATTTCCGAAGA |
| | AATCAACGAGGAGGATCTGAAAGAGTGCTTCTTCGACGATATGGAGTC |
| | GATACCCGCCGTGACGACTTGGAACACTTATCTGCGGTACATCACTGTG |
| | CACAAGTCATTGATCTTCGTGCTGATTTGGTGCCTGGTGATTTTCCTGG |
| | CCGAGGTCGCGGCCTCACTGGTGGTGCTCTGGCTGTTGGGAAACACGC |
| | CTCTGCAAGACAAGGGAAACTCCACGCACTCGAGAAACAACAGCTATG |
| | CCGTGATTATCACTTCCACCTCCTCTTATTACGTGTTCTACATCTACGTC |
| | GGAGTGGCGGATACCCTGCTCGCGATGGGTTTCTTCAGAGGACTGCCG |
| | CTGGTCCACACCTTGATCACCGTCAGCAAGATTCTTCACCACAAGATGT |
| | TGCATAGCGTGCTGCAGGCCCCCATGTCCACCCTCAACACTCTGAAGGC |
| | CGGAGGCATTCTGAACAGATTCTCCAAGGACATCGCTATCCTGGACGA |
| | TCTCCTGCCGCTTACCATCTTTGACTTCATCCAGCTGCTGCTGATCGTGA |
| | TTGGAGCAATCGCAGTGGTGGCGGTGCTGCAGCCTTACATTTTCGTGGC |
| | CACTGTGCCGGTCATTGTGGCGTTCATCATGCTGCGGGCCTACTTCCTC |
| | CAAACCAGCCAGCAGCTGAAGCAACTGGAATCCGAGGGACGATCCCCC |
| | ATCTTCACTCACCTTGTGACGTCGTTGAAGGGACTGTGGACCCTCCGGG |
| | CTTTCGGACGGCAGCCCTACTTCGAAACCCTCTTCCACAAGGCCCTGAA |
| | CCTCCACACCGCCAATTGGTTCCTGTACCTGTCCACCCTGCGGTGGTTC |
| | CAGATGCGCATCGAGATGATTTTCGTCATCTTCTTCATCGCGGTCACAT |
| | TCATCAGCATCCTGACTACCGGAGAGGGAGAGGGACGGGTCGGAATAA |
| | TCCTGACCCTCGCCATGAACATTATGAGCACCCTGCAGTGGGCAGTGA |
| | ACAGCTCGATCGACGTGGACAGCCTGATGCGAAGCGTCAGCCGCGTGT |
| | TCAAGTTCATCGACATGCCTACTGAGGGAAAACCCACTAAGTCCACTA |
| | AGCCCTACAAAAATGGCCAGCTGAGCAAGGTCATGATCATCGAAAACT |
| | CCCACGTGAAGAAGGACGATATTTGGCCCTCCGGAGGTCAAATGACCG |
| | TGAAGGACCTGACCGCAAAGTACACCGAGGGAGGAAACGCCATTCTCG |
| | AAAACATCAGCTTCTCCATTTCGCCGGGACAGCGGGTCGGCCTTCTCGG |
| | GCGGACCGGTTCCGGGAAGTCAACTCTGCTGTCGGCTTTCCTCCGGCTG |
| | CTGAATACCGAGGGGGAAATCCAAATTGACGGCGTGTCTTGGGATTCC |
| | ATTACTCTGCAGCAGTGGCGGAAGGCCTTCGGCGTGATCCCCCAGAAG |
| | GTGTTCATCTTCTCGGGTACCTTCCGGAAGAACCTGGATCCTTACGAGC |
| | AGTGGAGCGACCAAGAAATCTGGAAGGTCGCCGACGAGGTCGGCCTGC |
| | GCTCCGTGATTGAACAATTTCCTGGAAAGCTGGACTTCGTGCTCGTCGA |
| | CGGGGGATGTGTCCTGTCGCACGGACATAAGCAGCTCATGTGCCTCGC |
| | ACGGTCCGTGCTCTCCAAGGCCAAGATTCTGCTGCTGGACGAACCTTCG |
| | GCCCACCTGGATCCGGTCACCTACCAGATCATCAGGAGGACCCTGAAG |
| | CAGGCCTTTGCCGATTGCACCGTGATTCTCTGCGAGCACCGCATCGAGG |
| | CCATGCTGGAGTGCCAGCAGTTCCTGGTCATCGAGGAGAACAAGGTCC |
| | GCCAATACGACTCCATTCAAAAGCTCCTCAACGAGCGGTCGCTGTTCA |
| | GACAAGCTATTTCACCGTCCGATAGAGTGAAGCTCTTCCCGCATCGGA |
| | ACAGCTCAAAGTGCAAATCGAAGCCGCAGATCGCAGCCTTGAAGGAAG |
| | AGACTGAGGAAGAGGTGCAGGACACCCGGCTTTAA (SEQ ID NO: 46) |
| SEQ ID | ATGCAACGCTCTCCTCTTGAAAAGGCCTCGGTGGTGTCCAAGCTCTTCT |
| NO: 47 | TCTCGTGGACTAGACCCATCCTGAGAAAGGGGTACAGACAGCGCTTGG |
| | AGCTGTCCGATATCTATCAAATCCCTTCCGTGGACTCCGCGGACAACCT |
| | GTCCGAGAAGCTCGAGAGAGAATGGGACAGAGAACTCGCCTCAAAGA |
| | AGAACCCGAAGCTGATTAATGCGCTTAGGCGGTGCTTTTTCTGGCGGTT |
| | CATGTTCTACGGCATCTTCCTCTACCTGGGAGAGGTCACCAAGGCCGTG |
| | CAGCCCCTGTTGCTGGGACGGATTATTGCCTCCTACGACCCCGACAACA |
| | AGGAAGAAAGAAGCATCGCTATCTACTTGGGCATCGGTCTGTGCCTGC |
| | TTTTCATCGTCCGGACCCTCTTGTTGCATCCTGCTATTTTCGGCCTGCAT |
| | CACATTGGCATGCAGATGAGAATTGCCATGTTTTCCCTGATCTACAAGA |
| | AAACTCTGAAGCTCTCGAGCCGCGTGCTTGACAAGATTTCCATCGGCCA |
| | GCTCGTGTCCCTGCTCTCCAACAATCTGAACAAGTTCGACGAGGGCCTC |
| | GCCCTGGCCCACTTCGTGTGGATCGCCCCTCTGCAAGTGGCGCTTCTGA |
| | TGGGCCTGATCTGGGAGCTGCTGCAAGCCTCGGCATTCTGTGGGCTTGG |
| | ATTCCTGATCGTGCTGGCACTGTTCCAGGCCGGACTGGGGCGGATGAT |
| | GATGAAGTACAGGGACCAGAGAGCCGGAAAGATTTCCGAACGGCTGG |
| | TGATCACTTCGGAAATGATCGAAAACATCCAGTCAGTGAAGGCCTACT |
| | GCTGGGAAGAGGCCATGGAAAAGATGATTGAAAACCTCCGGCAAACC |
| | GAGCTGAAGCTGACCCGCAAGGCCGCTTACGTGCGCTATTTCAACTCGT |
| | CCGCTTTCTTCTTCTCCGGGTTCTTCGTGGTGTTTCTCTCCGTGCTCCCCT |
| | ACGCCCTGATTAAGGGAATCATCCTCAGGAAGATCTTCACCACCATTTC |
| | CTTCTGTATCGTGCTCCGCATGGCCGTGACCCGGCAGTTCCCATGGGCC |
| | GTGCAGACTTGGTACGACTCCCTGGGAGCCATTAACAAGATCCAGGAC |
| | TTCCTTCAAAAGCAGGAGTACAAGACCCTCGAGTACAACCTGACTACT |
| | ACCGAGGTCGTGATGGAAAACGTCACCGCCTTTTGGGAGGAGGGATTT |
| | GGCGAACTGTTCGAGAAGGCCAAGCAGAACAACAACAACCGCAAGAC |
| | CTCGAACGGTGACGACTCCCTCTTCTTTTCAAACTTCAGCCTGCTCGGG |
| | ACGCCCGTGCTGAAGGACATTAACTTCAAGATCGAAAGAGGACAGCTC |
| | CTGGCGGTGGCCGGATCGACCGGAGCCGGAAAGACTTCCCTGCTGATG |
| | GTGATCATGGGAGAGCTTGAACCTAGCGAGGGAAAGATCAAGCACTCC |
| | GGCCGCATCAGCTTCTGTAGCCAGTTTTCCTGGATCATGCCCGGAACCA |
| | TTAAGGAAAACATCATCTTCGGCGTGTCCTACGATGAATACCGCTACCG |
| | GTCCGTGATCAAAGCCTGCCAGCTGGAAGAGGATATTTCAAAGTTCGC |
| | GGAGAAAGATAACATCGTGCTGGGCGAAGGGGGTATTACCTTGTCGGG |
| | GGGCCAGCGGGCTAGAATCTCGCTGGCCAGAGCCGTGTATAAGGACGC |
| | CGACCTGTATCTCCTGGACTCCCCCTTCGGATACCTGGACGTCCTGACC |
| | GAAAAGGAGATCTTCGAATCGTGCGTGTGCAAGCTGATGGCTAACAAG |
| | ACTCGCATCCTCGTGACCTCCAAAATGGAGCACCTGAAGAAGGCAGAC |
| | AAGATTCTGATTCTGCATGAGGGGTCCTCCTACTTTTACGGCACCTTCT |
| | CGGAGTTGCAGAACTTGCAGCCCGACTTCTCATCGAAGCTGATGGGTT |
| | GCGACAGCTTCGACCAGTTCTCCGCCGAAAGAAGGAACTCGATCCTGA |
| | CGGAAACCTTGCACCGCTTCTCTTTGGAAGGCGACGCCCCTGTGTCATG |
| | GACCGAGACTAAGAAGCAGAGCTTCAAGCAGACCGGGGAATTCGGCG |
| | AAAAGAGGAAGAACAGCATCTTGAACCCCATTAACTCCATCCGCAAGT |
| | TCTCAATCGTGCAAAAGACGCCACTGCAGATGAACGGCATTGAGGAGG |
| | ACTCCGACGAACCCCTTGAGAGGCGCCTGTCCCTGGTGCCGGACAGCG |
| | AGCAGGGAGAAGCCATCCTGCCTCGGATTTCCGTGATCTCCACTGGTCC |
| | GACGCTCCAAGCCCGGCGGCGGCAGTCCGTGCTGAACCTGATGACCCA |
| | CAGCGTGAACCAGGGCCAAAACATTCACCGCAAGACTACCGCATCCAC |
| | CCGGAAAGTGTCCCTGGCACCTCAAGCGAATCTTACCGAGCTCGACAT |
| | CTACTCCCGGAGACTGTCGCAGGAAACCGGGCTCGAAATTTCCGAAGA |
| | AATCAACGAGGAGGATCTGAAAGAGTGCTTCTTCGACGATATGGAGTC |
| | GATACCCGCCGTGACGACTTGGAACACTTATCTGCGGTACATCACTGTG |
| | CACAAGTCATTGATCTTCGTGCTGATTTGGTGCCTGGTGATTTTCCTGG |
| | CCGAGGTCGCGGCCTCACTGGTGGTGCTCTGGCTGTTGGGAAACACGC |
| | CTCTGCAAGACAAGGGAAACTCCACGCACTCGAGAAACAACAGCTATG |
| | CCGTGATTATCACTTCCACCTCCTCTTATTACGTGTTCTACATCTACGTC |
| | GGAGTGGCGGATACCCTGCTCGCGATGGGTTTCTTCAGAGGACTGCCG |
| | CTGGTCCACACCTTGATCACCGTCAGCAAGATTCTTCACCACAAGATGT |
| | TGCATAGCGTGCTGCAGGCCCCCATGTCCACCCTCAACACTCTGAAGGC |
| | CGGAGGCATTCTGAACAGATTCTCCTGCGACATCGCTATCCTGGACGAT |
| | CTCCTGCCGCTTACCATCTTTGACTTCATCCAGCTGCTGCTGATCGTGAT |
| | TGGAGCAATCGCAGTGGTGGCGGTGCTGCAGCCTTACATTTTCGTGGCC |
| | ACTGTGCCGGTCATTGTGGCGTTCATCATGCTGCGGGCCTACTTCCTCC |
| | AAACCAGCCAGCAGCTGAAGCAACTGGAATCCGAGGGACGATCCCCCA |
| | TCTTCACTCACCTTGTGACGTCGTTGAAGGGACTGTGGACCCTCCGGGC |
| | TTTCGGACGGCAGCCCTACTTCGAAACCCTCTTCCACAAGGCCCTGAAC |
| | CTCCACACCGCCAATTGGTTCCTGTACCTGTCCACCCTGCGGTGGTTCC |
| | AGATGCGCATCGAGATGATTTTCGTCATCTTCTTCATCGCGGTCACATT |
| | CATCAGCATCCTGACTACCGGAGAGGGAGAGGGACGGGTCGGAATAAT |
| | CCTGACCCTCGCCATGAACATTATGAGCACCCTGCAGTGGGCAGTGAA |
| | CAGCTCGATCGACGTGGACAGCCTGATGCGAAGCGTCAGCCGCGTGTT |
| | CAAGTTCATCGACATGCCTACTGAGGGAAAACCCACTAAGTCCACTAA |
| | GCCCTACAAAAATGGCCAGCTGAGCAAGGTCATGATCATCGAAAACTC |
| | CCACGTGAAGAAGGACGATATTTGGCCCTCCGGAGGTCAAATGACCGT |
| | GAAGGACCTGACCGCAAAGTACACCGAGGGAGGAAACGCCATTCTCG |
| | AAAACATCAGCTTCTCCATTTCGCCGGGACAGCGGGTCGGCCTTCTCGG |
| | GCGGACCGGTTCCGGGAAGTCAACTCTGCTGTCGGCTTTCCTCCGGCTG |
| | CTGAATACCGAGGGGGAAATCCAAATTGACGGCGTGTCTTGGGATTCC |
| | ATTACTCTGCAGCAGTGGCGGAAGGCCTTCGGCGTGATCCCCCAGAAG |
| | GTGTTCATCTTCTCGGGTACCTTCCGGAAGAACCTGGATCCTTACGAGC |
| | AGTGGAGCGACCAAGAAATCTGGAAGGTCGCCGACGAGGTCGGCCTGC |
| | GCTCCGTGATTGAACAATTTCCTGGAAAGCTGGACTTCGTGCTCGTCGA |
| | CGGGGGATGTGTCCTGTCGCACGGACATAAGCAGCTCATGTGCCTCGC |
| | ACGGTCCGTGCTCTCCAAGGCCAAGATTCTGCTGCTGGACGAACCTTCG |
| | GCCCACCTGGATCCGGTCACCTACCAGATCATCAGGAGGACCCTGAAG |
| | CAGGCCTTTGCCGATTGCACCGTGATTCTCTGCGAGCACCGCATCGAGG |
| | CCATGCTGGAGTGCCAGCAGTTCCTGGTCATCGAGGAGAACAAGGTCC |
| | GCCAATACGACTCCATTCAAAAGCTCCTCAACGAGCGGTCGCTGTTCA |
| | GACAAGCTATTTCACCGTCCGATAGAGTGAAGCTCTTCCCGCATCGGA |
| | ACAGCTCAAAGTGCAAATCGAAGCCGCAGATCGCAGCCTTGAAGGAAG |
| | AGACTGAGGAAGAGGTGCAGGACACCCGGCTTTAA (SEQ ID NO: 47) |
| SEQ ID | ATGCAACGCTCTCCTCTTGAAAAGGCCTCGGTGGTGTCCAAGCTCTTCT |
| NO: 48 | TCTCGTGGACTAGACCCATCCTGAGAAAGGGGTACAGACAGCGCTTGG |
| | AGCTGTCCGATATCTATCAAATCCCTTCCGTGGACTCCGCGGACAACCT |
| | GTCCGAGAAGCTCGAGAGAGAATGGGACAGAGAACTCGCCTCAAAGA |
| | AGAACCCGAAGCTGATTAATGCGCTTAGGCGGTGCTTTTTCTGGCGGTT |
| | CATGTTCTACGGCATCTTCCTCTACCTGGGAGAGGTCACCAAGGCCGTG |
| | CAGCCCCTGTTGCTGGGACGGATTATTGCCTCCTACGACCCCGACAACA |
| | AGGAAGAAAGAAGCATCGCTATCTACTTGGGCATCGGTCTGTGCCTGC |
| | TTTTCATCGTCCGGACCCTCTTGTTGCATCCTGCTATTTTCGGCCTGCAT |
| | CACATTGGCATGCAGATGAGAATTGCCATGTTTTCCCTGATCTACAAGA |
| | AAACTCTGAAGCTCTCGAGCCGCGTGCTTGACAAGATTTCCATCGGCCA |
| | GCTCGTGTCCCTGCTCTCCAACAATCTGAACAAGTTCGACGAGGGCCTC |
| | GCCCTGGCCCACTTCGTGTGGATCGCCCCTCTGCAAGTGGCGCTTCTGA |
| | TGGGCCTGATCTGGGAGCTGCTGCAAGCCTCGGCATTCTGTGGGCTTGG |
| | ATTCCTGATCGTGCTGGCACTGTTCCAGGCCGGACTGGGGCGGATGAT |
| | GATGAAGTACAGGGACCAGAGAGCCGGAAAGATTTCCGAACGGCTGG |
| | TGATCACTTCGGAAATGATCGAAAACATCCAGTCAGTGAAGGCCTACT |
| | GCTGGGAAGAGGCCATGGAAAAGATGATTGAAAACCTCCGGCAAACC |
| | GAGCTGAAGCTGACCCGCAAGGCCGCTTACGTGCGCTATTTCAACTCGT |
| | CCGCTTTCTTCTTCTCCGGGTTCTTCGTGGTGTTTCTCTCCGTGCTCCCCT |
| | ACGCCCTGATTAAGGGAATCATCCTCAGGAAGATCTTCACCACCATTTC |
| | CTTCTGTATCGTGCTCCGCATGGCCGTGACCCGGCAGTTCCCATGGGCC |
| | GTGCAGACTTGGTACGACTCCCTGGGAGCCATTAACAAGATCCAGGAC |
| | TTCCTTCAAAAGCAGGAGTACAAGACCCTCGAGTACAACCTGACTACT |
| | ACCGAGGTCGTGATGGAAAACGTCACCGCCTTTTGGGAGGAGGGATTT |
| | GGCGAACTGTTCGAGAAGGCCAAGCAGAACAACAACAACCGCAAGAC |
| | CTCGAACGGTGACGACTCCCTCTTCTTTTCAAACTTCAGCCTGCTCGGG |
| | ACGCCCGTGCTGAAGGACATTAACTTCAAGATCGAAAGAGGACAGCTC |
| | CTGGCGGTGGCCGGATCGACCGGAGCCGGAAAGACTTCCCTGCTGATG |
| | GTGATCATGGGAGAGCTTGAACCTAGCGAGGGAAAGATCAAGCACTCC |
| | GGCCGCATCAGCTTCTGTAGCCAGTTTTCCTGGATCATGCCCGGAACCA |
| | TTAAGGAAAACATCATCTTCGGCGTGTCCTACGATGAATACCGCTACCG |
| | GTCCGTGATCAAAGCCTGCCAGCTGGAAGAGGATATTTCAAAGTTCGC |
| | GGAGAAAGATAACATCGTGCTGGGCGAAGGGGGTATTACCTTGTCGGG |
| | GGGCCAGCGGGCTAGAATCTCGCTGGCCAGAGCCGTGTATAAGGACGC |
| | CGACCTGTATCTCCTGGACTCCCCCTTCGGATACCTGGACGTCCTGACC |
| | GAAAAGGAGATCTTCGAATCGTGCGTGTGCAAGCTGATGGCTAACAAG |
| | ACTCGCATCCTCGTGACCTCCAAAATGGAGCACCTGAAGAAGGCAGAC |
| | AAGATTCTGATTCTGCATGAGGGGTCCTCCTACTTTTACGGCACCTTCT |
| | CGGAGTTGCAGAACTTGCAGCCCGACTTCTCATCGAAGCTGATGGGTT |
| | GCGACAGCTTCGACCAGTTCTCCGCCGAAAGAAGGAACTCGATCCTGA |
| | CGGAAACCTTGCACCGCTTCTCTTTGGAAGGCGACGCCCCTGTGTCATG |
| | GACCGAGACTAAGAAGCAGAGCTTCAAGCAGACCGGGGAATTCGGCG |
| | AAAAGAGGAAGAACAGCATCTTGAACCCCATTAACTCCATCCGCAAGT |
| | TCTCAATCGTGCAAAAGACGCCACTGCAGATGAACGGCATTGAGGAGG |
| | ACTCCGACGAACCCCTTGAGAGGCGCCTGTCCCTGGTGCCGGACAGCG |
| | AGCAGGGAGAAGCCATCCTGCCTCGGATTTCCGTGATCTCCACTGGTCC |
| | GACGCTCCAAGCCCGGCGGCGGCAGTCCGTGCTGAACCTGATGACCCA |
| | CAGCGTGAACCAGGGCCAAAACATTCACCGCAAGACTACCGCATCCAC |
| | CCGGAAAGTGTCCCTGGCACCTCAAGCGAATCTTACCGAGCTCGACAT |
| | CTACTCCCGGAGACTGTCGCAGGAAACCGGGCTCGAAATTTCCGAAGA |
| | AATCAACGAGGAGGATCTGAAAGAGTGCTTCTTCGACGATATGGAGTC |
| | GATACCCGCCGTGACGACTTGGAACACTTATCTGCGGTACATCACTGTG |
| | CACAAGTCATTGATCTTCGTGCTGATTTGGTGCCTGGTGATTTTCCTGG |
| | CCGAGGTCGCGGCCTCACTGGTGGTGCTCTGGCTGTTGGGAAACACGC |
| | CTCTGCAAGACAAGGGAAACTCCACGCACTCGAGAAACAACAGCTATG |
| | CCGTGATTATCACTTCCACCTCCTCTTATTACGTGTTCTACATCTACGTC |
| | GGAGTGGCGGATACCCTGCTCGCGATGGGTTTCTTCAGAGGACTGCCG |
| | CTGGTCCACACCTTGATCACCGTCAGCAAGATTCTTCACCACAAGATGT |
| | TGCATAGCGTGCTGCAGGCCCCCATGTCCACCCTCAACACTCTGAAGGC |
| | CGGAGGCATTCTGAACAGATTCTCCAAGGACATCGCTATCCTGGACGA |
| | TCTCCTGCCGCTTACCATCTTTGACTTCATCCAGCTGCTGCTGATCGTGA |
| | TTGGAGCAATCGCAGTGGTGGCGGTGCTGCAGCCTTACATTTTCGTGGC |
| | CACTGTGCCGGTCATTGTGGCGTTCATCATGCTGCGGGCCTACTTCCTC |
| | CAAACCAGCCAGCAGCTGAAGCAACTGGAATCCGAGGGACGATCCCCC |
| | ATCTTCACTCACCTTGTGACGTCGTTGAAGGGACTGTGGACCCTCCGGG |
| | CTTTCGGACGGCAGCCCTACTTCGAAACCCTCTTCCACAAGGCCCTGAA |
| | CCTCCACACCGCCAATTGGTTCCTGTACCTGTCCACCCTGCGGTGGTTC |
| | CAGATGCGCATCGAGATGATTTTCGTCATCTTCTTCATCGCGGTCACAT |
| | TCATCAGCATCCTGACTACCGGAGAGGGAGAGGGACGGGTCGGAATAA |
| | TCCTGACCCTCGCCATGAACATTATGAGCACCCTGCAGTGGGCAGTGA |
| | ACAGCTCGATCGACGTGGACAGCCTGATGCGAAGCGTCAGCCGCGTGT |
| | TCAAGTTCATCGACATGCCTACTGAGGGAAAACCCACTAAGTCCACTA |
| | AGCCCTACAAAAATGGCCAGCTGAGCAAGGTCATGATCATCGAAAACT |
| | CCCACGTGAAGAAGGACGATATTTGGCCCTCCGGAGGTCAAATGACCG |
| | TGAAGGACCTGACCGCAAAGTACACCGAGGGAGGAAACGCCATTCTCG |
| | AAAACATCAGCTTCTCCATTTCGCCGGGACAGCGGGTCGGCCTTCTCGG |
| | GCGGACCGGTTCCGGGAAGTCAACTCTGCTGTCGGCTTTCCTCCGGCTG |
| | CTGAATACCGAGGGGGAAATCCAAATTGACGGCGTGTCTTGGGATTCC |
| | ATTACTCTGCAGCAGTGGCGGAAGGCCTTCGGCGTGATCCCCCAGAAG |
| | GTGTTCATCTTCTCGGGTACCTTCCGGAAGAACCTGGATCCTTACGAGC |
| | AGTGGAGCGACCAAGAAATCTGGAAGGTCGCCGACGAGGTCGGCCTGC |
| | GCTCCGTGATTGAACAATTTCCTGGAAAGCTGGACTTCGTGCTCGTCGA |
| | CGGGGGATGTGTCCTGTCGCACGGACATAAGCAGCTCATGTGCCTCGC |
| | ACGGTCCGTGCTCTCCAAGGCCAAGATTCTGCTGCTGGACCAACCTTCG |
| | GCCCACCTGGATCCGGTCACCTACCAGATCATCAGGAGGACCCTGAAG |
| | CAGGCCTTTGCCGATTGCACCGTGATTCTCTGCGAGCACCGCATCGAGG |
| | CCATGCTGGAGTGCCAGCAGTTCCTGGTCATCGAGGAGAACAAGGTCC |
| | GCCAATACGACTCCATTCAAAAGCTCCTCAACGAGCGGTCGCTGTTCA |
| | GACAAGCTATTTCACCGTCCGATAGAGTGAAGCTCTTCCCGCATCGGA |
| | ACAGCTCAAAGTGCAAATCGAAGCCGCAGATCGCAGCCTTGAAGGAAG |
| | AGACTGAGGAAGAGGTGCAGGACACCCGGCTTTAA (SEQ ID NO: 48) |
| SEQ ID | ATGCAGCGCTCGCCTCTGGAAAAGGCGAGCGTCGTGTCACGGCTATTC |
| NO: 49 | TTTTCTTGGACCCGGCCCATTCTCAGGAAGGGCTACAGGCAGAGGCTG |
| Codon | GAGTTGAGCGACATCTATCAGATTCCTTCCGTGGACAGCGCCGACAAC |
| Optimized | CTGAGCGAGAAGCTGGAAAGGGAGTGGGACCGCGAACTGGCAAGCAA |
| hCFTR - | AAAGAACCCCAAGCTGATCAATGCCCTGAGAAGGTGTTTCTTTTGGAG |
| K14R | ATTCATGTTCTACGGGATCTTTCTGTATCTGGGCGAGGTTACAAAGGCT |
| E1371Q | GTGCAGCCCCTGCTGCTCGGCAGAATCATCGCCTCATACGATCCAGAC |
| | AACAAGGAAGAAAGAAGCATCGCCATCTACCTGGGCATTGGCCTCTGC |
| | CTCCTGTTTATTGTGCGGACTCTGCTGCTGCACCCAGCAATTTTCGGGTT |
| | GCATCATATTGGCATGCAGATGCGCATTGCTATGTTTTCCCTCATCTAC |
| | AAAAAGACACTGAAACTCAGCTCCCGGGTGCTGGACAAGATCTCCATC |
| | GGCCAACTGGTGTCTCTCCTGAGCAATAACTTGAATAAGTTCGACGAA |
| | GGGCTGGCCCTGGCACACTTCGTGTGGATTGCCCCCCTGCAGGTGGCCC |
| | TGCTGATGGGACTGATTTGGGAACTGCTGCAGGCTAGCGCTTTCTGCGG |
| | CCTGGGGTTCCTGATCGTGCTGGCACTGTTTCAGGCAGGCCTGGGCCGT |
| | ATGATGATGAAGTACAGAGACCAGAGGGCCGGGAAGATCTCCGAACG |
| | GCTCGTTATTACCTCTGAGATGATCGAGAACATTCAGTCTGTGAAAGCC |
| | TACTGCTGGGAGGAGGCTATGGAGAAGATGATCGAGAATCTGAGACAG |
| | ACCGAGCTGAAGCTGACCAGAAAGGCCGCCTACGTGAGGTACTTCAAC |
| | AGCAGTGCCTTCTTCTTCTCTGGCTTCTTCGTTGTGTTTCTGAGCGTGCT |
| | GCCATACGCTCTCATCAAAGGCATCATCCTGCGGAAGATCTTCACCACC |
| | ATCAGCTTTTGCATCGTGCTTAGAATGGCCGTGACCCGGCAGTTCCCAT |
| | GGGCCGTGCAAACTTGGTATGATTCCCTGGGCGCCATCAACAAAATCC |
| | AGGATTTCCTGCAGAAGCAGGAATACAAGACACTCGAATATAATCTCA |
| | CAACTACTGAGGTGGTTATGGAGAACGTGACTGCCTTCTGGGAGGAGG |
| | GGTTCGGAGAGCTTTTTGAGAAGGCAAAACAGAATAACAACAACCGCA |
| | AAACCAGCAACGGCGACGACAGCCTGTTCTTCTCCAATTTTTCTCTCCT |
| | GGGAACACCCGTCCTCAAAGACATCAACTTTAAGATCGAGAGGGGACA |
| | GCTGCTCGCAGTCGCCGGATCCACAGGCGCCGGCAAGACCTCTCTGCT |
| | GATGGTTATCATGGGCGAACTGGAGCCATCCGAGGGCAAGATTAAGCA |
| | CAGTGGAAGAATCTCCTTTTGTAGCCAGTTCAGTTGGATTATGCCCGGC |
| | ACTATTAAGGAGAATATCATTTTTGGGGTGAGCTATGATGAGTATCGGT |
| | ATCGGAGCGTTATCAAAGCCTGTCAGCTGGAGGAGGATATCAGCAAAT |
| | TCGCAGAGAAGGATAATATCGTGCTGGGGGAGGGGGGAATCACCCTGA |
| | GCGGAGGCCAGAGAGCCAGAATCTCACTGGCCCGGGCCGTCTACAAGG |
| | ACGCCGACCTTTACCTTCTGGACAGTCCCTTTGGATATCTGGATGTGCT |
| | GACTGAAAAGGAGATCTTCGAGTCTTGTGTGTGCAAGCTGATGGCTAA |
| | TAAGACCCGGATCCTAGTGACCAGTAAGATGGAGCACCTGAAGAAGGC |
| | AGACAAGATCTTGATTCTGCACGAGGGATCCTCTTACTTTTACGGCACC |
| | TTTAGCGAGCTGCAGAATCTCCAGCCCGATTTCTCATCTAAGCTGATGG |
| | GCTGTGATAGCTTCGACCAGTTCTCTGCCGAGCGCAGAAACAGCATCCT |
| | GACAGAGACACTGCACCGGTTTTCACTGGAGGGCGACGCCCCTGTCAG |
| | CTGGACCGAGACCAAAAAGCAGTCTTTCAAGCAGACAGGCGAGTTCGG |
| | CGAGAAGCGCAAAAACAGCATCCTGAATCCAATCAACTCTATAAGGAA |
| | GTTTAGCATCGTGCAGAAGACACCCCTCCAGATGAACGGCATCGAAGA |
| | GGACAGTGACGAGCCCCTGGAGCGGCGCCTGAGCCTCGTGCCTGACAG |
| | CGAACAGGGCGAGGCCATCCTGCCTAGGATCAGCGTGATTTCAACCGG |
| | GCCAACACTGCAGGCTAGGAGAAGACAGTCAGTGCTTAACCTGATGAC |
| | ACATAGCGTGAATCAGGGACAGAACATCCATCGAAAAACCACAGCCTC |
| | TACTCGCAAAGTGTCACTGGCTCCTCAGGCTAATCTGACAGAGCTGGA |
| | CATCTATAGCAGGAGGCTGAGCCAGGAGACAGGCCTGGAGATCAGTGA |
| | GGAGATCAACGAAGAGGACCTGAAGGAGTGCTTTTTCGATGACATGGA |
| | GAGTATCCCCGCCGTCACCACCTGGAATACCTACCTCCGGTACATCACA |
| | GTGCACAAGTCCCTCATCTTTGTGCTGATTTGGTGCCTCGTGATCTTTCT |
| | CGCAGAAGTGGCCGCCTCCCTGGTGGTGCTGTGGCTGTTGGGGAATAC |
| | TCCACTGCAGGACAAAGGCAATTCTACACACAGCAGGAATAATTCCTA |
| | TGCCGTGATTATCACCAGCACATCCTCTTACTACGTGTTCTACATCTAC |
| | GTGGGAGTGGCAGATACTCTGCTTGCAATGGGCTTCTTCAGGGGGCTG |
| | CCCCTGGTGCACACACTGATCACAGTGTCCAAGATCCTCCACCATAAA |
| | ATGCTCCACAGCGTGCTGCAGGCACCCATGAGCACCCTGAACACACTG |
| | AAGGCCGGCGGCATCCTGAATCGCTTTTCCAAAGACATCGCCATCCTCG |
| | ACGATCTCCTGCCACTGACCATCTTCGATTTTATCCAGCTGCTGCTGAT |
| | CGTGATCGGGGCCATCGCCGTGGTGGCCGTGCTGCAGCCATACATTTTC |
| | GTGGCTACAGTGCCCGTGATCGTTGCCTTTATCATGCTGAGAGCCTACT |
| | TCCTGCAGACTTCTCAGCAGCTGAAGCAGCTGGAGAGCGAAGGGAGAA |
| | GCCCCATCTTCACTCACCTGGTGACAAGCCTGAAGGGACTCTGGACCCT |
| | GAGAGCCTTCGGCCGGCAGCCCTATTTCGAGACCCTGTTTCACAAGGCC |
| | CTCAACCTGCACACAGCCAACTGGTTTCTCTACCTGTCCACCCTGAGGT |
| | GGTTCCAGATGAGGATTGAAATGATCTTCGTGATTTTTTTCATCGCCGT |
| | GACATTCATTAGCATTCTGACCACCGGCGAGGGGGAGGGGAGAGTGGG |
| | CATCATCCTGACCCTTGCCATGAACATTATGTCCACACTGCAGTGGGCC |
| | GTGAATAGTTCAATCGACGTGGACAGTCTGATGAGGTCCGTGAGCCGG |
| | GTGTTCAAGTTCATTGACATGCCCACAGAGGGGAAACCCACCAAAAGC |
| | ACCAAGCCCTACAAGAACGGGCAGCTGTCCAAGGTTATGATCATCGAG |
| | AACTCTCACGTGAAGAAGGACGACATTTGGCCCAGCGGCGGCCAGATG |
| | ACAGTGAAAGATCTGACCGCCAAATACACCGAGGGAGGCAACGCCATC |
| | CTCGAAAACATTAGCTTCTCTATCAGCCCTGGACAGAGGGTGGGCCTG |
| | CTGGGCCGGACAGGCTCAGGGAAGAGTACTCTGCTGTCAGCATTCCTG |
| | AGGCTCCTGAACACAGAGGGCGAGATCCAGATTGACGGCGTGTCCTGG |
| | GACTCCATCACCCTGCAGCAGTGGCGGAAGGCTTTCGGGGTGATCCCC |
| | CAGAAGGTGTTCATCTTTAGCGGCACTTTCAGAAAGAATCTGGACCCTT |
| | ATGAGCAGTGGAGTGACCAGGAGATCTGGAAAGTGGCCGATGAGGTC |
| | GGACTGAGGAGCGTGATCGAGCAGTTTCCAGGGAAGCTGGACTTTGTG |
| | CTGGTGGATGGCGGATGCGTGCTGTCTCACGGCCATAAACAGCTGATG |
| | TGTCTGGCCCGGTCCGTGCTGTCTAAGGCCAAGATCCTGCTGCTGGACC |
| | AACCCTCCGCCCACCTGGACCCCGTGACATACCAGATCATCAGGAGAA |
| | CTCTCAAGCAGGCCTTCGCCGACTGTACCGTGATTCTGTGCGAGCACCG |
| | CATTGAAGCTATGCTGGAGTGTCAGCAGTTCCTGGTGATCGAGGAAAA |
| | TAAGGTGAGGCAGTACGACAGCATCCAGAAGCTGCTGAACGAGCGCTC |
| | CCTGTTCCGCCAGGCTATCTCCCCATCAGACCGGGTGAAGCTCTTTCCC |
| | CACAGAAACTCCTCAAAGTGCAAGTCCAAGCCCCAGATCGCCGCCCTG |
| | AAGGAGGAGACCGAGGAGGAGGTGCAGGACACCAGGCTGTGA (SEQ |
| | ID NO: 49) |
| SEQ ID | ATGCAGCGCTCGCCTCTGGAAAAGGCGAGCGTCGTGTCACGGCTATTC |
| NO: 50 | TTTTCTTGGACCCGGCCCATTCTCAGGAAGGGCTACAGGCAGAGGCTG |
| Codon | GAGTTGAGCGACATCTATCAGATTCCTTCCGTGGACAGCGCCGACAAC |
| Optimized - | CTGAGCGAGAAGCTGGAAAGGGAGTGGGACCGCGAACTGGCAAGCAA |
| hCFTR- | AAAGAACCCCAAGCTGATCAATGCCCTGAGAAGGTGTTTCTTTTGGAG |
| 13E K14R | ATTCATGTTCTACGGGATCTTTCTGTATCTGGGCGAGGTTACAAAGGCT |
| | GTGCAGCCCCTGCTGCTCGGCAGAATCATCGCCTCATACGATCCAGAC |
| | AACAAGGAAGAAAGAAGCATCGCCATCTACCTGGGCATTGGCCTCTGC |
| | CTCCTGTTTATTGTGCGGACTCTGCTGCTGCACCCAGCAATTTTCGGGTT |
| | GCATCATATTGGCATGCAGATGCGCATTGCTATGTTTTCCCTCATCTAC |
| | AAAAAGACACTGAAACTCAGCTCCCGGGTGCTGGACAAGATCTCCATC |
| | GGCCAACTGGTGTCTCTCCTGAGCAATAACTTGAATAAGTTCGACGAA |
| | GGGCTGGCCCTGGCACACTTCGTGTGGATTGCCCCCCTGCAGGTGGCCC |
| | TGCTGATGGGACTGATTTGGGAACTGCTGCAGGCTAGCGCTTTCTGCGG |
| | CCTGGGGTTCCTGATCGTGCTGGCACTGTTTCAGGCAGGCCTGGGCCGT |
| | ATGATGATGAAGTACAGAGACCAGAGGGCCGGGAAGATCTCCGAACG |
| | GCTCGTTATTACCTCTGAGATGATCGAGAACATTCAGTCTGTGAAAGCC |
| | TACTGCTGGGAGGAGGCTATGGAGAAGATGATCGAGAATCTGAGACAG |
| | ACCGAGCTGAAGCTGACCAGAAAGGCCGCCTACGTGAGGTACTTCAAC |
| | AGCAGTGCCTTCTTCTTCTCTGGCTTCTTCGTTGTGTTTCTGAGCGTGCT |
| | GCCATACGCTCTCATCAAAGGCATCATCCTGCGGAAGATCTTCACCACC |
| | ATCAGCTTTTGCATCGTGCTTAGAATGGCCGTGACCCGGCAGTTCCCAT |
| | GGGCCGTGCAAACTTGGTATGATTCCCTGGGCGCCATCAACAAAATCC |
| | AGGATTTCCTGCAGAAGCAGGAATACAAGACACTCGAATATAATCTCA |
| | CAACTACTGAGGTGGTTATGGAGAACGTGACTGCCTTCTGGGAGGAGG |
| | GGTTCGGAGAGCTTTTTGAGAAGGCAAAACAGAATAACAACAACCGCA |
| | AAACCGAGAACGGCGACGACAGCCTGTTCTTCTCCAATTTTTCTCTCCT |
| | GGGAACACCCGTCCTCAAAGACATCAACTTTAAGATCGAGAGGGGACA |
| | GCTGCTCGCAGTCGCCGGATCCACAGGCGCCGGCAAGACCTCTCTGCT |
| | GATGGTTATCATGGGCGAACTGGAGCCATCCGAGGGCAAGATTAAGCA |
| | CAGTGGAAGAATCTCCTTTTGTAGCCAGTTCAGTTGGATTATGCCCGGC |
| | ACTATTAAGGAGAATATCATTTTTGGGGTGAGCTATGATGAGTATCGGT |
| | ATCGGAGCGTTATCAAAGCCTGTCAGCTGGAGGAGGATATCAGCAAAT |
| | TCGCAGAGAAGGATAATATCGTGCTGGGGGAGGGGGGAATCACCCTGA |
| | GCGGAGGCCAGAGAGCCAGAATCTCACTGGCCCGGGCCGTCTACAAGG |
| | ACGCCGACCTTTACCTTCTGGACAGTCCCTTTGGATATCTGGATGTGCT |
| | GACTGAAAAGGAGATCTTCGAGTCTTGTGTGTGCAAGCTGATGGCTAA |
| | TAAGACCCGGATCCTAGTGACCAGTAAGATGGAGCACCTGAAGAAGGC |
| | AGACAAGATCTTGATTCTGCACGAGGGATCCTCTTACTTTTACGGCACC |
| | TTTAGCGAGCTGCAGAATCTCCAGCCCGATTTCTCATCTAAGCTGATGG |
| | GCTGTGATAGCTTCGACCAGTTCTCTGCCGAGCGCAGAAACGAAATCC |
| | TGACAGAGACACTGCACCGGTTTGAGCTGGAGGGCGACGCCCCTGTCA |
| | GCTGGACCGAGACCAAAAAGCAGGAATTCAAGCAGGAGGGCGAGTTC |
| | GGCGAGAAGCGCAAAAACGAAATCCTGAATCCAATCAACTCTATAAGG |
| | AAGTTTGAAATCGTGCAGAAGACACCCCTCCAGATGAACGGCATCGAA |
| | GAGGACAGTGACGAGCCCCTGGAGCGGCGCCTGAGCCTCGTGCCTGAC |
| | AGCGAACAGGGCGAGGCCATCCTGCCTAGGATCGAGGTGATTTCAACC |
| | GGGCCAACACTGCAGGCTAGGAGAAGACAGTCAGTGCTTAACCTGATG |
| | ACACATAGCGTGAATCAGGGACAGAACATCCATCGAAAAGAAGAAGC |
| | CGAAACTCGCAAAGTGGAGCTGGCTCCTCAGGCTAATCTGACAGAGCT |
| | GGACATCTATAGCAGGAGGCTGGAACAGGAGACAGGCCTGGAGATCA |
| | GTGAGGAGATCAACGAAGAGGACCTGAAGGAGTGCTTTTTCGATGACA |
| | TGGAGAGTATCCCCGCCGTCACCACCTGGAATACCTACCTCCGGTACAT |
| | CACAGTGCACAAGTCCCTCATCTTTGTGCTGATTTGGTGCCTCGTGATC |
| | TTTCTCGCAGAAGTGGCCGCCTCCCTGGTGGTGCTGTGGCTGTTGGGGA |
| | ATACTCCACTGCAGGACAAAGGCAATTCTACACACAGCAGGAATAATT |
| | CCTATGCCGTGATTATCACCAGCACATCCTCTTACTACGTGTTCTACAT |
| | CTACGTGGGAGTGGCAGATACTCTGCTTGCAATGGGCTTCTTCAGGGG |
| | GCTGCCCCTGGTGCACACACTGATCACAGTGTCCAAGATCCTCCACCAT |
| | AAAATGCTCCACAGCGTGCTGCAGGCACCCATGAGCACCCTGAACACA |
| | CTGAAGGCCGGCGGCATCCTGAATCGCTTTTCCAAAGACATCGCCATCC |
| | TCGACGATCTCCTGCCACTGACCATCTTCGATTTTATCCAGCTGCTGCT |
| | GATCGTGATCGGGGCCATCGCCGTGGTGGCCGTGCTGCAGCCATACAT |
| | TTTCGTGGCTACAGTGCCCGTGATCGTTGCCTTTATCATGCTGAGAGCC |
| | TACTTCCTGCAGACTTCTCAGCAGCTGAAGCAGCTGGAGAGCGAAGGG |
| | AGAAGCCCCATCTTCACTCACCTGGTGACAAGCCTGAAGGGACTCTGG |
| | ACCCTGAGAGCCTTCGGCCGGCAGCCCTATTTCGAGACCCTGTTTCACA |
| | AGGCCCTCAACCTGCACACAGCCAACTGGTTTCTCTACCTGTCCACCCT |
| | GAGGTGGTTCCAGATGAGGATTGAAATGATCTTCGTGATTTTTTTCATC |
| | GCCGTGACATTCATTAGCATTCTGACCACCGGCGAGGGGGAGGGGAGA |
| | GTGGGCATCATCCTGACCCTTGCCATGAACATTATGTCCACACTGCAGT |
| | GGGCCGTGAATAGTTCAATCGACGTGGACAGTCTGATGAGGTCCGTGA |
| | GCCGGGTGTTCAAGTTCATTGACATGCCCACAGAGGGGAAACCCACCA |
| | AAAGCACCAAGCCCTACAAGAACGGGCAGCTGTCCAAGGTTATGATCA |
| | TCGAGAACTCTCACGTGAAGAAGGACGACATTTGGCCCAGCGGCGGCC |
| | AGATGACAGTGAAAGATCTGACCGCCAAATACACCGAGGGAGGCAAC |
| | GCCATCCTCGAAAACATTAGCTTCTCTATCAGCCCTGGACAGAGGGTG |
| | GGCCTGCTGGGCCGGACAGGCTCAGGGAAGAGTACTCTGCTGTCAGCA |
| | TTCCTGAGGCTCCTGAACACAGAGGGCGAGATCCAGATTGACGGCGTG |
| | TCCTGGGACTCCATCACCCTGCAGCAGTGGCGGAAGGCTTTCGGGGTG |
| | ATCCCCCAGAAGGTGTTCATCTTTAGCGGCACTTTCAGAAAGAATCTGG |
| | ACCCTTATGAGCAGTGGAGTGACCAGGAGATCTGGAAAGTGGCCGATG |
| | AGGTCGGACTGAGGAGCGTGATCGAGCAGTTTCCAGGGAAGCTGGACT |
| | TTGTGCTGGTGGATGGCGGATGCGTGCTGTCTCACGGCCATAAACAGCT |
| | GATGTGTCTGGCCCGGTCCGTGCTGTCTAAGGCCAAGATCCTGCTGCTG |
| | GACGAACCCTCCGCCCACCTGGACCCCGTGACATACCAGATCATCAGG |
| | AGAACTCTCAAGCAGGCCTTCGCCGACTGTACCGTGATTCTGTGCGAGC |
| | ACCGCATTGAAGCTATGCTGGAGTGTCAGCAGTTCCTGGTGATCGAGG |
| | AAAATAAGGTGAGGCAGTACGACAGCATCCAGAAGCTGCTGAACGAG |
| | CGCTCCCTGTTCCGCCAGGCTATCTCCCCATCAGACCGGGTGAAGCTCT |
| | TTCCCCACAGAAACTCCTCAAAGTGCAAGTCCAAGCCCCAGATCGCCG |
| | CCCTGAAGGAGGAGACCGAGGAGGAGGTGCAGGACACCAGGCTGTGA |
| | (SEQ ID NO: 50) |
| SEQ ID | ATGCAGCGCTCGCCTCTGGAAAAGGCGAGCGTCGTGTCACGGCTATTC |
| NO: 51 | TTTTCTTGGACCCGGCCCATTCTCAGGAAGGGCTACAGGCAGAGGCTG |
| Codon | GAGTTGAGCGACATCTATCAGATTCCTTCCGTGGACAGCGCCGACAAC |
| Optimized- | CTGAGCGAGAAGCTGGAAAGGGAGTGGGACCGCGAACTGGCAAGCAA |
| hCFTR- | AAAGAACCCCAAGCTGATCAATGCCCTGAGAAGGTGTTTCTTTTGGAG |
| 15E K14R | ATTCATGTTCTACGGGATCTTTCTGTATCTGGGCGAGGTTACAAAGGCT |
| | GTGCAGCCCCTGCTGCTCGGCAGAATCATCGCCTCATACGATCCAGAC |
| | AACAAGGAAGAAAGAAGCATCGCCATCTACCTGGGCATTGGCCTCTGC |
| | CTCCTGTTTATTGTGCGGACTCTGCTGCTGCACCCAGCAATTTTCGGGTT |
| | GCATCATATTGGCATGCAGATGCGCATTGCTATGTTTTCCCTCATCTAC |
| | AAAAAGACACTGAAACTCAGCTCCCGGGTGCTGGACAAGATCTCCATC |
| | GGCCAACTGGTGTCTCTCCTGAGCAATAACTTGAATAAGTTCGACGAA |
| | GGGCTGGCCCTGGCACACTTCGTGTGGATTGCCCCCCTGCAGGTGGCCC |
| | TGCTGATGGGACTGATTTGGGAACTGCTGCAGGCTAGCGCTTTCTGCGG |
| | CCTGGGGTTCCTGATCGTGCTGGCACTGTTTCAGGCAGGCCTGGGCCGT |
| | ATGATGATGAAGTACAGAGACCAGAGGGCCGGGAAGATCTCCGAACG |
| | GCTCGTTATTACCTCTGAGATGATCGAGAACATTCAGTCTGTGAAAGCC |
| | TACTGCTGGGAGGAGGCTATGGAGAAGATGATCGAGAATCTGAGACAG |
| | ACCGAGCTGAAGCTGACCAGAAAGGCCGCCTACGTGAGGTACTTCAAC |
| | AGCAGTGCCTTCTTCTTCTCTGGCTTCTTCGTTGTGTTTCTGAGCGTGCT |
| | GCCATACGCTCTCATCAAAGGCATCATCCTGCGGAAGATCTTCACCACC |
| | ATCAGCTTTTGCATCGTGCTTAGAATGGCCGTGACCCGGCAGTTCCCAT |
| | GGGCCGTGCAAACTTGGTATGATTCCCTGGGCGCCATCAACAAAATCC |
| | AGGATTTCCTGCAGAAGCAGGAATACAAGACACTCGAATATAATCTCA |
| | CAACTACTGAGGTGGTTATGGAGAACGTGACTGCCTTCTGGGAGGAGG |
| | GGTTCGGAGAGCTTTTTGAGAAGGCAAAACAGAATAACAACAACCGCA |
| | AAACCGAGAACGGCGACGACAGCCTGTTCTTCTCCAATTTTTCTCTCCT |
| | GGGAACACCCGTCCTCAAAGACATCAACTTTAAGATCGAGAGGGGACA |
| | GCTGCTCGCAGTCGCCGGATCCACAGGCGCCGGCAAGACCTCTCTGCT |
| | GATGGTTATCATGGGCGAACTGGAGCCATCCGAGGGCAAGATTAAGCA |
| | CAGTGGAAGAATCTCCTTTTGTAGCCAGTTCAGTTGGATTATGCCCGGC |
| | ACTATTAAGGAGAATATCATTTTTGGGGTGAGCTATGATGAGTATCGGT |
| | ATCGGAGCGTTATCAAAGCCTGTCAGCTGGAGGAGGATATCAGCAAAT |
| | TCGCAGAGAAGGATAATATCGTGCTGGGGGAGGGGGGAATCACCCTGA |
| | GCGGAGGCCAGAGAGCCAGAATCTCACTGGCCCGGGCCGTCTACAAGG |
| | ACGCCGACCTTTACCTTCTGGACAGTCCCTTTGGATATCTGGATGTGCT |
| | GACTGAAAAGGAGATCTTCGAGTCTTGTGTGTGCAAGCTGATGGCTAA |
| | TAAGACCCGGATCCTAGTGACCAGTAAGATGGAGCACCTGAAGAAGGC |
| | AGACAAGATCTTGATTCTGCACGAGGGATCCTCTTACTTTTACGGCACC |
| | TTTAGCGAGCTGCAGAATCTCCAGCCCGATTTCTCATCTAAGCTGATGG |
| | GCTGTGATAGCTTCGACCAGTTCTCTGCCGAGCGCAGAAACGAAATCC |
| | TGACAGAGACACTGCACCGGTTTGAGCTGGAGGGCGACGCCCCTGTCA |
| | GCTGGACCGAGACCAAAAAGCAGGAATTCAAGCAGGAGGGCGAGTTC |
| | GGCGAGAAGCGCAAAAACGAAATCCTGAATCCAATCAACTCTATAAGG |
| | AAGTTTGAAATCGTGCAGAAGACACCCCTCCAGATGAACGGCATCGAA |
| | GAGGACAGTGACGAGCCCCTGGAGCGGCGCCTGGAACTCGTGCCTGAC |
| | AGCGAACAGGGCGAGGCCATCCTGCCTAGGATCGAGGTGATTTCAACC |
| | GGGCCAACACTGCAGGCTAGGAGAAGACAGGAAGTGCTTAACCTGATG |
| | ACACATAGCGTGAATCAGGGACAGAACATCCATCGAAAAGAAGAAGC |
| | CGAAACTCGCAAAGTGGAGCTGGCTCCTCAGGCTAATCTGACAGAGCT |
| | GGACATCTATAGCAGGAGGCTGGAACAGGAGACAGGCCTGGAGATCA |
| | GTGAGGAGATCAACGAAGAGGACCTGAAGGAGTGCTTTTTCGATGACA |
| | TGGAGAGTATCCCCGCCGTCACCACCTGGAATACCTACCTCCGGTACAT |
| | CACAGTGCACAAGTCCCTCATCTTTGTGCTGATTTGGTGCCTCGTGATC |
| | TTTCTCGCAGAAGTGGCCGCCTCCCTGGTGGTGCTGTGGCTGTTGGGGA |
| | ATACTCCACTGCAGGACAAAGGCAATTCTACACACAGCAGGAATAATT |
| | CCTATGCCGTGATTATCACCAGCACATCCTCTTACTACGTGTTCTACAT |
| | CTACGTGGGAGTGGCAGATACTCTGCTTGCAATGGGCTTCTTCAGGGG |
| | GCTGCCCCTGGTGCACACACTGATCACAGTGTCCAAGATCCTCCACCAT |
| | AAAATGCTCCACAGCGTGCTGCAGGCACCCATGAGCACCCTGAACACA |
| | CTGAAGGCCGGCGGCATCCTGAATCGCTTTTCCAAAGACATCGCCATCC |
| | TCGACGATCTCCTGCCACTGACCATCTTCGATTTTATCCAGCTGCTGCT |
| | GATCGTGATCGGGGCCATCGCCGTGGTGGCCGTGCTGCAGCCATACAT |
| | TTTCGTGGCTACAGTGCCCGTGATCGTTGCCTTTATCATGCTGAGAGCC |
| | TACTTCCTGCAGACTTCTCAGCAGCTGAAGCAGCTGGAGAGCGAAGGG |
| | AGAAGCCCCATCTTCACTCACCTGGTGACAAGCCTGAAGGGACTCTGG |
| | ACCCTGAGAGCCTTCGGCCGGCAGCCCTATTTCGAGACCCTGTTTCACA |
| | AGGCCCTCAACCTGCACACAGCCAACTGGTTTCTCTACCTGTCCACCCT |
| | GAGGTGGTTCCAGATGAGGATTGAAATGATCTTCGTGATTTTTTTCATC |
| | GCCGTGACATTCATTAGCATTCTGACCACCGGCGAGGGGGAGGGGAGA |
| | GTGGGCATCATCCTGACCCTTGCCATGAACATTATGTCCACACTGCAGT |
| | GGGCCGTGAATAGTTCAATCGACGTGGACAGTCTGATGAGGTCCGTGA |
| | GCCGGGTGTTCAAGTTCATTGACATGCCCACAGAGGGGAAACCCACCA |
| | AAAGCACCAAGCCCTACAAGAACGGGCAGCTGTCCAAGGTTATGATCA |
| | TCGAGAACTCTCACGTGAAGAAGGACGACATTTGGCCCAGCGGCGGCC |
| | AGATGACAGTGAAAGATCTGACCGCCAAATACACCGAGGGAGGCAAC |
| | GCCATCCTCGAAAACATTAGCTTCTCTATCAGCCCTGGACAGAGGGTG |
| | GGCCTGCTGGGCCGGACAGGCTCAGGGAAGAGTACTCTGCTGTCAGCA |
| | TTCCTGAGGCTCCTGAACACAGAGGGCGAGATCCAGATTGACGGCGTG |
| | TCCTGGGACTCCATCACCCTGCAGCAGTGGCGGAAGGCTTTCGGGGTG |
| | ATCCCCCAGAAGGTGTTCATCTTTAGCGGCACTTTCAGAAAGAATCTGG |
| | ACCCTTATGAGCAGTGGAGTGACCAGGAGATCTGGAAAGTGGCCGATG |
| | AGGTCGGACTGAGGAGCGTGATCGAGCAGTTTCCAGGGAAGCTGGACT |
| | TTGTGCTGGTGGATGGCGGATGCGTGCTGTCTCACGGCCATAAACAGCT |
| | GATGTGTCTGGCCCGGTCCGTGCTGTCTAAGGCCAAGATCCTGCTGCTG |
| | GACGAACCCTCCGCCCACCTGGACCCCGTGACATACCAGATCATCAGG |
| | AGAACTCTCAAGCAGGCCTTCGCCGACTGTACCGTGATTCTGTGCGAGC |
| | ACCGCATTGAAGCTATGCTGGAGTGTCAGCAGTTCCTGGTGATCGAGG |
| | AAAATAAGGTGAGGCAGTACGACAGCATCCAGAAGCTGCTGAACGAG |
| | CGCTCCCTGTTCCGCCAGGCTATCTCCCCATCAGACCGGGTGAAGCTCT |
| | TTCCCCACAGAAACTCCTCAAAGTGCAAGTCCAAGCCCCAGATCGCCG |
| | CCCTGAAGGAGGAGACCGAGGAGGAGGTGCAGGACACCAGGCTGTGA |
| | (SEQ ID NO: 51) |
| SEQ ID | ATGCAGCGCTCGCCTCTGGAAAAGGCGAGCGTCGTGTCAAAGCTATTC |
| NO: 52 | TTTTCTTGGACCCGGCCCATTCTCAGGAAGGGCTACAGGCAGAGGCTG |
| Codon | GAGTTGAGCGACATCTATCAGATTCCTTCCGTGGACAGCGCCGACAAC |
| Optimized- | CTGAGCGAGAAGCTGGAAAGGGAGTGGGACCGCGAACTGGCAAGCAA |
| hCFTR- | AAAGAACCCCAAGCTGATCAATGCCCTGAGAAGGTGTTTCTTTTGGAG |
| 13E | ATTCATGTTCTACGGGATCTTTCTGTATCTGGGCGAGGTTACAAAGGCT |
| | GTGCAGCCCCTGCTGCTCGGCAGAATCATCGCCTCATACGATCCAGAC |
| | AACAAGGAAGAAAGAAGCATCGCCATCTACCTGGGCATTGGCCTCTGC |
| | CTCCTGTTTATTGTGCGGACTCTGCTGCTGCACCCAGCAATTTTCGGGTT |
| | GCATCATATTGGCATGCAGATGCGCATTGCTATGTTTTCCCTCATCTAC |
| | AAAAAGACACTGAAACTCAGCTCCCGGGTGCTGGACAAGATCTCCATC |
| | GGCCAACTGGTGTCTCTCCTGAGCAATAACTTGAATAAGTTCGACGAA |
| | GGGCTGGCCCTGGCACACTTCGTGTGGATTGCCCCCCTGCAGGTGGCCC |
| | TGCTGATGGGACTGATTTGGGAACTGCTGCAGGCTAGCGCTTTCTGCGG |
| | CCTGGGGTTCCTGATCGTGCTGGCACTGTTTCAGGCAGGCCTGGGCCGT |
| | ATGATGATGAAGTACAGAGACCAGAGGGCCGGGAAGATCTCCGAACG |
| | GCTCGTTATTACCTCTGAGATGATCGAGAACATTCAGTCTGTGAAAGCC |
| | TACTGCTGGGAGGAGGCTATGGAGAAGATGATCGAGAATCTGAGACAG |
| | ACCGAGCTGAAGCTGACCAGAAAGGCCGCCTACGTGAGGTACTTCAAC |
| | AGCAGTGCCTTCTTCTTCTCTGGCTTCTTCGTTGTGTTTCTGAGCGTGCT |
| | GCCATACGCTCTCATCAAAGGCATCATCCTGCGGAAGATCTTCACCACC |
| | ATCAGCTTTTGCATCGTGCTTAGAATGGCCGTGACCCGGCAGTTCCCAT |
| | GGGCCGTGCAAACTTGGTATGATTCCCTGGGCGCCATCAACAAAATCC |
| | AGGATTTCCTGCAGAAGCAGGAATACAAGACACTCGAATATAATCTCA |
| | CAACTACTGAGGTGGTTATGGAGAACGTGACTGCCTTCTGGGAGGAGG |
| | GGTTCGGAGAGCTTTTTGAGAAGGCAAAACAGAATAACAACAACCGCA |
| | AAACCGAGAACGGCGACGACAGCCTGTTCTTCTCCAATTTTTCTCTCCT |
| | GGGAACACCCGTCCTCAAAGACATCAACTTTAAGATCGAGAGGGGACA |
| | GCTGCTCGCAGTCGCCGGATCCACAGGCGCCGGCAAGACCTCTCTGCT |
| | GATGGTTATCATGGGCGAACTGGAGCCATCCGAGGGCAAGATTAAGCA |
| | CAGTGGAAGAATCTCCTTTTGTAGCCAGTTCAGTTGGATTATGCCCGGC |
| | ACTATTAAGGAGAATATCATTTTTGGGGTGAGCTATGATGAGTATCGGT |
| | ATCGGAGCGTTATCAAAGCCTGTCAGCTGGAGGAGGATATCAGCAAAT |
| | TCGCAGAGAAGGATAATATCGTGCTGGGGGAGGGGGGAATCACCCTGA |
| | GCGGAGGCCAGAGAGCCAGAATCTCACTGGCCCGGGCCGTCTACAAGG |
| | ACGCCGACCTTTACCTTCTGGACAGTCCCTTTGGATATCTGGATGTGCT |
| | GACTGAAAAGGAGATCTTCGAGTCTTGTGTGTGCAAGCTGATGGCTAA |
| | TAAGACCCGGATCCTAGTGACCAGTAAGATGGAGCACCTGAAGAAGGC |
| | AGACAAGATCTTGATTCTGCACGAGGGATCCTCTTACTTTTACGGCACC |
| | TTTAGCGAGCTGCAGAATCTCCAGCCCGATTTCTCATCTAAGCTGATGG |
| | GCTGTGATAGCTTCGACCAGTTCTCTGCCGAGCGCAGAAACGAAATCC |
| | TGACAGAGACACTGCACCGGTTTGAGCTGGAGGGCGACGCCCCTGTCA |
| | GCTGGACCGAGACCAAAAAGCAGGAATTCAAGCAGGAGGGCGAGTTC |
| | GGCGAGAAGCGCAAAAACGAAATCCTGAATCCAATCAACTCTATAAGG |
| | AAGTTTGAAATCGTGCAGAAGACACCCCTCCAGATGAACGGCATCGAA |
| | GAGGACAGTGACGAGCCCCTGGAGCGGCGCCTGAGCCTCGTGCCTGAC |
| | AGCGAACAGGGCGAGGCCATCCTGCCTAGGATCGAGGTGATTTCAACC |
| | GGGCCAACACTGCAGGCTAGGAGAAGACAGTCAGTGCTTAACCTGATG |
| | ACACATAGCGTGAATCAGGGACAGAACATCCATCGAAAAGAAGAAGC |
| | CGAAACTCGCAAAGTGGAGCTGGCTCCTCAGGCTAATCTGACAGAGCT |
| | GGACATCTATAGCAGGAGGCTGGAACAGGAGACAGGCCTGGAGATCA |
| | GTGAGGAGATCAACGAAGAGGACCTGAAGGAGTGCTTTTTCGATGACA |
| | TGGAGAGTATCCCCGCCGTCACCACCTGGAATACCTACCTCCGGTACAT |
| | CACAGTGCACAAGTCCCTCATCTTTGTGCTGATTTGGTGCCTCGTGATC |
| | TTTCTCGCAGAAGTGGCCGCCTCCCTGGTGGTGCTGTGGCTGTTGGGGA |
| | ATACTCCACTGCAGGACAAAGGCAATTCTACACACAGCAGGAATAATT |
| | CCTATGCCGTGATTATCACCAGCACATCCTCTTACTACGTGTTCTACAT |
| | CTACGTGGGAGTGGCAGATACTCTGCTTGCAATGGGCTTCTTCAGGGG |
| | GCTGCCCCTGGTGCACACACTGATCACAGTGTCCAAGATCCTCCACCAT |
| | AAAATGCTCCACAGCGTGCTGCAGGCACCCATGAGCACCCTGAACACA |
| | CTGAAGGCCGGCGGCATCCTGAATCGCTTTTCCAAAGACATCGCCATCC |
| | TCGACGATCTCCTGCCACTGACCATCTTCGATTTTATCCAGCTGCTGCT |
| | GATCGTGATCGGGGCCATCGCCGTGGTGGCCGTGCTGCAGCCATACAT |
| | TTTCGTGGCTACAGTGCCCGTGATCGTTGCCTTTATCATGCTGAGAGCC |
| | TACTTCCTGCAGACTTCTCAGCAGCTGAAGCAGCTGGAGAGCGAAGGG |
| | AGAAGCCCCATCTTCACTCACCTGGTGACAAGCCTGAAGGGACTCTGG |
| | ACCCTGAGAGCCTTCGGCCGGCAGCCCTATTTCGAGACCCTGTTTCACA |
| | AGGCCCTCAACCTGCACACAGCCAACTGGTTTCTCTACCTGTCCACCCT |
| | GAGGTGGTTCCAGATGAGGATTGAAATGATCTTCGTGATTTTTTTCATC |
| | GCCGTGACATTCATTAGCATTCTGACCACCGGCGAGGGGGAGGGGAGA |
| | GTGGGCATCATCCTGACCCTTGCCATGAACATTATGTCCACACTGCAGT |
| | GGGCCGTGAATAGTTCAATCGACGTGGACAGTCTGATGAGGTCCGTGA |
| | GCCGGGTGTTCAAGTTCATTGACATGCCCACAGAGGGGAAACCCACCA |
| | AAAGCACCAAGCCCTACAAGAACGGGCAGCTGTCCAAGGTTATGATCA |
| | TCGAGAACTCTCACGTGAAGAAGGACGACATTTGGCCCAGCGGCGGCC |
| | AGATGACAGTGAAAGATCTGACCGCCAAATACACCGAGGGAGGCAAC |
| | GCCATCCTCGAAAACATTAGCTTCTCTATCAGCCCTGGACAGAGGGTG |
| | GGCCTGCTGGGCCGGACAGGCTCAGGGAAGAGTACTCTGCTGTCAGCA |
| | TTCCTGAGGCTCCTGAACACAGAGGGCGAGATCCAGATTGACGGCGTG |
| | TCCTGGGACTCCATCACCCTGCAGCAGTGGCGGAAGGCTTTCGGGGTG |
| | ATCCCCCAGAAGGTGTTCATCTTTAGCGGCACTTTCAGAAAGAATCTGG |
| | ACCCTTATGAGCAGTGGAGTGACCAGGAGATCTGGAAAGTGGCCGATG |
| | AGGTCGGACTGAGGAGCGTGATCGAGCAGTTTCCAGGGAAGCTGGACT |
| | TTGTGCTGGTGGATGGCGGATGCGTGCTGTCTCACGGCCATAAACAGCT |
| | GATGTGTCTGGCCCGGTCCGTGCTGTCTAAGGCCAAGATCCTGCTGCTG |
| | GACGAACCCTCCGCCCACCTGGACCCCGTGACATACCAGATCATCAGG |
| | AGAACTCTCAAGCAGGCCTTCGCCGACTGTACCGTGATTCTGTGCGAGC |
| | ACCGCATTGAAGCTATGCTGGAGTGTCAGCAGTTCCTGGTGATCGAGG |
| | AAAATAAGGTGAGGCAGTACGACAGCATCCAGAAGCTGCTGAACGAG |
| | CGCTCCCTGTTCCGCCAGGCTATCTCCCCATCAGACCGGGTGAAGCTCT |
| | TTCCCCACAGAAACTCCTCAAAGTGCAAGTCCAAGCCCCAGATCGCCG |
| | CCCTGAAGGAGGAGACCGAGGAGGAGGTGCAGGACACCAGGCTGTGA |
| | (SEQ ID NO: 52) |

In some embodiments, a suitable mRNA sequence may be an mRNA sequence encoding a homolog or an analog of human CFTR (hCFTR) protein. For example, a homolog or an analog of hCFTR protein may be a modified hCFTR protein containing one or more amino acid substitutions, deletions, and/or insertions as compared to a wild-type or naturally-occurring hCFTR protein while retaining substantial hCFTR protein activity. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 3, SEQ ID NO: 41, SEQ ID NO: 42, SEQ ID NO: 43, or SEQ ID NO: 44. In some embodiments, an mRNA suitable for the present invention encodes a protein substantially identical to an engineered hCFTR protein. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO: 41, SEQ ID NO: 42, SEQ ID NO: 43, or SEQ ID NO: 44. In some embodiments, an mRNA suitable for the present invention encodes a fragment or a portion of an engineered hCFTR protein. In some embodiments, an mRNA suitable for the present invention encodes a fragment or a portion of an engineered hCFTR protein, wherein the fragment or portion of the protein still maintains CFTR activity similar to that of the full-length engineered protein. In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 1, SEQ ID NO: 7, SEQ ID NO: 8, SEQ ID NO: 45, SEQ ID NO: 46, SEQ ID NO: 47, or SEQ ID NO: 48.

In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical to any one of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 or SEQ ID NO: 40.

In some embodiments, a suitable mRNA encodes a fusion protein comprising a full length, fragment or portion of an engineered hCFTR protein fused to another protein (e.g., an N or C terminal fusion). In some embodiments, the protein fused to the mRNA encoding a full length, fragment or portion of an engineered hCFTR protein encodes a signal or a cellular targeting sequence.

(SEQ ID NO: 21)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 22)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCTGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATTTCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTTGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 23)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACACACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTTGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 24)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCAACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 25)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 26)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATTTCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCAACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 27)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCTGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCCGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAGCCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 28)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACACACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 29)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCCGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAGTCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 30)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCTGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATTTCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACACACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 31)
ATGCAGAGAAGCCCCCTGGAGAAGGCCTCTGTGGTGAGCAAGCTGTTCTTCAGCTG
GACCAGACCCATCCTGAGAAAGGGCTACAGACAGAGACTGGAGCTGTCTGACATCT
ACCAGATCCCCTCTGTGGACTCTGCCGACAACCTGTCTGAGAAGCTGGAGAGAGAG
TGGGACAGAGAGCTGGCCAGCAAGAAGAACCCCAAGCTGATCAATGCCCTGAGAA
GATGCTTCTTCTGGAGATTCATGTTCTATGGCATCTTCCTGTACCTGGGAGAGGTGAC
CAAGGCCGTGCAGCCCCTGCTGCTGGGCAGGATCATTGCCAGCTATGACCCTGACA
ACAAGGAGGAGAGAAGCATTGCCATCTACCTGGGCATTGGCCTGTGCCTGCTGTTCA
TTGTGAGAACCCTGCTGCTGCACCCTGCCATCTTTGGCCTGCACCACATTGGCATGC
AGATGAGAATTGCCATGTTCAGCCTGATCTACAAGAAGACCCTGAAGCTGAGCAGC
AGAGTGCTGGACAAGATCAGCATTGGCCAGCTGGTGAGCCTGCTGAGCAACAACCT
GAACAAGTTTGATGAGGGCCTGGCCCTGGCCCACTTTGTGTGGATTGCCCCCCTGCA
GGTGGCCCTGCTGATGGGCCTGATCTGGGAGCTGCTGCAGGCCTCTGCCTTCTGTGG
CCTGGGCTTCCTGATTGTGCTGGCCCTGTTCCAGGCCGGCCTGGGCAGAATGATGAT
GAAGTACAGAGACCAGAGAGCCGGCAAGATCTCTGAGAGACTGGTGATCACCTCTG
AGATGATTGAGAACATCCAGTCTGTGAAGGCCTACTGCTGGGAGGAGGCCATGGAG
AAGATGATTGAGAACCTGAGACAGACAGAGCTGAAGCTGACCAGGAAGGCCGCCTA
TGTGAGATACTTCAACAGCTCTGCCTTCTTCTTCTCTGGCTTCTTTGTGGTGTTCCTGT
CTGTGCTGCCCTATGCCCTGATCAAGGGCATCATCCTGAGGAAGATCTTCACCACCA
TCAGCTTCTGCATTGTGCTGAGGATGGCCGTGACCAGGCAGTTCCCCTGGGCCGTGC
AGACCTGGTATGACAGCCTGGGGGCCATCAACAAGATCCAGGACTTCCTGCAGAAG
CAGGAGTACAAGACCCTGGAGTACAACCTGACCACCACAGAGGTGGTGATGGAGAA
TGTGACAGCCTTCTGGGAGGAGGGCTTTGGAGAGCTGTTTGAGAAGGCCAAGCAGA
ACAACAACAACAGAAAGACCAGCAATGGAGATGACAGCCTGTTCTTCAGCAACTTC
AGCCTGCTGGGCACCCCTGTGCTGAAGGACATCAACTTCAAGATTGAGAGGGGCCA
GCTGCTGGCCGTGGCCGGCAGCACAGGAGCCGGCAAGACCAGCCTGCTGATGGTGA
TCATGGGAGAGCTGGAGCCCTCTGAGGGCAAGATCAAGCACTCTGGCAGAATCAGC
TTCTGCAGCCAGTTCAGCTGGATCATGCCTGGCACCATCAAGGAGAACATCATCTTT
GGGGTGAGCTATGATGAGTACAGGTACAGATCTGTGATCAAGGCCTGCCAGCTGGA
GGAGGACATCTCCAAGTTTGCCGAGAAGGACAACATTGTGCTGGGGGAGGGAGGCA
TCACCCTGTCTGGGGGCCAGAGAGCCAGAATCAGCCTGGCCAGAGCCGTGTACAAG
GATGCCGACCTGTACCTGCTGGACAGCCCCTTTGGCTACCTGGATGTGCTGACAGAG
AAGGAGATCTTTGAGAGCTGTGTGTGCAAGCTGATGGCCAACAAGACCAGGATCCT
GGTGACCAGCAAGATGGAGCACCTGAAGAAGGCCGACAAGATCCTGATCCTGCATG
AGGGCAGCAGCTACTTCTATGGCACCTTCTCTGAGCTGCAGAACCTGCAGCCTGACT
TCAGCAGCAAGCTGATGGGCTGTGACAGCTTTGACCAGTTCTCTGCTGAGAGAAGA
AACAGCATCCTGACAGAGACCCTGCACAGGTTCAGCCTGGAGGGGGATGCCCCTGT
GAGCTGGACAGAGACCAAGAAGCAGAGCTTCAAGCAGACAGGAGAGTTTGGGGAG
AAGAGGAAGAACAGCATCCTGAACCCCATCAACAGCATCAGGAAGTTCAGCATTGT
GCAGAAGACCCCCCTGCAGATGAATGGCATTGAGGAGGACTCTGATGAGCCCCTGG
AGAGAAGACTGAGCCTGGTGCCAGACTCTGAGCAGGGAGAGGCCATCCTGCCCAGG
ATCTCTGTGATCAGCACAGGCCCCACCCTGCAGGCCAGAAGAAGACAGTCTGTGCT
GAACCTGATGACCCACTCTGTGAACCAGGGCCAGAATATCCACAGAAAGACCACAG
CCAGCACCAGAAAGGTGAGCCTGGCCCCCCAGGCCAACCTGACAGAGCTGGACATC
TACAGCAGAAGGCTGAGCCAGGAGACAGGCCTGGAGATCTCTGAGGAGATCAATGA
GGAGGACCTGAAGGAGTGCTTCTTTGATGACATGGAGAGCATCCCTGCCGTGACCA
CCTGGAACACCTACCTGAGATACATCACAGTGCACAAGAGCCTGATCTTTGTGCTGA
TCTGGTGCCTGGTGATCTTCCTGGCCGAGGTGGCCGCCAGCCTGGTGGTGCTGTGGC
TGCTGGGCAACACCCCCCTGCAGGACAAGGGCAACAGCACCCACAGCAGAAACAAC
AGCTATGCTGTGATCATCACCAGCACCAGCAGCTACTATGTGTTCTACATCTATGTG
GGAGTGGCTGACACCCTGCTGGCCATGGGCTTCTTCAGAGGCCTGCCCCTGGTGCAC
ACCCTGATCACAGTGAGCAAGATCCTGCACCACAAGATGCTGCACTCTGTGCTGCAG
GCCCCCATGAGCACCCTGAACACCCTGAAGGCTGGAGGCATCCTGAACAGATTCAG
CAAGGACATTGCCATCCTGGATGACCTGCTGCCCCTGACCATCTTTGACTTCATCCA
GCTGCTGCTGATTGTGATTGGAGCCATTGCCGTGGTGGCCGTGCTGCAGCCCTACAT
CTTTGTGGCCACAGTGCCTGTGATTGTGGCCTTCATCATGCTGAGGGCCTACTTCCTG
CAGACCAGCCAGCAGCTGAAGCAGCTGGAGTCTGAGGGCAGAAGCCCCATCTTCAC
CCACCTGGTGACCAGCCTGAAGGGCCTGTGGACCCTGAGGGCCTTTGGCAGACAGC
CCTACTTTGAGACCCTGTTCCACAAGGCCCTGAACCTGCACACAGCCAACTGGTTCC
TGTACCTGAGCACCCTGAGATGGTTCCAGATGAGGATTGAGATGATCTTTGTGATCT
TCTTCATTGCCGTGACCTTCATCAGCATCCTGACCACAGGGGAGGGCGAGGGCAGA
GTGGGCATCATCCTGACCCTGGCCATGAACATCATGAGCACCCTGCAGTGGGCCGTG
AACAGCAGCATTGATGTGGACAGCCTGATGAGATCTGTGAGCAGAGTGTTCAAGTT
CATTGACATGCCCACAGAGGGCAAGCCCACCAAGAGCACCAAGCCCTACAAGAATG
GCCAGCTGAGCAAGGTGATGATCATTGAGAACAGCCATGTGAAGAAGGATGACATC
TGGCCCTCTGGAGGCCAGATGACAGTGAAGGACCTGACAGCCAAGTACACAGAGGG
GGGCAATGCCATCCTGGAGAACATCAGCTTCAGCATCAGCCCTGGCCAGAGGGTGG
GCCTGCTGGGCAGAACAGGCTCTGGCAAGAGCACCCTGCTGTCTGCCTTCCTGAGGC
TGCTGAACACAGAGGGAGAGATCCAGATTGATGGGGTGAGCTGGGACAGCATCACC
CTGCAGCAGTGGAGGAAGGCCTTTGGGGTGATCCCCCAGAAGGTGTTCATCTTCTCT
GGCACCTTCAGGAAGAACCTGGACCCCTATGAGCAGTGGTCTGACCAGGAGATCTG
GAAGGTGGCCGATGAGGTGGGCCTGAGATCTGTGATTGAGCAGTTCCCTGGCAAGC
TGGACTTTGTGCTGGTGGATGGAGGCTGTGTGCTGAGCCATGGCCACAAGCAGCTGA
TGTGCCTGGCCAGATCTGTGCTGAGCAAGGCCAAGATCCTGCTGCTGGATGAGCCCT
CTGCCCACCTGGACCCTGTGACCTACCAGATCATCAGAAGAACCCTGAAGCAGGCC
TTTGCCGACTGCACAGTGATCCTGTGTGAGCACAGAATTGAGGCCATGCTGGAGTGC
CAGCAGTTCCTGGTGATTGAGGAGAACAAGGTGAGGCAGTATGACAGCATCCAGAA
GCTGCTGAATGAGAGAAGCCTGTTCAGACAGGCCATCAGCCCCTCTGACAGAGTGA
AGCTGTTCCCCCACAGGAACAGCAGCAAGTGCAAGAGCAAGCCCCAGATTGCCGCC
CTGAAGGAGGAGACAGAGGAGGAGGTGCAGGACACCAGACTGTGA
(SEQ ID NO: 32)
ATGCAGAGGAGCCCCCTGGAGAAGGCCAGCGTGGTGAGCAAGCTGTTCTTCAGCTG
GACCAGGCCCATCCTGAGGAAGGGCTACAGGCAGAGGCTGGAGCTGAGCGACATCT
ACCAGATCCCCAGCGTGGACAGCGCCGACAACCTGAGCGAGAAGCTGGAGAGGGA
GTGGGACAGGGAGCTGGCCAGCAAGAAGAACCCCAAGCTGATCAACGCCCTGAGG
AGGTGCTTCTTCTGGAGGTTCATGTTCTACGGCATCTTCCTGTACCTGGGCGAGGTG
ACCAAGGCCGTGCAGCCCCTGCTGCTGGGCAGGATCATCGCCAGCTACGACCCCGA
CAACAAGGAGGAGAGGAGCATCGCCATCTACCTGGGCATCGGCCTGTGCCTGCTGT
TCATCGTGAGGACCCTGCTGCTGCACCCCGCCATCTTCGGCCTGCACCACATCGGCA
TGCAGATGAGGATCGCCATGTTCAGCCTGATCTACAAGAAGACCCTGAAGCTGAGC
AGCAGGGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGGATGAT
GATGAAGTACAGGGACCAGAGGGCCGGCAAGATCAGCGAGAGGCTGGTGATCACC
AGCGAGATGATCGAGAACATCCAGAGCGTGAAGGCCTACTGCTGGGAGGAGGCCAT
GGAGAAGATGATCGAGAACCTGAGGCAGACCGAGCTGAAGCTGACCAGGAAGGCC
GCCTACGTGAGGTACTTCAACAGCAGCGCCTTCTTCTTCAGCGGCTTCTTCGTGGTGT
TCCTGAGCGTGCTGCCCTACGCCCTGATCAAGGGCATCATCCTGAGGAAGATCTTCA
CCACCATCAGCTTCTGCATCGTGCTGAGGATGGCCGTGACCAGGCAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGGAAGACCAGCAACGGCGACGACAGCCTGTTCTTCAGC
AACTTCAGCCTGCTGGGCACCCCCGTGCTGAAGGACATCAACTTCAAGATCGAGAG
GGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
GATCAGCTTCTGCAGCCAGTTCAGCTGGATCATGCCCGGCACCATCAAGGAGAACA
TCATCTTCGGCGTGAGCTACGACGAGTACAGGTACAGGAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGGGCCAGGATCAGCCTGGCCAGGGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGGATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGGAGGAACAGCATCCTGACCGAGACCCTGCACAGGTTCAGCCTGGAGGGCGA
CGCCCCCGTGAGCTGGACCGAGACCAAGAAGCAGAGCTTCAAGCAGACCGGCGAGT
TCGGCGAGAAGAGGAAGAACAGCATCCTGAACCCCATCAACAGCATCAGGAAGTTC
AGCATCGTGCAGAAGACCCCCCTGCAGATGAACGGCATCGAGGAGGACAGCGACG
AGCCCCTGGAGAGGAGGCTGAGCCTGGTGCCCGACAGCGAGCAGGGCGAGGCCATC
CTGCCCAGGATCAGCGTGATCAGCACCGGCCCCACCCTGCAGGCCAGGAGGAGGCA
GAGCGTGCTGAACCTGATGACCCACAGCGTGAACCAGGGCCAGAACATCCACAGGA
AGACCACCGCCAGCACCAGGAAGGTGAGCCTGGCCCCCCAGGCCAACCTGACCGAG
CTGGACATCTACAGCAGGAGGCTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGGTACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGGAACAACAGCTACGCCGTGATCATCACCAGCACCAGCAGCTACTACGTGTTC
TACATCTACGTGGGCGTGGCCGACACCCTGCTGGCCATGGGCTTCTTCAGGGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGGTTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
GGCCTACTTCCTGCAGACCAGCCAGCAGCTGAAGCAGCTGGAGAGCGAGGGCAGGA
GCCCCATCTTCACCCACCTGGTGACCAGCCTGAAGGGCCTGTGGACCCTGAGGGCCT
TCGGCAGGCAGCCCTACTTCGAGACCCTGTTCCACAAGGCCCTGAACCTGCACACCG
CCAACTGGTTCCTGTACCTGAGCACCCTGAGGTGGTTCCAGATGAGGATCGAGATGA
TCTTCGTGATCTTCTTCATCGCCGTGACCTTCATCAGCATCCTGACCACCGGCGAGG
GCGAGGGCAGGGTGGGCATCATCCTGACCCTGGCCATGAACATCATGAGCACCCTG
CAGTGGGCCGTGAACAGCAGCATCGACGTGGACAGCCTGATGAGGAGCGTGAGCAG
GGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGGGTGGGCCTGCTGGGCAGGACCGGCAGCGGCAAGAGCACCCTGCTGAGC
GCCTTCCTGAGGCTGCTGAACACCGAGGGCGAGATCCAGATCGACGGCGTGAGCTG
GGACAGCATCACCCTGCAGCAGTGGAGGAAGGCCTTCGGCGTGATCCCCCAGAAGG
TGTTCATCTTCAGCGGCACCTTCAGGAAGAACCTGGACCCCTACGAGCAGTGGAGC
GACCAGGAGATCTGGAAGGTGGCCGACGAGGTGGGCCTGAGGAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGGAGCGTGCTGAGCAAGGCCAAGATCCT
GCTGCTGGACGAGCCCAGCGCCCACCTGGACCCCGTGACCTACCAGATCATCAGGA
GGACCCTGAAGCAGGCCTTCGCCGACTGCACCGTGATCCTGTGCGAGCACAGGATC
GAGGCCATGCTGGAGTGCCAGCAGTTCCTGGTGATCGAGGAGAACAAGGTGAGGCA
GTACGACAGCATCCAGAAGCTGCTGAACGAGAGGAGCCTGTTCAGGCAGGCCATCA
GCCCCAGCGACAGGGTGAAGCTGTTCCCCCACAGGAACAGCAGCAAGTGCAAGAGC
AAGCCCCAGATCGCCGCCCTGAAGGAGGAGACCGAGGAGGAGGTGCAGGACACCA
GGCTGTGA
(SEQ ID NO: 33)
ATGCAGAGATCCCCTCTGGAGAAGGCCTCAGTGGTGTCCAAGCTTTTCTTCTCCTGG
ACCAGGCCCATTTTAAGAAAGGGCTACAGGCAGAGACTTGAGCTGTCTGACATCTAT
CAGATCCCTTCTGTGGATTCTGCTGACAATCTTAGTGAAAAATTGGAAAGGGAGTGG
GACAGAGAGCTGGCAAGTAAAAAGAACCCCAAGCTGATTAATGCCCTGAGGCGCTG
CTTTTTTTGGAGATTCATGTTCTATGGCATATTCCTCTACCTTGGAGAAGTAACCAAA
GCTGTACAGCCTCTCCTCCTTGGCAGAATCATTGCCTCCTATGATCCTGATAACAAG
GAGGAGAGAAGCATAGCCATCTACCTGGGCATTGGGCTGTGCCTCTTGTTTATTGTG
AGGACCCTTCTCTTGCACCCTGCCATCTTTGGCCTTCATCACATTGGCATGCAAATGA
GAATAGCAATGTTTAGTCTTATTTACAAAAAAACATTAAAACTCTCTTCCAGGGTGT
TGGACAAGATCAGTATTGGACAACTGGTCAGCCTGCTGAGCAACAACCTGAACAAG
TTTGATGAAGGACTGGCCCTGGCCCACTTTGTCTGGATTGCCCCCCTTCAGGTGGCTC
TTTTGATGGGCCTGATCTGGGAACTCCTGCAGGCCTCTGCCTTCTGTGGGTTAGGCTT
CCTGATAGTGCTAGCTCTCTTTCAGGCAGGGTTGGGTAGAATGATGATGAAGTACAG
AGACCAGAGGGCTGGGAAGATATCTGAGAGGCTGGTCATTACTTCTGAAATGATAG
AAAACATCCAGTCTGTTAAAGCTTACTGCTGGGAGGAGGCTATGGAAAAGATGATT
GAGAACTTGAGGCAAACAGAGCTCAAGCTGACTAGGAAGGCAGCCTATGTCAGGTA
TTTCAACAGCAGTGCTTTCTTCTTCTCAGGCTTTTTCGTGGTCTTCTTGAGTGTTCTGC
CCTATGCCCTCATCAAGGGGATAATTTTGAGAAAGATTTTCACCACTATTTCCTTTTG
CATTGTCCTGAGGATGGCTGTCACCAGGCAATTCCCCTGGGCTGTGCAGACATGGTA
TGACTCTCTGGGGGCCATCAACAAAATCCAAGATTTCCTGCAGAAGCAGGAGTACA
AGACCCTGGAATACAACCTCACCACCACAGAAGTTGTGATGGAGAATGTGACTGCA
TTCTGGGAGGAAGGATTTGGGGAGCTGTTTGAGAAAGCAAAACAAAACAATAATAA
CAGGAAAACCAGCAATGGAGATGACTCCCTGTTCTTTTCCAACTTCTCTTTGTTGGG
CACCCCTGTCCTGAAAGATATAAACTTTAAAATTGAAAGAGGGCAGCTGTTGGCAGT
TGCTGGCTCCACAGGAGCTGGAAAAACTTCACTACTGATGGTGATCATGGGGGAGTT
AGAACCCTCTGAAGGGAAAATAAAACATTCTGGGAGGATTAGTTTCTGCAGCCAGT
TCAGCTGGATCATGCCTGGGACCATTAAAGAAAATATTATATTTGGAGTGAGCTATG
ATGAATATAGATATAGGAGTGTCATCAAAGCCTGTCAGTTGGAGGAAGACATCAGC
AAATTTGCAGAGAAAGACAACATTGTTCTGGGTGAAGGTGGCATCACCCTGTCAGG
AGGGCAAAGGGCCAGGATCAGCTTGGCCAGAGCAGTCTATAAAGATGCTGATCTGT
ACCTCCTGGATAGCCCTTTTGGCTATCTGGATGTTTTGACAGAGAAGGAAATTTTTG
AGTCCTGTGTCTGCAAGTTAATGGCAAATAAAACAAGGATACTTGTGACCTCAAAA
ATGGAACACCTGAAGAAGGCTGACAAAATTCTGATCCTGCATGAGGGCAGCAGCTA
CTTTTATGGAACATTTTCTGAACTGCAGAATTTGCAACCAGACTTTTCATCAAAGCTC
ATGGGATGTGACAGTTTTGATCAGTTTTCTGCAGAAAGGAGAAACTCCATTTTGACT
GAGACCCTGCACAGGTTCAGTCTGGAGGGGGATGCCCCAGTGAGTTGGACTGAGAC
AAAGAAACAGAGCTTCAAGCAGACTGGAGAGTTTGGAGAAAAGAGGAAAAACTCA
ATTCTCAATCCCATCAATAGCATCAGGAAGTTCAGCATAGTTCAGAAGACTCCTTTG
CAGATGAATGGGATTGAAGAGGACTCAGATGAGCCCCTGGAAAGGAGACTCTCCTT
GGTGCCAGATTCAGAGCAGGGGGAAGCCATACTGCCAAGGATCTCTGTGATTTCTAC
AGGGCCCACCCTCCAAGCAAGAAGGAGACAGTCAGTTTTAAACCTGATGACCCACT
CTGTCAACCAGGGACAGAACATTCATAGAAAGACAACAGCATCTACAAGAAAAGTT
TCACTGGCCCCTCAAGCCAATTTAACTGAACTAGATATCTACAGCAGGAGGCTCAGC
CAAGAAACAGGCCTGGAGATCTCAGAAGAAATAAATGAGGAGGATTTGAAGGAAT
GCTTCTTTGATGATATGGAGAGCATCCCAGCTGTCACAACCTGGAACACCTACCTGA
GATACATCACAGTGCACAAATCCCTCATCTTTGTACTTATATGGTGCCTTGTCATCTT
CTTAGCTGAGGTGGCTGCTTCCCTGGTGGTGCTGTGGCTGCTGGGAAACACACCCCT
CCAGGATAAAGGGAACTCTACTCACAGCAGGAACAACAGTTATGCTGTGATCATCA
CCAGTACCTCCTCCTACTATGTGTTCTACATTTATGTTGGAGTTGCAGACACATTGCT
TGCCATGGGTTTTTTTAGAGGACTCCCCCTGGTGCATACTCTCATCACTGTTTCCAAA
ATCCTTCACCACAAGATGCTGCACAGTGTACTACAGGCTCCCATGAGCACCCTCAAC
ACTCTTAAAGCAGGAGGAATCTTGAACAGATTTAGCAAGGACATTGCAATTCTTGAT
GACCTGCTTCCACTGACCATCTTTGACTTCATCCAGCTTCTGCTCATTGTAATTGGTG
CCATTGCTGTGGTAGCAGTGCTCCAGCCATATATTTTTGTGGCCACTGTGCCTGTTAT
TGTGGCCTTCATTATGTTGAGAGCCTACTTCCTGCAGACCTCTCAGCAGCTCAAGCA
ACTTGAAAGTGAGGGCAGGAGCCCCATATTTACACACTTGGTCACTTCCCTCAAAGG
CCTCTGGACACTCAGAGCTTTTGGAAGACAACCTTATTTTGAAACTCTCTTCCACAA
GGCTCTGAATCTCCACACAGCCAACTGGTTTCTGTATCTTTCAACACTGCGCTGGTTC
CAGATGAGGATTGAGATGATCTTTGTTATCTTCTTCATAGCTGTTACCTTCATCTCTA
TTCTGACAACTGGTGAGGGGGAAGGGAGAGTAGGCATCATCCTCACACTAGCCATG
AACATAATGTCTACCTTACAATGGGCCGTGAACAGCTCCATAGATGTGGACAGCCTC
ATGAGAAGTGTGTCAAGAGTTTTCAAATTCATTGACATGCCCACAGAAGGCAAACC
AACCAAGAGCACAAAACCCTACAAGAATGGCCAGCTGAGTAAGGTCATGATCATTG
AAAATTCTCATGTGAAGAAGGATGATATTTGGCCCAGTGGGGGCCAGATGACAGTC
AAGGACCTCACTGCCAAATACACAGAGGGTGGAAATGCTATCCTAGAGAACATCTC
CTTCTCCATCTCCCCAGGCCAAAGAGTTGGCTTGCTGGGCAGGACTGGCAGTGGCAA
GTCCACCTTGCTCTCAGCATTTCTCAGGCTTTTAAATACAGAGGGAGAGATTCAAAT
TGATGGGGTGTCTTGGGATAGTATAACACTTCAACAGTGGAGGAAAGCCTTTGGTGT
GATTCCTCAGAAAGTGTTTATCTTCTCTGGCACTTTCAGAAAAAATCTGGACCCCTAT
GAACAGTGGAGTGACCAGGAAATCTGGAAGGTGGCAGATGAAGTGGGCCTAAGATC
AGTCATAGAGCAGTTTCCTGGAAAGTTGGATTTTGTGCTTGTAGATGGAGGCTGTGT
GCTGTCCCATGGCCATAAACAGCTAATGTGCCTGGCTAGGTCAGTGCTGAGCAAGGC
CAAGATCCTGCTGTTAGATGAGCCTTCAGCCCATCTGGACCCTGTGACATACCAGAT
TATCAGAAGAACTCTGAAGCAGGCCTTTGCTGACTGCACTGTCATCCTGTGTGAGCA
CAGAATTGAGGCCATGCTGGAGTGCCAGCAGTTCCTTGTTATAGAAGAGAATAAGG
TTAGGCAGTATGACAGCATTCAGAAACTGCTAAATGAAAGATCTCTCTTCAGGCAAG
CTATTTCACCATCTGATAGAGTGAAACTTTTTCCCCACAGAAATTCCTCTAAATGTAA
ATCTAAGCCCCAGATAGCTGCCTTGAAAGAGGAGACTGAAGAAGAAGTCCAGGACA
CCAGACTGTGA
(SEQ ID NO: 34)
ATGCAGAGATCCCCGCTGGAGAAGGCATCTGTGGTGTCAAAACTGTTCTTTAGCTGG
ACAAGGCCCATCCTTAGGAAAGGGTACAGACAGAGGTTGGAGCTGTCAGACATATA
TCAGATCCCTTCAGTGGACTCTGCAGACAACCTCTCTGAAAAGCTGGAGAGGGAAT
GGGACAGGGAACTGGCCAGCAAAAAAAACCCTAAACTGATTAATGCCCTGAGGAGG
TGCTTCTTTTGGAGATTCATGTTCTATGGGATCTTCCTTTACCTGGGGGAGGTGACTA
AAGCTGTTCAGCCTCTTCTTCTGGGGAGGATTATTGCCTCCTATGACCCAGACAACA
AAGAAGAAAGAAGCATAGCCATTTACTTAGGCATAGGCCTCTGCTTGCTCTTCATAG
TTAGAACCCTCCTACTCCACCCAGCCATCTTTGGTCTCCACCACATAGGTATGCAGA
TGAGAATAGCAATGTTCTCCTTGATCTACAAGAAGACCCTCAAGCTGTCCAGCAGGG
TGCTGGACAAGATCTCCATAGGCCAGTTAGTCAGTCTACTGTCCAATAACTTAAATA
AGTTTGATGAGGGACTGGCACTGGCACATTTTGTGTGGATTGCCCCCCTCCAAGTGG
CCCTTCTTATGGGCCTTATCTGGGAGCTGTTGCAGGCCTCTGCTTTCTGTGGCCTGGG
TTTCCTCATAGTCCTAGCCTTATTCCAGGCTGGACTGGGCAGAATGATGATGAAGTA
TAGGGACCAAAGAGCAGGGAAGATTTCTGAAAGGCTGGTTATAACTTCTGAGATGA
TTGAGAACATTCAGTCAGTGAAAGCTTACTGCTGGGAAGAAGCTATGGAAAAAATG
ATTGAAAATCTCAGACAGACTGAATTAAAGTTGACCAGGAAAGCTGCTTATGTCAG
ATACTTCAACTCCTCAGCCTTCTTTTTTTCTGGCTTCTTTGTTGTATTCCTTTCAGTCC
TCCCCTATGCCCTGATTAAGGGCATTATCTTGAGGAAAATTTTCACAACCATCTCCTT
TTGTATTGTCCTCAGGATGGCTGTTACAAGGCAATTTCCTTGGGCTGTGCAAACTTG
GTATGATAGCCTTGGAGCAATCAACAAGATCCAGGATTTCCTGCAAAAGCAGGAGT
ACAAGACATTGGAATACAACCTTACCACCACTGAGGTGGTGATGGAAAATGTGACT
GCCTTCTGGGAGGAGGGGTTTGGAGAGCTGTTTGAGAAAGCCAAACAGAACAACAA
CAATAGAAAGACCTCTAATGGTGATGATTCCCTGTTCTTTTCTAACTTTAGTCTTCTG
GGGACCCCAGTTCTGAAAGATATTAACTTTAAAATTGAAAGGGGACAGTTGCTGGCT
GTGGCTGGGTCCACTGGGGCTGGGAAGACAAGCCTGCTCATGGTGATCATGGGAGA
GCTGGAACCCAGTGAAGGAAAGATCAAACACTCAGGCAGGATCTCCTTCTGCAGCC
AGTTCTCATGGATTATGCCAGGCACTATTAAAGAAAATATCATCTTTGGTGTAAGCT
ATGATGAGTACAGGTATAGATCTGTAATTAAAGCCTGCCAGCTGGAGGAAGACATC
TCTAAGTTTGCTGAGAAGGATAACATTGTGTTGGGGGAAGGGGGCATCACCCTTTCT
GGTGGGCAGAGGGCTAGGATCTCCCTTGCTAGGGCAGTATACAAGGATGCTGACTT
GTACCTCTTGGATAGTCCTTTTGGCTACCTAGATGTGCTGACAGAGAAAGAAATATT
TGAAAGCTGTGTGTGTAAGCTCATGGCTAACAAGACCAGGATCCTGGTCACCAGTA
AAATGGAACACCTCAAAAAAGCAGACAAGATCCTTATTCTCCATGAGGGCTCCTCCT
ACTTCTATGGGACCTTCAGTGAGCTGCAGAATCTGCAGCCAGACTTCTCCTCAAAAC
TTATGGGCTGTGACTCCTTTGACCAATTCTCTGCAGAAAGAAGGAATAGCATACTGA
CAGAAACACTGCATAGATTCTCCCTGGAAGGAGATGCCCCAGTGAGTTGGACAGAA
ACCAAAAAGCAGAGCTTCAAGCAGACTGGTGAGTTTGGTGAAAAGAGGAAGAATTC
TATCCTGAACCCCATCAATAGCATCAGGAAATTTAGCATAGTCCAAAAGACCCCCCT
CCAGATGAATGGAATAGAGGAGGATAGTGATGAGCCTCTTGAGAGAAGGCTGTCCC
TGGTTCCAGACAGTGAACAGGGTGAAGCCATTCTTCCGAGGATCAGTGTCATCTCCA
CTGGGCCCACATTGCAGGCCAGAAGAAGACAGTCTGTTCTGAATTTGATGACACATT
CTGTGAATCAAGGCCAGAATATCCATAGAAAAACCACTGCCAGCACCAGAAAAGTT
TCTCTAGCCCCCCAGGCTAACCTGACTGAGTTAGACATCTACAGCAGAAGGCTGAGC
CAAGAGACTGGCTTGGAAATATCTGAGGAGATCAATGAGGAGGACCTCAAGGAGTG
CTTCTTTGATGACATGGAGTCAATCCCTGCAGTCACTACATGGAACACTTACCTAAG
GTACATCACAGTTCATAAGAGCCTCATCTTTGTCCTCATATGGTGTCTGGTCATCTTT
TTAGCAGAAGTGGCTGCCAGCCTAGTTGTGCTGTGGTTACTGGGCAATACACCTCTT
CAGGACAAAGGCAATAGCACACACAGCAGAAACAACTCCTATGCAGTGATCATCAC
CTCTACAAGCTCTTACTATGTATTCTATATATATGTGGGAGTGGCAGATACTCTCCTG
GCCATGGGATTCTTCAGGGGATTACCTCTAGTTCACACATTGATCACAGTGTCAAAA
ATTCTCCACCACAAGATGTTACACAGTGTCCTGCAAGCCCCAATGTCTACTCTGAAC
ACACTTAAGGCAGGTGGAATTTTGAATAGGTTTAGCAAGGACATAGCTATCCTGGAT
GATCTCCTCCCTCTGACCATCTTTGACTTCATCCAGTTACTGCTCATTGTAATTGGAG
CCATTGCAGTGGTAGCAGTCCTACAGCCTTACATTTTTGTGGCTACTGTTCCTGTTAT
TGTGGCCTTCATTATGCTAAGAGCTTACTTCCTGCAAACAAGCCAACAGTTGAAACA
GCTAGAAAGTGAGGGAAGGTCCCCCATCTTCACCCACCTGGTGACATCACTCAAGG
GGCTATGGACTCTTAGGGCTTTTGGGAGACAGCCGTACTTTGAGACCTTATTCCATA
AGGCCCTTAACCTCCATACAGCAAACTGGTTCTTATACCTGAGTACTCTGAGGTGGT
TTCAAATGAGGATTGAAATGATTTTTGTGATCTTCTTCATTGCTGTGACCTTCATCTC
AATCTTGACCACAGGAGAGGGGGAGGGCAGGGTGGGCATCATACTGACCTTGGCCA
TGAACATTATGTCAACCCTGCAGTGGGCTGTCAATAGCTCCATTGATGTGGACAGTC
TGATGAGGAGTGTCTCCAGGGTCTTCAAGTTTATTGACATGCCAACTGAGGGCAAAC
CCACCAAAAGCACTAAGCCATATAAAAATGGCCAACTGTCCAAAGTGATGATCATT
GAAAATTCACATGTAAAGAAGGATGATATCTGGCCCTCTGGAGGACAGATGACAGT
GAAAGACCTGACTGCCAAGTACACAGAGGGTGGTAATGCCATTCTTGAGAACATTA
GTTTCAGTATTTCCCCGGGGCAAAGGGTGGGCCTCCTTGGCAGAACAGGCTCTGGCA
AGAGTACCCTGCTGTCAGCCTTTTTAAGACTGTTGAACACTGAGGGAGAAATTCAGA
TTGATGGTGTCTCCTGGGATAGCATCACCCTCCAGCAGTGGAGAAAAGCTTTTGGAG
TGATCCCGCAAAAGGTTTTCATCTTTTCAGGCACCTTCCGGAAGAACCTGGACCCCT
ATGAGCAGTGGTCTGACCAGGAAATATGGAAGGTAGCTGATGAAGTTGGGCTTAGG
TCAGTCATAGAGCAGTTCCCAGGCAAACTGGACTTTGTCCTGGTGGATGGTGGATGT
GTACTGAGTCATGGGCACAAACAGCTGATGTGCCTAGCCAGGTCTGTGCTCAGCAA
GGCAAAGATATTGCTGCTTGATGAACCCAGTGCCCATCTGGACCCAGTCACATATCA
GATCATCAGAAGAACATTGAAGCAGGCCTTTGCTGATTGCACAGTTATCCTCTGTGA
GCACAGGATTGAGGCCATGCTGGAGTGCCAGCAGTTTCTGGTGATTGAGGAGAATA
AAGTAAGGCAGTATGACTCCATCCAGAAGCTGCTCAATGAAAGAAGCCTCTTTAGA
CAAGCTATCTCCCCCTCAGACAGGGTCAAATTGTTCCCTCACAGAAACAGCAGCAA
GTGCAAGAGCAAGCCCCAAATTGCAGCCTTGAAAGAGGAGACAGAGGAAGAGGTG
CAGGACACCAGACTCTGA
(SEQ ID NO: 35)
ATGCAGAGAAGCCCCCTGGAGAAGGCCAGCGTGGTGAGCAAGCTGTTCTTCAGCTG
GACCAGACCCATCCTGAGAAAGGGCTACAGACAGAGACTGGAGCTGAGCGACATCT
ACCAGATCCCCAGCGTGGACAGCGCCGACAACCTGAGCGAGAAGCTGGAGAGAGA
GTGGGACAGAGAGCTGGCCAGCAAGAAGAACCCCAAGCTGATCAACGCCCTGAGA
AGATGCTTCTTCTGGAGATTCATGTTCTACGGCATCTTCCTGTACCTGGGCGAGGTG
ACCAAGGCCGTGCAGCCCCTGCTGCTGGGCAGAATCATCGCCAGCTACGACCCCGA
CAACAAGGAGGAGAGAAGCATCGCCATCTACCTGGGCATCGGCCTGTGCCTGCTGT
TCATCGTGAGAACCCTGCTGCTGCACCCCGCCATCTTCGGCCTGCACCACATCGGCA
TGCAGATGAGAATCGCCATGTTCAGCCTGATCTACAAGAAGACCCTGAAGCTGAGC
AGCAGAGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGAATGAT
GATGAAGTACAGAGACCAGAGAGCCGGCAAGATCAGCGAGAGACTGGTGATCACC
AGCGAGATGATCGAGAACATCCAGAGCGTGAAGGCCTACTGCTGGGAGGAGGCCAT
GGAGAAGATGATCGAGAACCTGAGACAGACCGAGCTGAAGCTGACCAGAAAGGCC
GCCTACGTGAGATACTTCAACAGCAGCGCCTTCTTCTTCAGCGGCTTCTTCGTGGTGT
TCCTGAGCGTGCTGCCCTACGCCCTGATCAAGGGCATCATCCTGAGAAAGATCTTCA
CCACCATCAGCTTCTGCATCGTGCTGAGAATGGCCGTGACCAGACAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGAAAGACCAGCAACGGCGACGACAGCCTGTTCTTCAGC
AACTTCAGCCTGCTGGGCACCCCCGTGCTGAAGGACATCAACTTCAAGATCGAGAG
AGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
AATCAGCTTCTGCAGCCAGTTCAGCTGGATCATGCCCGGCACCATCAAGGAGAACA
TCATCTTCGGCGTGAGCTACGACGAGTACAGATACAGAAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGAGCCAGAATCAGCCTGGCCAGAGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGAATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGAAGAAACAGCATCCTGACCGAGACCCTGCACAGATTCAGCCTGGAGGGCGA
CGCCCCCGTGAGCTGGACCGAGACCAAGAAGCAGAGCTTCAAGCAGACCGGCGAGT
TCGGCGAGAAGAGAAAGAACAGCATCCTGAACCCCATCAACAGCATCAGAAAGTTC
AGCATCGTGCAGAAGACCCCCCTGCAGATGAACGGCATCGAGGAGGACAGCGACG
AGCCCCTGGAGAGAAGACTGAGCCTGGTGCCCGACAGCGAGCAGGGCGAGGCCATC
CTGCCCAGAATCAGCGTGATCAGCACCGGCCCCACCCTGCAGGCCAGAAGAAGACA
GAGCGTGCTGAACCTGATGACCCACAGCGTGAACCAGGGCCAGAACATCCACAGAA
AGACCACCGCCAGCACCAGAAAGGTGAGCCTGGCCCCCCAGGCCAACCTGACCGAG
CTGGACATCTACAGCAGAAGACTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGATACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGAAACAACAGCTACGCCGTGATCATCACCAGCACCAGCAGCTACTACGTGTTC
TACATCTACGTGGGCGTGGCCGACACCCTGCTGGCCATGGGCTTCTTCAGAGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGATTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
AGCCTACTTCCTGCAGACCAGCCAGCAGCTGAAGCAGCTGGAGAGCGAGGGCAGAA
GCCCCATCTTCACCCACCTGGTGACCAGCCTGAAGGGCCTGTGGACCCTGAGAGCCT
TCGGCAGACAGCCCTACTTCGAGACCCTGTTCCACAAGGCCCTGAACCTGCACACCG
CCAACTGGTTCCTGTACCTGAGCACCCTGAGATGGTTCCAGATGAGAATCGAGATGA
TCTTCGTGATCTTCTTCATCGCCGTGACCTTCATCAGCATCCTGACCACCGGCGAGG
GCGAGGGCAGAGTGGGCATCATCCTGACCCTGGCCATGAACATCATGAGCACCCTG
CAGTGGGCCGTGAACAGCAGCATCGACGTGGACAGCCTGATGAGAAGCGTGAGCAG
AGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGAGTGGGCCTGCTGGGCAGAACCGGCAGCGGCAAGAGCACCCTGCTGAGC
GCCTTCCTGAGACTGCTGAACACCGAGGGCGAGATCCAGATCGACGGCGTGAGCTG
GGACAGCATCACCCTGCAGCAGTGGAGAAAGGCCTTCGGCGTGATCCCCCAGAAGG
TGTTCATCTTCAGCGGCACCTTCAGAAAGAACCTGGACCCCTACGAGCAGTGGAGC
GACCAGGAGATCTGGAAGGTGGCCGACGAGGTGGGCCTGAGAAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGAAGCGTGCTGAGCAAGGCCAAGATCCT
GCTGCTGGACGAGCCCAGCGCCCACCTGGACCCCGTGACCTACCAGATCATCAGAA
GAACCCTGAAGCAGGCCTTCGCCGACTGCACCGTGATCCTGTGCGAGCACAGAATC
GAGGCCATGCTGGAGTGCCAGCAGTTCCTGGTGATCGAGGAGAACAAGGTGAGACA
GTACGACAGCATCCAGAAGCTGCTGAACGAGAGAAGCCTGTTCAGACAGGCCATCA
GCCCCAGCGACAGAGTGAAGCTGTTCCCCCACAGAAACAGCAGCAAGTGCAAGAGC
AAGCCCCAGATCGCCGCCCTGAAGGAGGAGACCGAGGAGGAGGTGCAGGACACCA
GACTGTGA
(SEQ ID NO: 36)
ATGCAGCGCAGCCCCCTGGAGAAGGCCAGCGTGGTGAGCAAGCTGTTCTTCAGCTG
GACCCGCCCCATCCTGCGCAAGGGCTACCGCCAGCGCCTGGAGCTGAGCGACATCT
ACCAGATCCCCAGCGTGGACAGCGCCGACAACCTGAGCGAGAAGCTGGAGCGCGA
GTGGGACCGCGAGCTGGCCAGCAAGAAGAACCCCAAGCTGATCAACGCCCTGCGCC
GCTGCTTCTTCTGGCGCTTCATGTTCTACGGCATCTTCCTGTACCTGGGCGAGGTGAC
CAAGGCCGTGCAGCCCCTGCTGCTGGGCCGCATCATCGCCAGCTACGACCCCGACA
ACAAGGAGGAGCGCAGCATCGCCATCTACCTGGGCATCGGCCTGTGCCTGCTGTTCA
TCGTGCGCACCCTGCTGCTGCACCCCGCCATCTTCGGCCTGCACCACATCGGCATGC
AGATGCGCATCGCCATGTTCAGCCTGATCTACAAGAAGACCCTGAAGCTGAGCAGC
CGCGTGCTGGACAAGATCAGCATCGGCCAGCTGGTGAGCCTGCTGAGCAACAACCT
GAACAAGTTCGACGAGGGCCTGGCCCTGGCCCACTTCGTGTGGATCGCCCCCCTGCA
GGTGGCCCTGCTGATGGGCCTGATCTGGGAGCTGCTGCAGGCCAGCGCCTTCTGCGG
CCTGGGCTTCCTGATCGTGCTGGCCCTGTTCCAGGCCGGCCTGGGCCGCATGATGAT
GAAGTACCGCGACCAGCGCGCCGGCAAGATCAGCGAGCGCCTGGTGATCACCAGCG
AGATGATCGAGAACATCCAGAGCGTGAAGGCCTACTGCTGGGAGGAGGCCATGGAG
AAGATGATCGAGAACCTGCGCCAGACCGAGCTGAAGCTGACCCGCAAGGCCGCCTA
CGTGCGCTACTTCAACAGCAGCGCCTTCTTCTTCAGCGGCTTCTTCGTGGTGTTCCTG
AGCGTGCTGCCCTACGCCCTGATCAAGGGCATCATCCTGCGCAAGATCTTCACCACC
ATCAGCTTCTGCATCGTGCTGCGCATGGCCGTGACCCGCCAGTTCCCCTGGGCCGTG
CAGACCTGGTACGACAGCCTGGGCGCCATCAACAAGATCCAGGACTTCCTGCAGAA
GCAGGAGTACAAGACCCTGGAGTACAACCTGACCACCACCGAGGTGGTGATGGAGA
ACGTGACCGCCTTCTGGGAGGAGGGCTTCGGCGAGCTGTTCGAGAAGGCCAAGCAG
AACAACAACAACCGCAAGACCAGCAACGGCGACGACAGCCTGTTCTTCAGCAACTT
CAGCCTGCTGGGCACCCCCGTGCTGAAGGACATCAACTTCAAGATCGAGCGCGGCC
AGCTGCTGGCCGTGGCCGGCAGCACCGGCGCCGGCAAGACCAGCCTGCTGATGGTG
ATCATGGGCGAGCTGGAGCCCAGCGAGGGCAAGATCAAGCACAGCGGCCGCATCA
GCTTCTGCAGCCAGTTCAGCTGGATCATGCCCGGCACCATCAAGGAGAACATCATCT
TCGGCGTGAGCTACGACGAGTACCGCTACCGCAGCGTGATCAAGGCCTGCCAGCTG
GAGGAGGACATCAGCAAGTTCGCCGAGAAGGACAACATCGTGCTGGGCGAGGGCG
GCATCACCCTGAGCGGCGGCCAGCGCGCCCGCATCAGCCTGGCCCGCGCCGTGTAC
AAGGACGCCGACCTGTACCTGCTGGACAGCCCCTTCGGCTACCTGGACGTGCTGACC
GAGAAGGAGATCTTCGAGAGCTGCGTGTGCAAGCTGATGGCCAACAAGACCCGCAT
CCTGGTGACCAGCAAGATGGAGCACCTGAAGAAGGCCGACAAGATCCTGATCCTGC
ACGAGGGCAGCAGCTACTTCTACGGCACCTTCAGCGAGCTGCAGAACCTGCAGCCC
GACTTCAGCAGCAAGCTGATGGGCTGCGACAGCTTCGACCAGTTCAGCGCCGAGCG
CCGCAACAGCATCCTGACCGAGACCCTGCACCGCTTCAGCCTGGAGGGCGACGCCC
CCGTGAGCTGGACCGAGACCAAGAAGCAGAGCTTCAAGCAGACCGGCGAGTTCGGC
GAGAAGCGCAAGAACAGCATCCTGAACCCCATCAACAGCATCCGCAAGTTCAGCAT
CGTGCAGAAGACCCCCCTGCAGATGAACGGCATCGAGGAGGACAGCGACGAGCCCC
TGGAGCGCCGCCTGAGCCTGGTGCCCGACAGCGAGCAGGGCGAGGCCATCCTGCCC
CGCATCAGCGTGATCAGCACCGGCCCCACCCTGCAGGCCCGCCGCCGCCAGAGCGT
GCTGAACCTGATGACCCACAGCGTGAACCAGGGCCAGAACATCCACCGCAAGACCA
CCGCCAGCACCCGCAAGGTGAGCCTGGCCCCCCAGGCCAACCTGACCGAGCTGGAC
ATCTACAGCCGCCGCCTGAGCCAGGAGACCGGCCTGGAGATCAGCGAGGAGATCAA
CGAGGAGGACCTGAAGGAGTGCTTCTTCGACGACATGGAGAGCATCCCCGCCGTGA
CCACCTGGAACACCTACCTGCGCTACATCACCGTGCACAAGAGCCTGATCTTCGTGC
TGATCTGGTGCCTGGTGATCTTCCTGGCCGAGGTGGCCGCCAGCCTGGTGGTGCTGT
GGCTGCTGGGCAACACCCCCCTGCAGGACAAGGGCAACAGCACCCACAGCCGCAAC
AACAGCTACGCCGTGATCATCACCAGCACCAGCAGCTACTACGTGTTCTACATCTAC
GTGGGCGTGGCCGACACCCTGCTGGCCATGGGCTTCTTCCGCGGCCTGCCCCTGGTG
CACACCCTGATCACCGTGAGCAAGATCCTGCACCACAAGATGCTGCACAGCGTGCT
GCAGGCCCCCATGAGCACCCTGAACACCCTGAAGGCCGGCGGCATCCTGAACCGCT
TCAGCAAGGACATCGCCATCCTGGACGACCTGCTGCCCCTGACCATCTTCGACTTCA
TCCAGCTGCTGCTGATCGTGATCGGCGCCATCGCCGTGGTGGCCGTGCTGCAGCCCT
ACATCTTCGTGGCCACCGTGCCCGTGATCGTGGCCTTCATCATGCTGCGCGCCTACTT
CCTGCAGACCAGCCAGCAGCTGAAGCAGCTGGAGAGCGAGGGCCGCAGCCCCATCT
TCACCCACCTGGTGACCAGCCTGAAGGGCCTGTGGACCCTGCGCGCCTTCGGCCGCC
AGCCCTACTTCGAGACCCTGTTCCACAAGGCCCTGAACCTGCACACCGCCAACTGGT
TCCTGTACCTGAGCACCCTGCGCTGGTTCCAGATGCGCATCGAGATGATCTTCGTGA
TCTTCTTCATCGCCGTGACCTTCATCAGCATCCTGACCACCGGCGAGGGCGAGGGCC
GCGTGGGCATCATCCTGACCCTGGCCATGAACATCATGAGCACCCTGCAGTGGGCC
GTGAACAGCAGCATCGACGTGGACAGCCTGATGCGCAGCGTGAGCCGCGTGTTCAA
GTTCATCGACATGCCCACCGAGGGCAAGCCCACCAAGAGCACCAAGCCCTACAAGA
ACGGCCAGCTGAGCAAGGTGATGATCATCGAGAACAGCCACGTGAAGAAGGACGA
CATCTGGCCCAGCGGCGGCCAGATGACCGTGAAGGACCTGACCGCCAAGTACACCG
AGGGCGGCAACGCCATCCTGGAGAACATCAGCTTCAGCATCAGCCCCGGCCAGCGC
GTGGGCCTGCTGGGCCGCACCGGCAGCGGCAAGAGCACCCTGCTGAGCGCCTTCCT
GCGCCTGCTGAACACCGAGGGCGAGATCCAGATCGACGGCGTGAGCTGGGACAGCA
TCACCCTGCAGCAGTGGCGCAAGGCCTTCGGCGTGATCCCCCAGAAGGTGTTCATCT
TCAGCGGCACCTTCCGCAAGAACCTGGACCCCTACGAGCAGTGGAGCGACCAGGAG
ATCTGGAAGGTGGCCGACGAGGTGGGCCTGCGCAGCGTGATCGAGCAGTTCCCCGG
CAAGCTGGACTTCGTGCTGGTGGACGGCGGCTGCGTGCTGAGCCACGGCCACAAGC
AGCTGATGTGCCTGGCCCGCAGCGTGCTGAGCAAGGCCAAGATCCTGCTGCTGGAC
GAGCCCAGCGCCCACCTGGACCCCGTGACCTACCAGATCATCCGCCGCACCCTGAA
GCAGGCCTTCGCCGACTGCACCGTGATCCTGTGCGAGCACCGCATCGAGGCCATGCT
GGAGTGCCAGCAGTTCCTGGTGATCGAGGAGAACAAGGTGCGCCAGTACGACAGCA
TCCAGAAGCTGCTGAACGAGCGCAGCCTGTTCCGCCAGGCCATCAGCCCCAGCGAC
CGCGTGAAGCTGTTCCCCCACCGCAACAGCAGCAAGTGCAAGAGCAAGCCCCAGAT
CGCCGCCCTGAAGGAGGAGACCGAGGAGGAGGTGCAGGACACCCGCCTGTAA
(SEQ ID NO: 37)
ATGCAGAGAAGCCCCCTGGAGAAGGCCAGCGTGGTGAGCAAGCTGTTCTTCAGCTG
GACCAGACCCATCCTGAGAAAGGGCTACAGACAGAGACTGGAGCTGAGCGACATCT
ACCAGATCCCCAGCGTGGACAGCGCCGACAACCTGAGCGAGAAGCTGGAGAGAGA
GTGGGACAGAGAGCTGGCCAGCAAGAAGAACCCCAAGCTGATCAACGCCCTGAGA
AGATGCTTCTTCTGGAGATTCATGTTCTACGGCATCTTCCTGTACCTGGGCGAGGTG
ACCAAGGCCGTGCAGCCCCTGCTGCTGGGCAGAATCATCGCCAGCTACGACCCCGA
CAACAAGGAGGAGAGAAGCATCGCCATCTACCTGGGCATCGGCCTGTGCCTGCTGT
TCATCGTGAGAACCCTGCTGCTGCACCCCGCCATCTTCGGCCTGCACCACATCGGCA
TGCAGATGAGAATCGCCATGTTCAGCCTGATCTACAAGAAGACCCTGAAGCTGAGC
AGCAGAGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGAATGAT
GATGAAGTACAGGGACCAGAGAGCCGGCAAGATCAGCGAGAGACTGGTGATCACC
AGCGAGATGATCGAGAACATCCAGAGCGTGAAGGCCTACTGCTGGGAGGAGGCCAT
GGAGAAGATGATCGAGAACCTGAGACAGACCGAGCTGAAGCTGACCAGAAAGGCC
GCCTACGTGAGATACTTCAACAGCAGCGCCTTCTTCTTCAGCGGCTTCTTCGTGGTGT
TCCTGAGCGTGCTGCCCTACGCCCTGATCAAGGGCATCATCCTGAGAAAGATCTTCA
CCACCATCAGCTTCTGCATCGTGCTGAGAATGGCCGTGACCAGACAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGAAAGACCAGCAACGGCGACGACAGCCTGTTCTTCAGC
AACTTCAGCCTGCTGGGCACCCCCGTGCTGAAGGACATCAACTTCAAGATCGAGAG
AGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
AATCAGCTTCTGCAGCCAGTTCAGCTGGATCATGCCCGGCACCATCAAGGAGAACA
TCATCTTCGGCGTGAGCTACGACGAGTACAGATACAGAAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGAGCCAGAATCAGCCTGGCCAGAGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGAATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGAAGAAACAGCATCCTGACCGAGACCCTGCACAGATTCAGCCTGGAGGGCGA
CGCCCCCGTGAGCTGGACCGAGACCAAGAAGCAGAGCTTCAAGCAGACCGGCGAGT
TCGGCGAGAAGAGAAAGAACAGCATCCTGAACCCCATCAACAGCATCAGAAAGTTC
AGCATCGTGCAGAAGACCCCCCTGCAGATGAACGGCATCGAGGAGGACAGCGACG
AGCCCCTGGAGAGAAGACTGAGCCTGGTGCCCGACAGCGAGCAGGGCGAGGCCATC
CTGCCCAGAATCAGCGTGATCAGCACCGGCCCCACCCTGCAGGCCAGAAGAAGACA
GAGCGTGCTGAACCTGATGACCCACAGCGTGAACCAGGGCCAGAACATCCACAGAA
AGACCACCGCCAGCACCAGAAAGGTGAGCCTGGCCCCCCAGGCCAACCTGACCGAG
CTGGACATCTACAGCAGAAGACTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGATACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGAAACAACAGCTACGCCGTGATCATCACCAGCACCAGCAGCTACTACGTGTTC
TACATCTACGTGGGCGTGGCCGACACCCTGCTGGCCATGGGCTTCTTCAGAGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGATTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
AGCCTACTTCCTGCAGACCAGCCAGCAGCTGAAGCAGCTGGAGAGCGAGGGCAGGA
GCCCCATCTTCACCCACCTGGTGACCAGCCTGAAGGGCCTGTGGACCCTGAGAGCCT
TCGGCAGACAGCCCTACTTCGAGACCCTGTTCCACAAGGCCCTGAACCTGCACACCG
CCAACTGGTTCCTGTACCTGAGCACCCTGAGATGGTTCCAGATGAGAATCGAGATGA
TCTTCGTGATCTTCTTCATCGCCGTGACCTTCATCAGCATCCTGACCACCGGCGAGG
GCGAGGGCAGAGTGGGCATCATCCTGACCCTGGCCATGAACATCATGAGCACCCTG
CAGTGGGCCGTGAACAGCAGCATCGACGTGGACAGCCTGATGAGAAGCGTGAGCAG
AGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGAGTGGGCCTGCTGGGCAGAACCGGCAGCGGCAAGAGCACCCTGCTGAGC
GCCTTCCTGAGACTGCTGAACACCGAGGGCGAGATCCAGATCGACGGCGTGAGCTG
GGACAGCATCACCCTGCAGCAGTGGAGAAAGGCCTTCGGCGTGATCCCCCAGAAGG
TGTTCATCTTCAGCGGCACCTTCAGAAAGAACCTGGACCCCTACGAGCAGTGGAGC
GACCAGGAGATCTGGAAGGTGGCCGACGAGGTGGGCCTGAGAAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGAAGCGTGCTGAGCAAGGCCAAGATCCT
GCTGCTGGACGAGCCCAGCGCCCACCTGGACCCCGTGACCTACCAGATCATCAGAA
GAACCCTGAAGCAGGCCTTCGCCGACTGCACCGTGATCCTGTGCGAGCACAGAATC
GAGGCCATGCTGGAGTGCCAGCAGTTCCTGGTGATCGAGGAGAACAAGGTGAGACA
GTACGACAGCATCCAGAAGCTGCTGAACGAGAGAAGCCTGTTCAGACAGGCCATCA
GCCCCAGCGACAGAGTGAAGCTGTTCCCCCACAGAAACAGCAGCAAGTGCAAGAGC
AAGCCCCAGATCGCCGCCCTGAAGGAGGAGACCGAGGAGGAGGTGCAGGACACCA
GACTGTGA
(SEQ ID NO: 38)
ATGCAGAGGTCACCTCTGGAAAAGGCTAGCGTGGTCAGCAAGCTATTTTTTTCCTGG
ACCCGCCCGATACTCAGGAAGGGCTACCGACAGCGGCTGGAGCTGAGTGACATTTA
TCAGATTCCCTCCGTCGATTCCGCTGACAACCTGTCTGAGAAACTGGAGCGGGAATG
GGATAGGGAACTGGCGTCCAAAAAAAACCCCAAACTCATCAATGCACTCCGCAGAT
GCTTCTTCTGGCGGTTTATGTTTTATGGCATATTCCTGTATCTGGGGGAGGTGACGAA
AGCCGTGCAGCCGCTGCTGCTTGGTCGCATTATCGCGTCATACGATCCAGATAACAA
GGAGGAAAGAAGTATCGCTATCTATCTCGGGATAGGGCTGTGCCTGCTCTTCATTGT
GCGGACTCTTCTCTTGCACCCCGCCATTTTCGGTCTGCATCATATAGGTATGCAGATG
AGAATTGCGATGTTCTCATTGATTTACAAAAAAACGCTTAAGCTAAGTTCAAGGGTG
CTAGATAAGATATCGATCGGCCAGCTGGTGTCTCTGCTTAGCAACAACCTCAATAAA
TTCGACGAAGGCCTTGCACTGGCCCACTTCGTGTGGATCGCCCCTCTGCAGGTGGCT
CTGCTGATGGGGTTAATATGGGAGCTGTTGCAGGCCTCCGCTTTTTGTGGCCTGGGG
TTTCTCATCGTGTTGGCCTTGTTTCAGGCAGGGCTGGGACGTATGATGATGAAATAT
AGGGATCAGAGGGCTGGCAAAATCTCTGAGCGCCTGGTTATTACGAGTGAAATGAT
TGAGAACATCCAGTCAGTGAAGGCCTATTGCTGGGAGGAGGCCATGGAAAAAATGA
TTGAGAACCTACGCCAGACTGAGCTGAAGTTAACCAGAAAAGCCGCCTATGTGCGC
TACTTTAACAGTAGCGCATTTTTCTTCTCCGGTTTTTTCGTGGTGTTTCTTAGTGTGTT
GCCGTATGCCTTAATCAAGGGAATAATACTCCGGAAGATTTTCACTACCATCAGCTT
CTGTATCGTGTTGCGGATGGCCGTCACCCGGCAGTTTCCCTGGGCAGTACAGACTTG
GTACGATTCTCTCGGAGCAATTAACAAAATCCAAGACTTTCTACAAAAGCAGGAGT
ACAAGACCCTGGAGTACAATCTGACCACCACAGAAGTCGTAATGGAGAATGTAACT
GCCTTCTGGGAAGAGGGCTTTGGCGAACTCTTTGAAAAGGCCAAGCAGAACAATAA
CAACCGGAAGACCTCCAACGGGGACGACAGCTTATTTTTCAGCAATTTTTCTTTGCT
CGGGACCCCTGTACTGAAAGATATTAACTTTAAGATCGAGCGCGGACAACTCCTGGC
TGTCGCCGGCAGCACTGGAGCTGGAAAAACATCACTGCTTATGGTGATAATGGGAG
AACTCGAACCAAGCGAGGGAAAAATAAAGCACTCTGGACGGATTAGTTTTTGCTCC
CAGTTCTCGTGGATAATGCCTGGCACCATTAAGGAGAATATCATCTTTGGAGTGAGT
TACGACGAATACCGGTACCGGTCCGTTATCAAGGCTTGTCAACTCGAGGAGGACATT
TCTAAATTCGCCGAAAAAGATAATATAGTGCTGGGCGAAGGAGGCATTACACTGAG
CGGGGGTCAGAGAGCTCGAATTAGCCTCGCCCGAGCAGTCTATAAAGACGCCGATC
TTTACCTGCTGGATTCCCCTTTTGGGTATTTGGATGTTCTGACAGAGAAGGAAATCTT
TGAATCATGTGTCTGTAAACTGATGGCCAATAAGACTAGGATTCTAGTGACTTCGAA
AATGGAGCACCTGAAAAAAGCGGACAAAATTCTGATACTCCATGAAGGGTCTTCCT
ACTTCTACGGCACCTTCTCAGAGTTGCAGAACTTACAACCTGATTTTTCATCTAAGCT
TATGGGGTGCGACTCGTTTGACCAGTTCTCCGCTGAAAGACGAAACAGCATCTTAAC
GGAAACTCTTCACAGGTTCTCATTAGAGGGAGATGCGCCGGTGTCCTGGACAGAGA
CAAAAAAACAGTCTTTCAAACAGACAGGAGAGTTTGGCGAGAAGAGAAAAAACTC
AATCCTCAATCCCATCAATTCTATTAGAAAGTTTAGCATCGTCCAAAAAACACCATT
GCAGATGAATGGGATTGAGGAGGACAGTGATGAGCCTTTGGAACGAAGACTGTCCC
TGGTACCCGATAGCGAACAGGGTGAGGCCATCCTTCCTAGGATCTCGGTCATAAGTA
CAGGGCCCACACTGCAGGCCAGGCGACGTCAAAGTGTCCTCAATCTTATGACGCAC
AGTGTGAATCAGGGGCAGAACATCCATCGTAAGACGACAGCTTCAACTCGAAAGGT
CAGTCTAGCTCCACAAGCCAATCTTACAGAGCTGGACATTTATTCCCGCCGCCTCAG
TCAGGAGACCGGATTGGAAATATCAGAGGAAATTAATGAAGAGGATCTGAAGGAAT
GCTTCTTTGATGACATGGAATCGATCCCCGCTGTTACTACCTGGAACACATATCTGA
GATATATTACCGTCCATAAGAGCTTAATCTTTGTACTGATATGGTGCTTGGTGATTTT
CCTGGCAGAGGTTGCGGCGAGTTTGGTCGTGCTATGGCTCCTTGGAAACACTCCCCT
GCAGGATAAGGGGAACTCCACTCATAGCAGGAATAACAGCTATGCCGTGATCATCA
CCTCTACCTCCTCTTATTACGTGTTTTACATATACGTCGGTGTTGCGGATACCCTGTT
GGCAATGGGGTTCTTTAGAGGACTACCCCTAGTTCACACCCTGATCACCGTTTCGAA
GATCTTGCACCACAAGATGCTTCATAGCGTTCTCCAAGCTCCTATGAGCACCCTTAA
TACACTGAAAGCAGGAGGTATCCTTAACCGCTTTTCCAAAGACATCGCTATACTCGA
CGATTTGCTCCCATTGACCATCTTCGACTTCATTCAGCTGCTCCTCATTGTGATCGGC
GCCATTGCCGTGGTCGCAGTGTTACAGCCATATATTTTCGTAGCCACCGTGCCCGTC
ATCGTGGCATTTATCATGCTGCGCGCATATTTCTTACAGACATCTCAGCAACTGAAG
CAGCTGGAATCTGAGGGCAGATCTCCTATTTTTACACACCTGGTTACCAGCCTGAAG
GGCCTGTGGACCCTGCGTGCTTTCGGTCGCCAACCCTACTTTGAGACTCTCTTCCATA
AGGCTCTGAATTTACATACTGCCAATTGGTTCCTATACCTTAGTACCCTTCGGTGGTT
CCAGATGCGGATAGAAATGATCTTCGTGATTTTCTTCATCGCAGTCACTTTCATCTCT
ATTTTGACGACCGGTGAGGGCGAGGGCAGGGTGGGCATCATTCTGACTTTGGCCATG
AACATTATGTCAACACTCCAGTGGGCCGTTAATTCAAGCATTGATGTGGATTCCTTG
ATGCGTTCCGTCAGCAGGGTATTTAAATTCATAGACATGCCCACCGAGGGCAAGCCA
ACAAAATCTACCAAGCCATACAAAAATGGCCAACTAAGCAAGGTCATGATTATCGA
GAATTCTCATGTGAAAAAGGACGACATTTGGCCTTCCGGGGGTCAAATGACTGTAA
AGGACCTGACGGCTAAATACACTGAGGGCGGTAATGCTATCTTGGAGAACATCTCTT
TCAGCATCTCCCCTGGCCAGAGAGTGGGACTGCTCGGGCGGACAGGCTCCGGAAAG
TCTACGCTCCTTTCAGCATTCCTTAGACTTCTGAACACCGAAGGTGAGATTCAGATT
GACGGGGTCTCTTGGGACTCCATCACACTTCAGCAATGGAGGAAGGCATTCGGTGTA
ATCCCCCAAAAGGTTTTTATCTTCTCCGGAACATTTCGTAAGAATCTGGACCCGTAC
GAGCAGTGGTCAGATCAGGAGATCTGGAAAGTAGCAGACGAGGTCGGGCTACGGA
GCGTTATTGAACAGTTTCCTGGCAAACTGGACTTCGTTTTGGTGGACGGAGGCTGTG
TGCTGAGTCACGGCCATAAACAACTGATGTGCTTAGCTAGGTCTGTTCTCAGCAAGG
CAAAGATTTTACTGCTGGATGAACCAAGCGCCCACCTTGATCCAGTGACATATCAAA
TCATCAGAAGAACTCTTAAACAGGCGTTCGCCGACTGCACAGTGATCCTGTGTGAGC
ACAGAATAGAAGCCATGCTGGAATGTCAACAGTTTCTCGTGATTGAGGAGAACAAG
GTGCGCCAGTACGATAGCATCCAGAAGTTACTCAATGAAAGGTCACTCTTCAGGCA
GGCCATCTCACCCAGCGACCGCGTTAAGCTGTTTCCACACCGAAACAGTTCCAAGTG
CAAAAGTAAGCCACAGATTGCTGCACTGAAGGAAGAGACAGAAGAAGAAGTTCAG
GACACTCGGCTCTGA
(SEQ ID NO: 39)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA
(SEQ ID NO: 40)
ATGCAACGGAGTCCTCTGGAAAAAGCCTCTGTCGTATCTAAGCTTTTCTTCAGTTGG
ACACGCCCGATTTTGAGAAAGGGTTATCGGCAACGCTTGGAACTTAGTGACATCTAC
CAAATTCCAAGTGTAGACTCAGCCGATAACTTGAGCGAAAAGCTCGAACGAGAGTG
GGATCGAGAACTGGCTAGCAAAAAAAATCCCAAACTCATAAATGCCCTGCGACGCT
GTTTCTTTTGGCGATTTATGTTTTACGGTATTTTCCTTTATTTGGGTGAGGTCACGAA
GGCTGTACAGCCACTGCTGCTGGGTCGCATCATTGCCTCTTACGACCCTGACAACAA
AGAGGAGCGGTCAATAGCTATCTACCTTGGTATAGGACTTTGCTTGCTCTTCATAGT
CCGCACGTTGCTTCTCCACCCTGCTATATTTGGTCTCCATCACATTGGGATGCAAATG
CGGATCGCGATGTTCAGTCTTATATATAAAAAGACTCTTAAACTTTCCAGCCGGGTT
CTGGATAAGATCTCTATTGGTCAACTGGTATCTCTTTTGTCTAACAACCTGAATAAGT
TCGACGAGGGCCTTGCATTGGCCCATTTTGTATGGATTGCCCCTTTGCAAGTCGCCCT
CCTGATGGGATTGATCTGGGAACTCCTGCAAGCTAGTGCTTTTTGCGGATTGGGATT
CCTCATAGTCCTTGCGCTCTTTCAGGCGGGACTTGGACGCATGATGATGAAGTATCG
CGACCAACGAGCTGGCAAGATCAGTGAACGGCTTGTAATAACCAGTGAAATGATAG
AGAACATCCAGAGCGTAAAAGCTTACTGTTGGGAAGAAGCGATGGAAAAGATGATT
GAGAACCTTCGCCAGACAGAACTTAAACTTACACGAAAGGCCGCTTATGTCCGGTA
CTTCAACTCTTCAGCATTTTTTTTTAGTGGCTTCTTTGTAGTGTTCCTGTCCGTCCTTC
CGTATGCACTTATCAAGGGTATAATACTTAGGAAAATCTTCACAACAATCAGTTTTT
GCATAGTCCTTCGCATGGCAGTAACTCGCCAATTTCCCTGGGCAGTTCAGACGTGGT
ACGACTCACTTGGCGCAATTAACAAAATTCAAGATTTCCTCCAAAAGCAAGAGTATA
AAACCTTGGAATACAACCTTACCACCACAGAAGTTGTAATGGAAAATGTCACAGCC
TTCTGGGAGGAAGGTTTCGGCGAACTTTTTGAGAAGGCGAAGCAAAATAACAATAA
TCGGAAAACATCAAACGGTGACGATTCACTGTTCTTTTCTAACTTTAGCCTTCTTGGG
ACGCCCGTCCTGAAGGACATAAACTTTAAGATTGAACGGGGTCAACTTCTCGCGGTC
GCAGGGAGTACTGGAGCGGGGAAAACGAGCCTGCTGATGGTGATAATGGGGGAGTT
GGAGCCCTCAGAAGGCAAGATCAAGCATAGTGGTAGAATTAGCTTCTGCAGTCAAT
TTAGTTGGATTATGCCGGGCACGATCAAAGAAAATATAATCTTTGGGGTATCCTACG
ATGAATACAGGTACCGATCAGTGATAAAAGCGTGCCAGCTTGAAGAAGACATTTCA
AAGTTTGCTGAGAAGGATAATATCGTACTTGGAGAAGGAGGTATCACCCTGTCTGG
GGGTCAACGAGCGAGGATCTCCCTGGCACGCGCCGTCTACAAGGACGCGGACCTCT
ATCTGTTGGATTCACCGTTCGGATATTTGGACGTGCTTACGGAGAAAGAAATATTTG
AGAGCTGTGTTTGCAAGCTCATGGCAAATAAAACCAGAATATTGGTTACAAGCAAG
ATGGAGCATCTTAAGAAAGCAGATAAAATCCTGATATTGCACGAGGGCTCTTCATAC
TTCTACGGGACGTTTTCTGAGTTGCAGAACCTCCAGCCGGATTTCAGCTCTAAGCTG
ATGGGCTGTGATTCCTTTGATCAGTTTAGTGCGGAAAGACGAAACAGTATACTCACC
GAAACACTGCACAGGTTCTCTCTGGAGGGCGACGCCCCGGTTTCCTGGACAGAGAC
GAAGAAGCAGTCCTTCAAACAGACAGGCGAGTTTGGGGAGAAAAGGAAAAATAGC
ATACTCAACCCGATTAACAGCATTCGCAAGTTCAGTATAGTACAAAAGACCCCGTTG
CAGATGAACGGTATAGAGGAAGATTCTGATGAGCCACTGGAAAGACGGCTTTCTCT
CGTTCCGGACAGTGAACAGGGAGAGGCAATACTGCCTCGGATCAGCGTTATCTCTAC
AGGACCTACTTTGCAAGCTCGGCGCCGACAGTCAGTCTTGAATCTTATGACTCATAG
TGTTAATCAAGGCCAGAATATCCATCGCAAGACCACCGCAAGTACAAGGAAAGTGA
GCTTGGCACCTCAAGCAAACCTTACTGAACTTGATATCTACTCACGGCGACTTTCAC
AGGAGACCGGACTTGAAATTAGTGAAGAAATTAACGAGGAGGACCTCAAGGAGTGC
TTCTTCGATGACATGGAATCAATCCCCGCAGTCACAACCTGGAACACTTATCTGAGG
TATATAACAGTTCACAAGAGCCTCATTTTTGTACTTATTTGGTGTTTGGTAATTTTCC
TGGCGGAGGTTGCTGCTTCTTTGGTCGTCCTTTGGCTCCTCGGGAATACACCGCTCCA
AGACAAAGGCAACTCTACCCATAGTAGGAACAATTCATATGCAGTGATTATAACCA
GTACATCATCTTATTACGTTTTCTATATTTATGTCGGGGTAGCTGACACGCTGTTGGC
GATGGGCTTCTTTAGGGGCCTCCCCTTGGTACACACCCTTATCACGGTGAGTAAAAT
CCTGCATCACAAAATGCTTCATTCTGTACTCCAAGCGCCGATGAGTACGCTTAATAC
GCTGAAAGCAGGAGGGATACTGAATCGGTTCAGCAAGGACATCGCCATTCTGGATG
ACCTGCTTCCATTGACAATATTTGATTTCATTCAGCTCCTTCTCATAGTTATTGGAGC
CATAGCGGTGGTGGCTGTGCTTCAGCCTTATATATTCGTTGCCACAGTTCCCGTTATA
GTGGCATTTATAATGCTCAGGGCCTACTTTCTCCAGACTTCCCAGCAGTTGAAGCAA
CTCGAATCAGAAGGAAGGTCACCTATTTTCACACATCTTGTGACTTCCTTGAAGGGC
TTGTGGACGCTGCGGGCCTTCGGAAGACAACCATATTTTGAAACTCTCTTCCACAAA
GCTTTGAATCTTCATACTGCGAACTGGTTCCTGTATTTGAGTACTTTGCGCTGGTTCC
AGATGAGGATAGAAATGATATTCGTTATCTTCTTTATCGCGGTTACGTTCATAAGTA
TCCTCACTACGGGGGAGGGTGAGGGTAGAGTGGGCATAATACTGACCCTCGCCATG
AACATTATGTCCACCCTGCAGTGGGCGGTAAACAGCAGCATAGATGTGGATTCTTTG
ATGCGCAGTGTGAGCAGGGTTTTTAAGTTTATCGATATGCCGACGGAAGGAAAGCC
CACTAAAAGCACGAAACCCTATAAAAATGGACAGCTTAGCAAAGTAATGATAATCG
AGAATAGCCATGTGAAAAAGGATGACATATGGCCTTCCGGAGGCCAAATGACTGTT
AAAGATCTGACCGCTAAATATACCGAGGGCGGCAACGCAATACTCGAAAACATAAG
CTTTTCCATAAGCCCCGGCCAACGCGTGGGTCTTCTGGGGAGGACTGGCTCCGGAAA
ATCAACGTTGCTTAGCGCGTTTTTGCGGCTCCTTAACACTGAAGGTGAGATCCAAAT
AGATGGCGTTAGTTGGGACTCTATAACACTGCAACAATGGCGGAAAGCTTTCGGCGT
CATACCTCAGAAGGTGTTCATCTTTAGCGGAACGTTCAGGAAGAACTTGGATCCCTA
CGAACAATGGAGTGATCAAGAAATATGGAAAGTGGCAGATGAGGTAGGCTTGCGCA
GTGTCATTGAACAATTCCCAGGGAAACTCGACTTTGTACTGGTGGACGGCGGTTGCG
TCTTGTCACACGGGCACAAACAGTTGATGTGTTTGGCCCGCAGTGTTTTGTCTAAGG
CGAAGATTCTGTTGCTCGACGAACCGAGTGCTCATCTTGATCCCGTCACCTACCAAA
TCATCAGAAGGACGTTGAAGCAAGCTTTCGCCGACTGCACTGTAATCCTTTGTGAGC
ATAGGATCGAAGCAATGCTCGAGTGCCAACAGTTCTTGGTTATAGAGGAGAATAAG
GTTCGGCAATACGACTCAATACAGAAACTGCTTAATGAGCGGTCACTCTTTCGACAA
GCTATCTCTCCTAGTGACAGGGTAAAGCTTTTTCCTCATCGGAATTCCAGCAAGTGT
AAGAGTAAACCACAGATCGCCGCCCTTAAAGAGGAGACCGAAGAAGAGGTGCAGG
ATACGAGACTTTAG

Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

Exemplary Codon-Optimized Human Cystic Fibrosis Transmembrane Conductance Regulator (CFTR) mRNAs

Construct design:
X - SEQ ID NO: 1 - Y
5′ and 3′ UTR Sequences:
(SEQ ID NO: 4)
X (5′ UTR Sequence) =
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACG
(SEQ ID NO: 5)
Y (3′ UTR Sequence) =
CGGGUGGCAUCCCUGUGACCCCUCCCCAGUGCCUCUCCUGGCCCUGGAAG
UUGCCACUCCAGUGCCCACCAGCCUUGUCCUAAUAAAAUUAAGUUGCAUC
AAGCU
OR
(SEQ ID NO: 6)
GGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUCA
AAGCU

An exemplary codon-optimized human CFTR mRNA sequence includes SEQ ID NO: 1 as described in the detailed description section.

An exemplary full-length codon-optimized human CFTR mRNA sequence is shown below.

(SEQ ID NO: 7)
GGACAGAUCGCCUGGAGACGCCAUCCACGCUGUUUUGACCUCCAUAGAAGACACC
GGGACCGAUCCAGCCUCCGCGGCCGGGAACGGUGCAUUGGAACGCGGAUUCCCCG
UGCCAAGAGUGACUCACCGUCCUUGACACGAUGCAACGCUCUCCUCUUGAAAAGG
CCUCGGUGGUGUCCAAGCUCUUCUUCUCGUGGACUAGACCCAUCCUGAGAAAGGG
GUACAGACAGCGCUUGGAGCUGUCCGAUAUCUAUCAAAUCCCUUCCGUGGACUCC
GCGGACAACCUGUCCGAGAAGCUCGAGAGAGAAUGGGACAGAGAACUCGCCUCAA
AGAAGAACCCGAAGCUGAUUAAUGCGCUUAGGCGGUGCUUUUUCUGGCGGUUCA
UGUUCUACGGCAUCUUCCUCUACCUGGGAGAGGUCACCAAGGCCGUGCAGCCCCU
GUUGCUGGGACGGAUUAUUGCCUCCUACGACCCCGACAACAAGGAAGAAAGAAGC
AUCGCUAUCUACUUGGGCAUCGGUCUGUGCCUGCUUUUCAUCGUCCGGACCCUCU
UGUUGCAUCCUGCUAUUUUCGGCCUGCAUCACAUUGGCAUGCAGAUGAGAAUUG
CCAUGUUUUCCCUGAUCUACAAGAAAACUCUGAAGCUCUCGAGCCGCGUGCUUGA
CAAGAUUUCCAUCGGCCAGCUCGUGUCCCUGCUCUCCAACAAUCUGAACAAGUUC
GACGAGGGCCUCGCCCUGGCCCACUUCGUGUGGAUCGCCCCUCUGCAAGUGGCGC
UUCUGAUGGGCCUGAUCUGGGAGCUGCUGCAAGCCUCGGCAUUCUGUGGGCUUG
GAUUCCUGAUCGUGCUGGCACUGUUCCAGGCCGGACUGGGGCGGAUGAUGAUGA
AGUACAGGGACCAGAGAGCCGGAAAGAUUUCCGAACGGCUGGUGAUCACUUCGG
AAAUGAUCGAAAACAUCCAGUCAGUGAAGGCCUACUGCUGGGAAGAGGCCAUGG
AAAAGAUGAUUGAAAACCUCCGGCAAACCGAGCUGAAGCUGACCCGCAAGGCCGC
UUACGUGCGCUAUUUCAACUCGUCCGCUUUCUUCUUCUCCGGGUUCUUCGUGGUG
UUUCUCUCCGUGCUCCCCUACGCCCUGAUUAAGGGAAUCAUCCUCAGGAAGAUCU
UCACCACCAUUUCCUUCUGUAUCGUGCUCCGCAUGGCCGUGACCCGGCAGUUCCC
AUGGGCCGUGCAGACUUGGUACGACUCCCUGGGAGCCAUUAACAAGAUCCAGGAC
UUCCUUCAAAAGCAGGAGUACAAGACCCUCGAGUACAACCUGACUACUACCGAGG
UCGUGAUGGAAAACGUCACCGCCUUUUGGGAGGAGGGAUUUGGCGAACUGUUCG
AGAAGGCCAAGCAGAACAACAACAACCGCAAGACCUCGAACGGUGACGACUCCCU
CUUCUUUUCAAACUUCAGCCUGCUCGGGACGCCCGUGCUGAAGGACAUUAACUUC
AAGAUCGAAAGAGGACAGCUCCUGGCGGUGGCCGGAUCGACCGGAGCCGGAAAG
ACUUCCCUGCUGAUGGUGAUCAUGGGAGAGCUUGAACCUAGCGAGGGAAAGAUC
AAGCACUCCGGCCGCAUCAGCUUCUGUAGCCAGUUUUCCUGGAUCAUGCCCGGAA
CCAUUAAGGAAAACAUCAUCUUCGGCGUGUCCUACGAUGAAUACCGCUACCGGUC
CGUGAUCAAAGCCUGCCAGCUGGAAGAGGAUAUUUCAAAGUUCGCGGAGAAAGA
UAACAUCGUGCUGGGCGAAGGGGGUAUUACCUUGUCGGGGGGCCAGCGGGCUAG
AAUCUCGCUGGCCAGAGCCGUGUAUAAGGACGCCGACCUGUAUCUCCUGGACUCC
CCCUUCGGAUACCUGGACGUCCUGACCGAAAAGGAGAUCUUCGAAUCGUGCGUGU
GCAAGCUGAUGGCUAACAAGACUCGCAUCCUCGUGACCUCCAAAAUGGAGCACCU
GAAGAAGGCAGACAAGAUUCUGAUUCUGCAUGAGGGGUCCUCCUACUUUUACGG
CACCUUCUCGGAGUUGCAGAACUUGCAGCCCGACUUCUCAUCGAAGCUGAUGGGU
UGCGACAGCUUCGACCAGUUCUCCGCCGAAAGAAGGAACUCGAUCCUGACGGAAA
CCUUGCACCGCUUCUCUUUGGAAGGCGACGCCCCUGUGUCAUGGACCGAGACUAA
GAAGCAGAGCUUCAAGCAGACCGGGGAAUUCGGCGAAAAGAGGAAGAACAGCAU
CUUGAACCCCAUUAACUCCAUCCGCAAGUUCUCAAUCGUGCAAAAGACGCCACUG
CAGAUGAACGGCAUUGAGGAGGACUCCGACGAACCCCUUGAGAGGCGCCUGUCCC
UGGUGCCGGACAGCGAGCAGGGAGAAGCCAUCCUGCCUCGGAUUUCCGUGAUCUC
CACUGGUCCGACGCUCCAAGCCCGGCGGCGGCAGUCCGUGCUGAACCUGAUGACC
CACAGCGUGAACCAGGGCCAAAACAUUCACCGCAAGACUACCGCAUCCACCCGGA
AAGUGUCCCUGGCACCUCAAGCGAAUCUUACCGAGCUCGACAUCUACUCCCGGAG
ACUGUCGCAGGAAACCGGGCUCGAAAUUUCCGAAGAAAUCAACGAGGAGGAUCU
GAAAGAGUGCUUCUUCGACGAUAUGGAGUCGAUACCCGCCGUGACGACUUGGAA
CACUUAUCUGCGGUACAUCACUGUGCACAAGUCAUUGAUCUUCGUGCUGAUUUG
GUGCCUGGUGAUUUUCCUGGCCGAGGUCGCGGCCUCACUGGUGGUGCUCUGGCUG
UUGGGAAACACGCCUCUGCAAGACAAGGGAAACUCCACGCACUCGAGAAACAACA
GCUAUGCCGUGAUUAUCACUUCCACCUCCUCUUAUUACGUGUUCUACAUCUACGU
CGGAGUGGCGGAUACCCUGCUCGCGAUGGGUUUCUUCAGAGGACUGCCGCUGGUC
CACACCUUGAUCACCGUCAGCAAGAUUCUUCACCACAAGAUGUUGCAUAGCGUGC
UGCAGGCCCCCAUGUCCACCCUCAACACUCUGAAGGCCGGAGGCAUUCUGAACAG
AUUCUCCAAGGACAUCGCUAUCCUGGACGAUCUCCUGCCGCUUACCAUCUUUGAC
UUCAUCCAGCUGCUGCUGAUCGUGAUUGGAGCAAUCGCAGUGGUGGCGGUGCUG
CAGCCUUACAUUUUCGUGGCCACUGUGCCGGUCAUUGUGGCGUUCAUCAUGCUGC
GGGCCUACUUCCUCCAAACCAGCCAGCAGCUGAAGCAACUGGAAUCCGAGGGACG
AUCCCCCAUCUUCACUCACCUUGUGACGUCGUUGAAGGGACUGUGGACCCUCCGG
GCUUUCGGACGGCAGCCCUACUUCGAAACCCUCUUCCACAAGGCCCUGAACCUCC
ACACCGCCAAUUGGUUCCUGUACCUGUCCACCCUGCGGUGGUUCCAGAUGCGCAU
CGAGAUGAUUUUCGUCAUCUUCUUCAUCGCGGUCACAUUCAUCAGCAUCCUGACU
ACCGGAGAGGGAGAGGGACGGGUCGGAAUAAUCCUGACCCUCGCCAUGAACAUU
AUGAGCACCCUGCAGUGGGCAGUGAACAGCUCGAUCGACGUGGACAGCCUGAUGC
GAAGCGUCAGCCGCGUGUUCAAGUUCAUCGACAUGCCUACUGAGGGAAAACCCAC
UAAGUCCACUAAGCCCUACAAAAAUGGCCAGCUGAGCAAGGUCAUGAUCAUCGAA
AACUCCCACGUGAAGAAGGACGAUAUUUGGCCCUCCGGAGGUCAAAUGACCGUGA
AGGACCUGACCGCAAAGUACACCGAGGGAGGAAACGCCAUUCUCGAAAACAUCAG
CUUCUCCAUUUCGCCGGGACAGCGGGUCGGCCUUCUCGGGCGGACCGGUUCCGGG
AAGUCAACUCUGCUGUCGGCUUUCCUCCGGCUGCUGAAUACCGAGGGGGAAAUCC
AAAUUGACGGCGUGUCUUGGGAUUCCAUUACUCUGCAGCAGUGGCGGAAGGCCU
UCGGCGUGAUCCCCCAGAAGGUGUUCAUCUUCUCGGGUACCUUCCGGAAGAACCU
GGAUCCUUACGAGCAGUGGAGCGACCAAGAAAUCUGGAAGGUCGCCGACGAGGU
CGGCCUGCGCUCCGUGAUUGAACAAUUUCCUGGAAAGCUGGACUUCGUGCUCGUC
GACGGGGGAUGUGUCCUGUCGCACGGACAUAAGCAGCUCAUGUGCCUCGCACGGU
CCGUGCUCUCCAAGGCCAAGAUUCUGCUGCUGGACGAACCUUCGGCCCACCUGGA
UCCGGUCACCUACCAGAUCAUCAGGAGGACCCUGAAGCAGGCCUUUGCCGAUUGC
ACCGUGAUUCUCUGCGAGCACCGCAUCGAGGCCAUGCUGGAGUGCCAGCAGUUCC
UGGUCAUCGAGGAGAACAAGGUCCGCCAAUACGACUCCAUUCAAAAGCUCCUCAA
CGAGCGGUCGCUGUUCAGACAAGCUAUUUCACCGUCCGAUAGAGUGAAGCUCUUC
CCGCAUCGGAACAGCUCAAAGUGCAAAUCGAAGCCGCAGAUCGCAGCCUUGAAGG
AAGAGACUGAGGAAGAGGUGCAGGACACCCGGCUUUAACGGGUGGCAUCCCUGU
GACCCCUCCCCAGUGCCUCUCCUGGCCCUGGAAGUUGCCACUCCAGUGCCCACCA
GCCUUGUCCUAAUAAAAUUAAGUUGCAUCAAGCU

In another example, a full length codon-optimized human CFTR mRNA sequence is shown below:

(SEQ ID NO: 8)
GGACAGAUCGCCUGGAGACGCCAUCCACGCUGUUUUGACCUCCAUAGAAGACACC
GGGACCGAUCCAGCCUCCGCGGCCGGGAACGGUGCAUUGGAACGCGGAUUCCCCG
UGCCAAGAGUGACUCACCGUCCUUGACACGAUGCAACGCUCUCCUCUUGAAAAGG
CCUCGGUGGUGUCCAAGCUCUUCUUCUCGUGGACUAGACCCAUCCUGAGAAAGGG
GUACAGACAGCGCUUGGAGCUGUCCGAUAUCUAUCAAAUCCCUUCCGUGGACUCC
GCGGACAACCUGUCCGAGAAGCUCGAGAGAGAAUGGGACAGAGAACUCGCCUCAA
AGAAGAACCCGAAGCUGAUUAAUGCGCUUAGGCGGUGCUUUUUCUGGCGGUUCA
UGUUCUACGGCAUCUUCCUCUACCUGGGAGAGGUCACCAAGGCCGUGCAGCCCCU
GUUGCUGGGACGGAUUAUUGCCUCCUACGACCCCGACAACAAGGAAGAAAGAAGC
AUCGCUAUCUACUUGGGCAUCGGUCUGUGCCUGCUUUUCAUCGUCCGGACCCUCU
UGUUGCAUCCUGCUAUUUUCGGCCUGCAUCACAUUGGCAUGCAGAUGAGAAUUG
CCAUGUUUUCCCUGAUCUACAAGAAAACUCUGAAGCUCUCGAGCCGCGUGCUUGA
CAAGAUUUCCAUCGGCCAGCUCGUGUCCCUGCUCUCCAACAAUCUGAACAAGUUC
GACGAGGGCCUCGCCCUGGCCCACUUCGUGUGGAUCGCCCCUCUGCAAGUGGCGC
UUCUGAUGGGCCUGAUCUGGGAGCUGCUGCAAGCCUCGGCAUUCUGUGGGCUUG
GAUUCCUGAUCGUGCUGGCACUGUUCCAGGCCGGACUGGGGCGGAUGAUGAUGA
AGUACAGGGACCAGAGAGCCGGAAAGAUUUCCGAACGGCUGGUGAUCACUUCGG
AAAUGAUCGAAAACAUCCAGUCAGUGAAGGCCUACUGCUGGGAAGAGGCCAUGG
AAAAGAUGAUUGAAAACCUCCGGCAAACCGAGCUGAAGCUGACCCGCAAGGCCGC
UUACGUGCGCUAUUUCAACUCGUCCGCUUUCUUCUUCUCCGGGUUCUUCGUGGUG
UUUCUCUCCGUGCUCCCCUACGCCCUGAUUAAGGGAAUCAUCCUCAGGAAGAUCU
UCACCACCAUUUCCUUCUGUAUCGUGCUCCGCAUGGCCGUGACCCGGCAGUUCCC
AUGGGCCGUGCAGACUUGGUACGACUCCCUGGGAGCCAUUAACAAGAUCCAGGAC
UUCCUUCAAAAGCAGGAGUACAAGACCCUCGAGUACAACCUGACUACUACCGAGG
UCGUGAUGGAAAACGUCACCGCCUUUUGGGAGGAGGGAUUUGGCGAACUGUUCG
AGAAGGCCAAGCAGAACAACAACAACCGCAAGACCUCGAACGGUGACGACUCCCU
CUUCUUUUCAAACUUCAGCCUGCUCGGGACGCCCGUGCUGAAGGACAUUAACUUC
AAGAUCGAAAGAGGACAGCUCCUGGCGGUGGCCGGAUCGACCGGAGCCGGAAAG
ACUUCCCUGCUGAUGGUGAUCAUGGGAGAGCUUGAACCUAGCGAGGGAAAGAUC
AAGCACUCCGGCCGCAUCAGCUUCUGUAGCCAGUUUUCCUGGAUCAUGCCCGGAA
CCAUUAAGGAAAACAUCAUCUUCGGCGUGUCCUACGAUGAAUACCGCUACCGGUC
CGUGAUCAAAGCCUGCCAGCUGGAAGAGGAUAUUUCAAAGUUCGCGGAGAAAGA
UAACAUCGUGCUGGGCGAAGGGGGUAUUACCUUGUCGGGGGGCCAGCGGGCUAG
AAUCUCGCUGGCCAGAGCCGUGUAUAAGGACGCCGACCUGUAUCUCCUGGACUCC
CCCUUCGGAUACCUGGACGUCCUGACCGAAAAGGAGAUCUUCGAAUCGUGCGUGU
GCAAGCUGAUGGCUAACAAGACUCGCAUCCUCGUGACCUCCAAAAUGGAGCACCU
GAAGAAGGCAGACAAGAUUCUGAUUCUGCAUGAGGGGUCCUCCUACUUUUACGG
CACCUUCUCGGAGUUGCAGAACUUGCAGCCCGACUUCUCAUCGAAGCUGAUGGGU
UGCGACAGCUUCGACCAGUUCUCCGCCGAAAGAAGGAACUCGAUCCUGACGGAAA
CCUUGCACCGCUUCUCUUUGGAAGGCGACGCCCCUGUGUCAUGGACCGAGACUAA
GAAGCAGAGCUUCAAGCAGACCGGGGAAUUCGGCGAAAAGAGGAAGAACAGCAU
CUUGAACCCCAUUAACUCCAUCCGCAAGUUCUCAAUCGUGCAAAAGACGCCACUG
CAGAUGAACGGCAUUGAGGAGGACUCCGACGAACCCCUUGAGAGGCGCCUGUCCC
UGGUGCCGGACAGCGAGCAGGGAGAAGCCAUCCUGCCUCGGAUUUCCGUGAUCUC
CACUGGUCCGACGCUCCAAGCCCGGCGGCGGCAGUCCGUGCUGAACCUGAUGACC
CACAGCGUGAACCAGGGCCAAAACAUUCACCGCAAGACUACCGCAUCCACCCGGA
AAGUGUCCCUGGCACCUCAAGCGAAUCUUACCGAGCUCGACAUCUACUCCCGGAG
ACUGUCGCAGGAAACCGGGCUCGAAAUUUCCGAAGAAAUCAACGAGGAGGAUCU
GAAAGAGUGCUUCUUCGACGAUAUGGAGUCGAUACCCGCCGUGACGACUUGGAA
CACUUAUCUGCGGUACAUCACUGUGCACAAGUCAUUGAUCUUCGUGCUGAUUUG
GUGCCUGGUGAUUUUCCUGGCCGAGGUCGCGGCCUCACUGGUGGUGCUCUGGCUG
UUGGGAAACACGCCUCUGCAAGACAAGGGAAACUCCACGCACUCGAGAAACAACA
GCUAUGCCGUGAUUAUCACUUCCACCUCCUCUUAUUACGUGUUCUACAUCUACGU
CGGAGUGGCGGAUACCCUGCUCGCGAUGGGUUUCUUCAGAGGACUGCCGCUGGUC
CACACCUUGAUCACCGUCAGCAAGAUUCUUCACCACAAGAUGUUGCAUAGCGUGC
UGCAGGCCCCCAUGUCCACCCUCAACACUCUGAAGGCCGGAGGCAUUCUGAACAG
AUUCUCCAAGGACAUCGCUAUCCUGGACGAUCUCCUGCCGCUUACCAUCUUUGAC
UUCAUCCAGCUGCUGCUGAUCGUGAUUGGAGCAAUCGCAGUGGUGGCGGUGCUG
CAGCCUUACAUUUUCGUGGCCACUGUGCCGGUCAUUGUGGCGUUCAUCAUGCUGC
GGGCCUACUUCCUCCAAACCAGCCAGCAGCUGAAGCAACUGGAAUCCGAGGGACG
AUCCCCCAUCUUCACUCACCUUGUGACGUCGUUGAAGGGACUGUGGACCCUCCGG
GCUUUCGGACGGCAGCCCUACUUCGAAACCCUCUUCCACAAGGCCCUGAACCUCC
ACACCGCCAAUUGGUUCCUGUACCUGUCCACCCUGCGGUGGUUCCAGAUGCGCAU
CGAGAUGAUUUUCGUCAUCUUCUUCAUCGCGGUCACAUUCAUCAGCAUCCUGACU
ACCGGAGAGGGAGAGGGACGGGUCGGAAUAAUCCUGACCCUCGCCAUGAACAUU
AUGAGCACCCUGCAGUGGGCAGUGAACAGCUCGAUCGACGUGGACAGCCUGAUGC
GAAGCGUCAGCCGCGUGUUCAAGUUCAUCGACAUGCCUACUGAGGGAAAACCCAC
UAAGUCCACUAAGCCCUACAAAAAUGGCCAGCUGAGCAAGGUCAUGAUCAUCGAA
AACUCCCACGUGAAGAAGGACGAUAUUUGGCCCUCCGGAGGUCAAAUGACCGUGA
AGGACCUGACCGCAAAGUACACCGAGGGAGGAAACGCCAUUCUCGAAAACAUCAG
CUUCUCCAUUUCGCCGGGACAGCGGGUCGGCCUUCUCGGGCGGACCGGUUCCGGG
AAGUCAACUCUGCUGUCGGCUUUCCUCCGGCUGCUGAAUACCGAGGGGGAAAUCC
AAAUUGACGGCGUGUCUUGGGAUUCCAUUACUCUGCAGCAGUGGCGGAAGGCCU
UCGGCGUGAUCCCCCAGAAGGUGUUCAUCUUCUCGGGUACCUUCCGGAAGAACCU
GGAUCCUUACGAGCAGUGGAGCGACCAAGAAAUCUGGAAGGUCGCCGACGAGGU
CGGCCUGCGCUCCGUGAUUGAACAAUUUCCUGGAAAGCUGGACUUCGUGCUCGUC
GACGGGGGAUGUGUCCUGUCGCACGGACAUAAGCAGCUCAUGUGCCUCGCACGGU
CCGUGCUCUCCAAGGCCAAGAUUCUGCUGCUGGACGAACCUUCGGCCCACCUGGA
UCCGGUCACCUACCAGAUCAUCAGGAGGACCCUGAAGCAGGCCUUUGCCGAUUGC
ACCGUGAUUCUCUGCGAGCACCGCAUCGAGGCCAUGCUGGAGUGCCAGCAGUUCC
UGGUCAUCGAGGAGAACAAGGUCCGCCAAUACGACUCCAUUCAAAAGCUCCUCAA
CGAGCGGUCGCUGUUCAGACAAGCUAUUUCACCGUCCGAUAGAGUGAAGCUCUUC
CCGCAUCGGAACAGCUCAAAGUGCAAAUCGAAGCCGCAGAUCGCAGCCUUGAAGG
AAGAGACUGAGGAAGAGGUGCAGGACACCCGGCUUUAAGGGUGGCAUCCCUGUG
ACCCCUCCCCAGUGCCUCUCCUGGCCCUGGAAGUUGCCACUCCAGUGCCCACCAGC
CUUGUCCUAAUAAAAUUAAGUUGCAUCAAAGCU

In some embodiments, an activity of CFTR proteins is evaluated by an Ussing chamber assay. In some embodiments, duration of activity of CFTR proteins is evaluated by time-course Ussing assays. In some embodiments, protein expression and stability are evaluated by pulse-chase methods. In some embodiments, protein expression and stability are evaluated by surface biotinylation.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

### Codon Optimization

According to an increasing amount of research, mRNAs contain numerous layers of information that overlap the amino acid code. Traditionally, codon optimization has been used to remove rare codons which were thought to be rate-limiting for protein expression. While fast growing bacteria and yeast both exhibit strong codon bias in highly expressed genes, higher eukaryotes exhibit much less codon bias, making it more difficult to discern codons that may be rate-limiting. In addition, it has been found that codon bias per se does not necessarily yield high expression but requires other features.

For example, rare codons have been implicated in slowing translation and forming pause sites, which may be required for correct protein folding. Therefore, variations in codon usage may provide a mechanism to fine-tune the temporal pattern of elongation and thus increase the time available for a protein to take on its correct confirmation. Codon optimization can interfere with this fine-tuning mechanism, resulting in less efficient protein translation or an increased amount of incorrectly folded proteins. Similarly, codon optimization may disrupt the normal patterns of cognate and wobble tRNA usage, thereby affecting protein structure and function because wobble-dependent slowing of elongation may likewise have been selected as a mechanism for achieving correct protein folding.

Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid (so may be referred to as a one-to-one sequence). The increase in expression is not limited to cell cultures of mammalian cells but was also observed in vivo in a mouse model. It is expected that the observed improvement in expression of the codon-optimised CFTR coding sequence, either wild type or activated CFTR, will result in an improved, more cost-effective mRNA replacement therapy for patients in need thereof, because it does not require the use of modified nucleotides for the preparation of the mRNA and allows treatment with a reduced dose and/or at extended dosing intervals.

In some embodiments, codon-optimized mRNA is produced in accordance with methods known in the art.

In some embodiments, codon-optimized mRNA sequences according to the present invention were further codon-optimized by a new process: the process first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded protein antigen.

Synthesis of mRNA Using SP6 RNA Polymerase

In some embodiments, CFTR mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. Examples of such labeled ribonucleotides include biotin-, fluorescein-, digoxigenin-, aminoallyl-, and isotope-labeled nucleotides.

The sequence for bacteriophage SP6 RNA polymerase was initially described (GenBank: Y00105.1) as having the following amino acid sequence:

(SEQ ID NO: 9)
MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA.

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 9. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 9. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

An SP6 RNA polymerase suitable for the invention may be a commercially-available product, e.g., from Aldevron, Ambion, New England Biolabs (NEB), Promega, and Roche. The SP6 may be ordered and/or custom designed from a commercial source or a non-commercial source according to the amino acid sequence of SEQ ID NO: 9 or a variant of SEQ ID NO: 9 as described herein. The SP6 may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In certain embodiments, a His tag is located at SP6's N-terminus.

### SP6 Promoter

Any promoter that can be recognized by an SP6 RNA polymerase may be used in the present invention. Typically, an SP6 promoter comprises 5′ ATTTAGGTGACACTATAG-3′ (SEQ ID NO: 10). Variants of the SP6 promoter have been discovered and/or created to optimize recognition and/or binding of SP6 to its promoter. Non-limiting variants include but are not limited to: 5′-ATTTAGGGGACACTATAGAAGAG-3′; 5′-ATTTAGGGGACACTATAGAAGG-3′; 5′-ATTTAGGGGACACTATAGAAGGG-3′; 5′-ATTTAGGTGACACTATAGAA-3′; 5′-ATTTAGGTGACACTATAGAAGA-3′; 5′-ATTTAGGTGACACTATAGAAGAG-3′; 5′-ATTTAGGTGACACTATAGAAGG-3′; 5′-ATTTAGGTGACACTATAGAAGGG-3′; 5′-ATTTAGGTGACACTATAGAAGNG-3′; and 5′-CATACGATTTAGGTGACACTATAG-3′ (SEQ ID NO: 11 to SEQ ID NO: 20).

In addition, a suitable SP6 promoter for the present invention may be about 95%, 90%, 85%, 80%, 75%, or 70% identical or homologous to any one of SEQ ID NO: 10 to SEQ ID NO: 20. Moreover, an SP6 promoter useful in the present invention may include one or more additional nucleotides 5′ and/or 3′ to any of the promoter sequences described herein.

### DNA Template

Typically, a CFTR DNA template is either entirely double-stranded or mostly single-stranded with a double-stranded SP6 promoter sequence.

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription with SP6, provided that they contain a double-stranded SP6 promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which are described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Large-Scale mRNA Synthesis

The present invention relates to large-scale production of codon optimized CFTR mRNA. In some embodiments, a method according to the invention synthesizes mRNA at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount. Generally, a reaction mixture includes SP6 RNA polymerase, a linear DNA template, and an RNA polymerase reaction buffer (which may include ribonucleotides or may require addition of ribonucleotides).

According to the present invention, 1-100 mg of SP6 polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of SP6 polymerase is used per gram of mRNA produced. In some embodiments, about 5-20 mg of SP6 polymerase is used to produce about 1 gram of mRNA. In some embodiments, about 0.5 to 2 grams of SP6 polymerase is used to produce about 100 grams of mRNA. In some embodiments, about 5 to 20 grams of SP6 polymerase is used to about 1 kilogram of mRNA. In some embodiments, at least 5 mg of SP6 polymerase is used to produce at least 1 gram of mRNA. In some embodiments, at least 500 mg of SP6 polymerase is used to produce at least 100 grams of mRNA. In some embodiments, at least 5 grams of SP6 polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the SP6 RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the SP6 RNA polymerase is from about 10 to 50 nM, 20 to 50 nM, or 30 to 50 nM. A concentration of 100 to 10000 Units/ml of the SP6 RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and SP6 RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL SP6 polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an SP6 polymerase-specific promoter, SP6 RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and SP6 RNA polymerase at a concentration ranging from 0.01-0.1 mg/ml, e.g., the reaction mixture comprises NTPs at a concentration of 5 mM, the DNA template at a concentration of 0.1 mg/ml, and the SP6 RNA polymerase at a concentration of 0.05 mg/ml.

### Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“yU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp(5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in US 2016/0040154, US 2015/0376220, PCT application PCT/US18/19954 entitled “METHODS FOR PURIFICATION OF MESSENGER RNA” filed on Feb. 27, 2018, and PCT application PCT/US18/19978 entitled “METHODS FOR PURIFICATION OF MESSENGER RNA” filed on Feb. 27, 2018, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of mRNA

Full-length or abortive transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

mRNA Solution

In some embodiments, mRNA may be provided in a solution to be mixed with a lipid solution such that the mRNA may be encapsulated in lipid nanoparticles. A suitable mRNA solution may be any aqueous solution containing mRNA to be encapsulated at various concentrations. For example, a suitable mRNA solution may contain an mRNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable mRNA solution may contain an mRNA at a concentration ranging from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.09 mg/ml, 0.08 mg/ml, 0.07 mg/ml, 0.06 mg/ml, or 0.05 mg/ml.

Typically, a suitable mRNA solution may also contain a buffering agent and/or salt. Generally, buffering agents can include HEPES, ammonium sulfate, sodium bicarbonate, sodium citrate, sodium acetate, potassium phosphate and sodium phosphate. In some embodiments, suitable concentration of the buffering agent may range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM. In some embodiments, suitable concentration of the buffering agent is or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM.

Exemplary salts can include sodium chloride, magnesium chloride, and potassium chloride. In some embodiments, suitable concentration of salts in an mRNA solution may range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM. Salt concentration in a suitable mRNA solution is or greater than about 1 mM, 5 mM, 10 mM, 20 mM, 30 mM, 40 mM, 50 mM, 60 mM, 70 mM, 80 mM, 90 mM, or 100 mM.

In some embodiments, a suitable mRNA solution may have a pH ranging from about 3.5-6.5, 3.5-6.0, 3.5-5.5., 3.5-5.0, 3.5-4.5, 4.0-5.5, 4.0-5.0, 4.0-4.9, 4.0-4.8, 4.0-4.7, 4.0-4.6, or 4.0-4.5. In some embodiments, a suitable mRNA solution may have a pH of or no greater than about 3.5, 4.0, 4.1, 4.2, 4.3, 4.4, 4.5, 4.6, 4.7, 4.8, 4.9, 5.0, 5.2, 5.4, 5.6, 5.8, 6.0, 6.1, 6.3, and 6.5.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

### Delivery Vehicles

According to the present invention, mRNA encoding a CFTR protein (e.g., a full length, fragment, or portion of a CFTR protein) as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

In some embodiments, a delivery vehicle comprising CFTR mRNA is administered by pulmonary delivery, e.g., comprising nebulization. In these embodiments, the delivery vehicle may be in an aerosolized composition which can be inhaled. In some embodiments, the mRNA is expressed in the tissue in which the delivery vehicle was administered, e.g., nasal cavity, trachea, bronchi, bronchioles, and/or other pulmonary system-related cell or tissue. Additional teaching of pulmonary delivery and nebulization are described in the related international application PCT/US17/61100 filed Nov. 10, 2017 by Applicant entitled “NOVEL ICE-BASED LIPID NANOPARTICLE FORMULATION FOR DELIVERY OF MRNA”, and the U. S. Provisional Application U.S. Ser. No. 62/507,061, each of which is incorporated by reference in its entirety.

In some embodiments, mRNAs encoding a CFTR protein may be delivered via a single delivery vehicle. In some embodiments, mRNAs encoding a CFTR protein may be delivered via one or more delivery vehicles each of a different composition. According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

A delivery vehicle comprising CFTR mRNA may be administered and dosed in accordance with current medical practice, taking into account the clinical condition of the subject, the site and method of administration (e.g., local and systemic, including oral, pulmonary, and via injection), the scheduling of administration, the subject's age, sex, body weight, and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein production.

In some embodiments, a CFTR mRNA is administered in combination with one or more CFTR potentiators and/or correctors. Suitable CFTR potentiators and/or correctors include ivacaftor (trade name Kalydeco®), lumacaftor (trade name Orkambi®) or the combination of ivacaftor and lumacaftor. In some embodiments, a CFTR mRNA is administered in combination with one or more other CF treatment such as hormone replacement therapies, thyroid hormone replacement therapy, non-steroidal inflammatory drugs, and prescription dronabinol (Marinol®) during treatment.

In some embodiments, the human subject receives concomitant CFTR modulator therapy. In some embodiments, the concomitant CFTR modulator therapy comprises ivacaftor. In some embodiments, the concomitant CFTR modulator therapy comprises lumacaftor. In some embodiments, the concomitant CFTR modulator therapy comprises tezacaftor. In some embodiments, the concomitant CFTR modulator therapy is selected from ivacaftor, lumacaftor, tezacaftor, or a combination. In some embodiments, the concomitant CFTR modulator therapy comprises VX-659. In some embodiments, the concomitant CFTR modulator therapy comprises VX-445. In some embodiments, the concomitant CFTR modulator therapy comprises VX-152. In some embodiments, the concomitant CFTR modulator therapy comprises VX-440. In some embodiments, the concomitant CFTR modulator therapy comprises VX-371. In some embodiments, the concomitant CFTR modulator therapy comprises VX-561. In some embodiments, the concomitant CFTR modulator therapy comprises GLPG1837. In some embodiments, the concomitant CFTR modulator therapy comprises GLPG2222. In some embodiments, the concomitant CFTR modulator therapy comprises GLPG2737. In some embodiments, the concomitant CFTR modulator therapy comprises GLPG2451. In some embodiments, the concomitant CFTR modulator therapy comprises GLPG1837. In some embodiments, the concomitant CFTR modulator therapy comprises PTI-428. In some embodiments, the concomitant CFTR modulator therapy comprises PTI-801. In some embodiments, the concomitant CFTR modulator therapy comprises PTI-808. In some embodiments, the concomitant CFTR modulator therapy comprises eluforsen.

In some embodiments, the human subject is not eligible for treatment with one or more of ivacaftor, lumacaftor, tezacaftor, VX-659, VX-445, VX-152, VX-440, VX-371, VX-561, VX-659 or combinations thereof. In some embodiments, the human subject is not eligible for treatment with one or more of ivacaftor, lumacaftor, tezacaftor, VX-659, VX-445, VX-152, VX-440, VX-371, VX-561, VX-659, GLPG1837, GLPG2222, GLPG2737, GLPG2451, GLPG1837, PTI-428, PTI-801, PTI-808, eluforsen, or combinations thereof.

In some embodiments, delivery vehicles are formulated such that they are suitable for extended-release of the mRNA contained therein. Such extended-release compositions may be conveniently administered to a subject at extended dosing intervals.

### Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired mRNA to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids. In some embodiments, a liposome comprises no more than three distinct lipid components. In some embodiments, one distinct lipid component is a sterol-based cationic lipid.

### Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

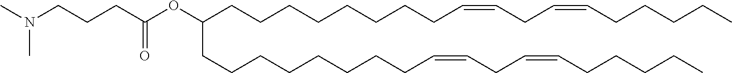 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

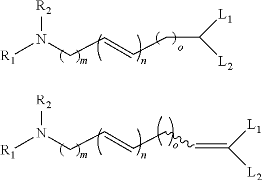

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C1-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

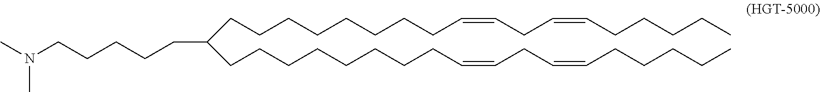 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

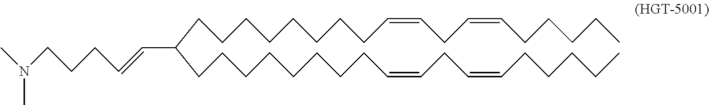 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

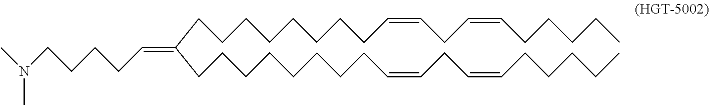 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

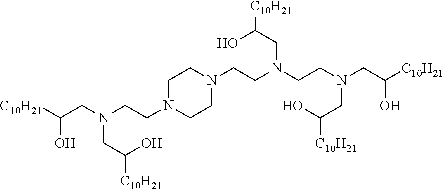

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

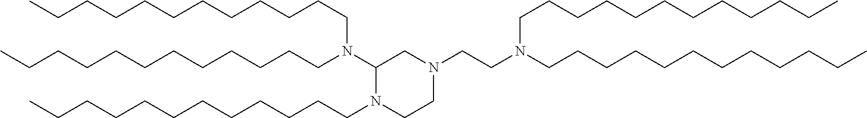 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

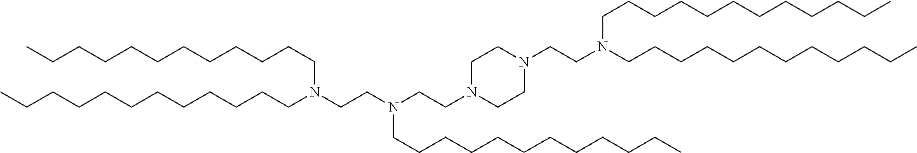 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

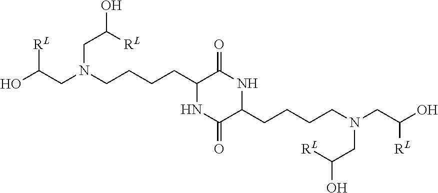

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

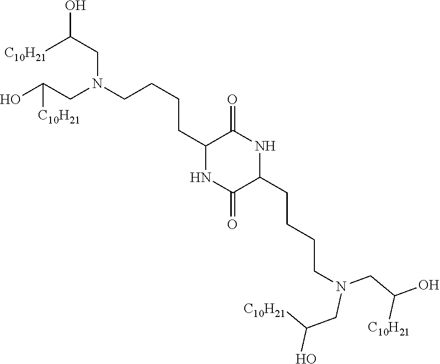

and pharmaceutically acceptable salts thereof. In certain embodiments, the compositions and methods of the present invention include a cationic lipid having a compound structure of:

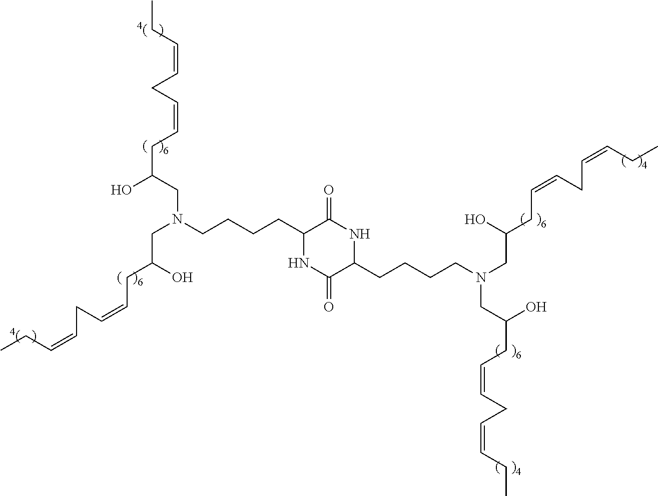

and pharmaceutically acceptable salts thereof. In certain embodiments, the compositions and methods of the present invention include a cationic lipid having a compound structure of:

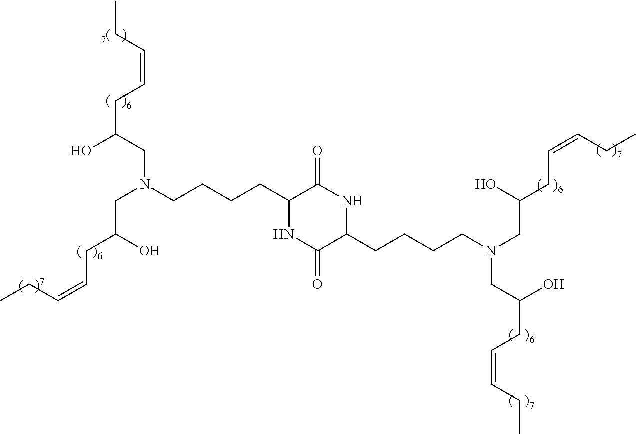

and pharmaceutically acceptable salts thereof. In certain embodiments, the compositions and methods of the present invention include a cationic lipid having a compound structure of.

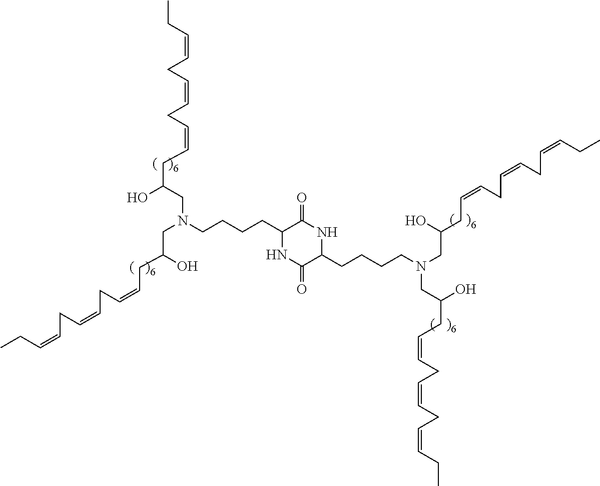

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

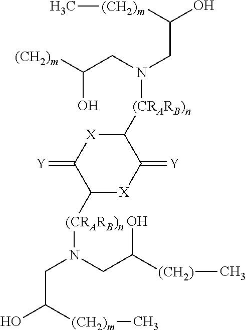

or a pharmaceutically acceptable salt thereof, wherein each X independently is O or S; each Y independently is O or S; each m independently is 0 to 20; each n independently is 1 to 6; each RA is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

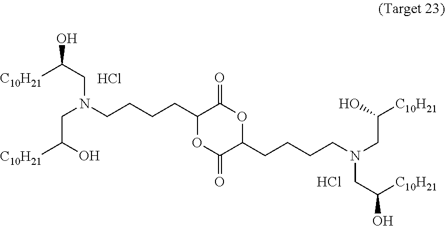

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

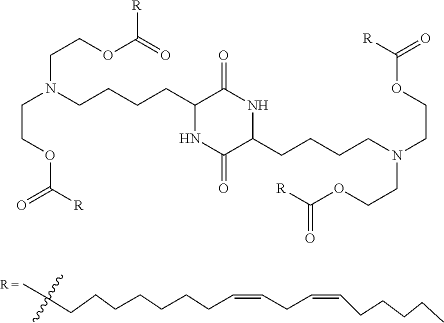

or a pharmaceutically acceptable salt thereof. In some embodiments, the compositions and methods of the present invention include a cationic lipid having the compound structure:

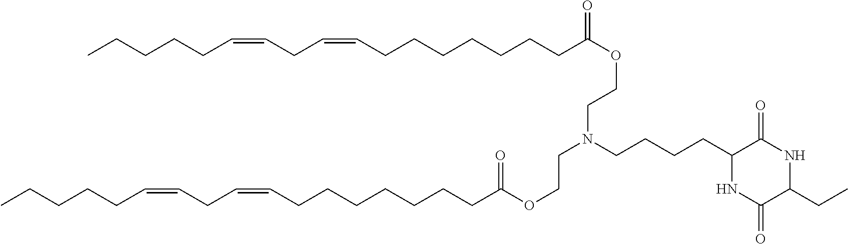 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

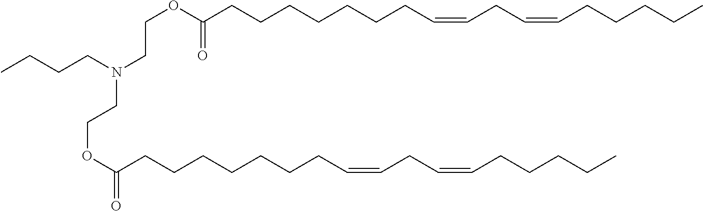 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

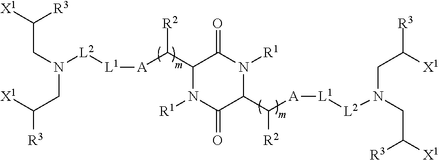

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

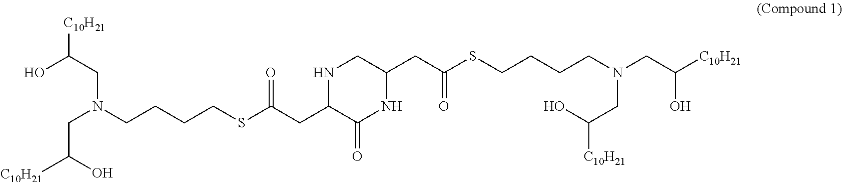

or a pharmaceutically acceptable salt thereof. In some embodiments, the compositions and methods of the present invention include a cationic lipid of the following formula:

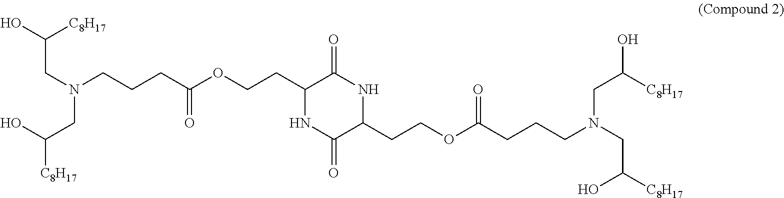

or a pharmaceutically acceptable salt thereof. In some embodiments, the compositions and methods of the present invention include a cationic lipid of the following formula:

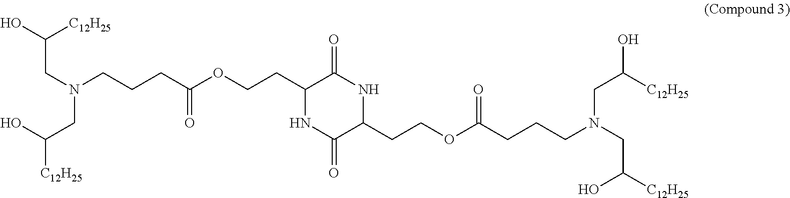

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

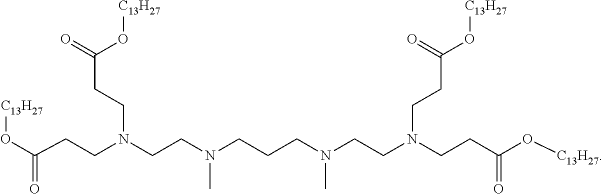

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

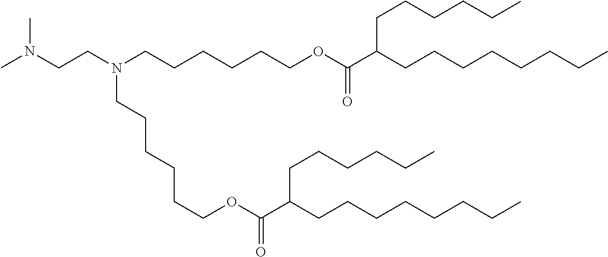 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

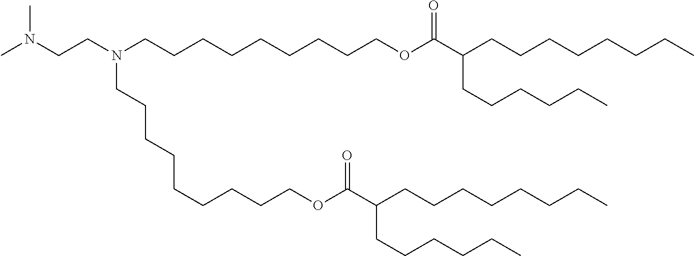 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

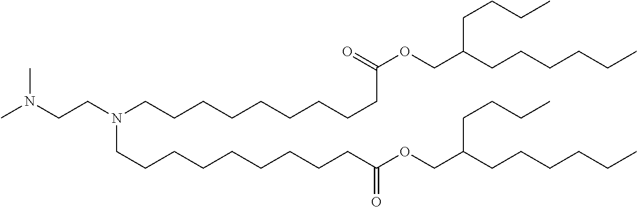 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

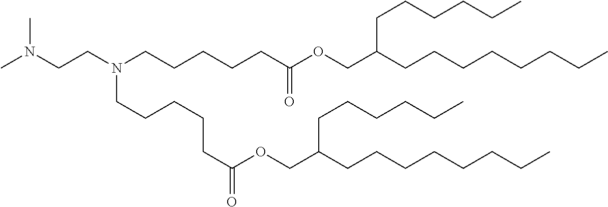 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

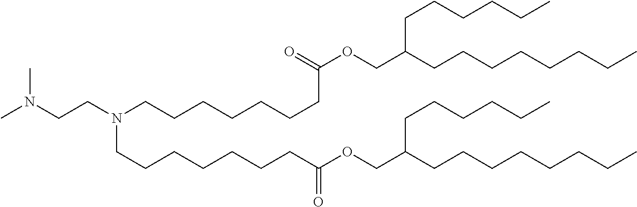 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

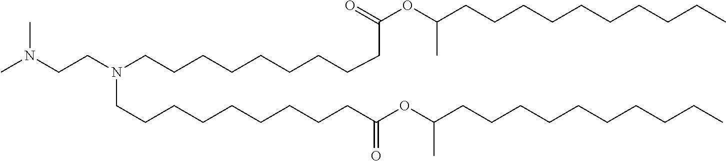 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

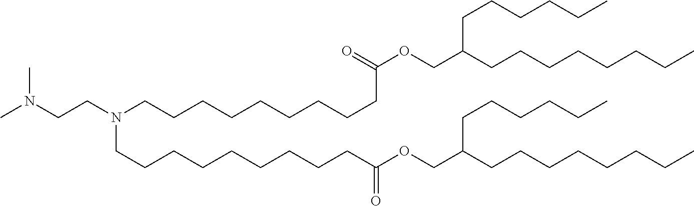 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

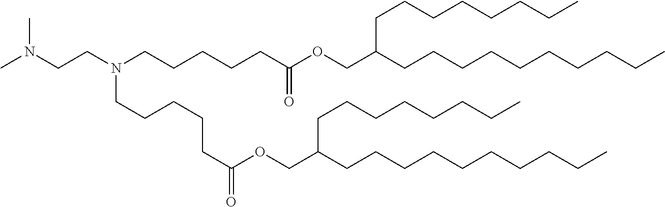 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

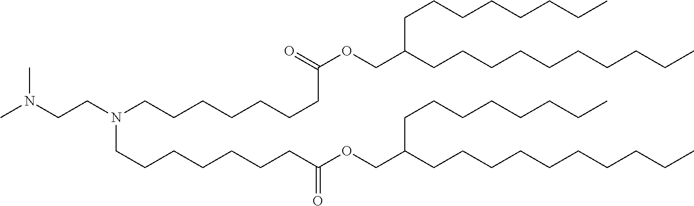 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

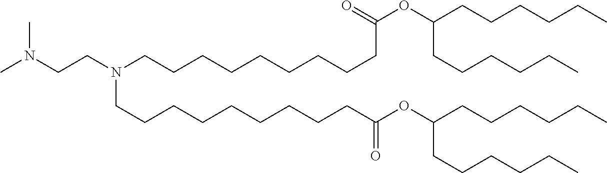 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

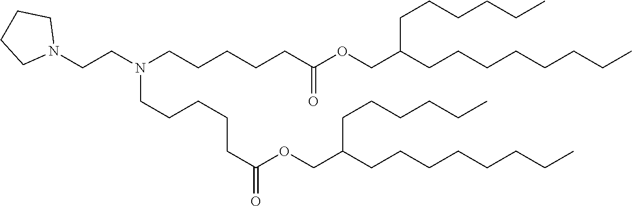 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

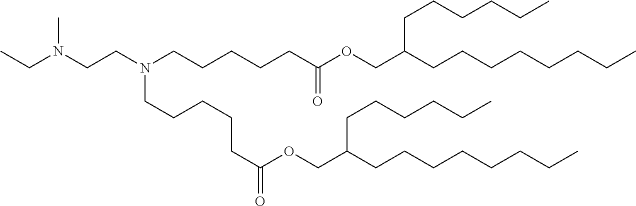 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

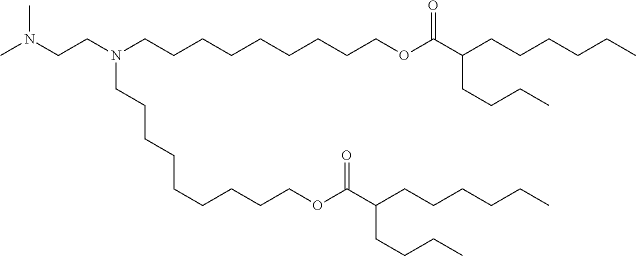 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

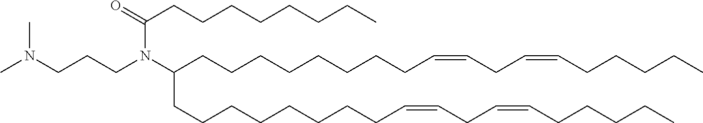 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

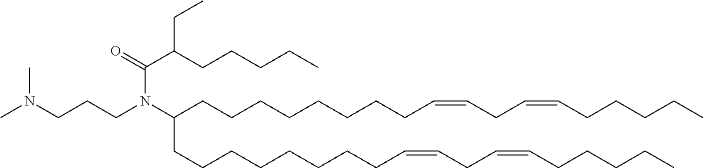 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

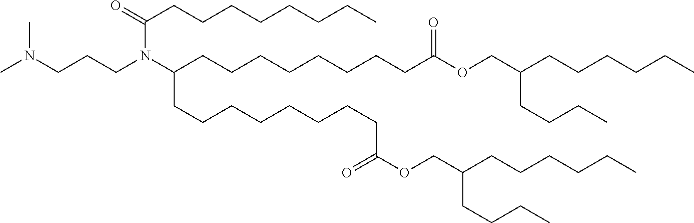 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

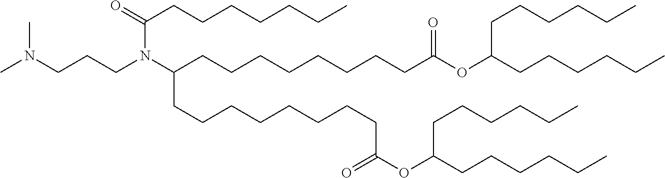 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

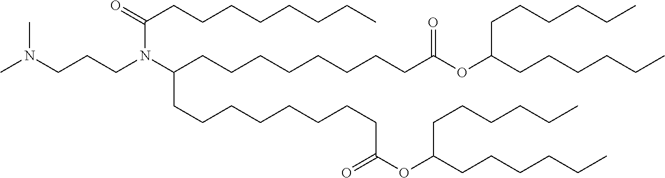 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

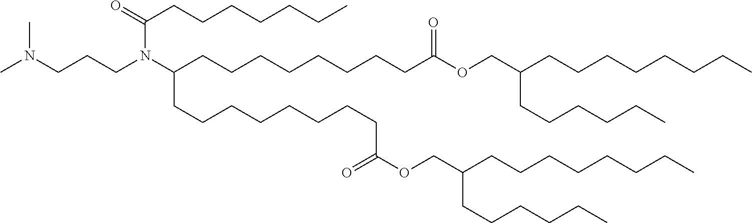 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

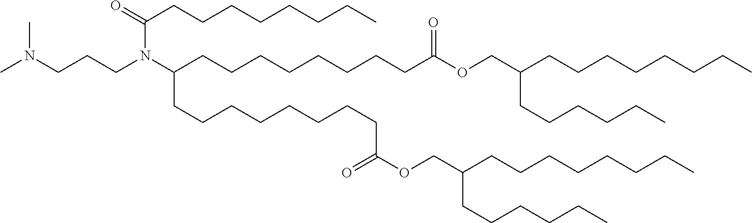 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

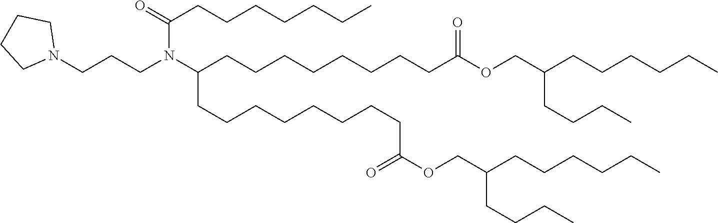 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

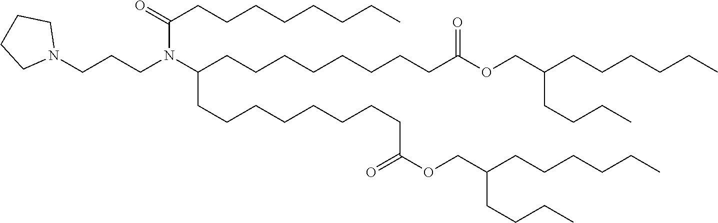 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

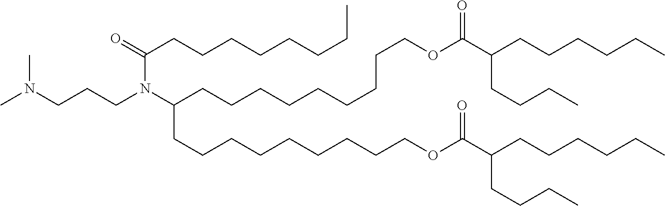 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

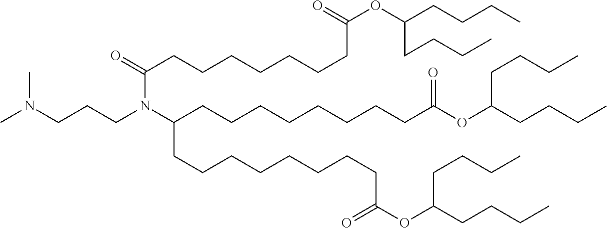 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

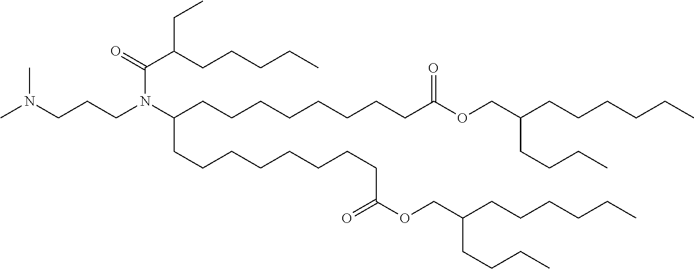 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

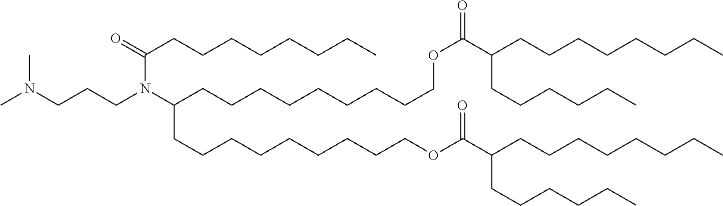 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

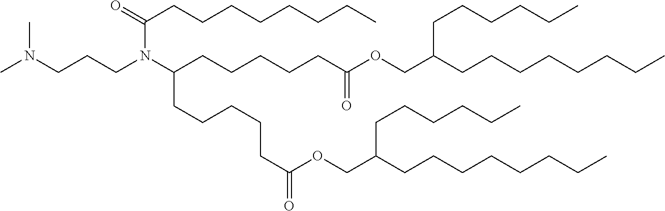 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

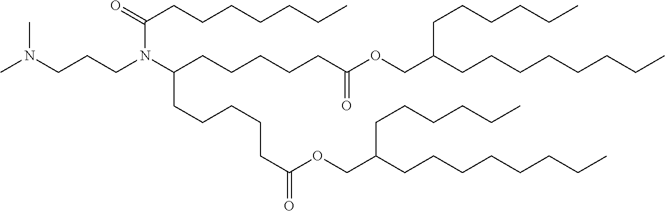 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

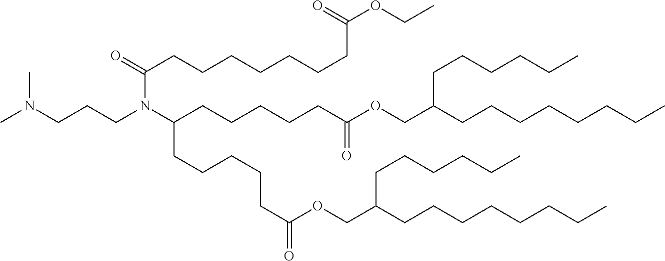 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

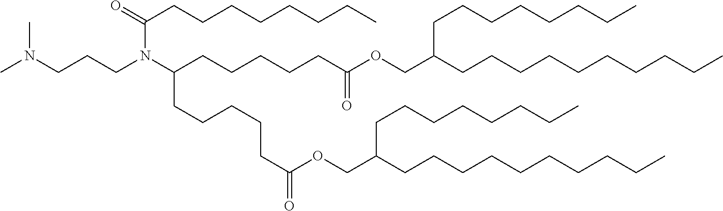 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

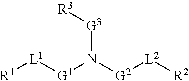

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; Ra is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

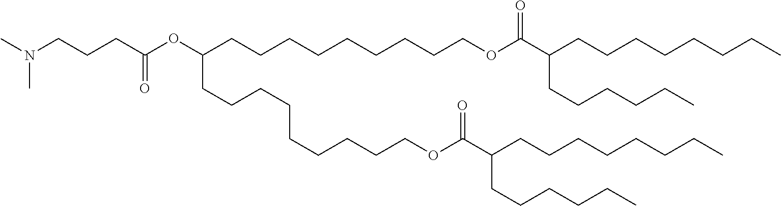 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

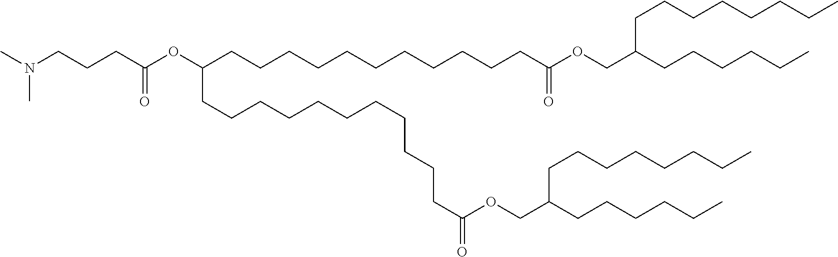 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

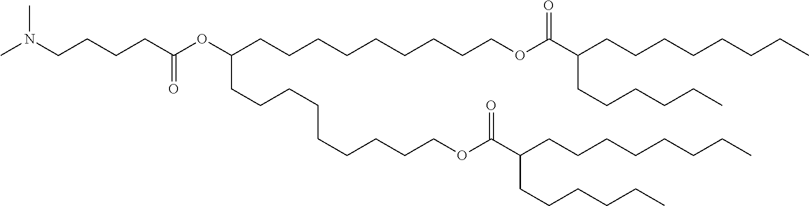 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

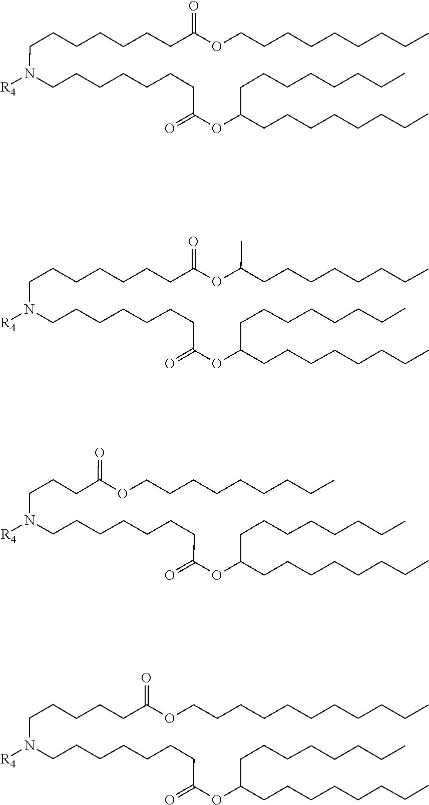

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

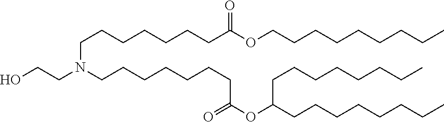 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

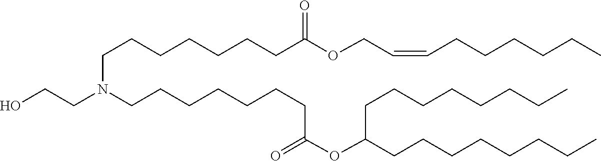 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

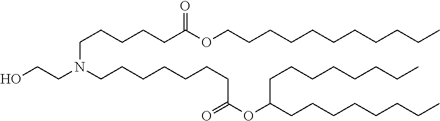 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

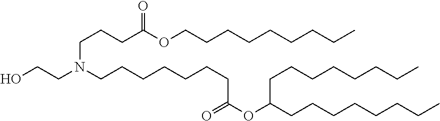 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

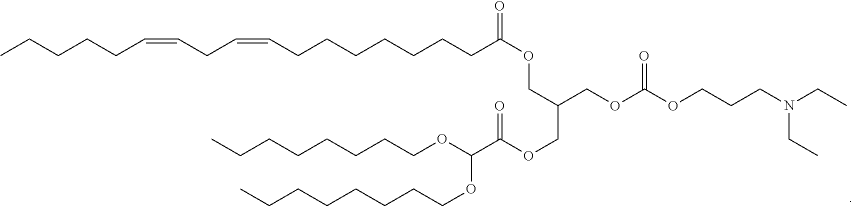 [SMILES: CCCCC/C=C\C/C=C\CCCCCCCC(=O)OCC(COC(=O)OCCCN(CC)CC)COC(=O)C(OCCCCCCCC)OCCCCCCCC]

and pharmaceutically acceptable salts thereof. In certain embodiments, the compositions and methods of the present invention include a cationic lipid having a compound structure of:

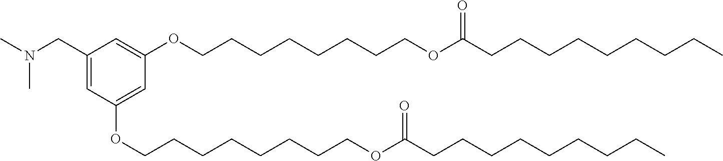 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

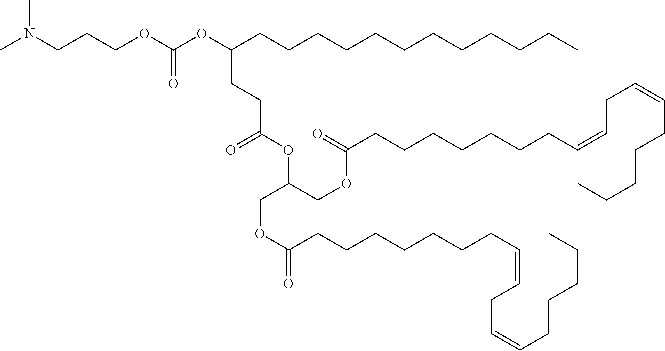 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

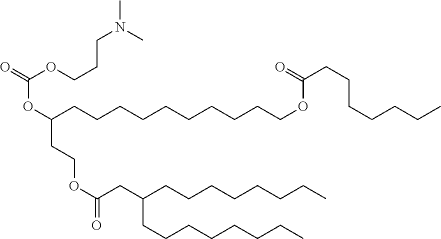 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

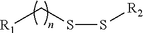

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

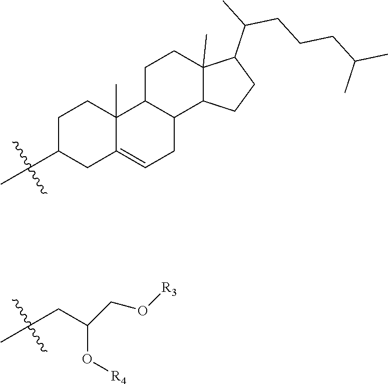

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

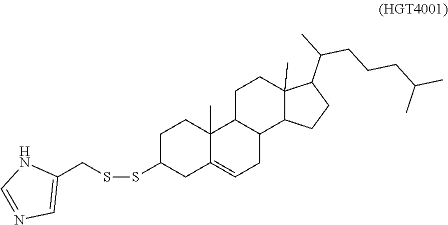 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002”, having a compound structure of:

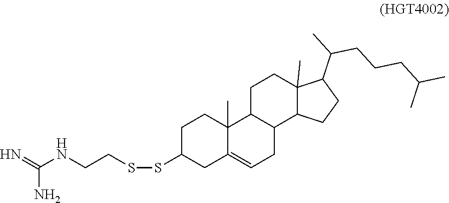 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003”, having a compound structure of:

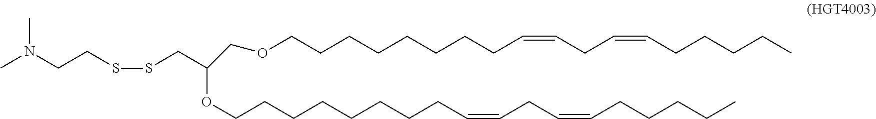 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004”, having a compound structure of:

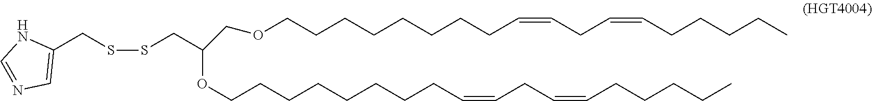 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005”, having a compound structure of:

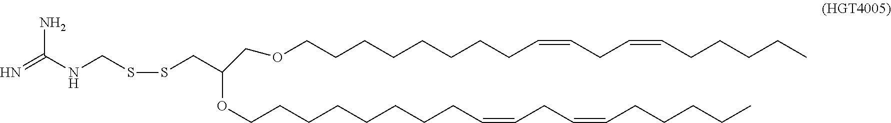 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in U.S. Provisional Application No. 62/672,194, filed May 16, 2018, and incorporated herein by reference. In certain embodiments, the compositions and methods of the present invention include a cationic lipid that is any of general formulas or any of structures (1a)-(21a) and (1b)-(21b) and (22)-(237) described in U.S. Provisional Application No. 62/672,194. In certain embodiments, the compositions and methods of the present invention include a cationic lipid that has a structure according to Formula (I′),

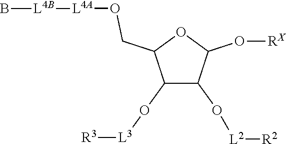

wherein:
   R^{X} is independently —H, -L¹-R¹, or -L^{5A}-L^{5B}-B′;
   each of L¹, L², and L³ is independently a covalent bond, —C(O)—, —C(O)O—, —C(O)S—, or —C(O)NR^{L}—;
   each L^{4A} and L^{5A} is independently —C(O)—, —C(O)O—, or —C(O)NR^{L}—;
   each L^{4B} and L^{5B} is independently C₁-C₂₀ alkylene; C₂-C₂₀ alkenylene; or C₂-C₂₀ alkynylene;
   each B and B′ is NR⁴R⁵ or a 5- to 10-membered nitrogen-containing heteroaryl;
   each R¹, R², and R³ is independently C₆-C₃₀ alkyl, C₆-C₃₀ alkenyl, or C₆-C₃₀ alkynyl;
   each R⁴ and R⁵ is independently hydrogen, C₁-C₁₀ alkyl; C₂-C₁₀ alkenyl; or C₂-C₁₀ alkynyl; and
   each R^{L} is independently hydrogen, C₁-C₂₀ alkyl, C₂-C₂₀ alkenyl, or C₂-C₂₀ alkynyl.

In certain embodiments, the compositions and methods of the present invention include a cationic lipid that is Compound (139) of 62/672,194, having a compound structure of:

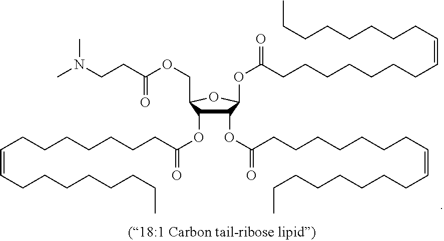

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′, 1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,1 2-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

### Non-Cationic Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

### Cholesterol-Based Lipids

In some embodiments, provided liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

### PEG-Modified Lipids

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the MCNA to be delivered. Additional considerations include, for example, the saturation of the alkyl chain, as well as the size, charge, pH, pKa, fusogenicity and toxicity of the selected lipid(s). Thus the molar ratios may be adjusted accordingly.

### Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

### Ratio of Distinct Lipid Components

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

(y+z)=100−x.

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Therapeutic Use of Compositions

In one aspect, the present invention, among other things, provide a method of inducing CFTR expression in vivo by administration of nucleic acids encoding a modified CFTR protein, or by administration of a modified CFTR protein. In some embodiments, a composition comprises nucleic acids encapsulated or complexed with a delivery vehicle. In some embodiments, the delivery vehicle is selected from the group consisting of liposomes, lipid nanoparticles, solid-lipid nanoparticles, polymers, viruses, sol-gels, and nanogels. In some embodiments, nucleic acids encoding a modified CFTR protein are packaged in a viral particle.

### Gene Therapy

In some embodiments, a pharmaceutical composition comprising nucleic acids encoding a modified CFTR protein is used to treat subjects in need thereof. In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. The pharmaceutical composition can be in a lyophilized form. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal routes. In one embodiment, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle or an inorganic nanoparticle.

In some embodiments, a rAAV is pseudotyped. A pseudotyped rAAV is an infectious virus comprising any combination of an AAV capsid protein and a rAAV genome. Pseudotyped rAAV are useful to alter the tissue or cell specificity of rAAV, and may be employed alone or in conjunction with non-pseudotyped rAAV to transfer one or more genes to a cell, e.g., a mammalian cell. For example, pseudotyped rAAV may be employed subsequent to administration with non-pseudotyped rAAV in a mammal which has developed an immune response to the non-pseudotyped rAAV. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 ITRs, each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ JTRs within a rAAV vector of the invention may be homologous, i.e., from the same serotype, heterologous, i.e., from different serotypes, or chimeric, i.e., an ITR which has ITR sequences from more than one AAV serotype.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV2. In some embodiments, the rAAV vector is AAV3. In some embodiments, the rAAV vector is AAV4. In some embodiments, the rAAV vector is AAV5. In some embodiments, the rAAV vector is AAV6. In some embodiments, the rAAV vector is AAV7. In some embodiments, the rAAV vector is AAV8. In some embodiments, the rAAV vector is AAV9. In some embodiments, the rAAV vector is AAV10. In some embodiments, the rAAV vector is AAV11. In some embodiments, the rAAV vector is sequence optimized. In some embodiments, the rAAV capsid is modified. For example, in some embodiments, the rAAV8 capsid is modified.

### Protein Replacement Therapy

The term “protein replacement” refers to the introduction of a non-native, purified protein into an individual having a deficiency in such protein. In some embodiments, a modified CFTR protein of the present invention can be used to treat subjects suffering from CFTR protein insufficiency.

A modified CFTR protein can be administered as a pure compound, but is advantageously presented in the form of a pharmaceutical preparation. In some embodiments, a modified CFTR protein is administered in a pharmaceutically acceptable carrier. In some embodiments, a modified CFTR protein can be formulated for oral, parenteral or rectal administration, or in forms suited to administrations by inhalation or insufflation (either via the mouth or nose).

For oral administrations, the pharmaceutical preparations are in the form of, for example, tablets or capsules prepared by known methods with pharmaceutically acceptable excipients such as binders (for example pregelatinized maize starch, polyvinylpyrrolidone, or methyl cellulose); fillers (for example lactose, microcrystalline cellulose or calcium hydrogen phosphate); additives (for example magnesium stearate, talc, silica); disintegrants (for example potato starch); and/or lubricants (for example sodium lauryl sulphate). The tablets can be coated using known methods. Liquid preparations for oral administration have the form, for example, of solutions, syrups or suspensions, or can be in the form of a dry product that can be dissolved in water or another liquid prior to use. Said preparations are prepared by known methods with pharmaceutically acceptable additives such as suspending agents (for example sorbitol, cellulose derivatives, edible hydrogenated fats); emulsifying agents (for example lecithin or acacia); non-aqueous liquids (for example almond oil, oily esters, ethyl alcohol or fractionated vegetable oils); and/or preservatives (for example methyl or propylhydroxybenzoates, sorbic acid or ascorbic acid). The preparations can also contain, in appropriate cases, buffering salts, colouring agents, flavouring agents and/or sweeteners.

Preparations for oral administration are formulated in a known manner, in order to provide a controlled release of the active compound.

In some embodiments, a modified CFTR protein is formulated, in a known manner, for parenteral administration, by injection or continuous administration. Formulations for injection are, advantageously, in the form of dosage units, for example in ampoules or multi-dose containers containing preservatives. The composition can be in the form of a suspension, in aqueous or oily liquids, and can contain elements of the formulation as dispersing and stabilizing agents. Alternatively, the active compound can be in powder form to be dissolved just before use in a liquid as needed, such as sterile water.

In some embodiments, a modified CFTR protein can be formulated for rectal administration as suppositories or enemas, for example, containing suppository excipients of known type such as cocoa butter or other glycerides.

In some embodiments, a modified CFTR protein is also formulated, in a known manner, in extended release compositions. These extended release compositions are, for example, administered by means of an implant (for example subcutaneous or intramuscular) or an intramuscular injection. Therefore, for example, a modified CFTR protein is formulated with suitable polymer or hydrophobic materials (such as an emulsion or an oil) or ion exchange resins, or relatively poorly soluble derivatives, such as relatively poorly soluble salts.

In some embodiments, a modified CFTR protein is administered by intranasal delivery. In some embodiments, a modified CFTR protein is formulated by administrations via a (known) device, such as in a powder with a suitable vehicle. In some embodiments, a modified CFTR protein is administered by pulmonary delivery.

In certain embodiments, a modified CFTR protein is administered on a suitable dosage schedule, for example, weekly, twice weekly, monthly, twice monthly, etc. In certain embodiments, the modified CFTR protein is administered once weekly by inhalation. The activated CFTR protein can be administered in any therapeutically effective amount.

### EXAMPLES

While certain compounds, compositions and methods of the present invention have been described with specificity in accordance with certain embodiments, the following examples serve only to illustrate the compounds of the invention and are not intended to limit the same.

### Example 1. Schematic Model of Regulation of Cystic Fibrosis Transmembrane Conductance Regulator (CFTR) Channel Activity

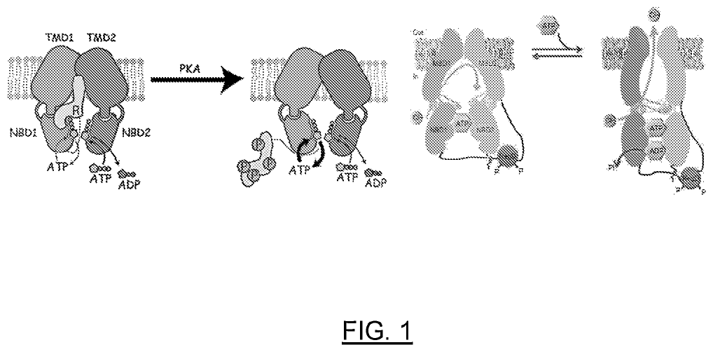

This example illustrates a schematic model for the regulation of CFTR channel activity (FIG. 1).

Briefly, CFTR is a chloride channel located in the apical membrane of epithelia and comprises 5 domains: two membrane spanning domains (TMD1 and TMD2) with six transmembrane segments each which form the chloride channel pore, two nucleotide binding domains (NBD1 and NBD2) that interact with and hydrolyze ATP, and a fifth unique unstructured regulatory domain called the R domain.

NBD1-NBD2 dimerization is promoted by cAMP-dependent Protein kinase A (PKA) phosphorylation of the unique R domain. Additionally, phosphorylation of the R domain regulates the CFTR activity through mechanisms independent of ATP-induced NBD dimerization.

CFTR is the only known ion channel in the ATP binding cassette (ABC) transporter superfamily. The binding of ATP to each of two sites in the nucleotide binding domains (NBD1 and NBD2 dimer interface) of CFTR drive conformational rearrangements of the transmembrane domains linked to cytosolic loops resulting in opening of the CFTR chloride channel (FIG. 1). ATP hydrolysis at the NBD2 site results in closing of the channel.

### Example 2. Engineering CFTR Activation/Stability Mutant Proteins

This example illustrates CFTR amino acid residues mutated in the CFTR gene that results in activation and opening of the CFTR chloride channel. For example, shown here are activating mutations that are R domain phosphomimetics, ATP-gating mutations and CFTR protein stability/expression-enhancing mutations.

The unstructured regulatory domain (R domain) of CFTR contains multiple protein kinase A (PKA) sites that when phosphorylated allow chloride channel gating by PKA.

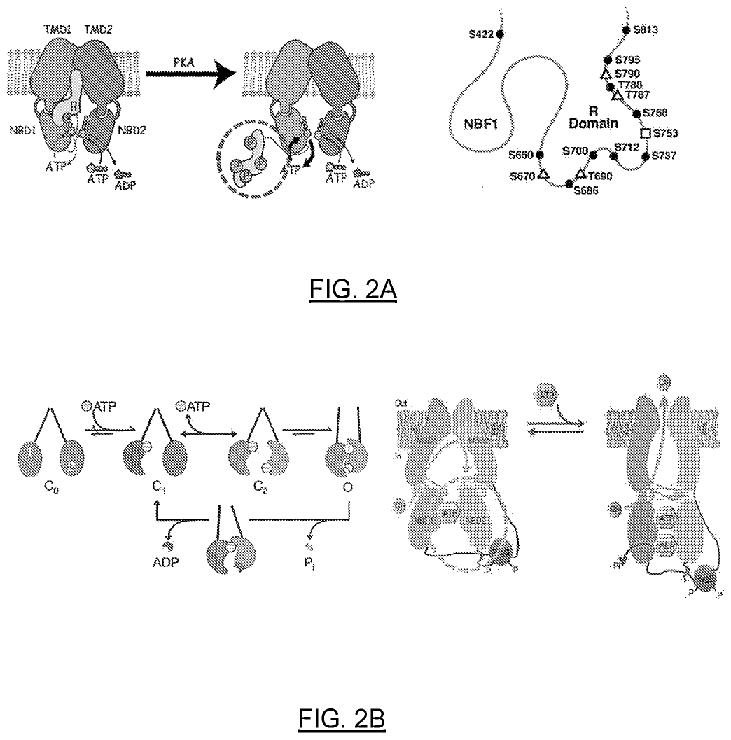

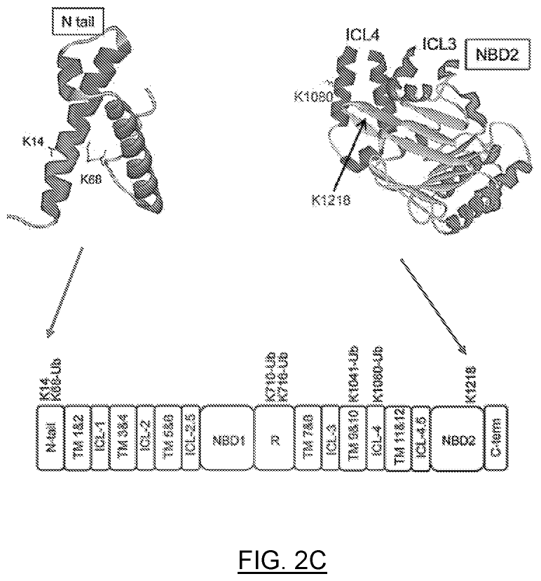

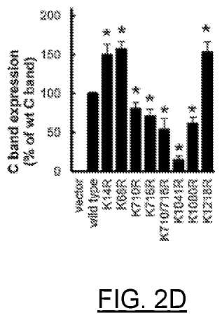

In this example, R domain phosphomimetics were generated by mutating residues described in Table 5 where X is any amino acid. R domain phosphomimetics included CFTR mutants such as S-Quad-D (S660D, S737D, S795D, S813D) and S-Hex-D (S660D, S685D, S700D, S737D, S795D, S813D), which are amino acid substitutions that mimic a phosphorylated CFTR, thereby resulting in activation (FIG. 2A, Table 3).

TABLE 5
CFTR Protein Activation Mutations: R-domain phosphomimetics

| | S422 | S660 | S670 | S686 | T690 | S700 | S712 | S737 | S753 | S768 | T787 | T788 | S790 | S795 | S813 |
|---|---|---|---|---|---|---|---|---|---|---|---|---|---|---|---|
| S-Quad-D | | D | | | | | | D | | | | | | D | D |
| (4D) | | | | | | | | | | | | | | | |
| S-Hex-D | | D | | D | | D | | D | | | | | | D | D |
| (6D) | | | | | | | | | | | | | | | |
| 8-D | | D | | D | | D | D | D | | D | | | | D | D |
| 15-D | D | D | D | D | D | D | D | D | D | D | D | D | D | D | D |
| 15-A | A | A | A | A | A | A | A | A | A | A | A | A | A | A | A |
| 13-D | D | D | D | D | D | D | D | | D | | D | D | D | D | D |
| S-Oct-D | | D | | D | | D | D | D | | D | | | | D | D |
| S-Quad-X | | X | | | | | | X | | | | | | X | X |
| S-Hex-X | | X | | X | | X | | X | | | | | | X | X |
| 15-X | X | X | X | X | X | X | X | X | X | X | X | X | X | X | X |
| 13-X | X | X | X | X | X | X | X | | X | | X | X | X | X | X |
| S-Oct-X | | X | | X | | X | X | X | | X | | | | X | X |

Another strategy used to engineer activation mutants of CFTR involved mutating residues involved in ATP gating to any other amino acid depicted by X as exemplified in Table 6. As one example, mutations K978C and E1371Q resulted in activation (FIG. 2B).

TABLE 6
CFTR Protein Activation Mutations: ATP-hydrolysis

| | S422 | S660 | S670 | S686 | T690 | S700 | S712 | S737 | S753 | S768 | T787 | T788 | S790 | S795 | S813 | K978 | E1371 |
|---|---|---|---|---|---|---|---|---|---|---|---|---|---|---|---|---|---|
| K978C | | | | | | | | | | | | | | | | C | |
| E1371Q | | | | | | | | | | | | | | | | | Q |
| 15-A | A | A | A | A | A | A | A | A | A | A | A | A | A | A | A | | Q |
| E1371Q | | | | | | | | | | | | | | | | | |
| 15-D | D | D | D | D | D | D | D | D | D | D | D | D | D | D | D | | Q |
| E1371Q | | | | | | | | | | | | | | | | | |
| K978X | | | | | | | | | | | | | | | | X | |
| E1371X | | | | | | | | | | | | | | | | | X |
| 15-X | X | X | X | X | X | X | X | X | X | X | X | X | X | X | X | | X |
| E1371X | | | | | | | | | | | | | | | | | |

CFTR proteins undergo ubiquitination at lysine residues. Amino acid mutations at lysine residues that result in a substitution of the lysine to another amino acid residue results in enhanced stability and protein expression of the CFTR protein (FIG. 2C). Exemplary mutant CFTR proteins were engineered as listed in Table 7. CFTR expression was measured as a proportion of WT CFTR (FIG. 2D)).

TABLE 7
Stability/Expression-enhancing engineered CFTR Proteins

| | K14 | K68 | K710 | K716 | K1041 | K1218 |
|---|---|---|---|---|---|---|
| 1 | R | | | | | |
| 2 | | R | | | | |
| 3 | R | R | | | | |
| 4 | | | | | | R |
| 5 | | R | | | | R |
| 6 | R | | | | | R |
| 7 | R | R | | | | R |
| 8 | | | R | | | |
| 9 | | | | R | | |
| 10 | | | | | R | |
| 11 | | | R | R | | |
| 12 | | | R | R | R | |
| 13 | X | | | | | |
| 14 | | X | | | | |
| 15 | X | X | | | | |
| 16 | | | | | | X |
| 17 | | X | | | | X |
| 18 | X | | | | | X |
| 19 | X | X | | | | X |
| 20 | | | X | | | |
| 21 | | | | X | | |
| 22 | | | | | X | |
| 23 | | | X | X | | |
| 23 | | | X | X | X | |

### Example 3. Evaluating Response of Activated CFTR Constructs in Ussing Chamber Assay at a High Concentration

In this example, mutant CFTR proteins were assessed for chloride ion transport using the Ussing assay. These assessments determined the activity of the mutant CFTR proteins.

TABLE 8
Activated CFTR Constructs

| Engineered | Activated CFTR Mutation | | | | | | | |
|---|---|---|---|---|---|---|---|---|
| CFTR | S660 | S686 | S700 | S737 | S795 | S813 | K978 | E1371 |
| A | | | | | | | | |
| B | D | | | D | D | D | | |
| C | D | D | D | D | D | D | | |
| D | | | | | | | C | |
| E | | | | | | | | Q |

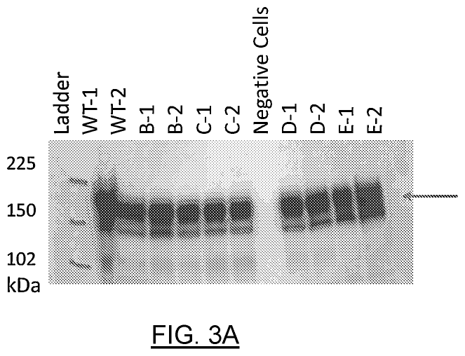

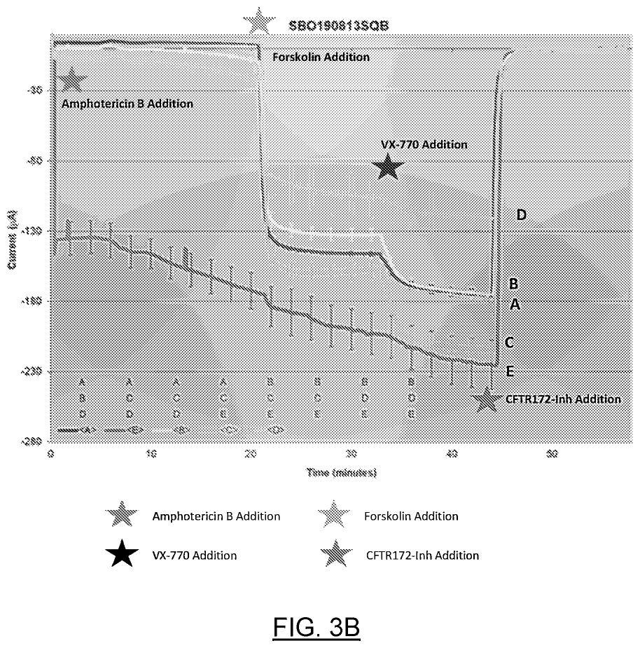

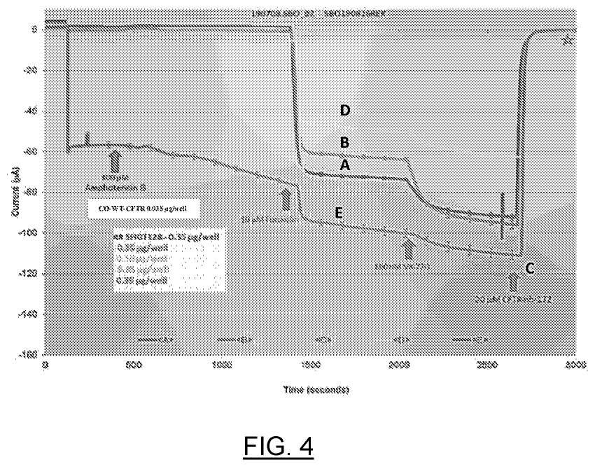

Wild type construct (CFTR Protein A in Table 8) and all mutant constructs (Engineered Proteins B-E in Table 8) were highly translated in BTEK293 cells and provided high CFTR expression as observed in a protein gel. The CFTR band is depicted by an arrow (FIG. 3A).

Chloride ion transport was measured by an Ussing chamber. This assay provides an indication of chloride flux based on the principle that the epithelium pumps ions from one side to another and ions leak back through tight junctions situated between epithelial cells. The voltage difference between two sides of the epithelium is measured using two voltage electrodes placed near the tissue/epithelium. The voltage is cancelled out by injecting current, using two other electrodes placed away from the epithelium. The short circuit current is measured as net chloride ion transport.

Traces in FIG. 3B show short-circuit current measurements measuring current over time, comparing WT CFTR (Protein A) relative to engineered CFTR mutant Protein B (R domain phosphomimetic S-Quad-D), engineered CFTR mutant Protein C (R domain phosphomimetic S-Hex-D), engineered CFTR mutant Protein D (K978C ATP independent activity), engineered CFTR mutant Protein E (E1371Q ATP hydrolysis deficient) at a concentration of 0.5 μg CFTR (“high concentration”).

Traces show short-circuit current measurements during sequential additions of forskolin, an activator of CFTR-mediated chloride secretion, and VX-770 (ivacaftor), a potentiator compound that directly increases phosphorylation-dependent CFTR channel opening on amphotericin B-permeabilized membranes. CFTR proteins A-D responded to forskolin activation resulting in increased chloride ion conductance. Forskolin activation was not observed for CFTR Protein E, the ATP hydrolysis deficient E1371Q mutant. Further addition of VX-770 increased conductance in CFTR Proteins B-D.

Conductance in all cases was lost upon inhibition of CFTR by treatment with the CFTR172-Inhibitor.

Varying profiles of response were observed at the high concentration of 0.5 μg of engineered CFTR proteins tested. (FIG. 3B)

### Example 4. Evaluating Response of Activated CFTR Constructs in Ussing Chamber Assay at a Low Concentration

In this example, activation of various engineered CFTR protein mutants (proteins B-E, discussed above) were compared with the activation of wild type CFTR (protein A) using the Ussing assay as described in Example 3. In this example the engineered CFTR protein activity was tested at a low concentration of 0.35 μg (FIG. 4).

Using a lower concentration (i.e., 0.35 μg) of the tested CFTR proteins showed improved reproducibility of current in the Ussing chamber analysis.

The results showed consistent trends in rank-ordered response to forskolin activation. Of the mutants tested, engineered CFTR mutant protein E showed forskolin activation at a lower dose in comparison to other constructs tested. Additionally, FIG. 4 demonstrate two distinct effects. Substitutions of cysteine for a particular lysine in cytosolic loop 3 (e.g., CFTR mutant D in Table 8) yielded a channel with overall reduced activity as compared to wild-type CFTR, though it did demonstrate slight conductance prior to forskolin addition. In contrast, substituting glutamine for a specific glutamic acid in NBD-2 (e.g., CFTR mutant E in Table 8) produced significant pre-forskolin conductance, as well as overall increased activity in FRT cells.

Furthermore, the data showed that addition of ivacaftor resulted in a stronger activation response in all of the constructs tested. Engineered CFTR mutant protein C had a more pronounced response to ivacaftor in comparison to the other constructs tested.

### Example 5. Dose Response Assessment of Engineered CFTR Protein E in Ussing Chamber Assay

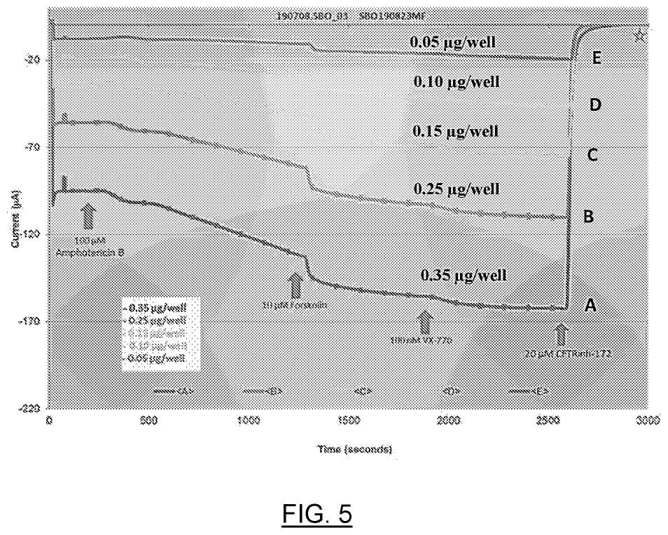

In this example, engineered CFTR protein E1371Q was tested for activation using various doses ranging from 0.05-0.35 μg/well using the Ussing assay (FIG. 5). FIG. 5 illustrates chloride flux from a cell expressing the mutant CFTR protein.

The data showed a consistent activity pattern across treatments at the different doses of tested.

### Example 6. Optimization of Escin Permeabilization for ATP Depletion Using WT CFTR at Different Doses

In this example, the effect of escin permeabilization for ATP depletion was assessed using wild type CFTR protein. The Ussing assay was used for these studies.

For these studies, the saponin escin was used as a perforating agent, which permits permeation of larger molecules including nucleotides and modulators of ion channel activity.

Ecsin treatment was carried out at doses of 10 μM, 15 μM, 20 μM, 25 μM in the presence and absence of ATP.

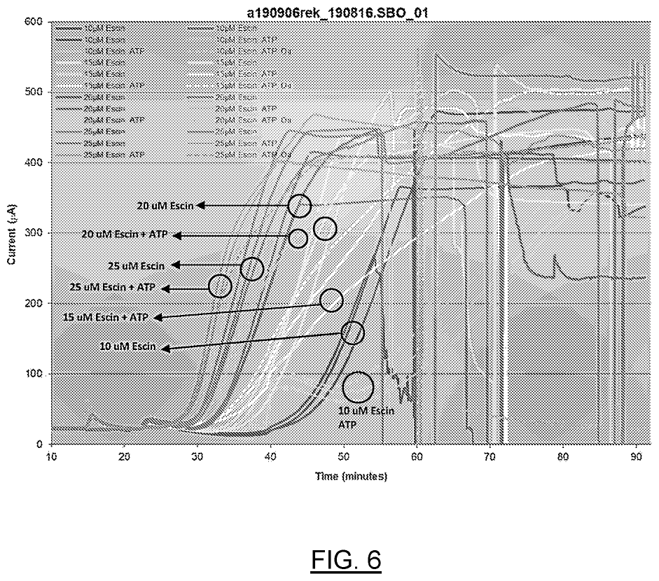

The results showed that ecsin permeabilization resulted in loss of membrane integrity at all levels tested in a dose and time dependent manner (FIG. 6). It was observed that that 10 μM ecsin yielded only a short window of time in which measurements with and without ATP could be compared, leading to additional optimizations.

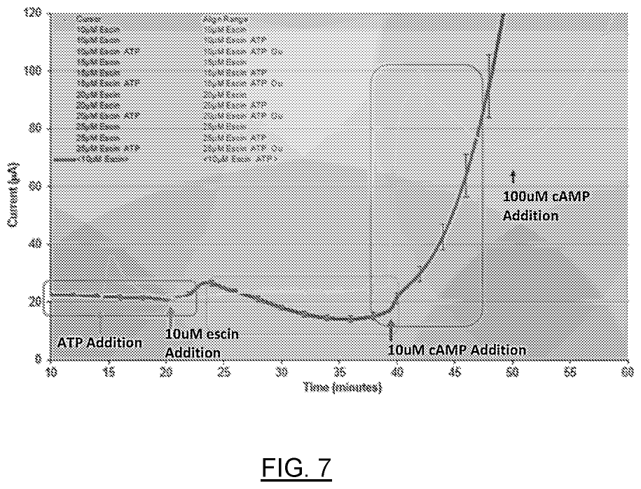

Further analysis was conducted to optimize ecsin permeabilization for ATP depletion (FIG. 7).

### Example 7. Evaluating Response of Phosphomimetic CFTR Constructs in Ussing Chamber Assay

In this example, activities of various engineered phosphomimetic CFTR protein mutants (Table. 9) were compared with the activity of wild-type CFTR using the Ussing chamber assay as described in Example 3.

TABLE 9
Phosphomimetic CFTR Constructs

| Engineered | |
|---|---|
| CFTR | Mutations |
| WT | Wild-type |
| 4D | S660D, S737D, S795D, S813D |
| 6D | S660D, S686D, S700D, S737D, S795D, S813D |
| 8D | S660D, S686D, S700D, S712D, S737D, S768D, S795D, |
| | S813D |
| 13D | S422D, S660D, S670D, S686D, T690D, S700D, S712D, |
| | S753D, T787D, T788D, T790D, S795D, S813D |
| 15D | S422D, S660D, S670D, S686D, T690D, S700D, S712D, |
| | S737D, S753D, S768D, T787D, T788D, T790D, S795D, |
| | S813D |

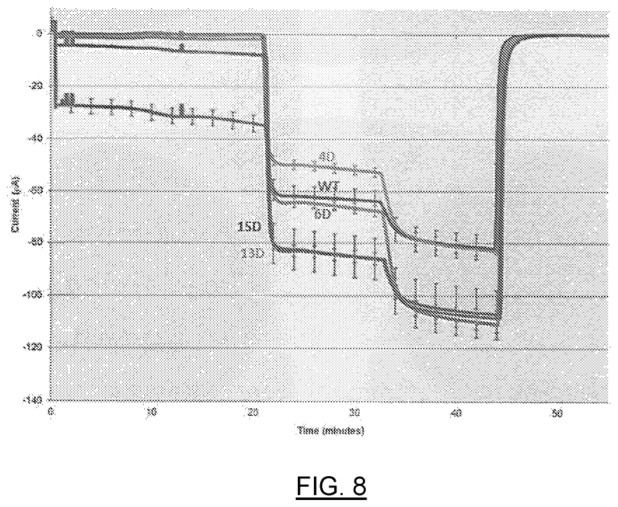

As shown in FIG. 8, combinations of S/T to D mutations in the R domain of CFTR impart various alterations to the channel functioning. 4D and 6D channels demonstrate greater responsiveness to VX-770, while 13D and 15D display forskolin-independent chloride transport, as well as increased overall activity as compared to wild-type CFTR channels. All phosphomimetic CFTR mutants, with an exception of the 4D mutant, showed higher overall activity as compared to the wild-type CFTR. The data also showed that increasing the number of phosphomimetic sites in the CFTR protein can increase the overall activity in Ussing chamber activity.

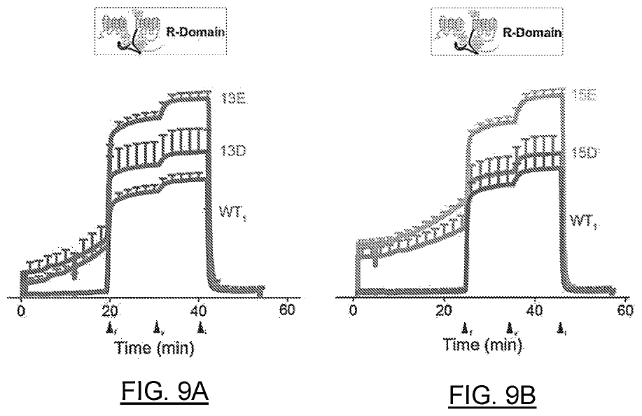

Next, several combinations of serine and threonine in the R-domain were replaced with negatively charged amino acids, either glutamic acid or aspartic acid, in order to mimic the electrostatic nature of phosphorylation. Two combinations were found to significantly impact chloride current—one containing 13 amino acid changes and the other with 15 amino acid changes. FIG. 9A shows a comparison of CFTR mutants containing 13 amino acid changes to either aspartate or glutamate in the R-domain. FIG. 9B shows comparison of CFTR mutants containing 15 amino acid changes to either aspartate or glutamate in the R-domain. In both cases, glutamate (E) substitutions were more effective than aspartate (D) substitutions.

The results showed that the phosphomimetic CFTR mutants were responsive to both forskolin and VX-700. The phosphomimetic CFTR mutants also showed increased current compared to the wild-type CFTR in the absence of forskolin (post-amphotericin) (FIG. 8, FIG. 9A and FIG. 9 B). Without being bound by a particular theory, this may be due a reduced threshold for cAMP gating, consistent with the expected phosphomimetic effect. While opening of wild-type channels depends on cAMP activation of PKA and downstream R-domain phosphorylation, the aspartate point mutations partially mimic constitutive phosphorylation such that the open-state conformation is adopted even in the absence of cAMP.

### Example 8. Evaluating Response of Stability/Trafficking CFTR Constructs in Ussing Chamber Assay

In this example, activities of various engineered stability/trafficking CFTR protein mutants (Table. 10) were compared with the activity of wild-type CFTR using the Ussing chamber assay as described in Example 3.

CFTR proteins undergo ubiquitination at multiple lysines in the protein. Ubiquitination is a reversible process due to the presence of deubiquitinating enzymes that can cleave ubiquitin from modified proteins. Ubiquitination is involved in regulation of both membrane trafficking and protein degradation. For example, the ubiquitinated K710, K716, and K1041 residues stabilize the wild-type CFTR, protecting it from proteolysis. Modifications of K14R and K68R lead to increased mature band C CFTR, which can be augmented by proteasomal inhibition allowing trafficking to the surface.

TABLE 10
Stability/Trafficking CFTR Constructs

| Engineered CFTR | Mutations |
|---|---|
| WT | Wild-type |
| A | K14R |
| B | K68R |
| C | K14R, K68R |
| D | K1218R |
| E | K68R, K1218R |
| F | K14R, K1218R |
| G | K14R, K68R, K1218R |

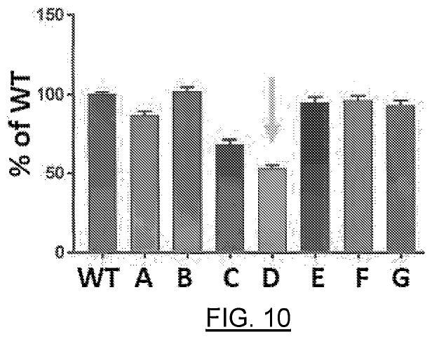

As shown in FIG. 10, no significant increase in activity was observed for the CFTR mutants in Table. 10, compared to the wild-type CFTR. The CFTR mutants A (K14R), C (K14R, K68R) and D (K1218R) showed decreased activity as compared to the wild-type CFTR as determined by Ussing chamber assay.

### Example 9. Evaluating Response of Phosphomimetic CFTR Constructs in Combination with K14R or E1371Q Mutation in Ussing Chamber Assay

In this example, activities of various engineered CFTR protein mutants (Table. 11) were compared using the Ussing chamber assay as described in Example 3. In particular, various combinations of K14R, E1371Q, and phosphomimetic mutations were tested.

To examine whether a synergistic effect can be achieved by combining phosphomimetic mutations with a trafficking mutation (K14R) or an ATP hydrolysis-deficient (E1371Q) mutation, CFTR mutant constructs shown in Table. 11 were prepared.

TABLE 11
Various CFTR Mutant Constructs

| Engineered | |
|---|---|
| CFTR | Mutations |
| WT | Wild-type |
| 13D | S422D, S660D, S670D, S686D, T690D, S700D, S712D, |
| | S753D, T787D, T788D, T790D, S795D, S813D |
| 13D/K14R | K14R, S422D, S660D, S670D, S686D, T690D, S700D, |
| | S712D, S753D, T787D, T788D, T790D, S795D, S813D |
| 13E | S422E, S660E, S670E, S686E, T690E, S700E, S712E, |
| | S753E, T787E, T788E, T790E, S795E, S813E |
| 13E/K14R | K14R, S422E, S660E, S670E, S686E, T690E, S700E, |
| | S712E, S753E, T787E, T788E, T790E, S795E, S813E |
| 15E | S422E, S660E, S670E, S686E, T690E, S700E, S712E, |
| | S737E, S753E, S768E, T787E, T788E, T790E, S795E, |
| | S813E |
| 15E/K14R | K14R, S422E, S660E, S670E, S686E, T690E, S700E, |
| | S712E, S737E, S753E, S768E, T787E, T788E, T790E, |
| | S795E, S813E |
| 15A | S422A, S660A, S670A, S686A, T690A, S700A, S712A, |
| | S737A, S753A, S768A, T787A, T788A, T790A, S795A, |
| | S813A |
| 15A/E1371Q | S422A, S660A, S670A, S686A, T690A, S700A, S712A, |
| | S737A, S753A, S768A, T787A, T788A, T790A, S795A, |
| | S813A, E1371Q |
| 15D/E1371Q | S422D, S660D, S670D, S686D, T690D, S700D, S712D, |
| | S737D, S753D, S768D, T787D, T788D, T790D, S795D, |
| | S813D, E1371Q |

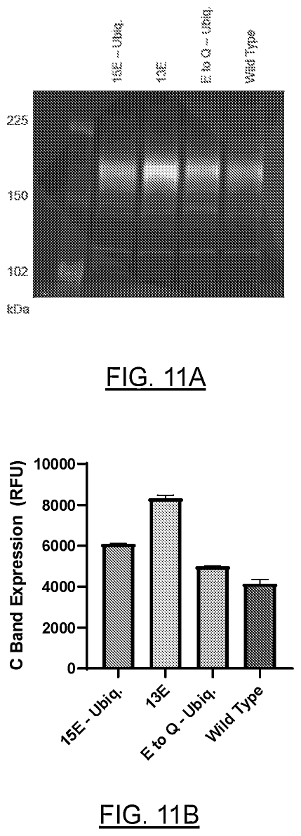

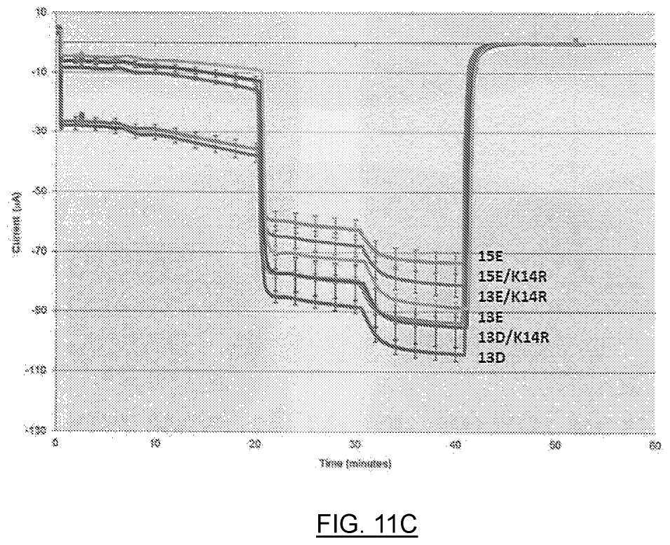

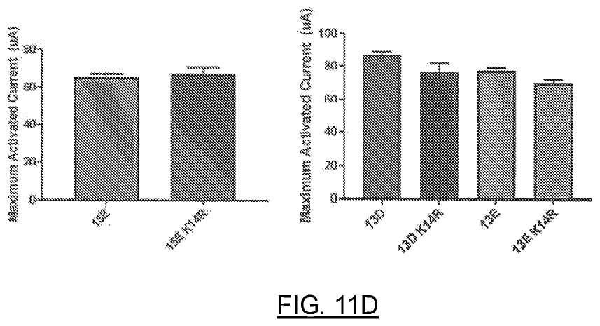

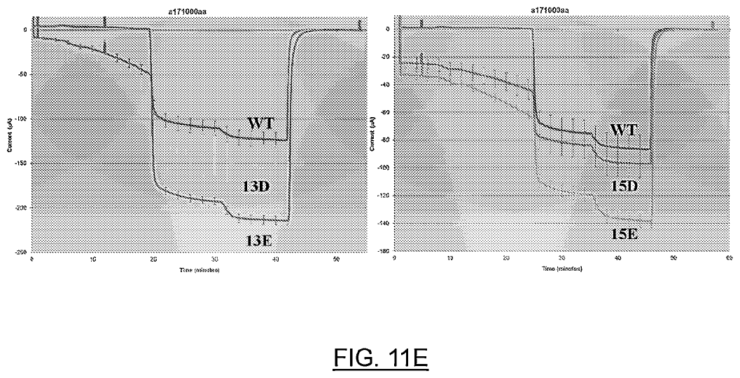

To examine the expression of CFTR mutants, expression of the wild-type CFTR, 15E, 13E, and E1371Q mutants was quantified as shown in FIG. 11A and FIG. 11B. Quantification of Western blot demonstrated an increased C-band expression from three variant CFTRs, with 13E showing a greater than 2-fold increase in protein.

As shown in FIG. 11C, the K14R mutation did not further increase the activity of the phosphomimetic CFTR mutants. FIG. 11D is a bar graph representation of maximum activated current for each mutant. FIG. 11D shows that when K14R mutation was combined with 15E, 13D, or 13E phosphomimetic mutants, there was no significant increase in the activity of the CFTR proteins.

Studies were also performed to assess whether there were any differences in CFTR activity following S to D amino acid substitutions in comparison to S to E amino acid substitutions. For these studies, the activity of CFTR mutant 13D was compared to the activity of 13E using Ussing chamber. The activity of CFTR mutant 15D was compared to the activity of 15E using the Ussing chamber. The data from these studies showed that E substitutions produced CFTR that had increased activity in comparison to those that had D substitutions (FIG. 11E). Thus, E amino acid substitutions can be used to further increase CFTR activity. For example, S13E and S15E substitutions have greater CFTR activity than S13D and S15D substitutions, respectively.

FIG. 12A and FIG. 12B show that the CFTR mutant protein 15A has a decreased activity compared to the wild-type CFTR. Analysis in Ussing chamber showed 85% reduction in short circuit current after forskolin addition when compared to wild-type CFTR, consistent with the known role of pKa-mediated channel activation. Additionally, CFTR mutant proteins (15A/E1371Q and 15D/E1371Q mutants) containing the E1371Q mutation have an increased pre-activation current. Surprisingly, this forskolin-independent effect was observed even more prominently with the E1371Q mutation (as compared to the phosphomimetic mutations), which falls in the ATP binding region of NBD2. The E1371Q mutation showed overall higher conductance than the wild-type CFTR, especially before forskolin addition. Additionally, a significant forskolin activation and an increased activity by the addition of VX-770 were not observed for the CFTR mutants containing E1371Q mutation. The minimal response to VX-770 likely reflects E1371Q's open probability having approached the theoretical, fully open maximum. The CFTR mutants of 15A, 15A/E1371Q, and 15D/E1371Q did not show an increased activity as compared to the wild-type CFTR protein.

### Example 10. Evaluating Response of E1371Q CFTR Construct in Combination with K14R Mutation in Ussing Chamber Assay

In this example, activity of E1371Q CFTR mutant was tested in combination with the K14R mutation using the Ussing chamber assay as described in Example 3.

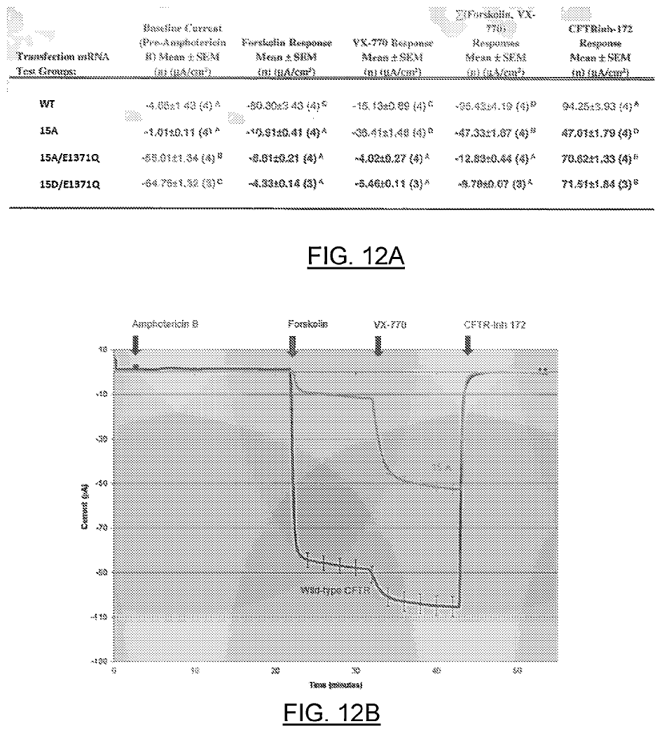

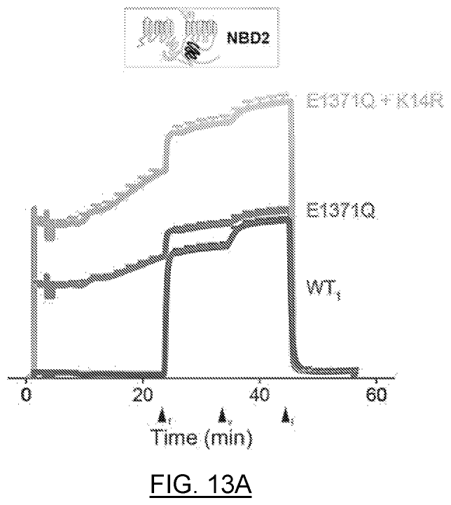

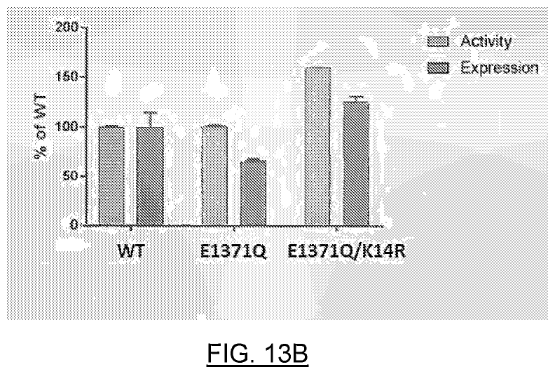

Our results also showed that E1371Q could be further augmented by introducing an additional variant, K14R, which represents a putative ubiquitination site (FIG. 13A). FIG. 13B shows that the E13Q/K14R CFTR mutant has an increased activity and an increased expression compared to both wild-type CFTR protein and the CFTR protein having only the E1371Q point mutation. These data suggest the possibility of generating a CFTR variant that is both more active (increased open probability) and more stable (increased copy number).

### Example 11. Evaluating Duration of Activity of CFTR Construct in Time-Course Ussing Chamber Assay

In this example, duration of activity of various phosphomimetic CFTR mutants were tested in combination with the K14R mutation using a time-course Ussing chamber assay as described in Example 3. The CFTR mutants tested in this example is listed in Table 12.

TABLE 12
CFTR Mutants tested in time-course Ussing Assay

| Engineered | |
|---|---|
| CFTR | Mutations |
| WT | Wild-type |
| 13E | S422E, S660E, S670E, S686E, T690E, S700E, S712E, |
| | S753E, T787E, T788E, T790E, S795E, S813E |
| 13E/K14R | K14R, S422E, S660E, S670E, S686E, T690E, S700E, |
| | S712E, S753E, T787E, T788E, T790E, S795E, S813E |
| 15E | S422E, S660E, S670E, S686E, T690E, S700E, S712E, |
| | S737E, S753E, S768E, T787E, T788E, T790E, S795E, |
| | S813E |
| 15E/K14R | K14R, S422E, S660E, S670E, S686E, T690E, S700E, |
| | S712E, S737E, S753E, S768E, T787E, T788E, T790E, |
| | S795E, S813E |

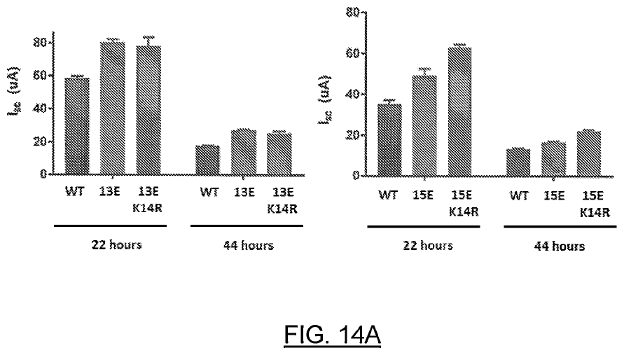

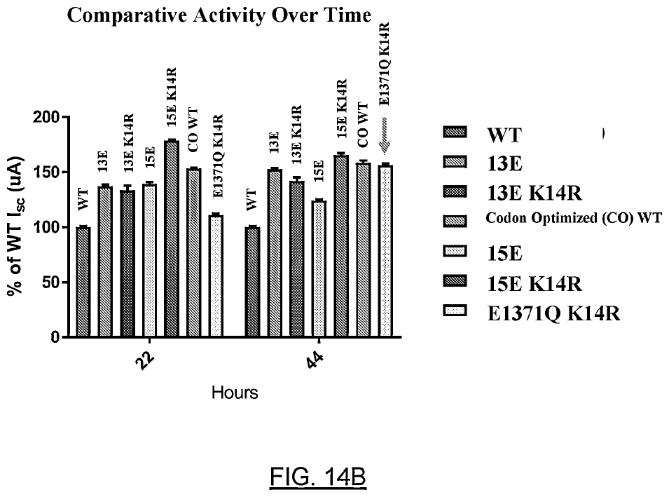

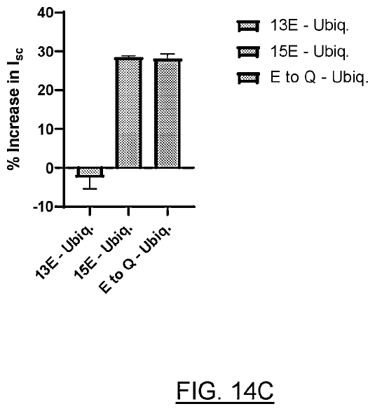

Activity of various CFTR proteins listed in Table 12 were measured at 22 and 44 hours. Short-circuit current (I_{sc}), the movement of ions as measured in the Ussing chamber from active transport, was plotted for each CFTR protein at 22 and 44 hours. FIG. 14A shows that highest remaining activity at 44 hours was observed with the 15E/K14R mutant and 13E mutant proteins. FIG. 14B shows that the E1371Q K14R mutant had high remaining activity at 44 hours.

Three engineered CFTR proteins (13E, 15E, and E to Q gating) were further designed to alter ubiquitination patterns in the N-terminus by substituting K14 with arginine. As shown in FIG. 14C, the additional substitution (K14R) had no effect on the chloride conductance when combined with the 13E phosphomimetic protein, in contrast with the 15E and E to Q gating variant proteins, both of which showed greater than about 25% increase in activity.

### Example 12. CFTR Mutations that Modulate the ATP Gating Cycle

In this example, mutations were made at CFTR amino acid residues that modulate the ATP gating cycle.

Mutations that destabilize or shorten the open state (e.g., mutations at W401, F409, F430, and/or K464) may modulate the effects of E1371Q to create an intermediate phenotype. Table 13, below, shows exemplary mutations that were assessed.

TABLE 13
Select CFTR Mutations that Modulate ATP Gating Cycle

| W401 | F409 | F430 | K464 | K978 | E1371 |
|---|---|---|---|---|---|
| | | | | C | |
| | | | | | Q |
| G | | | | | Q |
| G | G | | | | Q |
| G | G | G | | | Q |
| | | | A | | Q |

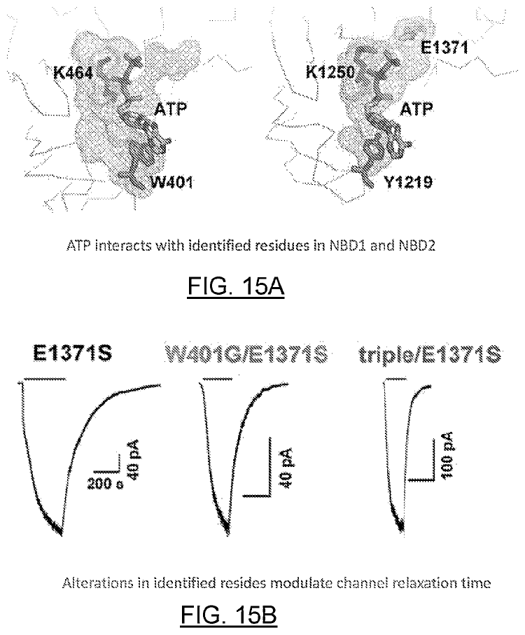

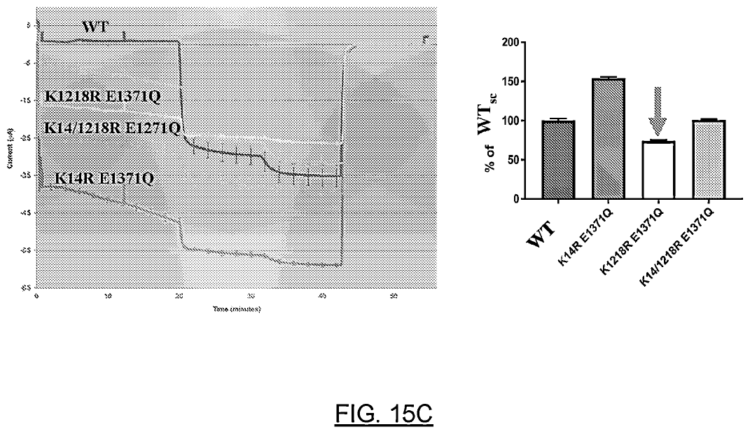

FIG. 15A shows a schematic of CFTR that contains mutations at the indicated residues. CFTR containing mutations that modulate the ATP gating cycle were assessed for CFTR channel relaxation times. The data shows that alterations in certain amino acid residues result in modulations of channel relation times (FIG. 15B).

Additional variants for E1371Q were assessed by measurement of short-circuit conductivity measured by Ussing chamber. The data showed that K1218R mutation decreases activity in both WT and E1371Q CFTR proteins (FIG. 15C).

### Example 13. Phosphomimetic CFTR mRNAs Show Varying Sensitivity to Forskolin Activation

In this example, various phosphomimetic CFTR mRNAs were tested for their sensitivity to forskolin activation.

In one study, varying numbers of phosphomimetic mutations were introduced into CFTR mRNAs. The number of phosphomimetic mutations that were introduced into CFTR mRNAs were 0, 4, 6, 8, 13 and 15. These CFTR mRNAs were then assessed for forskolin activation, by the successive addition of 10, 30, 100, 300, 1,000 and 10,000 nM forskolin. The activity of the different CFTR mRNAs having varying numbers of phosphomimetic mutations was assessed by measurement of short-circuit conductivity measured by Ussing chamber of FRT cells that had been transfected with CFTR mRNAs having a specific number of mutations.

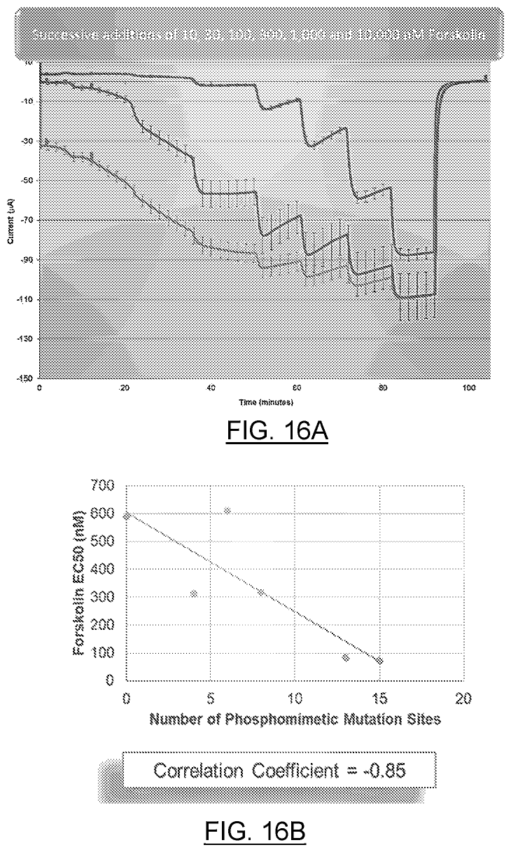

As shown in FIG. 16A, modifying typical Ussing chamber procedure to include successive escalating doses of forskolin demonstrates varying forskolin sensitivity of engineered CFTR variant channels. The data obtained from these experiments showed that with increasing amounts of phosphomimetic CFTR mutations, there was a corresponding increased sensitivity to forskolin activation (FIGS. 16A and 16B). The forskolin EC50 (nM) with respect to the number of mutations in the CFTR mRNA shown in Table 14 below.

TABLE 14
Phosphomimetic CFTR mRNAs have varying
sensitivity to forskolin activation

| Number of CFTR Mutations | Forskolin EC50 (NM) |
|---|---|
| 0 | 588.1 |
| 4 | 313.7 |
| 6 | 610.2 |
| 8 | 317.2 |
| 13 | 82.4 |
| 15 | 70.3 |

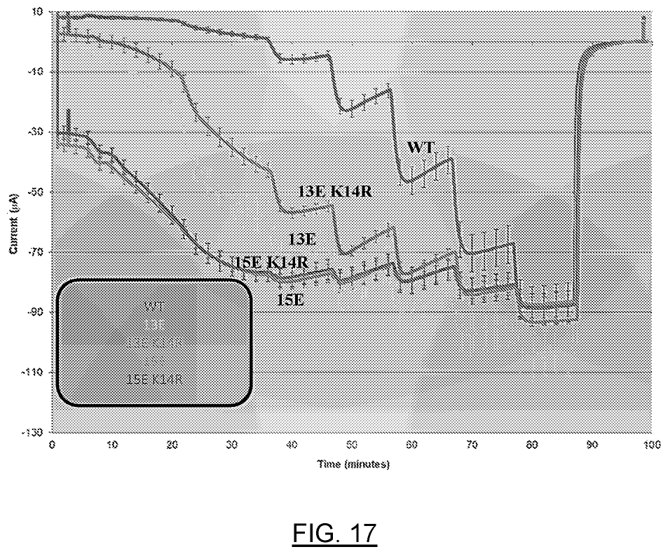

Studies were also conducted to determine whether amino acid substitutions 13S to 13D in comparison to 13 S to 13D showed any difference with regard to forskolin sensitivity. The data showed that both types of amino acid substitutions displayed similar changes in forskolin sensitivity as assessed by measurements of short-circuit conductivity measured by Ussing chamber of FRT cells that had been transfected with CFTR mRNAs having the specific amino acid substitutions (FIG. 17). The forskolin EC50 (nM) with these CFTR mutants is shown in Table 15 below.

TABLE 15
CFTR mRNA mutants 13S to 13D, and 13S to 13E showed
similar changes in forskolin sensitivity

| Construct | Forskolin EC50 (nM) |
|---|---|
| WT | 588.1 |
| 13D | 82.4 |
| 13E | 54.72 |
| 13E K14R | 54.84 |

### Example 14. In Silico Prediction of T Cell and B Cell Epitopes and In Vitro Translation and Tolerability

EpiQuest® software was used to predict T cell and B cell epitopes with regard to the CFTR mutants. Analysis using this software showed that all of the mutations assessed (i.e., K14R, 13D, 13E, 15D, 15E, K68R, K1218R, and E1371Q) were all outside of potential cytotoxic T lymphocyte (CTL) regions.

The data obtained from the assays showed that all assessed peptide sequences showed low or no immunogenicity potential. Furthermore, epitopes with humoral immunogenicity potential were not readily accessible and/or not on the surface. The data also showed that no mutations were predicted to create or enhance the potential of CTL epitopes. Moreover, ActivSite® software predicts that all mutations tested would fall outside critical regions of the CFTR molecule involved in functional and structural integrity.

Collectively, these data showed that the mutations would had low or no immunogenicity potential and that the mutations would not negatively impact CFTR functional and structural integrity.

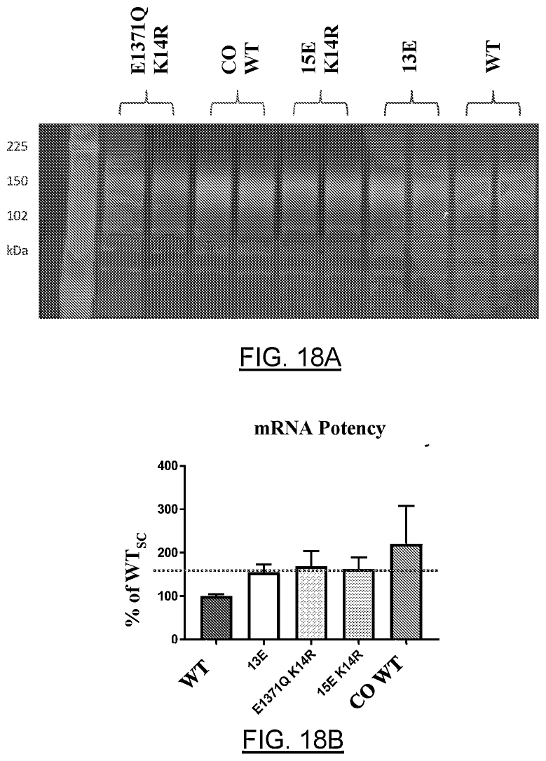

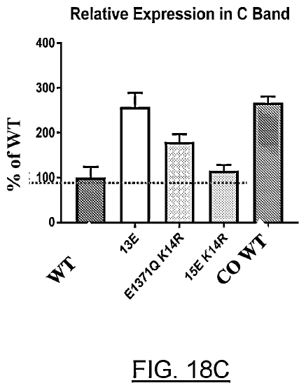

Studies were also performed to assess in vitro translation and tolerability of CFTR mutants. The data from these studies showed that codon optimized WT sequence, 13E and E1371Q K14R variants showed increased expression in the C band of HEK293 lysates (FIGS. 18A, 18B, and 18C). The “C band” refers to mature complex glycosylated form of CFTR.

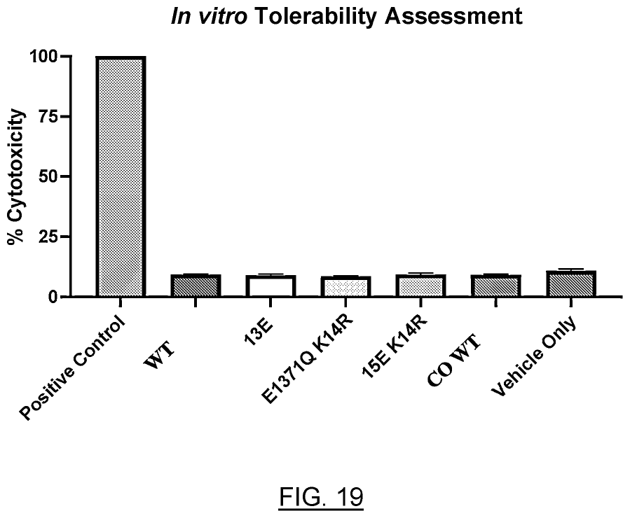

In vitro tolerability of the mutant CFTR mRNAs was also assessed in HEK293 cells using a commercially available cytotoxicity assay. The data from these studies showed that none of the CFTR variants demonstrated increased cytotoxicity when compared to vehicle control in HEK293 cells (FIG. 19).

### Example 15. CFTR Codon Optimization Increases Total Activity in Ussing Chamber for Tested CFTR Proteins

In this example, total activity of CFTR sequences that had been codon optimized (“CO”) in comparison to the non-codon optimized counterpart was assessed. A listing of codon-optimized CFTR sequences are shown in Table 4. For these assays, the following CFTR mutants were assessed WT, E1271Q/K14R, 13E/K14R, 15E/K14R, and 13E. The assessments included activity measurements between non-codon optimized CFTR mutants and codon-optimized CFTR mutants.

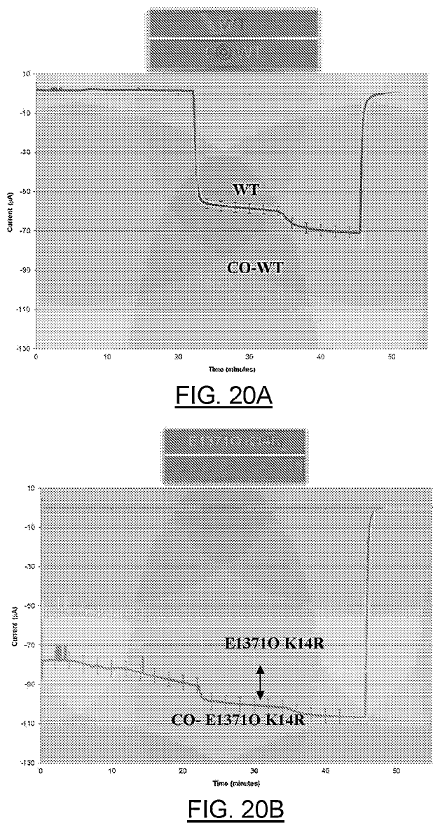

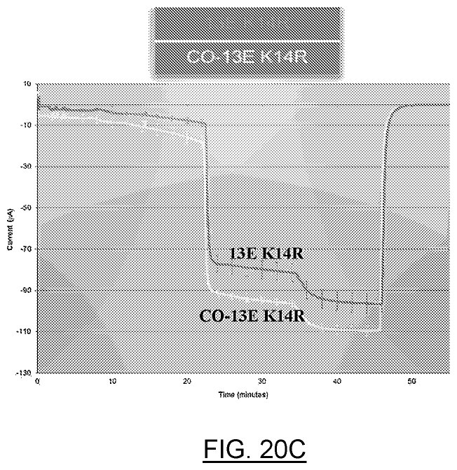

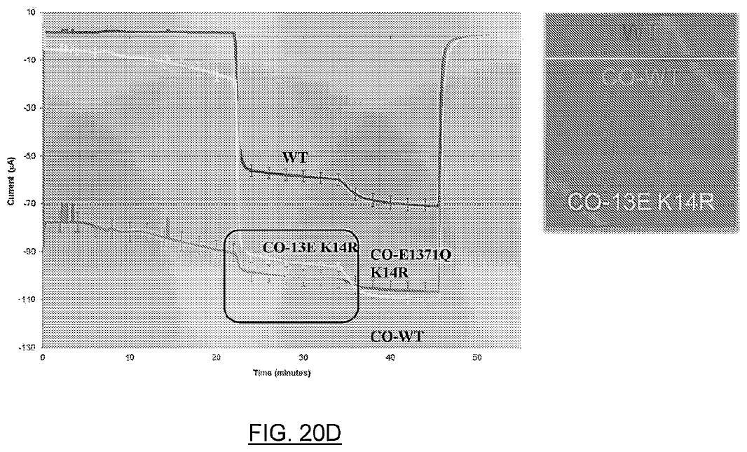

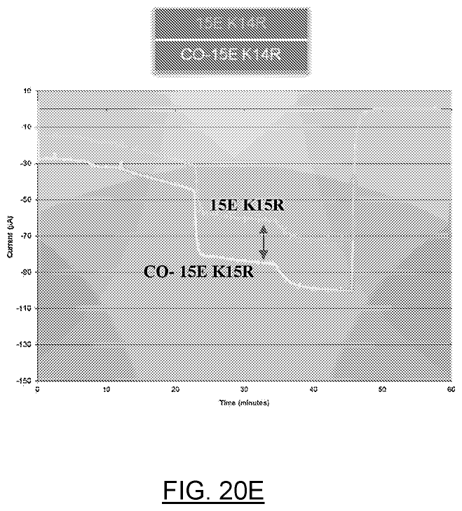

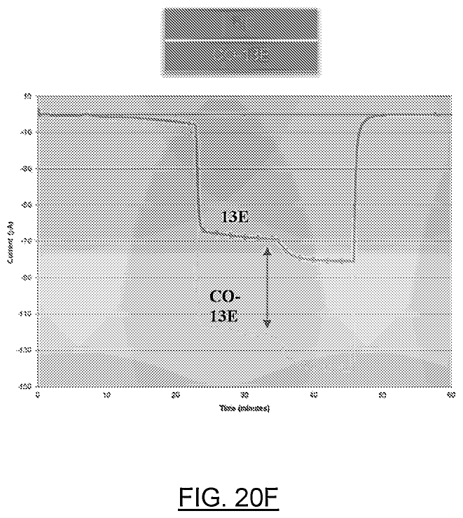

The data from these assays showed that codon-optimized constructs had increased activity in Ussing Chamber assays for all variants that were tested (FIGS. 20A, 20B, 20C, 20E and 20F) in comparison to the non-codon optimized counterpart.

In another study, the codon-optimized sequences each demonstrated comparable activity in comparison to other codon-optimized sequences regardless of the nature of the mutation contained in the codon-optimized sequence (FIG. 20D).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. (canceled)

2. A pharmaceutical composition for treating cystic fibrosis, comprising an mRNA encoding a Cystic Fibrosis Transmembrane Conductance Regulator (CFTR), wherein the CFTR comprises one or more mutations that produce an activated CFTR protein that is more stable than a wild type CFTR protein, wherein the mutation is an amino acid substitution.

3. The pharmaceutical composition of claim 2, wherein the one or more mutations are located within the CFTR regulatory domain, a membrane-spanning domain (MSD), and/or a nucleotide-binding domain, wherein the CFTR comprises at least 2 mutations in amino acid residues in comparison to wild type CFTR protein.

4.-5. (canceled)

6. The pharmaceutical composition of claim 3, wherein the CFTR comprises between 2 and 16 mutations in amino acid residues in comparison to wild type CFTR protein.

7. (canceled)

8. The pharmaceutical composition of claim 2, wherein:
(i) the one or more mutations are made at residues K14, K68, S422, S660, S670, S686, T690, S700, K710, 5712, K716, S737, S753, S768, T787, T788, 5790, S795, 5813, K978, K1041, K1080, K1218, E1371, and combinations thereof;
or
(ii) the one or more mutations are selected from K14R, K68R, K710R, K716R, K1041R, K1080R, K1218R, and combinations thereof;
or
(iii) the CFTR comprises mutations selected from K14R, K68R, K1218R, K14R/K68R, K14R/K1218R, K68R/K1218R, and K14R/K68R/K1218R;
or
(iv) the CFTR comprises mutations S660D/S737D/S795D/S813D, S660DD/S686D/S700D/S737D/S795D/S813D, S660D/S686D/S700D/S712D/S737D/S768D/S795D/S813D, K978C, or E13710;
or
(v) the CFTR comprises mutations S422D/S660D/S670D/S686D/T690D/S700D/S712D/S753D/T787D/T788D/S790D/S795D/S813 D;
or
(vi) the CFTR comprises mutations S422D/S660D/S670D/S686D/T690D/S700D/S712D/S737D/S753D/S768D/T787D/T788D/S790 D/S795D/S813D;
or
(vii) the one or more mutations made at residues S422, S660, S670, S686, T690, S700, S712, S737, S753, S768, T787, T788, S790, S795, S813 is an amino acid substitution to glutamic acid
or
(viii) the CFTR comprises mutations S422E/S660E/S670E/S686E/T690E/S700E/S712E/S753E/T787E/T788E/S790E/S795E/S813E
or
(ix) the CFTR comprises mutations S422E/S660E/S670E/S686E/T690E/S700E/S712E/S737E/S753E/S768E/T787E/T788E/S790E/S795E/S813E;
or
(x) wherein the CFTR further comprises K14R mutation and/or E1371Q mutation.

9.-17. (canceled)

18. The pharmaceutical composition of claim 2, wherein the mRNA encoding CFTR is codon optimized.

19. The pharmaceutical composition of claim 18, wherein the codon optimized CFTR mRNA further comprises a 5′ untranslated region (UTR) sequence of SEQ ID NO: 4 and/or a 3′ untranslated region (UTR) sequence of SEQ ID NO: 5 or SEQ ID NO: 6.

20. (canceled)

21. The pharmaceutical composition of claim 18, wherein the codon optimized CFTR mRNA is encapsulated within a nanoparticle.

22. (canceled)

23. The pharmaceutical composition of claim 21, wherein the nanoparticle comprises one or more cationic lipids, one or more non-cationic lipids, one or more cholesterol-based lipids and one or more PEG-modified lipids.

24.-25. (canceled)

26. The pharmaceutical composition of claim 23, wherein the nanoparticle has a size of less than about 100 nm.

27.-29. (canceled)

30. A polynucleotide comprising a sequence complementary to the sequence of the mRNA claim 2, wherein the polynucleotide is a linear or circular polynucleotide comprising deoxyribonucleotide residues.

31.-32. (canceled)

33. A method of:
(i) inducing CFTR expression in epithelial cells in a lung of a mammal, the method comprising contacting the epithelial cells in the lung of the mammal with a composition of claim 2 or,
(ii) treating cystic fibrosis comprising administering to a subject in need of treatment a pharmaceutical composition of claim 2.

34.-36. (canceled)

37. The method of claim 33, wherein the codon optimized CFTR mRNA is administered to the subject via pulmonary delivery.

38. (canceled)

39. The method of claim 33, wherein treating the subject is achieved at a lower therapeutically effective dose in comparison to treating the subject with an mRNA encoding a wild type CFTR and/or wherein treating the subject in need results in shorter nebulization times to administer a therapeutically effective dose in comparison to treating with an mRNA encoding wild type CFTR.

40. (canceled)

41. A nucleic acid encoding a modified Cystic Fibrosis Transmembrane Conductance Regulator (CFTR) protein, wherein the modified CFTR protein comprises one or more amino acid substitutions to glutamic acid.

42. The nucleic acid of claim 41, wherein the one or more amino acid substitutions occur at serine residues, or at threonine residues; and wherein the one or more amino acid substitutions are located within the CFTR regulatory domain.

43.-46. (canceled)

47. The nucleic acid of claim 41, wherein:
the modified CFTR protein comprises one or more mutations of S422E, S660E, S670E, S686E, T690E, S700E, S712E, S737E, S753E, S768E, T787E, T788E, S790E, S795E, S813E, and combinations thereof;
or
wherein the one or more amino acid substitutions are located at amino acid residues 440-820 or, wherein the modified CFTR protein comprises mutations S422E/S660E/S670E/S686E/T690E/S700E/S712E/S753E/T787E/T788E/S790E/S795E/S813E;
or
wherein the modified CFTR protein comprises mutations S422E/S660E/S670E/S686E/T690E/S700E/S712E/S737E/S753E/S768E/T787E/T788E/S790E/S795E/S813E;
or
wherein the modified CFTR protein further comprises K14R mutation and/or E1371Q mutation.

48.-53. (canceled)

54. The nucleic acid of claim 41, wherein the nucleic acid is DNA, cDNA, RNA, mRNA or PNA.

55.-67. (canceled)

68. A modified CFTR protein comprising one or more amino acid substitutions to glutamic acid or at serine residues or at threonine residues,
and
wherein the modified CFTR protein further comprises one or more mutations in the CFTR regulatory domain, a membrane-spanning domain (MSD), and/or a nucleotide-binding domain.

69.-73. (canceled)

74. The modified CFTR protein of claim 68, wherein:
(i) the one or more amino acid substitutions are located at:
(a) amino acid residues 440-820, or
(b) one or more amino acid substitutions occur at residue S422, S660, S670, S686, T690, S700, S712, S737, S753, S768, T787, T788, S790, S795, S813, and combinations thereof;
or
(ii) the modified CFTR protein comprises one or more mutations of S422E, S660E, S670E, S686E, T690E, S700E, S712E, S737E, S753E, S768E, T787E, T788E, S790E, S795E, S813E and combinations thereof;
or,
(iii) the CFTR protein comprises S422E/S660E/S670E/S686E/T690E/S700E/S712E/S753E/T787E/T788E/S790E/S795E/S813E;
or,
(iii) the modified CFTR protein comprises mutations S422E/S660E/S670E/S686E/T690E/S700E/S712E/S737E/S753E/S768E/T787E/T788E/S790E/S795E/S813E;
or
(iv) wherein the modified CFTR protein comprises K14R mutation and/or E1371Q mutation.

75.-80. (canceled)

81. A method of:
(i) inducing CFTR expression in epithelial cells in a lung of a mammal, or
(ii) treating cystic fibrosis comprising administering to a subject in need of treatment,
the method comprising contacting the epithelial cells in the lung of the mammal with the composition of claim 2.

82.-90. (canceled)
